(19) United States
(12) Patent Application Publication
(10) Pub. No.: US 2024/0392002 A1
(43) Pub. Date: Nov. 28, 2024

(54) ANTI-ROR ANTIBODY CONSTRUCTS

(71) Applicant: EXELIXIS, INC., Alameda, CA (US)

(72) Inventors: Lucas BAILEY, Madison, WI (US); Qufei LI, Middleton, WI (US); Malgorzata Agnieszka NOCULA-LUGOWSKA, Brighton, MA (US); Bryan GLASER, Madison, WI (US)

(73) Assignee: EXELIXIS, INC., Alameda, CA (US)

(21) Appl. No.: 18/801,013

(22) Filed: Aug. 12, 2024

Related U.S. Application Data
(62) Division of application No. 17/048,549, filed on Oct. 16, 2020, now Pat. No. 12,084,496.
(60) Provisional application No. 62/659,635, filed on Apr. 18, 2018.

(51) Int. Cl. C07K 16/28; A61K 39/00; A61K 45/06; A61P 35/00; C07K 16/46

(52) U.S. Cl. CPC C07K 16/2803; A61K 45/06; A61P 35/00; C07K 16/468; A61K 2039/505

(57) ABSTRACT

Anti-ROR antibody constructs, pharmaceutical compositions comprising the constructs, and methods of use thereof are presented.

### 1. CROSS-REFERENCE TO RELATED APPLICATIONS

This application is a divisional of U.S. patent application Ser. No. 17/048,549, filed Oct. 16, 2020, which is a U.S. National Stage Application under 35 U.S.C. § 371 of International Patent Application No. PCT/US2019/028051, filed Apr. 18, 2019, which claims the benefit of U.S. Provisional Patent Application No. 62/659,635 filed Apr. 18, 2018, the disclosure of each of which is incorporated by reference herein in its entirety.

### 2. SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14529-135-999_SUB_SEQ_LISTING”, was created on Aug. 15, 2024, and is 616,139 bytes in size.

### 3. BACKGROUND

The design and therapeutic use of multispecific antibodies—antibody-derived proteins engineered to recognize multiple targets—is an area of intensive research. Multispecific antibodies offer the promise of greater therapeutic control than is routinely provided by monospecific monoclonal antibodies. For example, multispecific antibodies can be engineered to provide greater target specificity than monospecific antibodies, reducing the off-target effects associated with many antibody therapies, particularly antibody-based immunotherapies. Multispecific antibodies also offer the promise of therapeutic strategies that are not possible with monospecific antibodies, such as synergistic targeting of multiple cell receptors, especially in immunotherapy. One such immunotherapy is the use of bispecific antibodies to recruit and redirect T cells to target and kill specific tumor cell populations through bispecific engagement of a T cell marker and a tumor cell marker. For example, the targeting of B cell lymphoma using CD3xCD19 bispecific antibodies, such as by the CD3xCD19 BiTE blinatumomab (Blincyto), is described in U.S. Pub. No. 2006/0193852.

There is, therefore, a need for improved multispecific antibodies that specifically bind to distinct cell populations, including tumor cell populations, with improvements including increased affinity or avidity, reduced off-target binding, and/or reduced unintended immune activation.

Various tumors can demonstrate cell-surface expression of tyrosine-protein kinase transmembrane receptor (ROR) antigens, as described in greater detail in Gentile, et al. (Cancer Res; 71(8) Apr. 15, 2011), Rebagay, et al. (Front. Oncol., 18 Apr. 2012), Zhang, et al. (American Journal ofPathology, Vol. 181, No. 6, December 2012), Henry, et al. (Oncotarget, Vol. 6, No. 37 2015), Zhang, et al. (PLoS ONE 7(3): e31127.), and Bainbridge, et al. (PLoS ONE 9(7): e102695.), each herein incorporated by reference in their entirety. In addition, ROR expression may not be expressed, or only demonstrate limited expression, in normal, i.e. non-cancerous, tissue as described in Balakrishnan et al. (Clin Cancer Res. 2017 Jun. 15; 23(12): 3061-3071), herein incorporated in its entirety. Thus ROR antigens can be used as a tumor-specific marker in certain tumors. Examples of tumors and cancers with demonstrated ROR expression include, but are not limited to, pancreatic cancer, ovarian cancer, breast cancer, lung cancer, gastric cancer, melanoma, Ewing sarcoma, chronic lymphocytic leukemia, mantle cell lymphoma, and B-ALL, as described in Gohil et al. (Oncoimmunology. 2017; 6(7): e1326437.), herein incorporated in its entirety. Other cancers include, but are not limited to, hematological cancer, prostate cancer, colon cancer, renal cancer, and uterine cancer. Use of ROR multispecific antibodies, formatted in various antibody platforms, to target tumors is described in Gohil, et al., international application WO 2017/053469, international application WO 2014/167022, U.S. Pub. No. 2017/0198045, international application WO 2016/094873, international application WO 2017/127499, and international application WO 2016/142768, each of which is herein incorporated by reference in its entirety.

ROR antigen binding molecules thus have therapeutic potential in treatment of cancer. Multispecific ROR binding molecules that bind T cell surface antigens in addition to a ROR antigen have potential to provide T cell redirected killing of ROR-expressing cancer cells.

There is, therefore, a need for ROR antigen binding molecules, including multispecific ROR antigen binding molecules. There is also a need for ROR antigen binding molecules that have improvements including increased affinity or avidity, reduced off-target binding, and/or reduced unintended immune activation. There is a particular need for a multispecific ROR antigen binding molecule that has improved manufacturability, and is readily purified.

### 4. SUMMARY

In a first aspect, antigen binding molecules are provided. In every embodiment, the antigen binding molecule includes at least an antigen binding site specific for a ROR antigen; the binding molecules are therefore termed ROR antigen binding molecules.

Described herein are tyrosine-protein kinase transmembrane receptor (ROR) antigen binding molecules comprising: A) a CDR1, a CDR2, and a CDR3 amino acid sequences of a light chain variable region (VL) from a ROR antigen binding site, wherein the CDR1, CDR2, and CDR3 VL sequences are selected from Table 6; and B) comprises a CDR1, a CDR2, and a CDR3 amino acid sequences of a heavy chain variable region (VH) from the ROR antigen binding site, wherein the CDR1, CDR2, and CDR3 VH sequences are selected from Table 6, wherein the ROR antigen binding site is a first antigen binding site and is specific for (i) ROR1 and ROR2, (ii) ROR1, or (iii) ROR2. In certain aspects, the ROR antigen binding molecule further comprises a second antigen binding site. In certain aspects, the second antigen binding site is the same as the first antigen binding site. In certain aspects, the second antigen binding site is specific for a second antigen different from the ROR antigen of the first antigen binding site. In certain aspects, the second antigen is a CD3 antigen.

Described herein are tyrosine-protein kinase transmembrane receptor (ROR) antigen binding molecules comprising: a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the ROR antigen binding molecule, wherein the interaction between the A domain and the F domain form a first antigen binding site specific for a ROR antigen.

In certain aspects, the ROR antigen is ROR1. In certain aspects, the ROR antigen is ROR2. In certain aspects, the ROR antigen is ROR1 and ROR2. In certain aspects, the ROR antigen is a domain selected from the group consisting of: a ROR1 Frizzle domain, ROR2 Frizzle domain, a ROR1 Ig-like domain, a ROR2 Ig-like domain, a ROR1 Kringle domain, and a ROR2 Kringle domain. In certain aspects, the ROR antigen comprises a human ROR antigen.

In certain aspects, Domain A comprises a CDR1, a CDR2, and a CDR3 amino acid sequences of a specific light chain variable region (VL) from a specific ROR antigen binding site, wherein the CDR1, CDR2, and CDR3 VL sequences are selected from Table 6, and Domain F comprises a CDR1, a CDR2, and a CDR3 amino acid sequences of a specific heavy chain variable region (VH) from the specific ROR antigen binding site, wherein the CDR1, CDR2, and CDR3 VH sequences are selected from Table 6. In certain aspects, the specific ROR antigen binding site is I2A-10, I2A-10 D54E Y55Q, or I2A-27 from Table 6.

In certain aspects, Domain A comprises a VL having one or two amino acid mutations as compared with a VL sequence of the antibody in Table 6, wherein the one or two amino acid mutations are in one or more CDR regions in the VL. In certain aspects, Domain F comprises a VH having one or two amino acid mutations as compared with a VH sequence of the antibody in Table 6, wherein the one or two amino acid mutations are in one or more CDR regions in the VH. In certain aspects, Domain A comprises a VL having the VL sequence of I2A-10 with one or more mutations in one or more CDR regions. In certain aspects, Domain A comprises a VL having the VL sequence of I2A-27 with one or more mutations in one or more CDR regions. In certain aspects, Domain F comprises a VL having the VL sequence of I2A-10 with one or more mutations in one or more CDR regions. In certain aspects, Domain F comprises a VL having the VL sequence of I2A-27 with one or more mutations in one or more CDR regions.

In certain aspects, the amino acid sequences of the B domain and the G domain are identical, wherein the sequence is an endogenous CH3 sequence.

In certain aspects, the amino acid sequences of the B domain and the G domain are different and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

In certain aspects, the orthogonal modifications of the B domain and the G domain comprise mutations that generate engineered disulfide bridges between the B domain and the G domain. In certain aspects, the mutations of the B domain and the G domain that generate engineered disulfide bridges are a S354C mutation in one of the B domain and G domain, and a 349C in the other domain.

In certain aspects, the orthogonal modifications of the B domain and the G domain comprise knob-in-hole mutations. In certain aspects, the knob-in hole mutations are a T366W mutation in one of the B domain and G domain, and a T366S, L368A, and aY407V mutation in the other domain.

In certain aspects, the orthogonal modifications of the B domain and the G domain comprise charge-pair mutations. In certain aspects, the charge-pair mutations are a T366K mutation in one of the B domain and G domain, and a L351D mutation in the other domain.

In certain aspects, the E domain has a CH3 amino acid sequence.

In certain aspects, the amino acid sequences of the E domain and the K domain are identical, wherein the sequence is an endogenous CH3 sequence.

In certain aspects, the amino acid sequences of the E domain and the K domain are different. In certain aspects, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification.

In certain aspects, the orthogonal modifications comprise mutations that generate engineered disulfide bridges between the E domain and the K domain. In certain aspects, the mutations that generate engineered disulfide bridges are a S354C mutation in one of the E domain and the K domain, and a 349C in the other domain.

In certain aspects, the orthogonal modifications in the E domain and the K domain comprise knob-in-hole mutations. In certain aspects, the knob-in hole mutations are a T366W mutation in one of the E domain or the K domain and a T366S, L368A, and aY407V mutation in the other domain.

In certain aspects, the orthogonal modifications in the E domain and the K domain comprise charge-pair mutations. In certain aspects, the charge-pair mutations are a T366K mutation in one of the E domain or the K domain and a corresponding L351D mutation in the other domain.

In certain aspects, the amino acid sequences of the E domain and the K domain are endogenous sequences of two different antibody domains, the domains selected to have a specific interaction that promotes the specific association between the first and the third polypeptides. In certain aspects, the two different amino acid sequences are a CH1 sequence and a CL sequence.

In certain aspects, the sequence that forms the junction between the A domain and the B domain is IKRTPREP (SEQ ID NO:57) or IKRTVREP (SEQ ID NO:58).

In certain aspects, the sequence that forms the junction between the F domain and the G domain is SSASPREP (SEQ ID NO:63).

In certain aspects, at least one CH3 amino acid sequence has a C-terminal tripeptide insertion connecting the CH3 amino acid sequence to a hinge amino acid sequence, wherein the tripeptide insertion is selected from the group consisting of PGK, KSC, and GEC.

In certain aspects, the sequences are human sequences.

In certain aspects, at least one CH3 amino acid sequence is an IgG sequence. In certain aspects, the IgG sequences are IgG1 sequences.

In certain aspects, at least one CH3 amino acid sequence has one or more isoallotype mutations. In certain aspects, the isoallotype mutations are D356E and L358M.

In certain aspects, the CL amino acid sequence is a Ckappa sequence.

In certain aspects, the CH2 sequences have one or more engineered mutations that reduce Fc effector function. In certain aspects, the one or more engineered mutations are at position L234, L235, and P329. In certain aspects, the one or more engineered mutations are L234A, L235A, and P329G. In certain aspects, the one or more engineered mutations are L234A, L235A, and P329K.

Also described herein are tyrosine-protein kinase transmembrane receptor (ROR) antigen binding molecules comprising: a first, second, third, and fourth polypeptide chain, wherein: (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the ROR antigen binding molecule, wherein the interaction between the A domain and the F domain form a first antigen binding site, the interaction between the H domain and the L domain form a second antigen binding site, and wherein the first antigen binding site, the second antigen binding site, or the first and the second antigen binding site are specific for a ROR antigen.

In certain aspects, the first antigen binding site is specific for the ROR antigen. In certain aspects, the second antigen binding site is specific for the ROR antigen. In certain aspects, the first and the second antigen binding site is specific for the ROR antigen.

In certain aspects, the ROR antigen is ROR1. In certain aspects, the ROR antigen is ROR2. In certain aspects, the ROR antigen is ROR1 and ROR2. In certain aspects, the ROR antigen is a domain selected from the group consisting of: a ROR1 Frizzle domain, ROR2 Frizzle domain, a ROR1 Ig-like domain, a ROR2 Ig-like domain, a ROR1 Kringle domain, and a ROR2 Kringle domain. In certain aspects, the ROR antigen comprises a human ROR antigen.

In certain aspects, Domain A comprises a CDR1, a CDR2, and a CDR3 amino acid sequences of a specific light chain variable region (VL) from a specific ROR antigen binding site, wherein the CDR1, CDR2, and CDR3 VL sequences are selected from Table 6, and Domain F comprises a CDR1, a CDR2, and a CDR3 amino acid sequences of a specific heavy chain variable region (VH) from the specific ROR antigen binding site, wherein the CDR1, CDR2, and CDR3 VH sequences are selected from Table 6. In certain aspects, the specific ROR antigen binding site is I2A-10, I2A-10 D54E Y55Q or I2A-27 from Table 6.

In certain aspects, Domain A comprises a VL having one or two amino acid mutations as compared with a VL sequence of the antibody in Table 6, wherein the one or two amino acid mutations are in one or more CDR regions in the VL. In certain aspects, Domain F comprises a VH having one or two amino acid mutations as compared with a VH sequence of the antibody in Table 6, wherein the one or two amino acid mutations are in one or more CDR regions in the VH. In certain aspects, Domain A comprises a VL having the VL sequence of I2A-10 with one or more mutations in one or more CDR regions. In certain aspects, Domain A comprises a VL having the VL sequence of I2A-27 with one or more mutations in one or more CDR regions. In certain aspects, Domain F comprises a VL having the VL sequence of I2A-10 with one or more mutations in one or more CDR regions. In certain aspects, Domain F comprises a VL having the VL sequence of I2A-27 with one or more mutations in one or more CDR regions.

In certain aspects, the second antigen binding site comprises: A) within the third polypeptide chain a specific light chain variable region (VL) amino acid sequence selected from the group consisting of: SEQ ID NO:69 and SEQ ID NO:73; and B) within the fourth polypeptide chain a specific heavy chain variable region (VH) amino acid sequence selected from the group consisting of: SEQ ID NO:68, SEQ ID NO:70, SEQ ID NO:71, and SEQ ID NO:72.

In certain aspects, the second antigen binding site comprises: A) within the fourth polypeptide chain a specific light chain variable region (VL) amino acid sequence selected from the group consisting of: SEQ ID NO:69 and SEQ ID NO:73; and B) within the third polypeptide chain a specific heavy chain variable region (VH) amino acid sequence selected from the group consisting of: SEQ ID NO:68, SEQ ID NO:70, SEQ ID NO:71, and SEQ ID NO:72.

In certain aspects, the amino acid sequences of the B domain and the G domain are identical, wherein the sequence is an endogenous CH3 sequence.

In certain aspects, the amino acid sequences of the B domain and the G domain are different and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

In certain aspects, the orthogonal modifications of the B domain and the G domain comprise mutations that generate engineered disulfide bridges between the B domain and the G domain. In certain aspects, the mutations of the B domain and the G domain that generate engineered disulfide bridges are a S354C mutation in one of the B domain and G domain, and a 349C in the other domain.

In certain aspects, the orthogonal modifications of the B domain and the G domain comprise knob-in-hole mutations. In certain aspects, the knob-in hole mutations are a T366W mutation in one of the B domain and G domain, and a T366S, L368A, and aY407V mutation in the other domain.

In certain aspects, the orthogonal modifications of the B domain and the G domain comprise charge-pair mutations. In certain aspects, the charge-pair mutations are a T366K mutation in one of the B domain and G domain, and a L351D mutation in the other domain.

In certain aspects, the E domain has a CH3 amino acid sequence.

In certain aspects, the amino acid sequences of the E domain and the K domain are identical, wherein the sequence is an endogenous CH3 sequence.

In certain aspects, the amino acid sequences of the E domain and the K domain are different. In certain aspects, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification.

In certain aspects, the orthogonal modifications comprise mutations that generate engineered disulfide bridges between the E domain and the K domain. In certain aspects, the mutations that generate engineered disulfide bridges are a S354C mutation in one of the E domain and the K domain, and a 349C in the other domain.

In certain aspects, the orthogonal modifications in the E domain and the K domain comprise knob-in-hole mutations. In certain aspects, the knob-in hole mutations are a T366W mutation in one of the E domain or the K domain and a T366S, L368A, and aY407V mutation in the other domain.

In certain aspects, the orthogonal modifications in the E domain and the K domain comprise charge-pair mutations. In certain aspects, the charge-pair mutations are a T366K mutation in one of the E domain or the K domain and a corresponding L351D mutation in the other domain.

In certain aspects, the amino acid sequences of the E domain and the K domain are endogenous sequences of two different antibody domains, the domains selected to have a specific interaction that promotes the specific association between the first and the third polypeptides. In certain aspects, the two different amino acid sequences are a CH1 sequence and a CL sequence.

In certain aspects, domain I has a CL sequence and domain M has a CH1 sequence.

In certain aspects, domain H has a VL sequence and domain L has a VH sequence.

In certain aspects, domain H has a VL amino acid sequence; domain I has a CL amino acid sequence; domain K has a CH3 amino acid sequence; domain L has a VH amino acid sequence; and domain M has a CH1 amino acid sequence.

In certain aspects, the ROR antigen binding molecule further comprises: a fifth polypeptide chain, wherein: (a) the first polypeptide chain further comprises a domain N and a domain 0, wherein the domains are arranged, from N-terminus to C-terminus, in a N-O-A-B-D-E orientation, and wherein domain N has a variable region domain amino acid sequence, domain O has a constant region domain amino acid sequence; (b) the ROR antigen binding molecule further comprises a fifth polypeptide chain, comprising: a domain P and a domain Q, wherein the domains are arranged, from N-terminus to C-terminus, in a P-Q orientation, and wherein domain P has a variable region domain amino acid sequence and domain Q has a constant region domain amino acid sequence; and (c) the first and the fifth polypeptides are associated through an interaction between the N and the P domains and an interaction between the O and the Q domains to form the ROR antigen binding molecule.

In certain aspects, (a) the amino acid sequences of domain N and domain A are identical, the amino acid sequences of domain H is different from the sequence of domain N and domain A, the amino acid sequences of domain 0 and domain B are identical, the amino acid sequences of domain I is different from the sequence of domain 0 and domain B, the amino acid sequences of domain P and domain F are identical, the amino acid sequences of domain L is different from the sequence of domain P and domain F, the amino acid sequences of domain Q and domain G are identical, the amino acid sequences of domain M is different from the sequence of domain Q and domain G; and (b) wherein the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for the first antigen. In certain aspects, the first antigen is the ROR antigen. In certain aspects, the second antigen is a CD3 antigen.

In certain aspects, (a) the amino acid sequences of domain N, domain A, and domain H are different, the amino acid sequences of domain 0, domain B, and domain I are different, the amino acid sequences of domain P, domain F, and domain L are different, and the amino acid sequences of domain Q, domain G, and domain M are different; (b) the interaction between the A domain and the F domain form a first antigen binding site specific for a first antigen, the interaction between the H domain and the L domain form a second antigen binding site specific for a second antigen, and the domain N and domain P form a third antigen binding site specific for a third antigen, and (c) the first, the second, or the third antigen is the ROR antigen.

In certain aspects, the ROR antigen binding molecule further comprises: a sixth polypeptide chain, wherein: (a) the third polypeptide chain further comprises a domain R and a domain S, wherein the domains are arranged, from N-terminus to C-terminus, in a R-S-H-I-J-K orientation, and wherein domain R has a variable region domain amino acid sequence and domain S has a constant domain amino acid sequence; (b) the ROR antigen binding molecule further comprises a sixth polypeptide chain, comprising: a domain T and a domain U, wherein the domains are arranged, from N-terminus to C-terminus, in a T-U orientation, and wherein domain T has a variable region domain amino acid sequence and domain U has a constant domain amino acid sequence; and (c) the third and the sixth polypeptides are associated through an interaction between the R and the T domains and an interaction between the S and the U domains to form the ROR antigen binding molecule.

In certain aspects: (a) the amino acid sequences of domain R and domain A are identical, the amino acid sequences of domain H is different from the sequence of domain R and domain A, the amino acid sequences of domain S and domain B are identical, the amino acid sequences of domain I is different from the sequence of domain S and domain B, the amino acid sequences of domain T and domain F are identical, the amino acid sequences of domain L is different from the sequence of domain T and domain F, the amino acid sequences of domain U and domain G are identical, the amino acid sequences of domain M is different from the sequence of domain U and domain G and (b) the interaction between the A domain and the F domain form a first antigen binding site specific for a first antigen, the interaction between the H domain and the L domain form a second antigen binding site specific for a second antigen; and the interaction between the R domain and the T domain form a third antigen binding site specific for the first antigen. In certain aspects, the first antigen is the ROR antigen. In certain aspects, the second antigen is a CD3 antigen.

In certain aspects, (a) the amino acid sequences of domain R and domain H are identical, the amino acid sequences of domain A is different from the sequence of domain R and domain H, the amino acid sequences of domain S and domain I are identical, the amino acid sequences of domain B is different from the sequence of domain S and domain I, the amino acid sequences of domain T and domain L are identical, the amino acid sequences of domain F is different from the sequence of domain T and domain L, the amino acid sequences of domain U and domain M are identical, the amino acid sequences of domain G is different from the sequence of domain U and domain M, and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for the second antigen. In certain aspects, the second antigen is the ROR antigen. In certain aspects, the first antigen is a CD3 antigen.

In certain aspects, (a) the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for a third antigen; and (c) the first, the second, or the third antigen is the ROR antigen.

In certain aspects, the ROR antigen binding molecule further comprises: a fifth and a sixth polypeptide chain, wherein: (a) the first polypeptide chain further comprises a domain N and a domain 0, wherein the domains are arranged, from N-terminus to C-terminus, in a N-O-A-B-D-E orientation; (b) the third polypeptide chain further comprises a domain R and a domain S, wherein the domains are arranged, from N-terminus to C-terminus, in a R-S-H-I-J-K orientation; (c) the ROR antigen binding molecule further comprises a fifth and a sixth polypeptide chain, wherein: the fifth polypeptide chain comprises a domain P and a domain Q, wherein the domains are arranged, from N-terminus to C-terminus, in a P-Q orientation, and the sixth polypeptide chain comprises a domain T and a domain U, wherein the domains are arranged, from N-terminus to C-terminus, in a T-U orientation; and (d) the first and the fifth polypeptides are associated through an interaction between the N and the P domains and an interaction between the O and the Q domains, and the third and the sixth polypeptides are associated through an interaction between the R and the T domains and an interaction between the S and the U domains to form the ROR antigen binding molecule.

In certain aspects, (a) the amino acid sequences of domain N and domain A are identical, the amino acid sequences of domain H and domain R are identical, the amino acid sequences of domain 0 and domain B are identical, the amino acid sequences of domain I and domain S are identical, the amino acid sequences of domain P and domain F are identical, the amino acid sequences of domain L and domain T are identical, the amino acid sequences of domain Q and domain G are identical, the amino acid sequences of domain M and domain U are identical; and (b) wherein the interaction between the A domain and the F domain form a first antigen binding site specific for a first antigen, the domain N and domain P form a second antigen binding site specific for the first antigen, the interaction between the H domain and the L domain form a third antigen binding site specific for a second antigen, and the interaction between the R domain and the T domain form a fourth antigen binding site specific for the second antigen.

In certain aspects, (a) the amino acid sequences of domain H and domain A are identical, the amino acid sequences of domain N and domain R are identical, the amino acid sequences of domain I and domain B are identical, the amino acid sequences of domain 0 and domain S are identical, the amino acid sequences of domain L and domain F are identical, the amino acid sequences of domain P and domain T are identical, the amino acid sequences of domain M and domain G are identical, the amino acid sequences of domain Q and domain U are identical; and (b) wherein the interaction between the A domain and the F domain form a first antigen binding site specific for a first antigen, the domain N and domain P form a second antigen binding site specific for a second antigen, the interaction between the H domain and the L domain form a third antigen binding site specific for the first antigen, and the interaction between the R domain and the T domain form a fourth antigen binding site specific for the second antigen.

In certain aspects, the sequence that forms the junction between the A domain and the B domain is IKRTPREP (SEQ ID NO:57) or IKRTVREP (SEQ ID NO:58).

In certain aspects, the sequence that forms the junction between the F domain and the G domain is SSASPREP (SEQ ID NO:63).

In certain aspects, at least one CH3 amino acid sequence has a C-terminal tripeptide insertion connecting the CH3 amino acid sequence to a hinge amino acid sequence, wherein the tripeptide insertion is selected from the group consisting of PGK, KSC, and GEC.

In certain aspects, the sequences are human sequences.

In certain aspects, at least one CH3 amino acid sequence is an IgG sequence. In certain aspects, the IgG sequences are IgG1 sequences.

In certain aspects, at least one CH3 amino acid sequence has one or more isoallotype mutations. In certain aspects, the isoallotype mutations are D356E and L358M.

In certain aspects, the CL amino acid sequence is a Ckappa sequence.

In certain aspects, the CH2 sequences have one or more engineered mutations that reduce Fc effector function. In certain aspects, the one or more engineered mutations are at position L234, L235, and P329. In certain aspects, the one or more engineered mutations are L234A, L235A, and P329G. In certain aspects, the one or more engineered mutations are L234A, L235A, and P329K.

Also described herein are tyrosine-protein kinase transmembrane receptor (ROR) antigen binding molecules comprising: a first antigen binding site specific for a ROR antigen, wherein the first antigen binding site comprises: A) a CDR1, a CDR2, and a CDR3 amino acid sequences of a specific light chain variable region (VL) from a specific ROR antigen binding site, wherein the CDR1, CDR2, and CDR3 VL sequences are selected from Table 6; and B) comprises a CDR1, a CDR2, and a CDR3 amino acid sequences of a specific heavy chain variable region (VH) from the specific ROR antigen binding site, wherein the CDR1, CDR2, and CDR3 VH sequences are selected from Table 6.

In certain aspects, the first antigen binding site comprises a VL having one or two amino acid mutations as compared with a VL sequence of the antibody in Table 6, wherein the one or two amino acid mutations are in one or more CDR regions in the VL. In certain aspects, the first antigen binding site comprises a VH having one or two amino acid mutations as compared with a VH sequence of the antibody in Table 6, wherein the one or two amino acid mutations are in one or more CDR regions in the VH.

In certain aspects, the first antigen binding site is specific for ROR1. In certain aspects, the first antigen binding site is specific for ROR2. In certain aspects, the first antigen binding site is specific for ROR1 and ROR2. In certain aspects, the ROR antigen is a domain selected from the group consisting of: a ROR1 Frizzle domain, ROR2 Frizzle domain, a ROR1 Ig-like domain, a ROR2 Ig-like domain, a ROR1 Kringle domain, and a ROR2 Kringle domain. In certain aspects, the ROR antigen comprises a human ROR antigen.

In certain aspects, the ROR antigen binding molecule further comprises a second antigen binding site. In certain aspects, the second antigen binding site is specific for the ROR antigen. In certain aspects, the second antigen binding site is specific for a second antigen different from the ROR antigen. In certain aspects, the second antigen is a CD3 antigen. In certain aspects, the antigen binding site is specific for an epitope of the CD3 antigen. In certain aspects, the second antigen binding site comprises: A) a specific light chain variable region (VL) amino acid sequence selected from the group consisting of: SEQ ID NO:69 and SEQ ID NO:73; and B) a specific heavy chain variable region (VH) amino acid sequence selected from the group consisting of: SEQ ID NO:68, SEQ ID NO:70, SEQ ID NO:71, and SEQ ID NO:72. In certain aspects, the second antigen binding site comprises: A) a specific light chain variable region (VL) amino acid sequence selected from the group consisting of: SEQ ID NO:69 and SEQ ID NO:73; and B) a specific heavy chain variable region (VH) amino acid sequence selected from the group consisting of: SEQ ID NO:68, SEQ ID NO:70, SEQ ID NO:71, and SEQ ID NO:72.

In certain aspects, the ROR antigen binding molecule comprises an antibody format selected from the group consisting of: full-length antibodies, Fab fragments, Fvs, scFvs, tandem scFvs, Diabodies, scDiabodies, DARTs, tandAbs, and minibodies. In certain aspects, the ROR antigen binding molecule comprises: a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence c) the first and the second polypeptides are associated through an interaction between the A and the F domain and an interaction between the B domain and the G domain to form the ROR antigen binding molecule, and wherein the interaction between the A domain and the F domain form a first antigen binding site.

In certain aspects, the ROR antigen binding molecule further comprises: a third and a fourth polypeptide chain, wherein: (a) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, and domains I, J, and K have a constant region domain amino acid sequence; (b) the fourth polypeptide chain comprises a domain L and a domain M, wherein the domains are arranged, from N-terminus to C-terminus, in a L-M orientation, and wherein domain L has a variable region domain amino acid sequence and domain M has a constant region amino acid sequence; (c) the third and the fourth polypeptides are associated through an interaction between the H and the L domains and an interaction between the I and the M domains; and (d) the first and the third polypeptides are associated through an interaction between the D domain and the J domain and an interaction between the E domain and the K domain to form the ROR antigen binding molecule, and wherein the interaction between the H domain and the L domain form a second antigen binding site. In certain aspects, the first antigen binding site is specific for the ROR antigen. In certain aspects, the second antigen binding site is specific for CD3.

In certain aspects, domain B and domain G have a CH3 amino acid sequence.

In certain aspects, the amino acid sequences of the B domain and the G domain are identical, wherein the sequence is an endogenous CH3 sequence.

In certain aspects, the amino acid sequences of the B domain and the G domain are different and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

In certain aspects, the orthogonal modifications of the B domain and the G domain comprise mutations that generate engineered disulfide bridges between the B domain and the G domain. In certain aspects, the mutations of the B domain and the G domain that generate engineered disulfide bridges are a S354C mutation in one of the B domain and G domain, and a 349C in the other domain.

In certain aspects, the orthogonal modifications of the B domain and the G domain comprise knob-in-hole mutations. In certain aspects, the knob-in hole mutations of the B domain and the G domain are a T366W mutation in one of the B domain and G domain, and a T366S, L368A, and aY407V mutation in the other domain.

In certain aspects, the orthogonal modifications of the B domain and the G domain comprise charge-pair mutations. In certain aspects, the charge-pair mutations of the B domain and the G domain are a T366K mutation in one of the B domain and G domain, and a L351D mutation in the other domain.

In certain aspects, domain B and domain G have an IgM CH2 amino acid sequence or an IgE CH2 amino acid sequence. In certain aspects, the IgM CH2 amino acid sequence or the IgE CH2 amino acid sequence comprise orthogonal modifications.

In certain aspects, domain I has a CL sequence and domain M has a CH1 sequence. In certain aspects, domain I has a CH1 sequence and domain M has a CL sequence. In certain aspects, the CH1 sequence and the CL sequence each comprise one or more orthogonal modifications, wherein a domain having the CH1 sequence does not significantly interact with a domain having a CL sequence lacking the orthogonal modification.

In certain aspects, the orthogonal modifications comprise mutations that generate engineered disulfide bridges between the at least one CH1 domain and a CL domain, the mutations selected from the group consisting of: an engineered cysteine at position 138 of the CH1 sequence and position 116 of the CL sequence; an engineered cysteine at position 128 of the CH1 sequence and position 119 of the CL sequence, and an engineered cysteine at position 129 of the CH1 sequence and position 210 of the CL sequence.

In certain aspects, the orthogonal modifications comprise mutations that generate engineered disulfide bridges between the at least one CH1 domain and a CL domain, wherein the mutations comprise and engineered cysteines at position 128 of the CH1 sequence and position 118 of a CL Kappa sequence.

In certain aspects, the orthogonal modifications comprise mutations that generate engineered disulfide bridges between the at least one CH1 domain and a CL domain, the mutations selected from the group consisting of: a F118C mutation in the CL sequence with a corresponding A141C in the CH1 sequence; a F1 18C mutation in the CL sequence with a corresponding L128C in the CH1 sequence; and a S162C mutations in the CL sequence with a corresponding P171C mutation in the CH1 sequence.

In certain aspects, the orthogonal modifications comprise charge-pair mutations between the at least one CH1 domain and a CL domain, the charge-pair mutations selected from the group consisting of: a F1 18S mutation in the CL sequence with a corresponding A141L in the CH1 sequence; a F1 18A mutation in the CL sequence with a corresponding A141L in the CH1 sequence; a F1 18V mutation in the CL sequence with a corresponding A141L in the CH1 sequence; and a T129R mutation in the CL sequence with a corresponding K147D in the CH1 sequence.

In certain aspects, the orthogonal modifications comprise charge-pair mutations between the at least one CH1 domain and a CL domain, the charge-pair mutations selected from the group consisting of: a N138K mutation in the CL sequence with a corresponding G166D in the CH1 sequence, and a N138D mutation in the CL sequence with a corresponding G166K in the CH1 sequence.

In certain aspects, domain A has a VL amino acid sequence and domain F has a VH amino acid sequence. In certain aspects, domain A has a VH amino acid sequence and domain F has a VL amino acid sequence.

In certain aspects, domain H has a VL amino acid sequence and domain L has a VH amino acid sequence. In certain aspects, domain H has a VH amino acid sequence and domain L has a VL amino acid sequence.

In certain aspects, domain D and domain J have a CH2 amino acid sequence.

In certain aspects, the E domain has a CH3 amino acid sequence.

In certain aspects, the amino acid sequences of the E domain and the K domain are identical, wherein the sequence is an endogenous CH3 sequence.

In certain aspects, the amino acid sequences of the E domain and the K domain are different. In certain aspects, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification.

In certain aspects, the orthogonal modifications comprise mutations that generate engineered disulfide bridges between the E domain and the K domain. In certain aspects, the mutations that generate engineered disulfide bridges are a S354C mutation in one of the E domain and the K domain, and a 349C in the other domain. In certain aspects, the orthogonal modifications in the E domain and the K domain comprise knob-in-hole mutations. In certain aspects, the knob-in hole mutations are a T366W mutation in one of the E domain or the K domain and a T366S, L368A, and aY407V mutation in the other domain. In certain aspects, the orthogonal modifications in the E domain and the K domain comprise charge-pair mutations. In certain aspects, the charge-pair mutations are a T366K mutation in one of the E domain or the K domain and a corresponding L351D mutation in the other domain.

In certain aspects, the amino acid sequences of the E domain and the K domain are endogenous sequences of two different antibody domains, the domains selected to have a specific interaction that promotes the specific association between the first and the third polypeptides. In certain aspects, the two different amino acid sequences are a CH1 sequence and a CL sequence.

In certain aspects, the ROR antigen binding molecule further comprises a third antigen binding site. In certain aspects, the third antigen binding site is specific for a ROR antigen. In certain aspects, the first antigen binding site and the third antigen binding site are specific for the same ROR antigen. In certain aspects, the first antigen binding site and the third antigen binding site are specific for different ROR antigens.

In certain aspects, ROR antigen binding molecule comprises a fifth polypeptide chain, wherein (a) the first polypeptide chain further comprises a domain N and a domain 0, wherein the domains are arranged, from N-terminus to C-terminus, in a N-O-A-B-D-E orientation, and wherein domain N has a variable region domain amino acid sequence, domain O has a constant region amino acid sequence; (b) the fifth polypeptide chain comprises a domain P and a domain Q, wherein the domains are arranged, from N-terminus to C-terminus, in a P-Q orientation, and wherein domain P has a variable region domain amino acid sequence and domain Q has a constant region amino acid sequence; and (c) the first and the fifth polypeptides are associated through an interaction between the N and the P domains and an interaction between the O and the Q domains to form the ROR antigen binding molecule.

In certain aspects, (a) the amino acid sequences of domain N and domain A are identical, the amino acid sequences of domain H is different from the sequence of domain N and domain A, the amino acid sequences of domain 0 and domain B are identical, the amino acid sequences of domain I is different from the sequence of domain 0 and domain B, the amino acid sequences of domain P and domain F are identical, the amino acid sequences of domain L is different from the sequence of domain P and domain F, the amino acid sequences of domain Q and domain G are identical, the amino acid sequences of domain M is different from the sequence of domain Q and domain G; and (b) wherein the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for the first antigen. In certain aspects, the first antigen is a ROR antigen. In certain aspects, the second antigen is a CD3 antigen.

In certain aspects, (a) the amino acid sequences of domain N, domain A, and domain H are different, the amino acid sequences of domain 0, domain B, and domain I are different, the amino acid sequences of domain P, domain F, and domain L are different, and the amino acid sequences of domain Q, domain G, and domain M are different; and (b) the interaction between the A domain and the F domain form a first antigen binding site specific for a first antigen, the interaction between the H domain and the L domain form a second antigen binding site specific for a second antigen, and the interaction between the N domain and the P domain form a third antigen binding site specific for a third antigen.

In certain aspects, the ROR antigen binding molecule comprises a sixth polypeptide chain, wherein: (a) the third polypeptide chain further comprises a domain R and a domain S, wherein the domains are arranged, from N-terminus to C-terminus, in a R-S-H-I-J-K orientation, and wherein domain R has a variable region amino acid sequence and domain S has a constant domain amino acid sequence; (b) the sixth polypeptide chain comprises: a domain T and a domain U, wherein the domains are arranged, from N-terminus to C-terminus, in a T-U orientation, and wherein domain T has a variable region amino acid sequence and domain U has a constant domain amino acid sequence; and (c) the third and the sixth polypeptides are associated through an interaction between the R and the T domains and an interaction between the S and the U domains to form the ROR antigen binding molecule.

In certain aspects, (a) the amino acid sequences of domain R and domain A are identical, the amino acid sequences of domain H is different from the sequence of domain R and domain A, the amino acid sequences of domain S and domain B are identical, the amino acid sequences of domain I is different from the sequence of domain S and domain B, the amino acid sequences of domain T and domain F are identical, the amino acid sequences of domain L is different from the sequence of domain T and domain F, the amino acid sequences of domain U and domain G are identical, the amino acid sequences of domain M is different from the sequence of domain U and domain G, and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for the first antigen. In certain aspects, the first antigen is a ROR antigen. In certain aspects, the second antigen is a CD3 antigen.

In certain aspects, (a) the amino acid sequences of domain R and domain H are identical, the amino acid sequences of domain A is different from the sequence of domain R and domain H, the amino acid sequences of domain S and domain I are identical, the amino acid sequences of domain B is different from the sequence of domain S and domain I, the amino acid sequences of domain T and domain L are identical, the amino acid sequences of domain F is different from the sequence of domain T and domain L, the amino acid sequences of domain U and domain M are identical, the amino acid sequences of domain G is different from the sequence of domain U and domain M, and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for the second antigen. In certain aspects, the second antigen is a ROR antigen. In certain aspects, the first antigen is a CD3 antigen.

In certain aspects, (a) the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and (b) the interaction between the A domain and the F domain form a first antigen binding site specific for a first antigen, the interaction between the H domain and the L domain form a second antigen binding site specific for a second antigen, and the interaction between the R domain and the T domain form a third antigen binding site specific for a third antigen.

Also described herein are purified ROR antigen binding molecules, the purified ROR antigen binding molecules comprising any of the ROR antigen binding molecule described herein. In certain aspects, the purified ROR antigen binding molecules are purified by a purification method comprising a CH1 affinity purification step. In certain aspects, the purification method is a single-step purification method.

Also described herein are pharmaceutical compositions comprising any of the ROR antigen binding molecules described herein and a pharmaceutically acceptable diluent.

Also described herein are methods for treating a subject with cancer, the methods comprising administering a therapeutically effective amount of any of the pharmaceutical compositions described herein. In certain aspects, the cancer is selected from the group consisting of: pancreatic cancer, ovarian cancer, breast cancer, lung cancer, gastric cancer, melanoma, Ewing sarcoma, chronic lymphocytic leukemia, mantle cell lymphoma, B-ALL, hematological cancer, prostate cancer, colon cancer, renal cancer, and uterine cancer.

### 5. BRIEF DESCRIPTION OF THE DRAWINGS

FIG. 1 shows an alignment of the CH3-CH3 IgG1 dimer pair with CH1-CL. The quaternary structures align with an RMSD of ˜1.6 Å².

FIG. 2 presents schematic architectures, with respective naming conventions, for various binding molecules (also called antibody constructs) described herein.

FIG. 3 presents a higher resolution schematic of polypeptide chains and their domains, with respective naming conventions, for the bivalent 1×1 antibody constructs described herein.

FIG. 4 shows the architecture of an exemplary bivalent, monospecific, construct.

FIG. 5 shows data from a biolayer interferometry (BLI) experiment, described in Example 1, in which a bivalent monospecific binding molecule having the architecture illustrated in FIG. 4 [polypeptide 1: VL—CH3(Knob)—CH2—CH3/polypeptide 2: VH-CH3(Hole)] was assayed. The antigen binding site was specific for TNFα. The BLI response from binding molecule immobilization and TNFα binding to the immobilized construct demonstrates robust, specific, bivalent binding to the antigen. The data are consistent with a molecule having a high percentage of intended pairing of polypeptide 1 and polypeptide 2.

FIG. 6 illustrates features of an exemplary bivalent 1×1 bispecific binding molecule, “BC1”.

FIG. 7A shows size exclusion chromatography (SEC) analysis of “BC1”, demonstrating that a single-step CH1 affinity purification step (CaptureSelect™ CH1 affinity resin) yields a single, monodisperse peak via gel filtration in which>98% is unaggregated bivalent protein. FIG. 7B shows comparative literature data of SEC analysis of a CrossMab bivalent antibody construct [data from Schaefer et al. (Proc Natl Acad Sci USA. 2011 Jul. 5; 108(27):11187-92)].

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification.

FIG. 9 shows nonreducing SDS-PAGE gels of “BC1” at various stages of purification.

FIGS. 10A and 10B compare SDS-PAGE gels of “BC1” after single-step CH1-affinity purification under both non-reducing and reducing conditions (FIG. 10A) with SDS-PAGE gels of a CrossMab bispecific antibody under non-reducing and reducing conditions as published in the referenced literature (FIG. 10B).

FIGS. 11A and 11B show mass spectrometry analysis of “BC1”, demonstrating two distinct heavy chains (FIG. 11A) and two distinct light chains (FIG. 11B) under reducing conditions.

FIG. 12 presents a mass spectrometry analysis of purified “BC1” under non-reducing conditions, confirming the absence of incomplete pairing after purification.

FIG. 13 presents accelerated stability testing data demonstrating stability of “BC1” over 8 weeks at 40° C., compared to two IgG control antibodies.

FIG. 14 illustrates features of an exemplary bivalent 1×1 bispecific binding molecule, “BC6”, further described in Example 3.

FIG. 15A presents size exclusion chromatography (SEC) analysis of “BC6” following one-step purification using the CaptureSelect™ CH1 affinity resin, demonstrating that the single step CH1 affinity purification yields a single monodisperse peak and the absence of non-covalent aggregates. FIG. 15B shows a SDS-PAGE gel of “BC6” under non-reducing conditions.

FIG. 16 illustrates features of an exemplary bivalent bispecific binding molecule, “BC28”, further described in Example 4.

FIG. 17 shows SDS-PAGE analysis under non-reducing conditions following single-step CH1 affinity purification of “BC28”, “BC29”, “BC30”, “BC31”, and “BC32”.

FIG. 18 shows SEC analysis of “BC28” and “BC30”, each following one-step purification using the CaptureSelect™ CH1 affinity resin.

FIG. 19 illustrates features of an exemplary bivalent bispecific binding molecule, “BC44”, further described in Example 5.

FIGS. 20A and 20B show size exclusion chromatography (SEC) data of two bivalent binding molecules, “BC15” and “BC16”, respectively, under accelerated stability testing conditions. “BC15” and “BC16” have different variable region-CH3 junctions.

FIG. 21 presents a schematic of five polypeptide chains and their domains, with respective naming conventions, for the trivalent 2x1 antibody constructs described herein, wherein according to the naming convention, chain 5 is named “₅th polypeptide chain” in the schematic.

FIG. 22 illustrates features of an exemplary trivalent 2x1 bispecific binding molecule, “BC1-2x1”, further described in Example 7.

FIG. 23 shows non-reducing SDS-PAGE of “BC1” and “BC1-2x1” protein expressed using the ThermoFisher Expi293 transient transfection system, at various stages of purification.

FIG. 24 compares the avidity of the bivalent 1×1 construct “BC1” to the avidity of the trivalent 2x1 construct “BC1-2x1” using an Octet (Pall ForteBio) biolayer interferometry analysis.

FIG. 25 illustrates salient features of a trivalent 2x1 construct, “TB111.”

FIG. 26 presents a schematic of five polypeptide chains and their domains, with respective naming conventions, for the trivalent 1x2 antibody constructs described herein, wherein according to the naming convention, chain 5 is named “6^{th} polypeptide chain” in the schematic.

FIG. 27 illustrates features of an exemplary trivalent 1×2 construct “CTLA4-4× Nivo x CTLA4-4”, further described in Example 10.

FIG. 28 is a SDS-PAGE gel in which the lanes showing the trivalent 1×2 construct “CTLA4-4× Nivo x CTLA4-4” construct under non-reducing (“-DTT”) and reducing (“+DTT”) conditions have been boxed.

FIG. 29 shows a comparison of antigen binding between two antibodies: bivalent 1×1 construct “CTLA4-4× OX40-8” and the trivalent 1×2 construct “CTLA4-4× Nivo x CTLA4-4.” “CTLA4-4× OX40-8” binds to CTLA4 monovalently, while “CTLA4-4× Nivo x CTLA4-4” binds to CTLA4 bivalently.

FIG. 30 illustrates features of an exemplary trivalent 1×2 trispecific construct, “BC28-1x1x1a”, further described in Example 11.

FIG. 31 shows size exclusion chromatography of “BC28-1x1x1a” following transient expression and single step CH1 affinity resin purification, demonstrating a single well-defined peak.

FIG. 32 shows SDS-PAGE results with bivalent and trivalent constructs, each after transient expression and one-step purification using the CaptureSelect™ CH1 affinity resin, under non-reducing and reducing conditions, as further described in Example 12.

FIGS. 33A-33C show Octet binding analyses to 3 antigens: PD1, Antigen “A”, and CTLA4. As further described in Example 13, FIG. 33A shows binding of “BC1” to PD1 and Antigen “A”; FIG. 33B shows binding of a bivalent bispecific construct “CTLA4-4× OX40-8” to CTLA4, Antigen “A”, and PD1; FIG. 33C shows binding of trivalent trispecific “BC28-1x1x1a” to PD1, Antigen “A”, and CTLA4.

FIG. 34 presents a schematic of six polypeptide chains and their domains, with respective naming conventions, for certain tetravalent 2×2 constructs described herein.

FIG. 35 illustrates certain salient features of the exemplary tetravalent 2×2 construct, “BC22-2×2” further described in Example 14.

FIG. 36 is a non-reducing SDS-PAGE gel comparing the 2×2 tetravalent “BC22-2×2” construct to a 1×2 trivalent construct “BC12-1×2” and a 2×1 trivalent construct “BC21-2×1” at different stages of purification.

FIG. 37 provides architecture for an exemplary tetravalent 2×2 construct.

FIG. 38 presents a schematic of six polypeptide chains and their domains, with respective naming conventions, for certain tetravalent constructs described herein, wherein according to the naming convention, chain 5 is named “7^{th} polypeptide chain” and chain 6 is named “8^{th} polypeptide chain” in the schematic.

FIG. 39 provides exemplary architecture of a bispecific tetravalent construct.

FIG. 40 provides exemplary architecture for a trispecific tetravalent construct utilizing a common light chain strategy.

FIG. 41 shows bispecific antigen engagement by the tetravalent construct schematized in FIG. 39, demonstrating that this construct was capable of simultaneous engagement. The biolayer interferometry (BLI) response from B-Body immobilization and TNFα binding to the immobilized construct are consistent with a molecule with a high percentage of intended chain pairing.

FIG. 42 provides flow cytometry analysis of B-Body binding to cell-surface antigen. Cross-hatched signal indicates cells without antigen; dotted signal indicates transiently transfected cells with surface antigen.

FIG. 43 provides exemplary architecture of a trivalent construct.

FIG. 44 provides exemplary architecture of a trivalent construct.

FIG. 45 shows SDS-PAGE results with bivalent and trivalent constructs, each after transient expression and one-step purification using the CaptureSelect™ CH1 affinity resin, under non-reducing and reducing conditions, as further described in Example 17.

FIG. 46 shows differences in the thermal transitions for “BC24jv”, “BC26jv”, and “BC28jv” measured to assess pairing stability of junctional variants.

FIG. 47 demonstrates Octet (Pall ForteBio) biolayer interferometry analysis of a two-fold serial dilution (200-12.5 nM) used to determine binding affinity to CD3 for a non-mutagenized SP34-89 monovalent B-Body.

FIGS. 48A-48B demonstrate Octet (Pall ForteBio) biolayer interferometry analysis of a two-fold serial dilution (200-12.5 nM) used to determine binding affinity to ROR1 for two ROR antigen binding site candidates (FIG. 48A clone I2-A10; FIG. 48B clone I2-A27).

FIG. 49 shows that RORxCD3 bispecific 1×1 and 1×2 B-bodies resulted in activation of reporter T cells when mixed with ROR1 expressing tumor lines (HOP-92), but no activation when mixed with tumor lines that do not express ROR1 (B16).

FIG. 50 shows the RORxCD3 bispecific I2A-10 1×2 B-body resulted in cytotoxic T cell mediated killing when mixed with ROR1 expressing tumor lines (MDA-MD-231), but did not result in cytotoxicity when a CD3 bispecific B-body having an irrelevant tumor ABS (e.g., a tumor antigen not expressed in MDA-MD-231) was added to the mixture.

FIGS. 51A-51E show that the RORxCD3 bispecific I2A-3 1×1 and 1×2 B-bodies resulted in activation of the reporter T cells when mixed with ROR1 expressing tumor lines HOP-92 (FIG. 51A), A549 (FIG. 51B), MDA-MD-231 (FIG. 51C), JeKo-1 (FIG. 51D), and RPMI-8226 (FIG. 51E), but no activation when mixed with tumor lines that do not express ROR1 (B16).

FIG. 52 shows that the RORxCD3 bispecific 1×2 B-bodies I2A-1, 12A-3, 12A-10, 12A-14, 12A-22, and I2A-27 resulted in cytotoxic T cell mediated killing when mixed with ROR1 expressing tumor lines (MDA-MD-231), but 1×2 B-bodies I2A-16 and 12A-22 did not result in potent cytotoxicity.

FIG. 53A illustrates published ROR1 expression data for the MDA-MD-231 and RPMI-8226 tumor lines FIG. 53B and FIG. 53C demonstrate that the cytotoxicity efficacy correlates with ROR1 in MDA-MD-231 and RPMI-8226 tumor cell lines.

FIGS. 54A-54F show that I2A-10 and I2A-27 B-bodies activated CD8+ T cells in a PBMC population as determined by quantifying CD25 (FIG. 54A), CD69 (FIG. 54C), and both CD25 and CD69 (FIG. 54E), as well as activated CD4+ T cells in a PBMC population as determined by quantifying CD25 (FIG. 54B), CD69 (FIG. 54D), and both CD25 and CD69 (FIG. 54F).

FIG. 55 shows that 26% and 36% of candidates I2A-10 (top panel) and I2A-27 (bottom panel) were internalized following a 2 hour incubation with MDA-MB-231 cells, respectively.

FIG. 56 shows size exclusion chromatography (SEC) analysis, demonstrating that a single-step CH1 affinity purification step yields single, monodisperse peaks via gel filtration in which>98% is unaggregated protein for 1×2 B-body candidates I2A-10 (top panel) and I2A-27 (bottom panel).

FIG. 57A shows non-reducing SDS-PAGE gels of 1×2 B-body candidates I2A-10 (left panel) and I2A-27 (right panel), demonstrating a major band of fully assembled constructs (high-migrating 250 kDa band).

FIG. 57B shows Bioanalyzer (Agilent) analysis of non-reduced samples for 1×2 B-body candidates 12A-10 and I2A-27 demonstrating a major band of fully assembled constructs.

FIG. 58 shows SDS-PAGE analysis of bispecific antibodies comprising standard knob-hole orthogonal mutations introduced into CH3 domains found in their native positions within the Fc portion of the bispecific antibody that have been purified using a single-step CH1 affinity purification step (CaptureSelect™ CH1 affinity resin).

FIGS. 59A-59B show Octet (Pall ForteBio) biolayer interferometry analysis demonstrating FcγRIa binding to trastuzumab (FIG. 59A “WT IgG1”), but not sFc10 (FIG. 59B).

FIG. 60 shows killing by trastuzumab (Herceptin, “WT-IgG1”) but not by sFc7 or sFc10 in an ADCC assay.

FIG. 61 shows C1q binding by trastuzumab (Herceptin, “WT-IgG1”) but not by sFc1, sFc7, or sFc10 in a C1q ELISA.

FIGS. 62A-62C show tumor volume monitored for mice engrafted with tumor cells, humanized with PBMCs (left solid arrow), then subsequently treated IV (right dashed arrow) with PBS (FIG. 62A), 1×2 B-body candidate I2-A10 (FIG. 62B), or 1×2 B-body candidate I2-A27 (FIG. 62C).

FIG. 63 shows tumor volume at the conclusion of the study for each of the mice, with mean and standard deviation for each group shown. The open square for the I2-A27 group was removed from the analysis due to probable non-humanization by PMBCs.

FIG. 64A shows binding of SP34-89 to Jurkat and cynomolgus T cells. FIG. 64B shows binding of an I2-A27 1×2 B-body™ bispecific antibody to cynomolgus CD3 delta and epsilon heterodimer.

FIG. 65A shows binding of an I2-A27 IgG antibody to ROR1 in a monovalent binding assay. FIG. 65B shows minimal binding of an I2-A27 IgG antibody to ROR2. FIG. 65C shows binding of an I2-A27 1×2 B-body™ to ROR1 in a monovalent binding assay.

FIG. 65D shows minimal binding of an I2-A27 1×2 B-body™ to ROR2. FIG. 65E shows binding of an I2-A27 1×2 B-body™ to ROR1 in a bivalent binding assay.

FIG. 66A shows SEC analysis of an I2-A27 1×2 B-body™. FIG. 66B shows SMAC analysis of an I2-A27 1×2 B-body™. FIG. 66C shows HIC analysis of an I2-A27 1×2 B-body™

FIG. 67A shows activity of an I2-A27 1×2 B-body™ in a Jurkat assay with MDA-MB-231 (ROR1 expressing) cells. FIG. 67B shows activity of an I2-A27 1×2 B-body™ in a Jurkat assay with RPMI-8226 (ROR1 and ROR2 expressing) cells. FIG. 67C shows inactivity of an I2-A27 1×2 B-body™ in a Jurkat assay with K562 (ROR2 expressing) cells.

FIG. 67D shows inactivity of an I2-A27 1×2 B-body™ in a Jurkat assay in the absence of a target cell line.

FIG. 68A shows CD69 expression with various concentrations of an I2-A27 1×2 B-body™ and ROR1 expressing MDA-MB-231 cells. FIG. 68B shows CD69 expression with various concentrations of an I2-A27 1×2 B-body™ and ROR1 expressing RPMI-8226 cells.

FIG. 68C shows CD25 expression with various concentrations of an I2-A27 1×2 B-body™ and ROR1 expressing MDA-MB-231 cells. FIG. 68D shows CD25 expression with various concentrations of an I2-A27 1×2 B-body™ and ROR1 expressing RPMI-8226 cells.

FIG. 69A shows LDH release with various concentrations of an I2-A27 1×2 B-body™ and ROR1 expressing MDA-MB-231 cells. FIG. 69B shows LDH release with various concentrations of an I2-A27 1×2 B-body™ and ROR1 expressing RPMI-8226 cells.

FIG. 70A shows Granzyme B with various concentrations of an I2-A27 1×2 B-body™ and ROR1 expressing MDA-MB-231 cells. FIG. 70B shows Granzyme B with various concentrations of an I2-A27 1×2 B-body™ and ROR1 expressing RPMI-8226 cells.

FIG. 70C shows TNFα secretion with various concentrations of an I2-A27 1×2 B-body™ and ROR1 expressing MDA-MB-231 cells. FIG. 70D shows TNFα secretion with various concentrations of an I2-A27 1×2 B-body™ and ROR1 expressing RPMI-8226 cells. FIG. 70E shows IFNγ release with various concentrations of an I2-A27 1×2 B-body™ and ROR1 expressing MDA-MB-231 cells. FIG. 70F shows IFNγ release with various concentrations of an I2-A27 1×2 B-body™ and ROR1 expressing RPMI-8226 cells.

FIG. 71A shows activity for I2-A27 1×2 B-body™ samples stored in human serum at 4° C. or 37° C. for 1 week. FIG. 71B shows inactivity of samples in a Jurkat assay in the absence of ROR1 expressing cells.

FIG. 72 shows stability assays of I2-A27 1×2 B-body™ samples under accelerated conditions.

FIG. 73 shows stability assays of I2-A27 1×2 B-body™ samples under real time conditions.

FIG. 74 shows acid stability assays of an I2-A27 1×2 B-body™

FIG. 75 shows binding of an I2-A10 D54E Y55Q 1×2 B-body™ to cynomolgus CD3 delta and epsilon heterodimer.

FIG. 76A shows binding of an I2-A10 D54E Y55Q IgG antibody to ROR1 in an monovalent binding assay. FIG. 76B shows binding of an I2-A10 D54E Y55Q IgG to ROR2 in a monovalent binding assay. FIG. 76C shows binding of an I2-A10 D54E Y55Q 1×2 B-body™ to ROR1 in a monovalent binding assay. FIG. 76D shows binding of an I2-A10 D54E Y55Q 1×2 B-body™ to ROR2 in a monovalent binding assay. FIG. 76E shows binding of an I2-A10 D54E Y55Q 1×2 B-body™ to ROR1 in a bivalent binding assay. FIG. 76F shows binding of an I2-A10D54E Y55Q 1×2 B-body™ to ROR2 in a bivalent binding assay.

FIG. 77 shows binding of an I2-A10D54E Y55Q 1×2 B-body™ to Ig-like domain of ROR1.

FIG. 78A shows SEC analysis of an I2-A10D54E Y55Q 1×2 B-body™. FIG. 78B shows SMAC analysis of an I2-A10D54E Y55Q 1×2 B-body™. FIG. 78C shows HIC analysis of an I2-A10D54E Y55Q 1×2 B-body™

FIG. 79A shows activity of an I2-A10D54E Y55Q 1×2 B-body™ in Jurkat assay with MDA-MB-231 (ROR1 expressing) cells. FIG. 79B shows activity of an I2-A10 D54E Y55Q 1×2 B-body™ in a Jurkat assay with RPMI-8226 (ROR1 and ROR2 expressing) cells.

FIG. 79C shows activity of an I2-A10D54E Y55Q 1×2 B-body™ with K562 (ROR2 expressing) cells. FIG. 79D shows inactivity of an I2-A10D54E Y55Q 1×2 B-body™ in a Jurkat assay in the absence of a target cell line.

FIG. 80A shows CD69 expression with various concentrations of an I2-A10D54E Y55Q 1×2 B-body™ and ROR1 expressing MDA-MB-231 cells. FIG. 80B shows CD69 expression with various concentrations of an I2-A10D54E Y55Q 1×2 B-body™ and ROR1 and ROR2 expressing RPMI-8226 cells. FIG. 80C shows CD25 expression with various concentrations of an I2-A10D54E Y55Q 1×2 B-body™ and ROR1 expressing MDA-MB-231 cells. FIG. 80D shows CD25 expression with various concentrations of an I2-A10D54E Y55Q 1×2 B-body™ and ROR1 and ROR2 expressing RPMI-8226 cells.

FIG. 81A shows LDH release with various concentrations of an I2-A10D54E Y55Q 1×2 B-body™ and ROR1 expressing MDA-MB-231 cells. FIG. 81B shows LDH release with various concentrations of an I2-A10D54E Y55Q 1×2 B-body™ and ROR1 and ROR2 expressing RPMI-8226 cells.

FIG. 82A shows Granzyme B with various concentrations of an I2-A10D54E Y55Q 1×2 B-body™ and ROR1 expressing MDA-MB-231 cells. FIG. 82B shows Granzyme B with various concentrations of an I2-A10D54E Y55Q 1×2 B-body™ and ROR1 and ROR2 expressing RPMI-8226 cells. FIG. 82C shows TNFα secretion with various concentrations of an I2-A10D54E Y55Q 1×2 B-body and ROR1 expressing MDA-MB-231 cells. FIG. 82D shows TNFα secretion with various concentrations of an I2-A10D54E Y55Q 1×2 B-body™ and ROR1 and ROR2 expressing RPMI-8226 cells. FIG. 82E shows IFNγ secretion with various concentrations of an I2-A10D54E Y55Q 1×2 B-body™ and ROR1 expressing MDA-MB-231 cells. FIG. 82F shows IFNγ secretion with various concentrations of an I2-A10 D54E Y55Q 1×2 B-body™ and ROR1 and ROR2 expressing RPMI-8226 cells.

FIG. 83A shows activity of an I2-A10 D54E Y55Q 1×2 B-body™ samples stored in human serum at 4° C. or 37° C. for 1 week. FIG. 83B shows inactivity of samples in a Jurkat assay in the absence of ROR1 expressing cells.

FIG. 84 shows stability assays of I2-A10 D54E Y55Q 1×2 B-body™ samples under accelerated conditions.

FIG. 85 shows stability assays of I2-A10 D54E Y55Q 1×2 B-body™ samples under real time conditions.

FIG. 86 shows acid stability assays of an I2-A10 D54E Y55Q 1×2 B-body™

FIG. 87 shows in vivo efficacy of an I2-27 1×2 B-body™ and an I2-A10 D54E Y55Q 1×2 B-body™ to reduce tumor volume (mm³) in a mouse model of cancer.

FIG. 88 shows in vivo efficacy of multiple doses of an I2-27 1×2 B-body™ and an I2-A10 D54E Y55Q 1×2 B-body™ to reduce tumor size (mm³) in a mouse model of cancer.

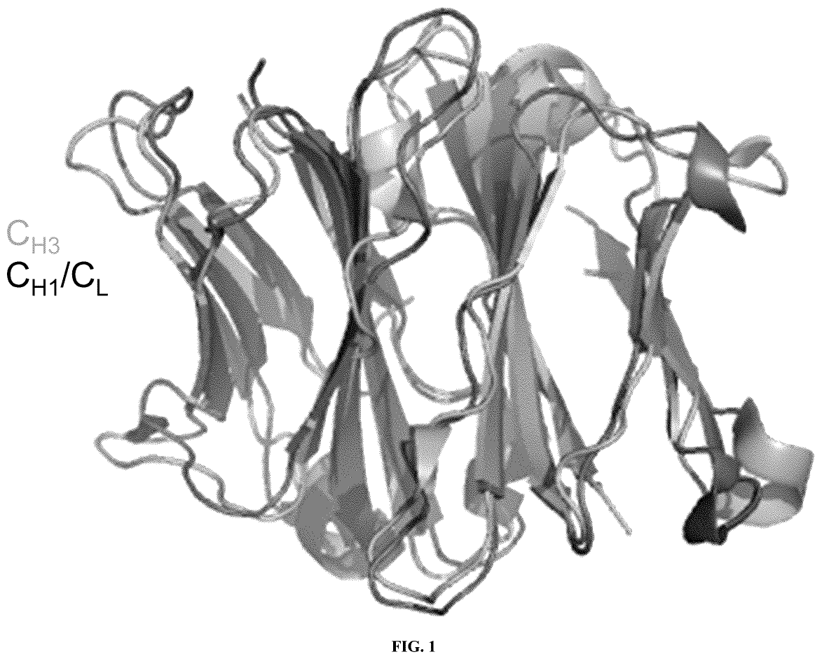

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs.

As used herein, the following terms have the meanings ascribed to them below.

By “antigen binding site” is meant a region of a ROR binding molecule that specifically recognizes or binds to a given antigen or epitope.

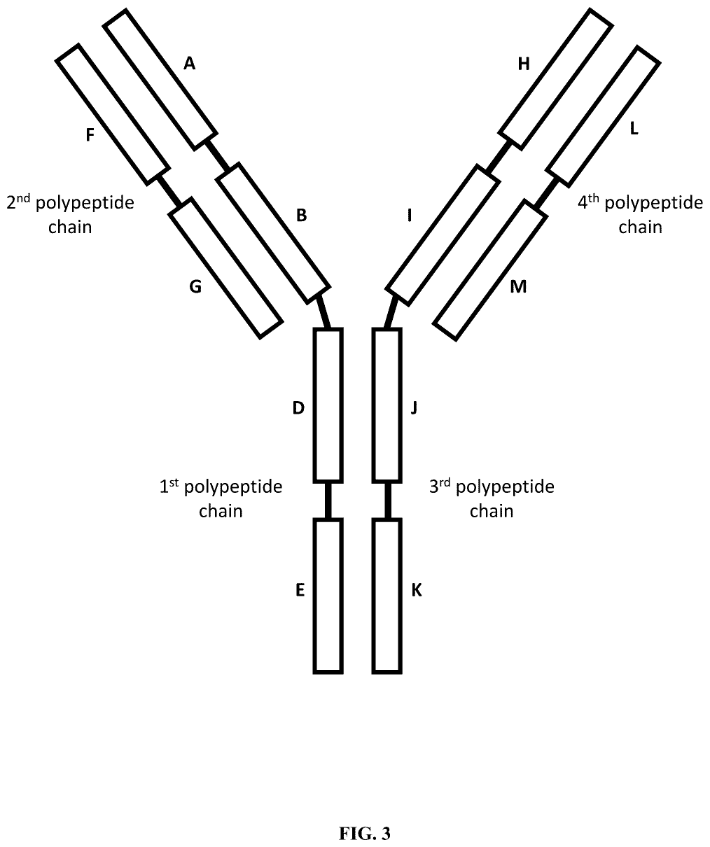

“B-Body,” as used herein and with reference to FIG. 3, refers to binding molecules comprising the features of a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the binding molecule. B-bodies are described in more detail in International Patent Application No. PCT/US2017/057268, herein incorporated by reference in its entirety.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, arthritis, or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

### 6.2. Other Interpretational Conventions

Unless otherwise specified, all references to sequences herein are to amino acid sequences.

Unless otherwise specified, antibody constant region residue numbering is according to the Eu index as described at

www.imgt.org/IMGTScientificChart/Numbering/Hu_IGHGnber.html #refs (accessed Aug. 22, 2017) and in Edelman et al., Proc. Natl. Acad. USA, 63:78-85 (1969), which are hereby incorporated by reference in their entireties, and identifies the residue according to its location in an endogenous constant region sequence regardless of the residue's physical location within a chain of the ROR binding molecules described herein. By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Ranges provided herein are understood to be shorthand for all of the values within the range, inclusive of the recited endpoints. For example, a range of 1 to 50 is understood to include any number, combination of numbers, or sub-range from the group consisting of 1, 2, 3,4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or apparent from context, as used herein the term “or” is understood to be inclusive. Unless specifically stated or apparent from context, as used herein, the terms “a”, “an”, and “the” are understood to be singular or plural.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### 6.3. ROR Antigen Binding Molecules

In a first aspect, antigen binding molecules are provided. In every embodiment, the antigen binding molecule includes at least a first antigen binding site specific for a ROR antigen; the binding molecules are therefore termed ROR antigen binding molecules.

The ROR binding molecules described herein bind specifically to ROR antigens.

As used herein, “ROR antigens” refer to members of the tyrosine-protein kinase transmembrane receptor (ROR) family, including members ROR1 and ROR2. In certain embodiments, the ROR binding molecule has antigen binding sites that specifically bind to ROR1 only. In other embodiments, the ROR binding molecule has antigen binding sites that specifically bind to ROR2 only. In still other embodiments, the ROR binding molecule has antigen binding sites that are cross-reactive and specifically bind to both ROR1 and ROR2.

ROR1 and ROR2 proteins typically consist of at least four protein domains: three extracellular domains—Ig-like, FZ, and Kringle domains—as well as the intracellular Protein Kinase domain. In some embodiments, the ROR binding molecule has antigen binding sites that specifically bind to the extracellular portion of the ROR antigen. In certain embodiments, the ROR binding molecule has antigen binding sites that specifically bind to the Ig-like domain. In other embodiments, the ROR binding molecule has antigen binding sites that specifically bind to the FZ domain. In still other embodiments, the ROR binding molecule has antigen binding sites that specifically bind to the Kringle domain. In particular embodiments, the ROR binding molecule has antigen binding sites that specifically bind to at least a portion of a single ROR domain. In particular embodiments, the ROR binding molecule has antigen binding sites that specifically bind to at least a portion of more than one ROR domain, such as the junction between a first and a second ROR domain. The ROR domains can refer to ROR1 domains or ROR2 domains.

In specific embodiments, the ROR antigen is human. UniProt accession #Q01973 describes a canonical human ROR1 protein, including its sequences and domain features, and is hereby incorporated by reference in its entirety. SEQ ID NO:94 provides the full-length ROR1 protein sequence. With reference to the full-length sequence from the N-terminus to C-terminus, the Ig-Like domain is defined as amino acids 42-147, the FZ domain as amino acids 165-299, and the Kringle domain as amino acids 312-391. UniProt accession #Q01974 describes a canonical human ROR2 protein, including its sequences and domain features, and is hereby incorporated by reference in its entirety. SEQ ID NO:95 provides the full-length ROR2 protein sequence. With reference to the full-length sequence from the N-terminus to C-terminus, the Ig-Like domain is defined as amino acids 55-145, the FZ domain as amino acids 169-303, and the Kringle domain as amino acids 316-394.

Various tumors can demonstrate cell-surface expression of ROR antigens, as described in greater detail in Gentile, et al. (Cancer Res; 71(8) Apr. 15, 2011), Rebagay, et al. (Front. Oncol., 18 Apr. 2012), Zhang, et al. (American Journal ofPathology, Vol. 181, No. 6, December 2012), Henry, et al. (Oncotarget, Vol. 6, No. 37 2015), Zhang, et al. (PLoS ONE 7(3): e31127.), and Bainbridge, et al. (PLoS ONE 9(7): e102695.), each herein incorporated by reference in their entirety. In addition, ROR expression may not be expressed, or only demonstrate limited expression, in normal, i.e. non-cancerous, tissue as described in Balakrishnan et al. (Clin Cancer Res. 2017 Jun. 15; 23(12): 3061-3071), herein incorporated in its entirety. Thus ROR antigens can be used as a tumor-specific marker in certain tumors. Examples of tumors and cancers with demonstrated ROR expression include, but are not limited to, pancreatic cancer, ovarian cancer, breast cancer, lung cancer, gastric cancer, melanoma, Ewing sarcoma, chronic lymphocytic leukemia, mantle cell lymphoma, and B-ALL, as described in Gohil et al. (Oncoimmunology. 2017; 6(7): e1326437.), herein incorporated in its entirety. Other cancers include, but are not limited to, hematological cancer, prostate cancer, colon cancer, renal cancer, and uterine cancer.

In various embodiments, the ROR binding molecule additionally binds specifically to at least one antigen additional to a ROR antigen.

In a specific embodiment, the ROR binding molecule is a bispecific bivalent molecule. In another embodiment, the ROR binding molecule is a bispecific trivalent molecule. In particular embodiments, the ROR binding molecule has antigen binding sites that specifically bind the ROR antigen and a T cell surface expressed molecule. In a specific embodiment, the ROR binding molecule has antigen binding sites that specifically bind the ROR antigen and the T cell surface expressed protein CD3. Without wishing to be bound by theory, the ROR binding molecule that specifically binds the ROR antigen and the T cell surface expressed molecule (i.e., CD3) can direct T cell mediated killing (cytotoxicity) of cells expressing the ROR antigen through redirecting T cells to the ROR expressing cells (i.e., target cells). T cell mediated killing using bispecific anti-CD3 molecules is described in detail in U.S. Pub. No. 2006/0193852, herein incorporated by reference in its entirety. In some embodiments, the T cell surface expressed molecule is selected from any molecule capable of redirecting T cells to a target cell.

With reference to FIG. 3, in a series of embodiments, the ROR binding molecules comprise a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a CL amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and wherein the fourth polypeptide chain comprises the CH1 domains and domain M is the CH1 domain, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the ROR binding molecule.

In a series of embodiments, (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein the third polypeptide chain comprises the CH1 domain and domain I is the CH1 domain, or portion thereof, domain H has a variable region domain amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and wherein domain M has a CL amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the ROR binding molecule.

### 6.3.1. Domain A (Variable Region)

In the ROR binding molecules, domain A has a variable region domain amino acid sequence. Variable region domain amino acid sequences, as described herein, are variable region domain amino acid sequences of an antibody including VL and VH antibody domain sequences. VL and VH sequences are described in greater detail below in Sections 6.3.1.1 and 6.3.1.4, respectively. In a preferred embodiment, domain A has a VL antibody domain sequence and domain F has a VH antibody domain sequence.

### 6.3.1.1.VL Regions

The VL amino acid sequences useful in the ROR binding molecules described herein are antibody light chain variable domain sequences. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail below in Sections 6.3.1.2 and 6.3.1.3.

In various embodiments, VL amino acid sequences are mutated sequences of naturally occurring sequences. In certain embodiments, the VL amino acid sequences are lambda (k) light chain variable domain sequences. In certain embodiments, the VL amino acid sequences are kappa (x) light chain variable domain sequences. In a preferred embodiment, the VL amino acid sequences are kappa (x) light chain variable domain sequences.

In the ROR binding molecules described herein, the C-terminus of domain A is connected to the N-terminus of domain B. In certain embodiments, domain A has a VL amino acid sequence that is mutated at its C-terminus at the junction between domain A and domain B, as described in greater detail below in Section 6.3.19.1 and in Example 6.

### 6.3.1.2. Complementarity Determining Regions

The VL amino acid sequences comprise highly variable sequences termed “complementarity determining regions” (CDRs), typically three CDRs (CDR1, CD2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries.

### 6.3.1.3. Framework Regions and CDR Grafting

The VL amino acid sequences comprise “framework region” (FR) sequences. FRs are generally conserved sequence regions that act as a scaffold for interspersed CDRs (see Section 6.3.1.2.), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences.

Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.3.1.4.VH Regions

The VH amino acid sequences in the ROR binding molecules described herein are antibody heavy chain variable domain sequences. In a typical antibody arrangement in both nature and in the ROR binding molecules described herein, a specific VH amino acid sequence associates with a specific VL amino acid sequence to form an antigen-binding site.

In various embodiments, VH amino acid sequences are mammalian sequences, including human sequences, synthesized sequences, or combinations of non-human mammalian, mammalian, and/or synthesized sequences, as described in further detail above in Sections 6.3.1.2 and 6.3.1.3. In various embodiments, VH amino acid sequences are mutated sequences of naturally occurring sequences.

### 6.3.2. Domain B (Constant Region)

In the ROR binding molecules, Domain B has a constant region domain sequence. Constant region domain amino acid sequences, as described herein, are sequences of a constant region domain of an antibody.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype. In preferred specific embodiments, the constant region sequence is a CH3 sequence. CH3 sequences are described in greater detail below in Section 6.3.2.1. In other preferred embodiments, the constant region sequence is an orthologous CH2 sequence. Orthologous CH2 sequences are described in greater detail below in Section 6.3.2.2.

In particular embodiments, the constant region sequence has been mutated to include one or more orthogonal mutations. In a preferred embodiment, domain B has a constant region sequence that is a CH3 sequence comprising knob-hole (synonymously, “knob-in-hole,” “KIH”) orthogonal mutations, as described in greater detail below in Section 6.3.14.2, and either a S354C or a Y349C mutation that forms an engineered disulfide bridge with a CH3 domain containing an orthogonal mutation, as described in in greater detail below in Section 6.3.14.1. In some preferred embodiments, the knob-hole orthogonal mutation is a T366W mutation.

### 6.3.2.1.CH3 Regions

CH3 amino acid sequences, as described herein, are sequences of the C-terminal domain of an antibody heavy chain.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype or CH4 sequences from an IgE or IgM isotype. In a specific embodiment, the CH3 sequences are from an IgG isotype. In a preferred embodiment, the CH3 sequences are from an IgG1 isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a ROR binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail below in Sections 6.3.14.1-6.3.14.3.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1 ml allotype are replaced. In a preferred embodiment, isoallotype mutations D356E and L358M are made in the CH3 sequence.

In a preferred embodiment, domain B has a human IgG1 CH3 amino acid sequence with the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In certain embodiments, domain B has a human IgG1 CH3 sequence with a 447C mutation incorporated into an otherwise endogenous CH3 sequence.

In the ROR binding molecules described herein, the N-terminus of domain B is connected to the C-terminus of domain A. In certain embodiments, domain B has a CH3 amino acid sequence that is mutated at its N-terminus at the junction between domain A and domain B, as described in greater detail below in Section 6.3.19.1 and Example 6.

In the ROR binding molecules, the C-terminus of domain B is connected to the N-terminus of domain D. In certain embodiments, domain B has a CH3 amino acid sequence that is extended at the C-terminus at the junction between domain B and domain D, as described in greater detail below in Section 6.3.19.3.

### 6.3.2.2. Orthologous CH2 Regions

CH2 amino acid sequences, as described herein, are sequences of the third domain of an antibody heavy chain, with reference from the N-terminus to C-terminus. CH2 amino acid sequences, in general, are discussed in more detail below in section 6.3.3. In a series of embodiments, a ROR binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the ROR binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the ROR binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the ROR binding molecules are described in more detail in international PCT applications WO2017/011342 and WO2017/106462, herein incorporated by reference in their entirety.

### 6.3.3. Domain D (Constant Region)

In the ROR binding molecules described herein, domain D has a constant region amino acid sequence. Constant region amino acid sequences are described in more detail in Section 6.3.2.

In a preferred series of embodiments, domain D has a CH2 amino acid sequence. CH2 amino acid sequences, as described herein, are CH2 amino acid sequences of the third domain of a native antibody heavy chain, with reference from the N-terminus to C-terminus. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from an IgA1, IgA2, IgD, IgE, IgG1, IgG2, IgG3, IgG4, or IgM isotype. In a preferred embodiment, the CH2 sequences are from an IgG1 isotype.

In certain embodiments, the CH2 sequences are endogenous sequences. In particular embodiments, the sequence is UniProt accession number P01857 amino acids 111-223. In a preferred embodiment, the CH2 sequences have an N-terminal hinge region peptide that connects the N-terminal variable domain-constant domain segment to the CH2 domain, as discussed in more detail below in Section 6.3.19.3.

In the ROR binding molecules, the N-terminus of domain D is connected to the C-terminus of domain B. In certain embodiments, domain B has a CH3 amino acid sequence that is extended at the C-terminus at the junction between domain D and domain B, as described in greater detail below in Section 6.3.19.3.

### 6.3.4. Domain E (Constant Region)

In the ROR binding molecules, domain E has a constant region domain amino acid sequence. Constant region amino acid sequences are described in more detail in Section 6.3.2.

In certain embodiments, the constant region sequence is a CH3 sequence. CH3 sequences are described in greater detail above in Section 6.3.2.1.. In particular embodiments, the constant region sequence has been mutated to include one or more orthogonal mutations. In a preferred embodiment, domain E has a constant region sequence that is a CH3 sequence comprising knob-hole (synonymously, “knob-in-hole,” “KIH”) orthogonal mutations, as described in greater detail below in Section 6.3.14.2, and either a S354C or a Y349C mutation that forms an engineered disulfide bridge with a CH3 domain containing an orthogonal mutation, as described in in greater detail below in Section 6.3.14.1.

In some preferred embodiments, the knob-hole orthogonal mutation is a T366W mutation. In certain embodiments, the constant region domain sequence is a CH1 sequence. In particular embodiments, the CH1 amino acid sequence of domain E is the only CH1 amino acid sequence in the ROR binding molecule. In certain embodiments, the N-terminus of the CH1 domain is connected to the C-terminus of a CH2 domain, as described in greater detail below in 6.3.19.5. In certain embodiments, the constant region sequence is a CL sequence. In certain embodiments, the N-terminus of the CL domain is connected to the C-terminus of a CH2 domain, as described in greater detail below in 6.3.19.5. CH1 and CL sequences are described in further detail in Section 6.3.8.1.

### 6.3.5. Domain F (Variable Region)

In the ROR binding molecules, domain F has a variable region domain amino acid sequence. Variable region domain amino acid sequences, as discussed in greater detail in Section 6.3.1, are variable region domain amino acid sequences of an antibody including VL and VH antibody domain sequences. VL and VH sequences are described in greater detail above in Sections 6.3.1.1 and 6.3.1.4, respectively. In a preferred embodiment, domain F has a VH antibody domain sequence.

### 6.3.6. Domain G (Constant Region)

In the ROR binding molecules, domain G has a constant region amino acid sequence. Constant region amino acid sequences are described in more detail in Section 6.3.2.

In preferred specific embodiments, the constant region sequence is a CH3 sequence. CH3 sequences are described in greater detail below in Section 6.3.2.1. In other preferred embodiments, the constant region sequence is an orthologous CH2 sequence. Orthologous CH2 sequences are described in greater detail below in Section 6.3.2.2.

In certain preferred embodiments, domain G has a human IgG1 CH3 sequence with the following mutational changes: S354C; and a tripeptide insertion, 445P, 446G, 447K. In some preferred embodiments, domain G has a human IgG1 CH3 sequence with the following mutational changes: S354C; and 445P, 446G, 447K tripeptide insertion. In some preferred embodiments, domain G has a human IgG1 CH3 sequence with the following changes: L351D, and a tripeptide insertion of 445G, 446E, 447C.

### 6.3.7. Domain H (Variable Region)

In the ROR binding molecules, domain L has a variable region domain amino acid sequence. Variable region domain amino acid sequences, as discussed in greater detail in Section 6.3.1, are variable region domain amino acid sequences of an antibody including VL and VH antibody domain sequences. VL and VH sequences are described in greater detail above in Sections 6.3.1.1. and 6.3.1.4, respectively. In a preferred embodiment, domain H has a VL antibody domain sequence.

### 6.3.8. Domain I (Constant Region)

In the ROR binding molecules, domain I has a constant region domain amino acid sequence. Constant region domain amino acid sequences are described in greater detail above in Section 6.3.2. In a series of preferred embodiments of the ROR binding molecules, domain I has a CL amino acid sequence. In another series of embodiments, domain I has a CH1 amino acid sequence. CH1 and CL amino acid sequences are described in further detail in Section 6.3.8.1.

### 6.3.8.1.CH1 and CL Regions

CH1 amino acid sequences, as described herein, are sequences of the second domain of an antibody heavy chain, with reference from the N-terminus to C-terminus. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The CL amino acid sequences useful in the ROR binding molecules described herein are antibody light chain constant domain sequences. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (k) light chain constant domain sequences. In particular embodiments, the CL amino acid sequences are human lambda light chain constant domain sequences. In preferred embodiments, the lambda (k) light chain sequence is UniProt accession number P0CG04.

In certain embodiments, the CL amino acid sequences are kappa (x) light chain constant domain sequences. In a preferred embodiment, the CL amino acid sequences are human kappa (x) light chain constant domain sequences. In a preferred embodiment, the kappa light chain sequence is UniProt accession number P01834.

In certain embodiments, the CH1 sequence and the CL sequences are both endogenous sequences. In certain embodiments, the CH1 sequence and the CL sequences separately comprise respectively orthogonal modifications in endogenous CH1 and CL sequences, as discussed below in greater detail in Section 6.3.8.2. It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the ROR binding molecule having a portion of the CH1 sequences described above can be bound by the CH1 binding reagent.

Without wishing to be bound by theory, the CH1 domain is also unique in that it's folding is typically the rate limiting step in the secretion of IgG (Feige et al. Mol Cell. 2009 Jun. 12; 34(5):569-79; herein incorporated by reference in its entirety). Thus, purifying the ROR binding molecules based on the rate limiting component of CH1 comprising polypeptide chains can provide a means to purify complete complexes from incomplete chains, e.g., purifying complexes having a limiting CH1 domain from complexes only having one or more non-CH1 comprising chains.

While the CH1 limiting expression may be a benefit in some aspects, as discussed, there is the potential for CH1 to limit overall expression of the complete ROR binding molecules. Thus, in certain embodiments, the expression of the polypeptide chain comprising the CH1 sequence(s) is adjusted to improve the efficiency of the ROR binding molecules forming complete complexes. In an illustrative example, the ratio of a plasmid vector constructed to express the polypeptide chain comprising the CH1 sequence(s) can be increased relative to the plasmid vectors constructed to express the other polypeptide chains.

In another illustrative example, the polypeptide chain comprising the CH1 sequence(s) when compared to the polypeptide chain comprising the CL sequence(s) can be the smaller of the two polypeptide chains. In another specific embodiment, the expression of the polypeptide chain comprising the CH1 sequence(s) can be adjusted by controlling which polypeptide chain has the CH1 sequence(s). For example, engineering the ROR binding molecule such that the CH1 domain is present in a two-domain polypeptide chain (e.g., the 4^{th} polypeptide chain described herein), instead of the CH1 sequence's native position in a four-domain polypeptide chain (e.g., the 3^{rd} polypeptide chain described herein), can be used to control the expression of the polypeptide chain comprising the CH1 sequence(s). However, in other aspects, a relative expression level of CH1 containing chains that is too high compared to the other chains can result in incomplete complexes the have the CH1 chain, but not each of the other chains. Thus, in certain embodiments, the expression of the polypeptide chain comprising the CH1 sequence(s) is adjusted to both reduce the formation incomplete complexes without the CH1 containing chain, and to reduce the formation incomplete complexes with the CH1 containing chain but without the other chains present in a complete complex.

### 6.3.8.2. CH1 and CL Orthogonal Modifications

In certain embodiments, the CH1 sequence and the CL sequences separately comprise respectively orthogonal modifications in endogenous CH1 and CL sequences.

Orthogonal mutations, in general, are described in more detail below in Sections 6.3.14.1-6.3.14.3.

In particular embodiments, the orthogonal modifications in endogenous CH1 and CL sequences are an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In a preferred embodiment, the engineered cysteines are at position 128 of the CH1 sequence and position 118 of the CL Kappa sequence, as numbered by the Eu index.

In a series of preferred embodiments, the mutations that provide non-endogenous cysteine amino acids are a F1 18C mutation in the CL sequence with a corresponding A141C in the CH1 sequence, or a F118C mutation in the CL sequence with a corresponding L128C in the CH1 sequence, or a S162C mutations in the CL sequence with a corresponding P171C mutation in the CH1 sequence, as numbered by the Eu index.

In a variety of embodiments, the orthogonal mutations in the CL sequence and the CH1 sequence are charge-pair mutations. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches. In a series of preferred embodiments the charge-pair mutations are a N138K mutation in the CL sequence with a corresponding G166D in the CH1 sequence, or a N138D mutation in the CL sequence with a corresponding G166K in the CH1 sequence, as numbered by the Eu index.

### 6.3.9. Domain J (CH2)

In the ROR binding molecules, domain J has a CH2 amino acid sequence. CH2 amino acid sequences are described in greater detail above in Section 6.3.3. In a preferred embodiment, the CH2 amino acid sequence has an N-terminal hinge region that connects domain J to domain I, as described in more detail below in Section 6.3.19.4.

In the ROR binding molecules, the C-terminus of domain J is connected to the N-terminus of domain K. In particular embodiments, domain J is connected to the N-terminus of domain K that has a CH1 amino acid sequence or CL amino acid sequence, as described in further detail below in Section 6.3.19.5.

### 6.3.10. Domain K (Constant Region)

In the ROR binding molecules, domain K has a constant region domain amino acid sequence. Constant region domain amino acid sequences are described in greater detail above in Section 6.3.2. In a preferred embodiment, domain K has a constant region sequence that is a CH3 sequence comprising knob-hole orthogonal mutations, as described in greater detail below in Section 6.3.14.2; isoallotype mutations, as described in more detail above in 6.3.2.1.; and either a S354C or a Y349C mutation that forms an engineered disulfide bridge with a CH3 domain containing an orthogonal mutation, as described in in greater detail below in Section 6.3.14.1. In some preferred embodiments, the knob-hole orthogonal mutations combined with isoallotype mutations are the following mutational changes: D356E, L358M, T366S, L368A, and Y407V.

In certain embodiments, the constant region domain sequence is a CH1 sequence. In particular embodiments, the CH1 amino acid sequence of domain K is the only CH1 amino acid sequence in the ROR binding molecule. In certain embodiments, the N-terminus of the CH1 domain is connected to the C-terminus of a CH2 domain, as described in greater detail below in 6.3.19.5. In certain embodiments, the constant region sequence is a CL sequence. In certain embodiments, the N-terminus of the CL domain is connected to the C-terminus of a CH2 domain, as described in greater detail below in 6.3.19.5. CH1 and CL sequences are described in further detail in Section 6.3.8.1.

### 6.3.11. Domain L (Variable Region)

In the ROR binding molecules, domain L has a variable region domain amino acid sequence. Variable region domain amino acid sequences, as discussed in greater detail in Section 6.3.1, are variable region domain amino acid sequences of an antibody including VL and VH antibody domain sequences. VL and VH sequences are described in greater detail above in Sections 6.3.1.1.and 6.3.1.4, respectively. In a preferred embodiment, domain L has a VH antibody domain sequence.

### 6.3.12. Domain M (Constant Region)

In the ROR binding molecules, domain M has a constant region domain amino acid sequence. Constant region domain amino acid sequences are described in greater detail above in Section 6.3.2. In a series of preferred embodiments of the ROR binding molecules, domain I has a CH1 amino acid sequence. In another series of preferred embodiments, domain I has a CL amino acid sequence. CH1 and CL amino acid sequences are described in further detail in Section 6.3.8.1.

### 6.3.13. Pairing of Domains A & F

In the ROR binding molecules, a domain A VL or VH amino acid sequence and a cognate domain F VL or VH amino acid sequence are associated and form an antigen binding site (ABS). The A:F antigen binding site (ABS) is capable of specifically binding an epitope of an antigen. Antigen binding by an ABS is described in greater detail below in Section 6.3.13.1.

In a variety of multivalent embodiments, the ABS formed by domains A and F (A:F) is identical in sequence to one or more other ABSs within the ROR binding molecule and therefore has the same recognition specificity as the one or more other sequence-identical ABSs within the ROR binding molecule.

In a variety of multivalent embodiments, the A:F ABS is non-identical in sequence to one or more other ABSs within the ROR binding molecule. In certain embodiments, the A:F ABS has a recognition specificity different from that of one or more other sequence-non-identical ABSs in the ROR binding molecule. In particular embodiments, the A:F ABS recognizes a different antigen from that recognized by at least one other sequence-non-identical ABS in the ROR binding molecule. In particular embodiments, the A:F ABS recognizes a different epitope of an antigen that is also recognized by at least one other sequence-non-identical ABS in the ROR binding molecule. In these embodiments, the ABS formed by domains A and F recognizes an epitope of antigen, wherein one or more other ABSs within the ROR binding molecule recognizes the same antigen but not the same epitope.

### 6.3.13.1. Binding of Antigen by ABS

An ABS, and the ROR binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g. Octet/FORTEBIO®), surface plasmon resonance (SPR) technology (e.g. Biacore®), and cell binding assays. For purposes herein, affinities are dissociation equilibrium constants measured by bio-layer interferometry using Octet/FORTEBIO®.

“Specific binding,” as used herein, refers to an affinity between an ABS and its cognate antigen or epitope in which the K_{D} value is below 10⁻⁶M, 10⁻⁷M, 10⁻⁸M, 10⁻⁹M, or 10⁻¹⁰M.

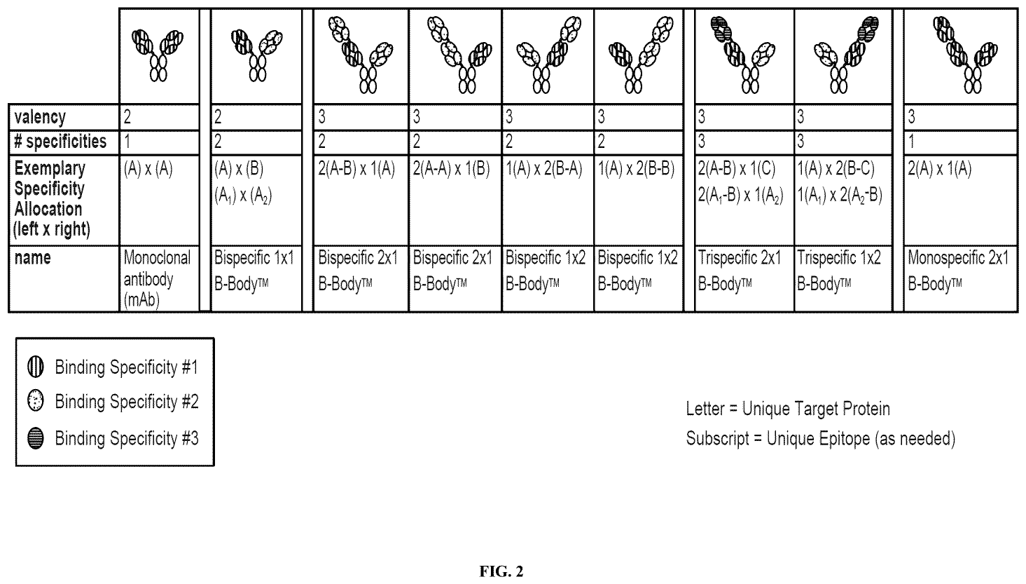

The number of ABSs in a ROR binding molecule as described herein defines the “valency” of the ROR binding molecule, as schematized in FIG. 2. A ROR binding molecule having a single ABS is “monovalent”. A ROR binding molecule having a plurality of ABSs is said to be “multivalent”. A multivalent ROR binding molecule having two ABSs is “bivalent.” A multivalent ROR binding molecule having three ABSs is “trivalent.” A multivalent ROR binding molecule having four ABSs is “tetravalent.”

In various multivalent embodiments, all of the plurality of ABSs have the same recognition specificity. As schematized in FIG. 2, such a ROR binding molecule is a “monospecific” “multivalent” binding construct. In other multivalent embodiments, at least two of the plurality of ABSs have different recognition specificities. Such ROR binding molecules are multivalent and “multispecific”. In multivalent embodiments in which the ABSs collectively have two recognition specificities, the ROR binding molecule is “bispecific.” In multivalent embodiments in which the ABSs collectively have three recognition specificities, the ROR binding molecule is “trispecific.”

In multivalent embodiments in which the ABSs collectively have a plurality of recognition specificities for different epitopes present on the same antigen, the ROR binding molecule is “multiparatopic.” Multivalent embodiments in which the ABSs collectively recognize two epitopes on the same antigen are “biparatopic.”

In various multivalent embodiments, multivalency of the ROR binding molecule improves the avidity of the ROR binding molecule for a specific target. As described herein, “avidity” refers to the overall strength of interaction between two or more molecules, e.g. a multivalent ROR binding molecule for a specific target, wherein the avidity is the cumulative strength of interaction provided by the affinities of multiple ABSs. Avidity can be measured by the same methods as those used to determine affinity, as described above. In certain embodiments, the avidity of a ROR binding molecule for a specific target is such that the interaction is a specific binding interaction, wherein the avidity between two molecules has a K_{D} value below 10⁻⁶M, 10⁻⁷M, 10⁻⁸M, 10⁻⁹M, or 10⁻⁰M. In certain embodiments, the avidity of a ROR binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

### 6.3.14. Pairing of Domains B & G

In the ROR binding molecules described herein, a domain B constant region amino acid sequence and a domain G constant region amino acid sequence are associated. Constant region domain amino acid sequences are described in greater detail above in Section 6.3.2.

In a series of preferred embodiments, domain B and domain G have CH3 amino acid sequences. CH3 sequences are described in greater detail above in Section 6.3.2.1. In various embodiments, the amino acid sequences of the B and the G domains are identical. In certain of these embodiments, the sequence is an endogenous CH3 sequence.

In a variety of embodiments, the amino acid sequences of the B and the G domains are different, and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

“Orthogonal modifications” or synonymously “orthogonal mutations” as described herein are one or more engineered mutations in an amino acid sequence of an antibody domain that increase the affinity of binding of a first domain having orthogonal modification for a second domain having a complementary orthogonal modification. In certain embodiments, the orthogonal modifications decrease the affinity of a domain having the orthogonal modifications for a domain lacking the complementary orthogonal modifications. In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail below in Sections 6.3.14.1-6.3.14.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail above in Section 6.3.2.1.

### 6.3.14.1. Orthogonal Engineered Disulfide Bridges

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

### 6.3.14.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681)), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and aY407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and aY407V mutation in a second domain.

### 6.3.14.3. Orthogonal Charge-pair Mutations

In a variety of embodiments, orthogonal modifications are charge-pair mutations. As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

### 6.3.15. Pairing of Domains E & K

In various embodiments, the E domain has a CH3 amino acid sequence.

In various embodiments, the K domain has a CH3 amino acid sequence.

In a variety of embodiments, the amino acid sequences of the E and K domains are identical, wherein the sequence is an endogenous CH3 sequence.

In a variety of embodiments, the sequences of the E and K domains are different. In a variety of embodiments, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In certain embodiments, the orthogonal modifications include, but are not limited to, engineered disulfide bridges, knob-in-hole mutations, and charge-pair mutations, as described in greater detail above in sections 6.3.14.1-6.3.14.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations.

### 6.3.16. Pairing of Domains I & M and Domains H & L

In a variety of embodiments, domain I has a CL sequence and domain M has a CH1 sequence. In a variety of embodiments, domain H has a VL sequence and domain L has a VH sequence. In a preferred embodiment, domain H has a VL amino acid sequence, domain I has a CL amino acid sequence, domain L has a VH amino acid sequence, and domain M has a CH1 amino acid sequence. In another preferred embodiment, domain H has a VL amino acid sequence, domain I has a CL amino acid sequence, domain L has a VH amino acid sequence, domain M has a CH1 amino acid sequence, and domain K has a CH3 amino acid sequence.

In a variety of embodiments, the amino acid sequences of the I domain and the M domain separately comprise respectively orthogonal modifications in an endogenous sequence, wherein the I domain interacts with the M domain, and wherein neither the I domain nor the M domain significantly interacts with a domain lacking the orthogonal modification. In a series of embodiments, the orthogonal mutations in the I domain are in a CL sequence and the orthogonal mutations in the M domain are in CH1 sequence. Orthogonal mutations are in CH1 and CL sequences are described in more detail above in Section 6.3.8.2.

In a variety of embodiments, the amino acid sequences of the H domain and the L domain separately comprise respectively orthogonal modifications in an endogenous sequence, wherein the H domain interacts with the L domain, and wherein neither the H domain nor the L domain significantly interacts with a domain lacking the orthogonal modification. In a series of embodiments, the orthogonal mutations in the H domain are in a VL sequence and the orthogonal mutations in the L domain are in VH sequence. In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

In certain embodiments, the interaction between the A domain and the F domain form a first antigen binding site specific for a first antigen, and the interaction between the H domain and the L domain form a second antigen binding site specific for a second antigen. In certain embodiments, the interaction between the A domain and the F domain form a first antigen binding site specific for a first antigen, and the interaction between the H domain and the L domain form a second antigen binding site specific for the first antigen.

6.3.17. Trivalent ROR binding molecules

In another series of embodiments, the ROR binding molecules have three antigen binding sites and are therefore termed “trivalent.”

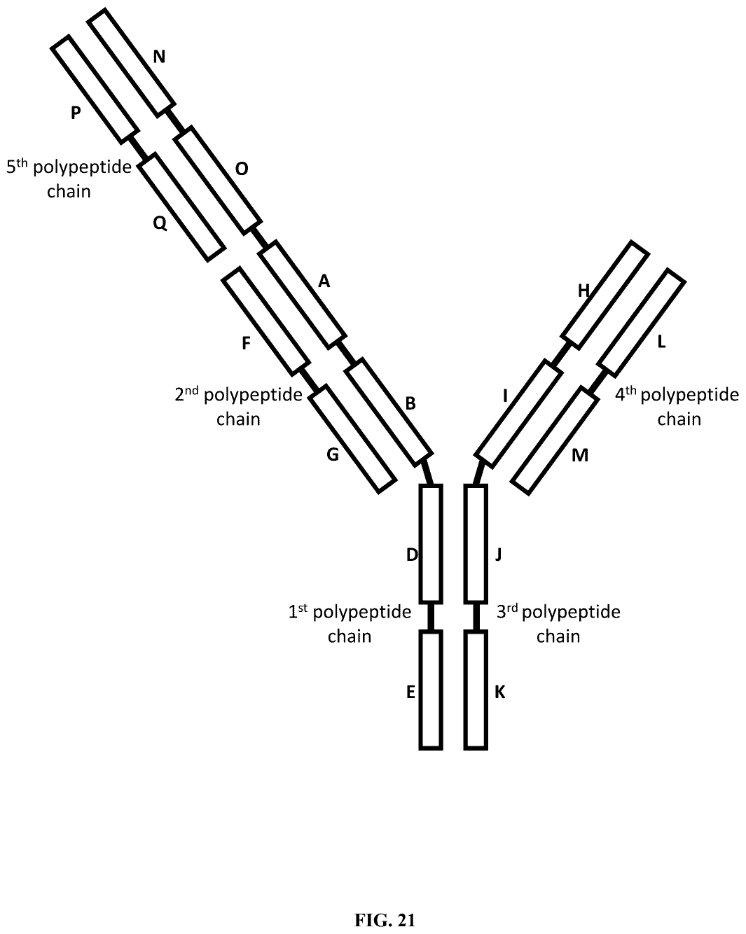

With reference to FIG. 21, in various trivalent embodiments the ROR binding molecules further comprise a fifth polypeptide chain, wherein (a) the first polypeptide chain further comprises a domain N and a domain O, wherein the domains are arranged, from N-terminus to C-terminus, in a N-O-A-B-D-E orientation, and wherein domain N has a VL amino acid sequence, domain O has a constant region amino acid sequence; (b) the ROR binding molecule further comprises a fifth polypeptide chain, comprising: a domain P and a domain Q, wherein the domains are arranged, from N-terminus to C-terminus, in a P-Q orientation, and wherein domain P has a VH amino acid sequence and domain Q has a constant amino acid sequence; and (c) the first and the fifth polypeptides are associated through an interaction between the N and the P domains and an interaction between the O and the Q domains to form the ROR binding molecule. As schematized in FIG. 2, these trivalent embodiments are termed “2×1” trivalent constructs.

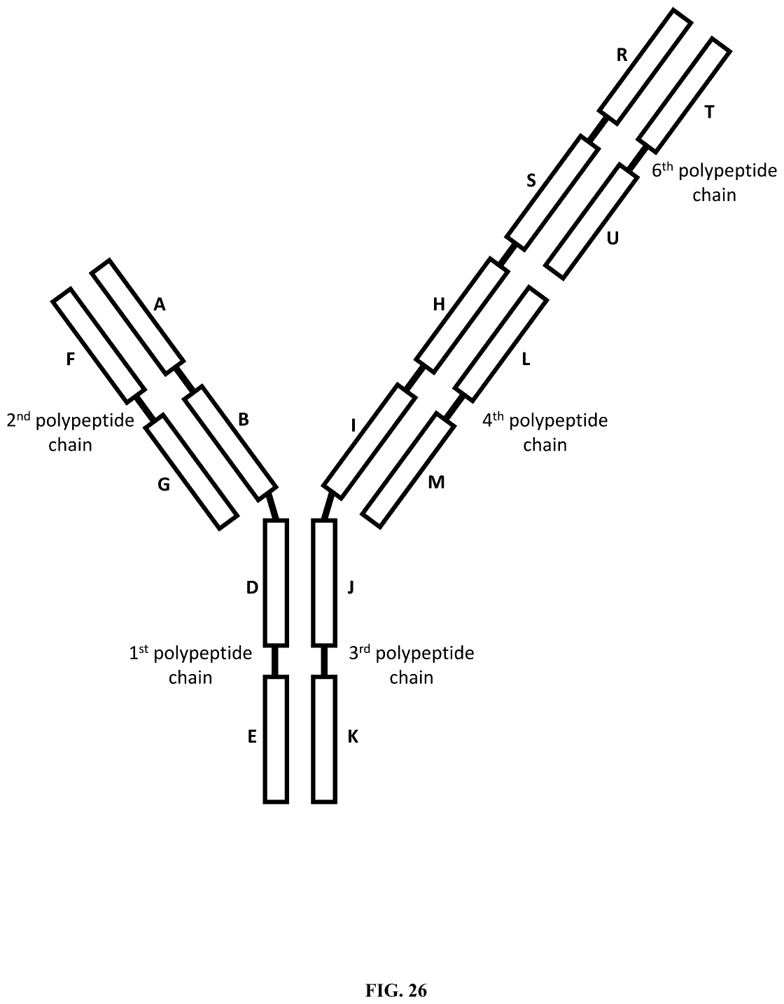

With reference to FIG. 26, in a further series of trivalent embodiments, the ROR binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a VL amino acid sequence and domain S has a constant domain amino acid sequence; (b) the ROR binding molecule further comprises a sixth polypeptide chain, comprising: a domain T and a domain U, wherein the domains are arranged, from N-terminus to C-terminus, in a T-U orientation, and wherein domain T has a VH amino acid sequence and domain U has a constant domain amino acid sequence; and (c) the third and the sixth polypeptides are associated through an interaction between the R and the T domains and an interaction between the S and the U domains to form the ROR binding molecule. As schematized in FIG. 2, these trivalent embodiments are termed “1×2” trivalent constructs.

In a variety of embodiments, the domain O is connected to domain A through a peptide linker. In a variety of embodiments, the domain S is connected to domain H through a peptide linker. In a preferred embodiment, the peptide linker connecting either domain O to domain A or connecting domain S to domain H is a 6 amino acid GSGSGS (SEQ ID NO:541) peptide sequence, as described in more detail in Section 6.3.19.6.

### 6.3.17.1. Trivalent 2×1 Bispecific Constructs [2(A-A)×1(B)]

With reference to FIG. 21, in a variety of embodiments the amino acid sequences of domain N and domain A are identical, the amino acid sequences of domain H is different from domains N and A, the amino acid sequences of domain 0 and domain B are identical, the amino acid sequences of domain I is different from domains O and B, the amino acid sequences of domain P and domain F are identical, the amino acid sequences of domain L is different from domains P and F, the amino acid sequences of domain Q and domain G are identical, the amino acid sequences of domain M is different from domains Q and G; and the interaction between the A domain and the F domain form a first antigen binding site specific for a first antigen, the interaction between the H domain and the L domain form a second antigen binding site specific for a second antigen, and the domain N and domain P form a third antigen binding site specific for the first antigen.

### 6.3.17.2. Trivalent 2×1 Bispecific Constructs [2(A-B)×1(A)]

With reference to FIG. 21, in a variety of embodiments the amino acid sequences of domain N and domain H are identical, the amino acid sequences of domain A is different from domains N and H, the amino acid sequences of domain 0 and domain I are identical, the amino acid sequences of domain B is different from domains O and I, the amino acid sequences of domain P and domain L are identical, the amino acid sequences of domain F is different from domains P and L, the amino acid sequences of domain Q and domain M are identical, the amino acid sequences of domain G is different from domains Q and M; and the interaction between the A domain and the F domain form a first antigen binding site specific for a first antigen, the interaction between the H domain and the L domain form a second antigen binding site specific for a second antigen, and the domain N and domain P form a third antigen binding site specific for the second antigen.

### 6.3.17.3. Trivalent 2×1 Trispecific Constructs [2(A-B)×1(C)]

With reference to FIG. 21, in a variety of embodiments, the amino acid sequences of domain N, domain A, and domain H are different, the amino acid sequences of domain 0, domain B, and domain I are different, the amino acid sequences of domain P, domain F, and domain L are different, and the amino acid sequences of domain Q, domain G, and domain M are different; and the interaction between the A domain and the F domain form a first antigen binding site specific for a first antigen, the interaction between the H domain and the L domain form a second antigen binding site specific for a second antigen, and the domain N and domain P form a third antigen binding site specific for a third antigen.

In certain embodiments, domain O has a constant region sequence that is a CL from a kappa light chain and domain Q has a constant region sequence that is a CH1 from an IgG1 isotype, as discussed in more detail in Section 6.3.8.1. In a preferred embodiment, domain 0 and domain Q have CH3 sequences such that they specifically associate with each other, as discussed in more detail above in Section 6.3.14.

### 6.3.17.4. Trivalent 1×2 Bispecific Constructs [1(A)×2(B-A)]

With reference to FIG. 26, in a variety of embodiments, the amino acid sequences of domain R and domain A are identical, the amino acid sequences of domain H is different from domain R and A, the amino acid sequences of domain S and domain B are identical, the amino acid sequences of domain I is different from domain S and B, the amino acid sequences of domain T and domain F are identical, the amino acid sequences of domain L is different from domain T and F, the amino acid sequences of domain U and domain G are identical, the amino acid sequences of domain M is different from domain U and G and the interaction between the A domain and the F domain form a first antigen binding site specific for a first antigen, the interaction between the H domain and the L domain form a second antigen binding site specific for a second antigen, and the domain R and domain T form a third antigen binding site specific for the first antigen.

### 6.3.17.5. Trivalent 1×2 Bispecific Constructs [1(A)×2(B-B)]

In a variety of embodiments, the ROR binding molecule further comprises a second CH1 domain, or portion thereof. With reference to FIG. 26, in specific embodiments, the amino acid sequences of domain R and domain H are identical, the amino acid sequences of domain A is different from domain R and H, the amino acid sequences of domain S and domain I are identical, the amino acid sequences of domain B is different from domain S and I, the amino acid sequences of domain T and domain L are identical, the amino acid sequences of domain F is different from domain T and L, the amino acid sequences of domain U and domain M are identical, the amino acid sequences of domain G is different from domain U and M and the interaction between the A domain and the F domain form a first antigen binding site specific for a first antigen, the interaction between the H domain and the L domain form a second antigen binding site specific for a second antigen, and the domain R and domain T form a third antigen binding site specific for the second antigen.

In particular embodiments, the amino acid sequences of domain S and domain I are CH1 sequences. In particular embodiments, the amino acid sequences of domain U and domain M are CH1 sequences.

### 6.3.17.6. Trivalent 1×2 Trispecific Constructs [1(A)×2(B-C)]

With reference to FIG. 26, in a variety of embodiments, the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and the interaction between the A domain and the F domain form a first antigen binding site specific for a first antigen, the interaction between the H domain and the L domain form a second antigen binding site specific for a second antigen, and the domain R and domain T form a third antigen binding site specific for a third antigen.

In particular embodiments, domain S has a constant region sequence that is a CL from a kappa light chain and domain U has a constant region sequence that is a CH1 from an IgG1 isotype, as discussed in more detail in Section 6.3.8.1. In a preferred embodiment, domain S and domain U have CH3 sequences such that they specifically associate with each other, as discussed in more detail above in Section 6.3.14.

In certain embodiments, the ROR binding molecule further comprises a second CH1 domain, or portion thereof. In particular embodiments, the amino acid sequences of domain S and domain I are CH1 sequences. In particular embodiments, the amino acid sequences of domain U and domain M are CH1 sequences.

6.3.18. Tetravalent 2×2 ROR binding molecules

In a variety of embodiments, the ROR binding molecules have 4 antigen binding sites and are therefore termed “tetravalent.”

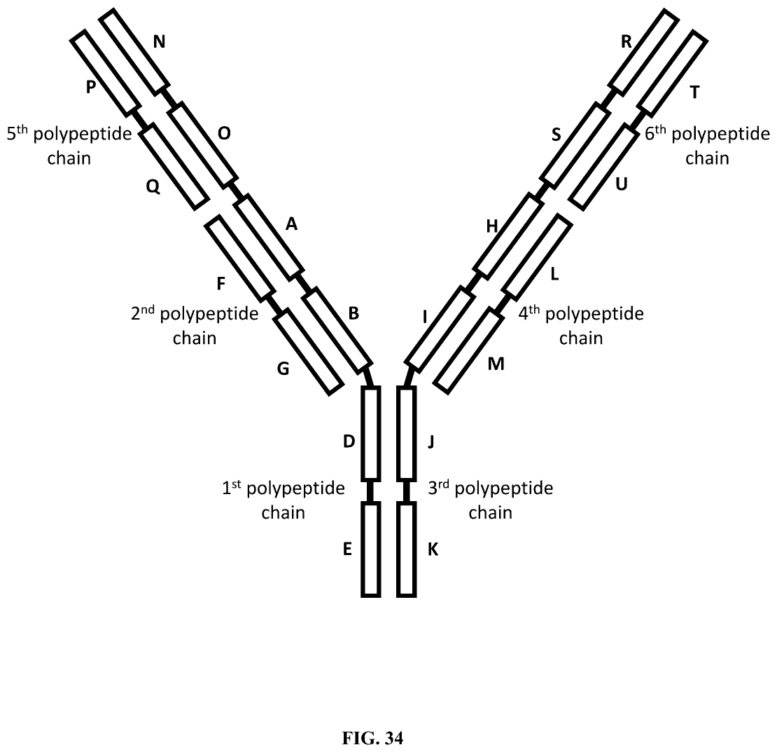

With reference to FIG. 34, in a further series of embodiments, the ROR binding molecules further comprise a fifth and a sixth polypeptide chain, wherein (a) the first polypeptide chain further comprises a domain N and a domain O, wherein the domains are arranged, from N-terminus to C-terminus, in a N-O-A-B-D-E orientation; (b) the third polypeptide chain further comprises a domain R and a domain S, wherein the domains are arranged, from N-terminus to C-terminus, in a R-S-H-I-J-K orientation; (c) the ROR binding molecule further comprises a fifth and a sixth polypeptide chain, wherein the fifth polypeptide chain comprises a domain P and a domain Q, wherein the domains are arranged, from N-terminus to C-terminus, in a P-Q orientation, and the sixth polypeptide chain comprises a domain T and a domain U, wherein the domains are arranged, from N-terminus to C-terminus, in a T-U orientation; and (d) the first and the fifth polypeptides are associated through an interaction between the N and the P domains and an interaction between the O and the Q domains, and the third and the sixth polypeptides are associated through an interaction between the Rand the T domains and an interaction between the S and the U domains to form the ROR binding molecule.

In a variety of embodiments, the domain O is connected to domain A through a peptide linker and the domain S is connected to domain H through a peptide linker. In a preferred embodiment, the peptide linker connecting domain O to domain A and connecting domain S to domain H is a 6 amino acid GSGSGS (SEQ ID NO:541) peptide sequence, as described in more detail in Section 6.3.19.6.

### 6.3.18.1. Tetravalent 2×2 Bispecific Constructs

With reference to FIG. 34, in a series of tetravalent 2×2 bispecific ROR binding molecules, the amino acid sequences of domain N and domain A are identical, the amino acid sequences of domain H and domain R are identical, the amino acid sequences of domain 0 and domain B are identical, the amino acid sequences of domain I and domain S are identical, the amino acid sequences of domain P and domain F are identical, the amino acid sequences of domain L and domain T are identical, the amino acid sequences of domain Q and domain G are identical, the amino acid sequences of domain M and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for a second antigen, and the interaction between the R domain and the T domain form a fourth antigen binding site specific for the second antigen.

With reference to FIG. 34, in another series of tetravalent 2×2 bispecific ROR binding molecules, the amino acid sequences of domain H and domain A are identical, the amino acid sequences of domain N and domain R are identical, the amino acid sequences of domain I and domain B are identical, the amino acid sequences of domain 0 and domain S are identical, the amino acid sequences of domain L and domain F are identical, the amino acid sequences of domain P and domain T are identical, the amino acid sequences of domain M and domain G are identical, the amino acid sequences of domain Q and domain U are identical; and wherein the interaction between the A domain and the F domain form a first antigen binding site specific for a first antigen, the domain N and domain P form a second antigen binding site specific for a second antigen, the interaction between the H domain and the L domain form a third antigen binding site specific for the first antigen, and the interaction between the R domain and the T domain form a fourth antigen binding site specific for the second antigen.

### 6.3.19. Domain Junctions

### 6.3.19.1. Junctions Connecting VL and CH3 Domains

In a variety of embodiments, the amino acid sequence that forms a junction between the C-terminus of a VL domain and the N-terminus of a CH3 domain is an engineered sequence. In certain embodiments, one or more amino acids are deleted or added in the C-terminus of the VL domain. In certain embodiments, the junction connecting the C-terminus of a VL domain and the N-terminus of a CH3 domain is one of the sequences described in Table 2 below in Section 6.13.7. In particular embodiments, A111 is deleted in the C-terminus of the VL domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In certain embodiments, one or more amino acids are deleted or added to both the C-terminus of the VL domain and the N-terminus of the CH3 domain. In particular embodiments, A111 is deleted in the C-terminus of the VL domain and P343 is deleted in the N-terminus of the CH3 domain. In a preferred embodiment, A111 and V110 are deleted in the C-terminus of the VL domain. In another preferred embodiment, A111 and V110 are deleted in the C-terminus of the VL domain and the N-terminus of the CH3 domain has a P343V mutation.

### 6.3.19.2. Junctions Connecting VH and CH3 Domains

In a variety of embodiments, the amino acid sequence that forms a junction between the C-terminus of a VH domain and the N-terminus of a CH3 domain is an engineered sequence. In certain embodiments, one or more amino acids are deleted or added in the C-terminus of the VH domain. In certain embodiments, the junction connecting the C-terminus of a VH domain and the N-terminus of the CH3 domain is one of the sequences described in Table 3 below in Section 6.13.7. In particular embodiments, K117 and G118 are deleted in the C-terminus of the VH domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In particular embodiments, P343, R344, and E345 are deleted in the N-terminus of the CH3 domain. In certain embodiments, one or more amino acids are deleted or added to both the C-terminus of the VH domain and the N-terminus of the CH3 domain. In a preferred embodiment, T116, K117, and G118 are deleted in the C-terminus of the VH domain.

### 6.3.19.3. Junctions Connecting CH3 C-Terminus to CH2 N-Terminus (Hinge)

In the ROR binding molecules described herein, the N-terminus of the CH2 domain has a “hinge” region amino acid sequence. As used herein, hinge regions are sequences of an antibody heavy chain that link the N-terminal variable domain-constant domain segment of an antibody and a CH2 domain of an antibody. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains). As used herein, the hinge region amino acid sequence is SEQ ID NO: 56.

In a variety of embodiments, a CH3 amino acid sequence is extended at the C-terminus at the junction between the C-terminus of the CH3 domain and the N-terminus of a CH2 domain. In certain embodiments, a CH3 amino acid sequence is extended at the C-terminus at the junction between the C-terminus of the CH3 domain and a hinge region, which in turn is connected to the N-terminus of a CH2 domain. In a preferred embodiment, the CH3 amino acid sequence is extended by inserting a PGK tripeptide sequence followed by the DKTHT (SEQ ID NO:542) motif of an IgG1 hinge region.

In a particular embodiment, the extension at the C-terminus of the CH3 domain incorporates amino acid sequences that can form a disulfide bond with orthogonal C-terminal extension of another CH3 domain. In a preferred embodiment, the extension at the C-terminus of the CH3 domain incorporates a KSC tripeptide sequence that is followed by the DKTHT (SEQ ID NO:542) motif of an IgG1 hinge region that forms a disulfide bond with orthogonal C-terminal extension of another CH3 domain that incorporates a GEC motif of a kappa light chain.

### 6.3.19.4. Junctions Connecting CL C-Terminus and CH2 N-Terminus (Hinge)

In a variety of embodiments, a CL amino acid sequence is connected through its C-terminus to a hinge region, which in turn is connected to the N-terminus of a CH2 domain.

Hinge region sequences are described in more detail above in Section 6.3.19.3. In a preferred embodiment, the hinge region amino acid sequence is SEQ ID NO:56.

### 6.3.19.5. Junctions Connecting CH2 C-terminus to Constant Region Domain

In a variety of embodiments, a CH2 amino acid sequence is connected through its C-terminus to the N-terminus of a constant region domain. Constant regions are described in more detail above in Section 6.3.4. In a preferred embodiment, the CH2 sequence is connected to a CH3 sequence via its endogenous sequence. In other embodiments, the CH2 sequence is connected to a CH1 or CL sequence. Examples discussing connecting a CH2 sequence to a CH1 or CL sequence are described in more detail in U.S. Pat. No. 8,242,247, which is hereby incorporated in its entirety.

### 6.3.19.6. Junctions Connecting Domain O to Domain A or Domain S to Domain H on Trivalent and Tetravalent Molecules

In a variety of embodiments, heavy chains of antibodies (e.g. the first and third polypeptide chains) are extended at their N-terminus to include additional domains that provide additional ABSs. With reference to FIG. 21, FIG. 26, and FIG. 34, in certain embodiments, the C-terminus of the constant region domain amino acid sequence of a domain 0 and/or a domain S is connected to the N-terminus of the variable region domain amino acid sequence of a domain A and/or a domain H, respectively. In some preferred embodiments, the constant region domain is a CH3 amino acid sequence and the variable region domain is a VL amino acid sequence. In some preferred embodiments, the constant region domain is a CL amino acid sequence and the variable region domain is a VL amino acid sequence. In certain embodiments, the constant region domain is connected to the variable region domain through a peptide linker. In a preferred embodiment, the peptide linker is a 6 amino acid GSGSGS (SEQ ID NO:541) peptide sequence.

In a variety of embodiments, light chains of antibodies (e.g. the second and fourth polypeptide chains) are extended at their N-terminus to include additional variable domain-constant domain segments of an antibody. In certain embodiments, the constant region domain is a CH1 amino acid sequence and the variable region domain is a VH amino acid sequence.

6.4. Specific Bivalent ROR binding molecules

In a further aspect, bivalent ROR binding molecules are provided.

With reference to FIG. 3, in a series of embodiments the ROR binding molecules comprise a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the ROR binding molecule.

In a preferred embodiment, domain E has a CH3 amino acid sequence, domain H has a VL amino acid sequence, domain I has a CL amino acid sequence, domain K has a CH3 amino acid sequence, domain L has a VH amino acid sequence, and domain M has a CH1 amino acid sequence.

In certain embodiments, the interaction between the A domain and the F domain form a first antigen binding site specific for a first antigen, and the interaction between the H domain and the L domain form a second antigen binding site specific for a second antigen, and the ROR binding molecule is a bispecific bivalent ROR binding molecule. In certain embodiments, the interaction between the A domain and the F domain form a first antigen binding site specific for a first antigen, and the interaction between the H domain and the L domain form a second antigen binding site specific for the first antigen, and the ROR binding molecule is a monospecific bivalent ROR binding molecule.

### 6.4.1. Bivalent Bispecific B-Body “BC1”

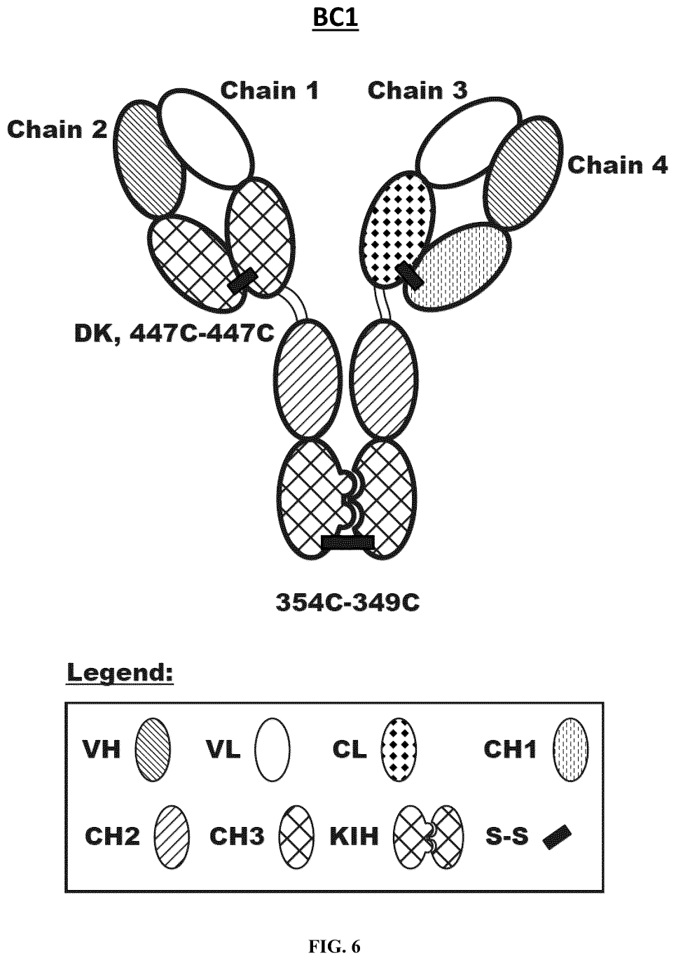

With reference to FIG. 3 and FIG. 6, in a series of embodiments, the ROR binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a T366K mutation and a C-terminal extension incorporating a KSC tripeptide sequence that is followed by the DKTHT (SEQ ID NO:542) motif of an IgG1 hinge region, domain D has a human IgG1 CH2 amino acid sequence, and domain E has human IgG1 CH3 amino acid with a S354C and T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a L351D mutation and a C-terminal extension incorporating a GEC amino acid disulfide motif, (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the ROR binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:8, the second polypeptide chain has the sequence SEQ ID NO:9, the third polypeptide chain has the sequence SEQ ID NO:10, and the fourth polypeptide chain has the sequence SEQ ID NO:11.

### 6.4.2. Bivalent Bispecific B-Body “BC6”

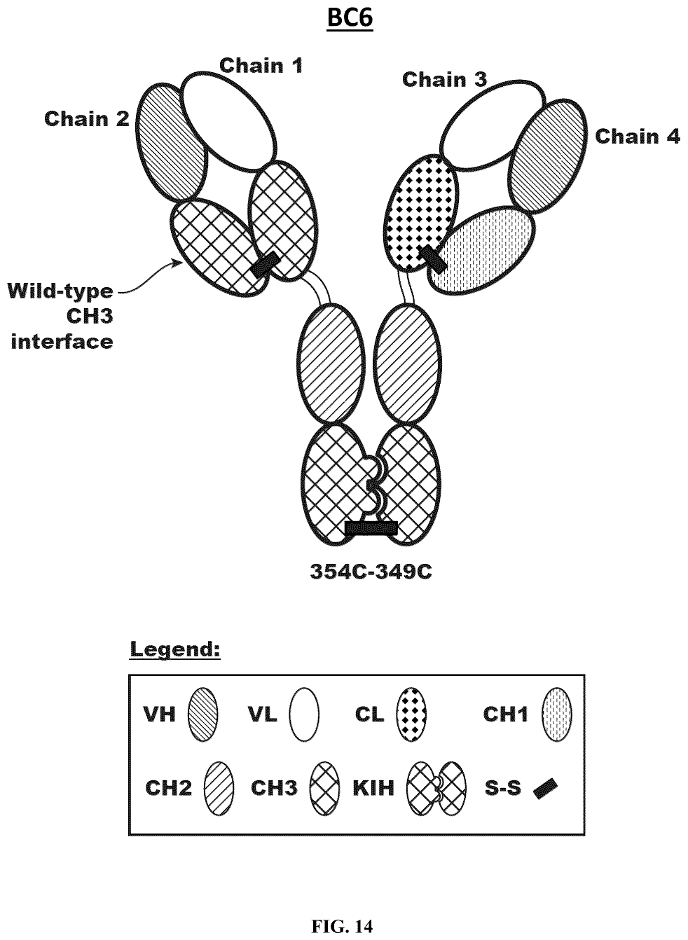

With reference to FIG. 3 and FIG. 14, in a series of embodiments, the ROR binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a C-terminal extension incorporating a KSC tripeptide sequence that is followed by the DKTHT (SEQ ID NO:542) motif of an IgG1 hinge region, domain D has a human IgG1 CH2 amino acid sequence, and domain E has human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a C-terminal extension incorporating a GEC amino acid disulfide motif, (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the ROR binding molecule; (h) domain A and domain F form a first antigen binding site specific for a first antigen; and (i) domain H and domain L form a second antigen binding site specific for a second antigen.

### 6.4.3. Bivalent Bispecific B-Body “BC28”

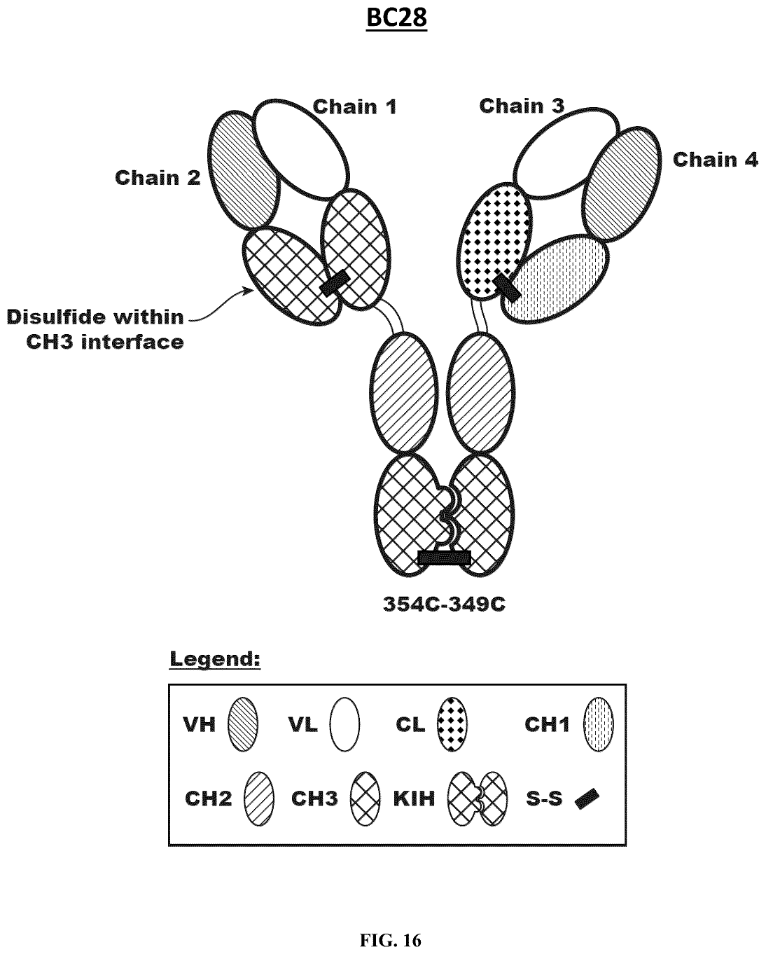

With reference to FIG. 3 and FIG. 16, in a series of embodiments, the ROR binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation and a C-terminal extension incorporating a PGK tripeptide sequence that is followed by the DKTHT (SEQ ID NO:542) motif of an IgG1 hinge region, domain D has a human IgG1 CH2 amino acid sequence, and domain E has a human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the ROR binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:24, the second polypeptide chain has the sequence SEQ ID NO:25, the third polypeptide chain has the sequence SEQ ID NO:10, and the fourth polypeptide chain has the sequence SEQ ID NO:11.

### 6.4.4. Bivalent Bispecific B-Body “BC44”

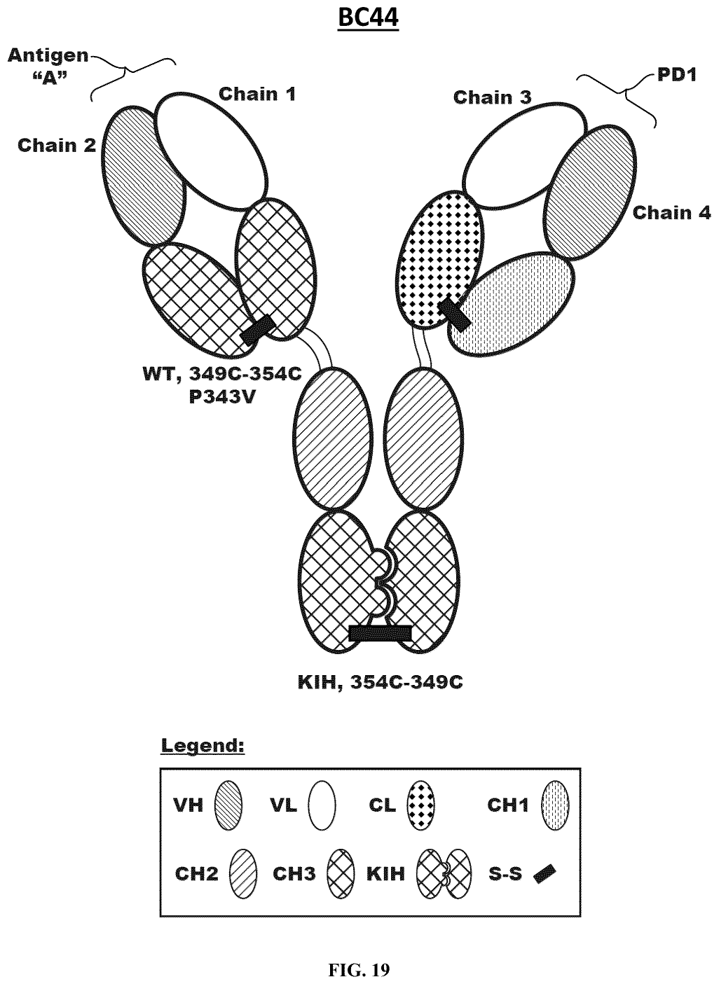

With reference to FIG. 3 and FIG. 19, in a series of embodiments, the ROR binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation, a P343V mutation, and a C-terminal extension incorporating a PGK tripeptide sequence that is followed by the DKTHT (SEQ ID NO:542) motif of an IgG1 hinge region, domain D has a human IgG1 CH2 amino acid sequence, and domain E has human IgG1 CH3 amino acid with a S354C mutation and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, T366S, L368A, and aY407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the ROR binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:32, the second polypeptide chain has the sequence SEQ ID NO:25, the third polypeptide chain has the sequence SEQ ID NO:10, and the fourth polypeptide chain has the sequence SEQ ID NO:11.

6.5. Specific Trivalent ROR binding molecules

### 6.5.1. Trivalent 1×2 Bispecific B-Body “BC28-1×2”

With reference to Section 6.4.3. and FIG. 26, in a series of embodiments, the ROR binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has the first VL amino acid sequence and domain S has a human IgG1 CH3 amino acid sequence with a Y349C mutation and a C-terminal extension incorporating a PGK tripeptide sequence that is followed by GSGSGS (SEQ ID NO:541) linker peptide connecting domain S to domain H; (b) the ROR binding molecule further comprises a sixth polypeptide chain, comprising: a domain T and a domain U, wherein the domains are arranged, from N-terminus to C-terminus, in a T-U orientation, and wherein domain T has the first VH amino acid sequence and domain U has a human IgG1 CH3 amino acid sequence with a S354C mutation and a C-terminal extension incorporating a PGK tripeptide sequence; (c) the third and the sixth polypeptides are associated through an interaction between the R and the T domains and an interaction between the S and the U domains to form the ROR binding molecule, and (d) domain R and domain T form a third antigen binding site specific for the first antigen.

In preferred embodiments, the first polypeptide chain has the sequence SEQ ID NO:24, the second polypeptide chain has the sequence SEQ ID NO:25, the third polypeptide chain has the sequence SEQ ID NO:37, the fourth polypeptide chain has the sequence SEQ ID NO:11, and the sixth polypeptide chain has the sequence SEQ ID NO:25.

### 6.5.2. Trivalent 1×2 Trispecific B-Body “BC28-1×1x1a”

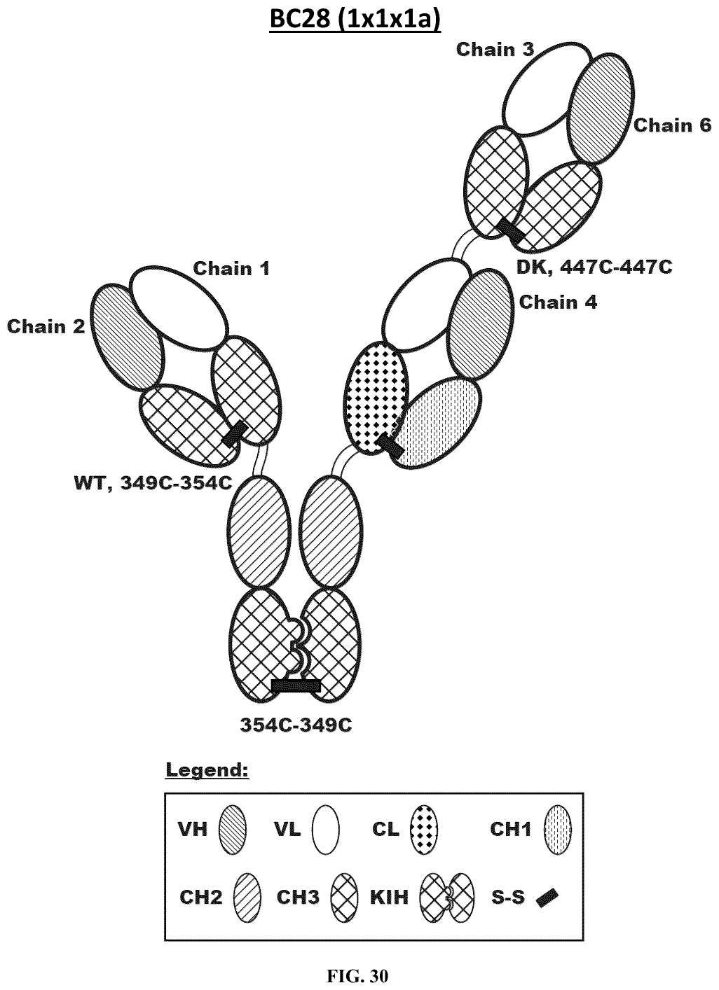

With reference to Section 6.4.3. and FIG. 26 and FIG. 30, in a series of embodiments, the ROR binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a third VL amino acid sequence and domain S has a human IgG1 CH3 amino acid sequence with a T366K mutation and a C-terminal extension incorporating a KSC tripeptide sequence that is followed by GSGSGS (SEQ ID NO:541) linker peptide connecting domain S to domain H; (b) the ROR binding molecule further comprises a sixth polypeptide chain, comprising: a domain T and a domain U, wherein the domains are arranged, from N-terminus to C-terminus, in a T-U orientation, and wherein domain T has a third VH amino acid sequence and domain U has a human IgG1 CH3 amino acid sequence with a L351D mutation and a C-terminal extension incorporating a GEC amino acid disulfide motif, and (c) the third and the sixth polypeptides are associated through an interaction between the R and the T domains and an interaction between the S and the U domains to form the ROR binding molecule, and (d) domain R and domain T form a third antigen binding site specific for a third antigen.

In preferred embodiments, the first polypeptide chain has the sequence SEQ ID NO:24, the second polypeptide chain has the sequence SEQ ID NO:25, the third polypeptide chain has the sequence SEQ ID NO:45, the fourth polypeptide chain has the sequence SEQ ID NO:11, and the sixth polypeptide chain has the sequence SEQ ID NO: 53.

6.6. Other ROR binding molecule platforms

The various antibody platforms described above are not limiting. The antigen binding sites described herein, including specific CDR subsets, can be formatted into any binding molecule platform including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

6.7. Antigen specificities

Other antigens to which a ROR binding molecule as described herein can specifically bind, in addition to a ROR antigen, may be chosen from a wide variety of molecular targets. For example, an antigen binding site or sites may specifically bind E-Cad, CLDN7, FGFR2b, N-Cad, Cad-11, FGFR2c, ERBB2, ERBB3, FGFR1, FOLR1, IGF-Ira, GLP1R, PDGFRa, PDGFRb, EPHB6, ABCG2, CXCR4, CXCR7, Integrin-avb3, SPARC, VCAM, ICAM, Annexin, ROR1, ROR2, TNFα, CD137, angiopoietin 2, angiopoietin 3, BAFF, beta amyloid, C5, CA-125, CD147, CD125, CD147, CD152, CD19, CD20, CD22, CD23, CD24, CD25, CD274, CD28, CD3, CD30, CD33, CD37, CD4, CD40, CD44, CD44v4, CD44v6, CD44v7, CD50, CD51, CD52, CEA, CSF1R, CTLA-2, DLL4, EGFR, EPCAM, HER3, GD2 ganglioside, GDF-8, Her2/neu, CD2221, IL-17A, IL-12, IL-23, IL-13, IL-6, IL-23, an integrin, CD11a, MUC1, Notch, TAG-72, TGFβ, TRAIL-R2, VEGF-A, VEGFR-1, VEGFR2, VEGFc, hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-P2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, Fas, CD27, CD30, and 4-1BBL); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)); in embodiments relating to bispecific antibodies, the antibody may for example bind two of these targets. Furthermore, the Fc portion of the heavy chain of an antibody may be used to target Fc receptor-expressing cells such as the use of the Fc portion of an IgE antibody to target mast cells and basophils.

Other antigens to which a ROR binding molecule as described herein can specifically bind, in addition to a ROR antigen, may be chosen that specifically binds the TNF family of receptors including, but not limited to, TNFR1 (also known as CD120a and TNFRSFlA), TNFR2 (also known as CD120b and TNFRSFIB), TNFRSF3 (also known as LTOR), TNFRSF4 (also known as OX40 and CD134), TNFRSF5 (also known as CD40), TNFRSF6 (also known as FAS and CD95), TNFRSF6B (also known as DCR3), TNFRSF7 (also known as CD27), TNFRSF8 (also known as CD30), TNFRSF9 (also known as 4-1iB), TNFRSF1OA (also known as TRAILR1, DR4, and CD26), TNFRSF1OB (also known as TRAILR2, DR5, and CD262), TNFRSF1OC (also known as TRAILR3, DCR1, CD263), TNFRSF1OD (also known as TRAILR4, DCR2, and CD264), TNFRSF11 A (also known as RANK and CD265), TNFRSF1 IB (also known as OPG), TNFRSF12A (also known as FN14, TWEAKR, and CD266), TNFRSF13B (also known as TACI and CD267), TNFRSF13C (also known as BAFFR, BR3, and CD268), TNFRSF14 (also known as HVEM and CD270), TNFRSF16 (also known as NGFR, p75NTR, and CD271), or TNFRSF17 (also known as BCMA and CD269), TNFRSF18 (also known as GITR and CD357), TNFRSF19 (also known as TROY, TAJ, and TRADE), TNFRSF21 (also known as CD358), TNFRSF25 (also known as Apo-3, TRAMP, LARD, or WS-1), EDA2R (also known as XEDAR).

Other antigens to which a ROR binding molecule as described herein can specifically bind, in addition to a ROR antigen, may be chosen from immuno-oncology targets including, but not limited to, checkpoint inhibitor targets such as PD1, PDL1, CTLA-4, PDL2, n7-H3, n7-H4, BTLA, TIM3, GAL9, LAG3, VISTA, KIR, 2n4, BY55, and CGEN-15049.

### 6.8. Further Modifications

In a further series of embodiments, the ROR binding molecule has additional modifications.

### 6.8.1. Antibody-Drug Conjugates

In various embodiments, the ROR binding molecule is conjugated to a therapeutic agent (e.g., drug) to form a ROR binding molecule-drug conjugate. Therapeutic agents include, but are not limited to, chemotherapeutic agents, imaging agents (e.g., radioisotopes), immune modulators (e.g., cytokines, chemokines, or checkpoint inhibitors), and toxins (e.g., cytotoxic agents). In certain embodiments, the therapeutic agents are attached to the ROR binding molecule through a linker peptide, as discussed in more detail below in Section 6.8.3.

Methods of preparing antibody-drug conjugates (ADCs) that can be adapted to conjugate drugs to the ROR binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 Nov-Dec; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc NatlAcad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (JAm Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### 6.8.2. Additional Binding Moieties

In various embodiments, the ROR binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the ROR binding molecule. In certain embodiments, the one or more additional binding moieties are specific for the same antigen or epitope of the ABSs within the ROR binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the ROR binding molecule using in vitro methods including, but not limited to, reactive chemistry and affinity tagging systems, as discussed in more detail below in Section 6.8.3. In certain embodiments, the one or more additional binding moieties are attached to the ROR binding molecule through Fc-mediated binding (e.g. Protein A/G). In certain embodiments, the one or more additional binding moieties are attached to the ROR binding molecule using recombinant DNA techniques, such as encoding the nucleotide sequence of the fusion product between the ROR binding molecule and the additional binding moieties on the same expression vector (e.g. plasmid).

### 6.8.3. Functional/Reactive Groups

In various embodiments, the ROR binding molecule has modifications that comprise functional groups or chemically reactive groups that can be used in downstream processes, such as linking to additional moieties (e.g. drug conjugates and additional binding moieties, as discussed in more detail above in Sections 6.8.1. and 6.8.2.) and downstream purification processes.

In certain embodiments, the modifications are chemically reactive groups including, but not limited to, reactive thiols (e.g. maleimide based reactive groups), reactive amines (e.g. N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g. HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178.), the entirety of which is hereby incorporated by reference for all it teaches.

### 6.8.4. Reduced Effector Function

In certain embodiments, the ROR binding molecule has one or more engineered mutations in an amino acid sequence of an antibody domain that reduce the effector functions naturally associated with antibody binding. Effector functions include, but are not limited to, cellular functions that result from an Fc receptor binding to an Fc portion of an antibody, such as antibody-dependent cellular cytotoxicity (ADCC, also referred to as antibody-dependent cell-mediated cytotoxicity), complement fixation (e.g. C1q binding), antibody dependent cellular-mediated phagocytosis (ADCP), and opsonization. Engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each herein incorporated by reference in its entirety.

In specific embodiments, the ROR binding molecule has one or more engineered mutations in an amino acid sequence of an antibody domain that reduce binding of an Fc portion of the ROR binding molecule by FcR receptors. In some embodiments, the FcR receptors are FcRγ receptors. In particular embodiments, the FcR receptors are FcγRIIa and/or FcγRIIIA receptors.

In specific embodiments, the one or more engineered mutations that reduce effector function are mutations in a CH2 domain of an antibody. In various embodiments, the one or more engineered mutations are at position L234 and L235 of the CH2 domain. In particular embodiments, the one or more engineered mutations are L234A and L235A of the CH2 domain. In other embodiments, the one or more engineered mutations are at position L234, L235, and P329 of the CH2 domain. In particular embodiments, the one or more engineered mutations are L234A, L235A, and P329G of the CH2 domain. In preferred embodiments, the one or more engineered mutations are L234A, L235A, and P329K of the CH2 domain.

### 6.9. Methods of Purification

A method of purifying a ROR binding molecule comprising a B-body platform is provided herein.

In a series of embodiments, the method comprises the steps of: i) contacting a sample comprising the ROR binding molecule with a CH1 binding reagent, wherein the ROR binding molecule comprises at least a first, a second, a third, and a fourth polypeptide chain associated in a complex, wherein the complex comprises at least one CH1 domain, or portion thereof, and wherein the number of CH1 domains in the complex is at least one fewer than the valency of the complex, and wherein the contacting is performed under conditions sufficient for the CH1 binding reagent to bind the CH1 domain, or portion thereof, and ii) purifying the complex from one or more incomplete complexes, wherein the incomplete complexes do not comprise the first, the second, the third, and the fourth polypeptide chain.

In a typical, naturally occurring, antibody, two heavy chains are associated, each of which has a CH1 domain as the second domain, numbering from N-terminus to C-terminus.

Thus, a typical antibody has two CH1 domains. CH1 domains are described in more detail in Section 6.3.8.1. In a variety of the ROR binding molecules described herein, the CH1 domain typically found in the protein has been substituted with another domain, such that the number of CH1 domains in the protein is effectively reduced. In a non-limiting illustrative example, the CH1 domain of a typical antibody can be substituted with a CH3 domain, generating an antigen-binding protein having only a single CH1 domain.

ROR binding molecules can also refer to molecules based on antibody architectures that have been engineered such that they no longer possess a typical antibody architecture. For example, an antibody can be extended at its N or C terminus to increase the valency (described in more detail in Section 6.3.13.1) of the antigen-binding protein, and in certain instances the number of CH1 domains is also increased beyond the typical two CH1 domains. Such molecules can also have one or more of their CH1 domains substituted, such that the number of CH1 domains in the protein is at least one fewer than the valency of the antigen-binding protein. In some embodiments, the number of CH1 domains that are substituted by other domains generates a ROR binding molecule having only a single CH1 domain. In other embodiments, the number of CH1 domains substituted by another domain generates a ROR binding molecule having two or more CH1 domains, but at least one fewer than the valency of the antigen-binding protein. In particular embodiments, where a ROR binding molecule has two or more CH1 domains, the multiple CH1 domains can all be in the same polypeptide chain. In other particular embodiments, where a ROR binding molecule has two or more CH1 domains, the multiple CH1 domains can be a single CH1 domain in multiple copies of the same polypeptide chain present in the complete complex.

### 6.9.1. CH1 Binding Reagents

In exemplary non-limiting methods of purifying ROR binding molecules, a sample comprising the ROR binding molecules is contacted with CH1 binding reagents. CH1 binding reagents, as described herein, can be any molecule that specifically binds a CH1 epitope. The various CH1 sequences that provide the CH1 epitope are described in more detail in Section 6.3.8.1, and specific binding is described in more detail in Section 6.3.13.1.

In some embodiments, CH1 binding reagents are derived from immunoglobulin proteins and have an antigen binding site (ABS) that specifically binds the CH1 epitope. In particular embodiments, the CH1 binding reagent is an antibody, also referred to as an “anti-CH1 antibody.” The anti-CH1 antibody can be derived from a variety of species. In particular embodiments, the anti-CH1 antibody is a mammalian antibody, including, but not limited to mouse, rat, hamster, rabbit, camel, donkey, goat, and human antibodies. In specific embodiments, the anti-CH1 antibody is a single-domain antibody. Single-domain antibodies, as described herein, have a single variable domain that forms the ABS and specifically binds the CH1 epitope. Exemplary single-domain antibodies include, but are not limited to, heavy chain antibodies derived from camels and sharks, as described in more detail in international application WO 2009/011572, herein incorporated by reference for all it teaches. In a preferred embodiment, the anti-CH1 antibody is a camel derived antibody (also referred to as a “camelid antibody”). Exemplary camelid antibodies include, but are not limited to, human IgG-CH1 CaptureSelect™ (ThermoFisher, #194320010) and human IgA-CH1 (ThermoFisher, #194311010). In some embodiments, the anti-CH1 antibody is a monoclonal antibody. Monoclonal antibodies are typically produced from cultured antibody-producing cell lines. In other embodiments, the anti-CH1 antibody is a polyclonal antibody, i.e., a collection of different anti-CH1 antibodies that each recognize the CH1 epitope. Polyclonal antibodies are typically produced by collecting the antibody containing serum of an animal immunized with the antigen of interest, or fragment thereof, here CH1.

In some embodiments, CH1 binding reagents are molecules not derived from immunoglobulin proteins. Examples of such molecules include, but are not limited to, aptamers, peptoids, and affibodies, as described in more detail in Perret and Boschetti (Biochimie, Feb. 2018, Vol 145:98-112).

### 6.9.2. Solid Supports

In exemplary non-limiting methods of purifying ROR binding molecules, the CH1 binding reagent can be attached to a solid support in various embodiments of the invention. Solid supports, as described herein, refers to a material to which other entities can be attached or immobilized, e.g., the CH1 binding reagent. Solid supports, also referred to as “carriers,” are described in more detail in international application WO 2009/011572.

In specific embodiments, the solid support comprises a bead or nanoparticle. Examples of beads and nanoparticles include, but are not limited to, agarose beads, polystyrene beads, magnetic nanoparticles (e.g., Dynabeads™, ThermoFisher), polymers (e.g., dextran), synthetic polymers (e.g., Sepharose™), or any other material suitable for attaching the CH1 binding reagent. In particular embodiments, the solid support is modified to enable attachment of the CH1 binding reagent. Example of solid support modifications include, but are not limited to, chemical modifications that form covalent bonds with proteins (e.g., activated aldehyde groups) and modifications that specifically pair with a cognate modification of a CH1 binding reagent (e.g., biotin-streptavidin pairs, disulfide linkages, polyhistidine-nickel, or “click-chemistry” modifications such as azido-alkynyl pairs).

In certain embodiments, the CH1 binding reagent is attached to the solid support prior to the CH1 binding reagent contacting the ROR binding molecules, herein also referred to as an “anti-CH1 resin.” In some embodiments, anti-CH1 resins are dispersed in a solution.

In other embodiments, anti-CH1 resins are “packed” into a column. The anti-CH1 resin is then contacted with the ROR binding molecules and the CH1 binding reagents specifically bind the ROR binding molecules.

In other embodiments, the CH1 binding reagent is attached to the solid support after the CH1 binding reagent contacts the ROR binding molecules. As a non-limiting illustration, a CH1 binding reagent with a biotin modification can be contacted with the ROR binding molecules, and subsequently the CH1 binding reagent/ROR binding molecule mixture can be contacted with streptavidin modified solid support to attach the CH1 binding reagent to the solid support, including CH1 binding reagents specifically bound to the ROR binding molecules.

In methods wherein the CH1 binding reagents are attached to solid supports, in a variety of embodiments, the bound ROR binding molecules are released, or “eluted,” from the solid support forming an eluate having the ROR binding molecules. In some embodiments, the bound ROR binding molecules are released through reversing the paired modifications (e.g., reduction of the disulfide linkage), adding a reagent to compete off the ROR binding molecules (e.g., adding imidazole that competes with a polyhistidine for binding to nickel), cleaving off the ROR binding molecules (e.g., a cleavable moiety can be included in the modification), or otherwise interfering with the specific binding of the CH1 binding reagent for the ROR binding molecule. Methods that interfere with specific binding include, but are not limited to, contacting ROR binding molecules bound to CH1 binding reagents with a low-pH solution. In preferred embodiment, the low-pH solution comprises 0.1 M acetic acid pH 4.0. In other embodiments, the bound ROR binding molecules can be contacted with a range of low-pH solutions, i.e., a “gradient.”

### 6.9.3. Further Purification

In some embodiments of the exemplary non-limiting methods, a single iteration of the method using the steps of contacting the ROR binding molecules with the CH1 binding reagents, followed by eluting the ROR binding molecules, is used to purify the ROR binding molecules from the one or more incomplete complexes. In particular embodiments, no other purifying step is performed. In other embodiments, one or more additional purification steps are performed to further purify the ROR binding molecules from the one or more incomplete complexes. The one or more additional purification steps include, but are not limited to, purifying the ROR binding molecules based on other protein characteristics, such as size (e.g., size exclusion chromatography), charge (e.g., ion exchange chromatography), or hydrophobicity (e.g., hydrophobicity interaction chromatography). In a preferred embodiment, an additional cation exchange chromatograph is performed. Additionally, the ROR binding molecules can be further purified repeating contacting the ROR binding molecules with the CH1 binding reagents as described above, as well as modifying the CH1 purification method between iterations, e.g., using a step elution for the first iteration and a gradient elution for a subsequent elution.

### 6.9.4. Assembly and Purity of Complexes

In the embodiments of the present invention, at least four distinct polypeptide chains associate together to form a complete complex, e.g., the ROR binding molecule. However, incomplete complexes can also form that do not contain the at least four distinct polypeptide chains. For example, incomplete complexes may form that only have one, two, or three of the polypeptide chains. In other examples, an incomplete complex may contain more than three polypeptide chains, but does not contain the at least four distinct polypeptide chains, e.g., the incomplete complex inappropriately associates with more than one copy of a distinct polypeptide chain. The method of the invention purifies the complex, e.g., the completely assembled ROR binding molecule, from incomplete complexes.

Methods to assess the efficacy and efficiency of the purification steps are well known to those skilled in the art and include, but are not limited to, SDS-PAGE analysis, ion exchange chromatography, size exclusion chromatography, and mass spectrometry. Purity can also be assessed according to a variety of criteria. Examples of criterion include, but are not limited to: 1) assessing the percentage of the total protein in an eluate that is provided by the completely assembled ROR binding molecule, 2) assessing the fold enrichment or percent increase of the method for purifying the desired products, e.g., comparing the total protein provided by the completely assembled ROR binding molecule in the eluate to that in a starting sample, 3) assessing the percentage of the total protein or the percent decrease of undesired products, e.g., the incomplete complexes described above, including determining the percent or the percent decrease of specific undesired products (e.g., unassociated single polypeptide chains, dimers of any combination of the polypeptide chains, or trimers of any combination of the polypeptide chains). Purity can be assessed after any combination of methods described herein. For example, purity can be assessed after a single iteration of using the anti-CH1 binding reagent, as described herein, or after additional purification steps, as described in more detail in Section 6.9.3. The efficacy and efficiency of the purification steps may also be used to compare the methods described using the anti-CH1 binding reagent to other purification methods known to those skilled in the art, such as Protein A purification.

### 6.10. Methods of Manufacturing

The ROR binding molecules described herein can readily be manufactured by expression using standard cell free translation, transient transfection, and stable transfection approaches currently used for antibody manufacture. In specific embodiments, Expi293 cells (ThermoFisher) can be used for production of the ROR binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

As further described in the Examples below, the expressed proteins can be readily separated from undesired proteins and protein complexes using a CH1 affinity resin, such as the CaptureSelect CH1 resin and provided protocol from ThermoFisher. Other purification strategies include, but are not limited to, use of Protein A, Protein G, or Protein A/G reagents. Further purification can be affected using ion exchange chromatography as is routinely used in the art.

### 6.11. Pharmaceutical Compositions

In another aspect, pharmaceutical compositions are provided that comprise a ROR binding molecule as described herein and a pharmaceutically acceptable carrier or diluent. In typical embodiments, the pharmaceutical composition is sterile.

In various embodiments, the pharmaceutical composition comprises the ROR binding molecule at a concentration of 0.1 mg/ml-100 mg/ml. In specific embodiments, the pharmaceutical composition comprises the ROR binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the ROR binding molecule at a concentration of more than 10 mg/ml. In certain embodiments, the ROR binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the ROR binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

### 6.12. Methods of Treatment

In another aspect, methods of treatment are provided, the methods comprising administering a ROR binding molecule (e.g., antibody) as described herein to a subject in an amount effective to treat the subject. Such ROR antigen binding molecules are useful in the treatment of ROR expressing cancers, including cancers that express an ROR1 antigen, cancers that express an ROR2 antigen, and/or cancers that express both an ROR1 antigen and an ROR2 antigen.

In some embodiments, an antibody of the present disclosure may be used to treat a variety of cancers. The cancer may be a cancer from the bladder, blood (myeloid leukemia [acute and chronic], acute lymphoblastic leukemia, chronic lymphocytic leukemia, myeloproliferative diseases, multiple myeloma, myelodysplastic syndrome), bone, bone marrow, brain (astrocytoma, medulloblastoma, glioma, ependymoma, germinoma [pinealoma], glioblastoma multiform, oligodendroglioma, schwannoma, retinoblastoma, congenital tumors), breast, colon, esophagus (squamous cell carcinoma, adenocarcinoma, leiomyosarcoma, lymphoma), gastrointestine, gum, head, kidney (adenocarcinoma, Wilm's tumor [nephroblastoma], lymphoma, leukemia, renal cell carcinoma), liver, lung, nasopharynx, neck, ovary, prostate (adenocarcinoma, sarcoma, castrate resistant prostate cancer), skin, stomach (carcinoma, lymphoma, leiomyosarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), tongue, or uterus. In some embodiments, the cancer may be a neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma (angiosarcoma, fibrosarcoma, rhabdomyosarcoma, liposarcoma); fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma (reticulum cell sarcoma); hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; myxoma; rhabdomyoma; fibroma; squamous cell carcinomas of the head and neck; laryngeal and hypopharyngeal cancer; nasal cavity and paranasal sinus cancer; nasopharyngeal cancer; salivary gland cancer; oral; orppharyngeal cancer; bronchogenic carcinoma (squamous cell, undifferentiated small cell, undifferentiated large cell, adenocarcinoma, non-small cell lung cancer); alveolar (bronchiolar) carcinoma; bronchial adenoma; chondromatous hamartoma; colorectal cancer; gastrointestinal stromal tumors; carcinoids; Turcot Syndrome; gastric cancer; gastroesophageal junction adenocarcinoma; pancreas (ductal adenocarcinoma, insulinoma, glucagonoma, gastrinoma, carcinoid tumors, vipoma); small bowel (adenocarcinoma, lymphoma, carcinoid tumors, Karposi's sarcoma, leiomyoma, hemangioma, lipoma, neurofibroma, fibroma); large bowel (adenocarcinoma, tubular adenoma, villous adenoma, hamartoma, leiomyoma); metastatic breast cancer; ductal carcinoma in situ; invasive ductal carcinoma; tubular carcinoma; mucinous carcinoma; lobular carcinoma in situ; triple negative breast cancer; bladder and urethra (squamous cell carcinoma, transitional cell carcinoma, adenocarcinoma, urothelial carcinoma); clear cell carcinoma; hepatoma (hepatocellular carcinoma); angiosarcoma; hepatocellular adenoma; hemangioma; osteogenic sarcoma (osteosarcoma); malignant fibrous histiocytoma; malignant giant cell tumor chordoma; osteochrondroma (osteocartilaginous exostoses); benign chondroma; chondromyxofibroma; osteoid osteoma; giant cell tumors; medullary thyroid cancer; differentiated thyroid cancer; papillary thyroid cancer; follicular thyroid cancer; hurthle cell cancer; anaplastic thyroid cancer; skull (osteoma, hemangioma, granuloma, xanthoma, osteitis deformans); meninges (meningioma, meningiosarcoma, gliomatosis); spinal cord (neurofibroma, meningioma, glioma, sarcoma); uterus (clear); cervix (cervical carcinoma, pre-tumor cervical dysplasia); ovaries (ovarian carcinoma [serous cystadenocarcinoma, mucinous cystadenocarcinoma, unclassified carcinoma], granulosa-thecal cell tumors, Sertoli—Leydig cell tumors, dysgerminoma, malignant teratoma); vulva (squamous cell carcinoma, intraepithelial carcinoma, adenocarcinoma, fibrosarcoma, melanoma); vagina (clear cell carcinoma, squamous cell carcinoma); botryoid sarcoma (embryonal rhabdomyosarcoma); fallopian tubes (carcinoma); non-Hodgkin's lymphoma [malignant lymphoma]; Karposi's sarcoma; moles dysplastic nevi; angioma; dermatofibroma; keloids; psoriasis; neuroblastoma.; adrenocortical carcinoma; pheochromocytomas; paragangliomas; merkel cell carcinoma; pancreatic neuroendocrine and carcinoid tumors; neuroendocrine tumors; carcinoid tumors; pancreatic cancers; gastroesophageal; clear cell renal cell carcinoma; and primary peritoneal cancer.

An antibody of the present disclosure may be administered to a subject per se or in the form of a pharmaceutical composition for the treatment of, e.g., cancer, autoimmunity, transplantation rejection, post-traumatic immune responses, graft-versus-host disease, ischemia, stroke, and infectious diseases (e.g., by targeting viral antigens, such as gp120 of HIV).

In another aspect, a ROR binding molecule (e.g., antibody) as described herein may be used in a method of treating a subject with cancer in combination with one or more additional therapies. The additional therapies that may be used in combination with an ROR antigen binding molecule (e.g., antibody) described herein include but are not limited to: (i) surgery; (ii) radiotherapy; (iii) endocrine therapy; (iv) immunotherapy (including adjuvant therapy and cell therapy such as CAR T-cell therapy); and (v) chemotherapy, including cytotoxic agents and chemotherapeutic agents.

Any therapy that has an activity against a cancer may be used in combination with an ROR antigen binding molecule (e.g., antibody) provided herein. Examples of such agents for cancer treatment can be found, for instance, at https://www.cancer.gov/about-cancer/treatment/drugs (last visited Jan. 22, 2019) and in publically available sources such as Cancer Principles and Practice of Oncology by V. T. Devita and S. Hellman (editors), 11^{th} edition (2018), Lippincott Williams & Wilkins Publishers. A person of ordinary skill in the art would be able to discern which combinations of agents would be useful based on the particular characteristics of the drugs and the type of cancer involved.

In certain embodiments, the additional therapy is a radiotherapy including, for example, gamma radiation, neutron beam radiotherapy, electron beam radiotherapy, proton therapy, brachytherapy, and systemic radioactive isotopes. Radiotherapy may comprise radiation or associated administration of radiopharmaceuticals. The source of radiation may be either external or internal to the subject being treated (radiation treatment may, for example, be in the form of external beam radiation therapy (EBRT) or brachytherapy (BT)). Exemplary radioactive elements include, e.g., radium, cesium-137, iridium-192, americium-241, gold-198, cobalt-57, copper-67, technetium-99, iodide-123, iodide-131, and indium-111.

In certain embodiments, the additional therapy is an immunotherapy. Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies, including immunotherapeutic agents, stimulate the body's own immune system (e.g., vaccines) while passive immunotherapies, including immunotherapeutic agents, generally use immune system components created outside of the body (e.g., antibodies), antibodies conjugated with drugs, toxins, or radionuclides, and targeted therapeutics.

Exemplary immunotherapeutic agents include immune checkpoint inhibitors. In some embodiments, the immune checkpoint inhibitor used in methods of treatment can totally or partially reduce, inhibit, interfere with, or modulate one or more checkpoint proteins which regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). Immune checkpoint inhibitors include antibodies or are derived from antibodies.

In certain embodiments, the checkpoint inhibitor is an OX40 (CD134) agonist. In some embodiments, the checkpoint inhibitor is an anti-OX40 antibody. In some embodiments, the anti-OX40 antibody is anti-OX-40. In some embodiments, the anti-OX40 antibody is MEDI6469.

In certain embodiments, the checkpoint inhibitor is a CD40 agonist. In some embodiments, the checkpoint inhibitor is an anti-CD40 antibody. In some embodiments, the anti-CD40 antibody is CF-870,893.

In certain embodiments, the checkpoint inhibitor is a CTLA-4 inhibitor. In some embodiments, the CTLA-4 inhibitor is an anti-CTLA-4 antibody. Examples of anti CTLA 4 antibodies include, but are not limited to, those described in U.S. Pat. Nos. 5,811,097; 5,811,097; 5,855,887; 6,051,227; 6,207,157; 6,682,736; 6,984,720; and 7,605,238. In some embodiments, the anti-CTLA-4 antibody is tremelimumab (also known as ticilimumab or CP-675,206). In some embodiments, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™

In certain embodiments, the checkpoint inhibitor is a PD-1/PD-L1 inhibitor. Examples of PD-l/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699.

In certain embodiments, the checkpoint inhibitor is a PD-1 inhibitor. In some embodiments, the PD-1 inhibitor is an anti-PD-1 antibody. In some embodiments, the anti-PD-1 antibody is BGB-A317, nivolumab (also known as ONO-4538, BMS-936558, or MDX1106) or pembrolizumab (also known as MK-3475, SCH 900475, or lambrolizumab). In some embodiments, the anti-PD-1 antibody is nivolumab. Nivolumab is a human IgG4 anti-PD-1 monoclonal antibody, and is marketed under the trade name Opdivo™. In some embodiments, the anti-PD-1 antibody is pembrolizumab. Pembrolizumab is a humanized monoclonal IgG4 antibody and is marketed under the trade name Keytruda™. In some embodiments, the anti-PD-1 antibody is CT-011, a humanized antibody. CT-011 administered alone has failed to show response in treating acute myeloid leukemia (AML) at relapse. In some embodiments, the anti-PD-1 antibody is AMP-224, a fusion protein. In some embodiments, the PD-1 antibody is BGB-A317. BGB-A317 is a monoclonal antibody in which the ability to bind Fe gamma receptor I is specifically engineered out, and which has a unique binding signature to PD-1 with high affinity and superior target specificity.

In certain embodiments, the checkpoint inhibitor is a PD-L1 inhibitor. In one embodiments, the PD-L1 inhibitor is an anti-PD-L1 antibody. In some embodiments, the anti-PD-L1 antibody is MEDI4736 (durvalumab). In some embodiments, the anti-PD-L1 antibody is BMS-936559 (also known as MDX-1105-01). In some embodiments, the PD-L1 inhibitor is atezolizumab (also known as MPDL3280A, and Tecentriq®).

In certain embodiments, the checkpoint inhibitor is a PD-L2 inhibitor. In some embodiments, the PD-L2 inhibitor is an anti-PD-L2 antibody. In some embodiments, the anti-PD-L2 antibody is rHIgM12B7A.

In certain embodiments, the checkpoint inhibitor is a lymphocyte activation gene-3 (LAG-3) inhibitor. In some embodiments, the LAG-3 inhibitor is IMP321, a soluble Ig fusion protein (Brignone et al., J. Immunol., 2007, 179, 4202-4211). In some embodiments, the LAG-3 inhibitor is BMS-986016.

In certain embodiments, the checkpoint inhibitor is a B7 inhibitor. In some embodiments, the B7 inhibitor is a B7-H3 inhibitor or a B7-H4 inhibitor. In some embodiments, the B7-H3 inhibitor is MGA271, an anti-B7-H3 antibody (Loo et al., Clin. Cancer Res., 2012, 3834).

In certain embodiments, the checkpoint inhibitor is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In certain embodiments, the checkpoint inhibitor is a GITR agonist. In some embodiments, the checkpoint inhibitor is an anti-GITR antibody. In some embodiments, the anti-GITR antibody is TRX518.

In certain embodiments, the checkpoint inhibitor is a CD137 agonist. In some embodiments, the checkpoint inhibitor is an anti-CD137 antibody. In some embodiments, the anti-CD137 antibody is urelumab. In some embodiments, the anti-CD137 antibody is PF-05082566.

In certain embodiments, the checkpoint inhibitor is recombinant human interleukin-15 (rhIL-15).

In certain embodiments, the checkpoint inhibitor is an IDO inhibitor. In some embodiments, the IDO inhibitor is INCB024360. In some embodiments, the IDO inhibitor is indoximod.

Other exemplary immunotherapies include adjuvant therapies, including immunotherapeutic agents such as cytokines, chemokines, interferons, interleukins, or lymphokines. Examples include cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of, interleukins, for example, IL-2 with other cytokines, such as IFN-alpha.

Other exemplary immunotherapies include cell therapies, for example, a population of immune cells, such as leukocytes (nucleated white blood cells), comprising (e.g., expressing) a receptor that binds to an antigen of interest. A leukocyte of the present disclosure may be, for example, a neutrophil, eosinophil, basophil, lymphocyte or a monocyte. In some embodiments, a leukocyte is a lymphocyte. Examples of lymphocytes include T cells, B cells, Natural Killer (NK) cells or NKT cells. In some embodiments, a T-cell is a CD4+ Th (T helper) cell, a CD8+ cytotoxic T cell, a T6T cell or a regulatory (suppressor) T cell. In some embodiments, an immune cell is a dendritic cell. In some embodiments, the cell therapies are CAR-T cell therapies. In some embodiments, a bispecific CAR is comprised of two distinct antigen recognition domains present in tandem on a single transgenic receptor (referred to as a TanCAR; see, e.g., Grada Z et al. Molecular Therapy Nucleic Acids 2013; 2:e105, incorporated herein by reference in its entirety). Thus, methods, in some embodiments, comprise delivering to a tumor a combination comprising a ROR antigen binding molecule (e.g., antibody) and an immunotherapeutic agent, wherein the immunotherapeutic agent is an engineered nucleic acid that encodes an antigen, or delivering to a tumor an engineered nucleic acid that induces expression of a self-antigen, and delivering to the tumor an immune cell expressing a bispecific CAR that binds to two antigens, one of which is encoded by the engineered nucleic acid.

Other exemplary immunotherapies include immunotherapeutic agents such as cancer vaccines, which can be used to illicit an immune response in a subject against a cancer antigen. An exemplary method involves administering to a subject a RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, in combination with administering a ROR antigen binding molecule (e.g., antibody) either in the same composition or a separate composition, administered at the same time, or sequentially dosed, wherein the anti-antigenic polypeptide antibody titer in the subject is increased following vaccination relative to anti-antigenic polypeptide antibody titer in a subject vaccinated with a prophylactically effective dose of a traditional vaccine against the cancer.

In certain embodiments, the additional therapies include chemotherapy such as one or more cytotoxic agents or one or more chemotherapeutic agents. An cytotoxic agent can inhibit or prevent a cellular function and/or causes cell death or destruction. Cytotoxic agents include, but are not limited to, radioactive isotopes (e.g., At²¹¹, J¹³¹ I¹²⁵ Y⁹⁰, Re¹⁸⁶, Re¹⁸⁸ Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

In certain embodiments, the additional therapy includes one or more chemotherapeutic agents. Chemotherapeutic agents include chemical compounds useful in the treatment of cancer. Chemotherapeutic agents include (i) anti-hormonal agents that act to regulate or inhibit hormone action on tumors such as anti-estrogens and selective estrogen receptor modulators; (ii) aromatase inhibitors that inhibit the enzyme aromatase, which regulates estrogen production in the adrenal glands; (iii) anti-androgens; (iv) protein kinase inhibitors; (v) lipid kinase inhibitors; (vi) antisense oligonucleotides, including those which inhibit expression of genes in signaling pathways implicated in aberrant cell proliferation; (viii) vaccines such as gene therapy vaccines. Chemotherapeutic agents can also include antibodies.

Exemplary kinase inhibitors include erlotinib (Tarceva®), gefitinib (Iressa®), dasatinib (Sprycel®), nilotinib (Tasigna®), crizotinib (Xalkori®), ruxolitinib (Jakafi®), vemurafenib (Zelboraf®), vandetanib (Caprelsa®), pazopanib (Votrient®), afatinib, alisertib, amuvatinib, axitinib, baricitinib, bosutinib, brivanib, canertinib, cabozantinib (Cabometyx®), cediranib, ceritinib, crenolanib, dabrafenib, dacomitinib, danusertib, dovitinib, foretinib, ganetespib, ibrutinib, idelalisib, imatinib, iniparib, lapatinib, lenvatinib, linifanib, linsitinib, masitinib, momelotinib, motesanib, neratinib, nintedanib, niraparib, oprozomib, olaparib, palbociclib, pictilisib, pirfenidone, ponatinib, quizartinib, regorafenib, rigosertib, rucaparib, saracatinib, saridegib, sorafenib, sunitinib, tandutinib, tasocitinib, telatinib, tivantinib, tivozanib, tofacitinib, trametinib, veliparib, vismodegib, volasertib, cobimetinib (Cotellic®), XL-147, XL-765, XL-499, XL-880, and others. In some embodiments, a ROR antigen binding molecule (e.g., antibody) can be used in combination with a HSP90 inhibitor (e.g., XL888), liver X receptor (LXR) modulators, retinoid-related orphan receptor gamma (RORy) modulators, a CK1 inhbitor, a CK1l-a inhibitor, a Wnt pathway inhibitor (e.g., SST-215), or a mineralocorticoid receptor inhibitor, (e.g., esaxerenone or XL-550) for the treatment of cancer.

Kinase inhibitors can be tyrosine kinase inhibitors, such as the EGFR inhibitors; small molecule HER2 tyrosine kinase inhibitor such as Mubritonib (TAK165, Takeda); CP-724.714, (Axon Medchem BV, an oral selective inhibitor of the ErbB2 receptor tyrosine kinase); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase 1 inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; antisense molecules (e.g. those that bind to HER-encoding nucleic acid); quinoxalines (U.S. Pat. No. 5,804,396); tryphostins (U.S. Pat. No. 5,804,396); Affinitac (ISIS 3521; Isis/Lilly); PKI166 (Novartis); Semaxinib (Pfizer); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Combined treatment with the ROR antigen binding molecule (e.g., antibody) provided herein and the additional therapy, such as a therapeutic agent, may be simultaneous, separate or sequential, in any order. For combinations of therapeutic agents such as an ROR antigen binding providing molecule and another therapeutic agent such as an immunotherapeutic agent or a chemotherapeutic agent, simultaneous administration, the therapeutics agents may be administered as one composition or as separate compositions, as appropriate.

### 6.13. Examples

The following examples are provided by way of illustration, not limitation.

### 6.13.1. Methods

Non-limiting, illustrative methods for the purification of the various antigen-binding proteins and their use in various assays are described in more detail below.

### 6.13.1.1. Expi293 Expression

The various antigen-binding proteins tested were expressed using the Expi293 transient transfection system according to manufacturer's instructions. Briefly, four plasmids coding for four individual chains were mixed at 1:1:1:1 mass ratio, unless otherwise stated, and transfected with ExpiFectamine 293 transfection kit to Expi 293 cells. Cells were cultured at 37° C. with 8% C02, 100% humidity and shaking at 125 rpm. Transfected cells were fed once after 16-18 hours of transfections. The cells were harvested at day 5 by centrifugation at 2000 g for 10 minutes. The supernatant was collected for affinity chromatography purification.

### 6.13.1.2. Protein A and Anti-CH1 Purification

Cleared supernatants containing the various antigen-binding proteins were separated using either a Protein A (ProtA) resin or an anti-CH1 resin on an AKTA Purifier FPLC. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1 M acetic acid pH 4.0. The elution was monitored by absorbance at 280 nm and the elution peaks were pooled for analysis. For anti-CH1 purification, a 1 mL CaptureSelect™ XL column (ThermoFisher) was equilibrated with PBS. The sample was loaded onto the column at 5 ml/min. The sample was eluted using 0.1 M acetic acid pH 4.0. The elution was monitored by absorbance at 280 nm and the elution peaks were pooled for analysis.

### 6.13.1.3. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 95° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

### 6.13.1.4. IEX Chromatography

Samples containing the various separated antigen-binding proteins were analyzed by cation exchange chromatography for the ratio of complete product to incomplete product and impurities. Cleared supernatants were analyzed with a 5-ml MonoS (GE Lifesciences) on an AKTA Purifier FPLC. The MonoS column was equilibrated with buffer A 10 mM MES pH 6.0. The samples were loaded onto the column at 2 ml/min. The sample was eluted using a 0-30% gradient with buffer B (10 mM MES pH 6.0, 1 M sodium chloride) over 6 CV. The elution was monitored by absorbance at 280 nm and the purity of the samples were calculated by peak integration to identify the abundance of the monomer peak and contaminants peaks. The monomer peak and contaminant peaks were separately pooled for analysis by SDS-PAGE as described above.

### 6.13.1.5. Analytical SEC Chromatography

Samples containing the various separated antigen-binding proteins were analyzed by analytical size exclusion chromatography for the ratio of monomer to high molecular weight product and impurities. Cleared supernatants were analyzed with an industry standard TSK G3000SWxl column (Tosoh Bioscience) on an Agilent 1100 HPLC. The TSK column was equilibrated with PBS. 25 μL of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

### 6.13.1.6. Mass Spectrometry

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were both tested in the reduced format to specifically identify each chain by molecular weight. Samples were all tested under non-reducing conditions to identify the molecular weights of all complexes in the samples. Mass spec analysis was used to identify the number of unique products based on molecular weight. 6.13.1.7. Antibody discovery by phage display

Phage display of human Fab libraries was carried out using standard protocols. Biotinylated extracellular domains of human ROR1 and ROR2 protein were purchased from Acro Biosystems and biotinylated with EZ-Link NHS biotin (Thermo Scientific Cat. No. 20217). Phage clones were screened for the ability to bind the extracellular domains of ROR1 (Acro Cat. No. RO1-H522γ) and ROR2 (Acro Cat. No. R02-H52E5) by phage ELISA using standard protocols. Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain-pIII fusion are expressed as separate polypeptides and assemble in the bacterial periplasm, where the redox potential enables disulfide bond formation, to form the phage display antibody containing the candidate ABS.

The library was created using sequences derived from a specific human heavy chain variable domain (VH3-23) and a specific human light chain variable domain (Vk-1). Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:74) and light chain (SEQ ID NO:75) scaffolds used in the library are listed below, where a lower case “x” represents CDR amino acids that were varied to create the library, and bold italic represents the CDR sequences that were constant.

Diversity was created through Kunkel mutagenesis using primers to introduce diversity into VL CDR3 and VH CDR1 (H1), CDR2 (H2) and CDR3 (H3) to mimic the diversity found in the natural antibody repertoire, as described in more detail in Kunkel, TA (PNAS Jan. 1, 1985. 82 (2) 488-492), herein incorporated by reference in its entirety.

Briefly, single-stranded DNA were prepared from isolated phage using standard procedures and Kunkel mutagenesis carried out. Chemically synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to ˜5×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀˜0.6. After 20 minutes, infected cells were sub-cultured in 25 mL 2xYT+Ampicillin and M13K07 helper phage and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for the target antigen. Target-specific enrichment was confirmed using polyclonal and monoclonal phage ELISA. DNA sequencing was used to determine isolated Fab clones containing a candidate ABS.

To measure binding affinity in ROR binder discovery campaigns, the VL and VH domains identified in the phage screen described above were formatted into a bivalent monospecific native human full-length IgG1 architecture and immobilized to a biosensor on an Octet (Pall ForteBio) biolayer interferometer. Soluble ROR antigens, including the extracellular domains of ROR1 (Acro Cat. No. RO1-H522γ) and ROR2 (Acro Cat. No. R02-H52E5), as well as the individual ROR1 Frizzled (Acro Cat. No. RO1-H5222), Ig-like (Acro Cat. No. RO1-H5221), and Kringle (Acro Cat. No. RO1-H5223) domains, were then added to the system and binding measured.

For experiments performed using the B-Body format, VL variable regions of individual clones were formatted into Domain A and/or H, and VH region into Domain F and/or L of a bivalent 1×1 B-Body “BC1” scaffold shown below and with reference to FIG. 3.

“BC1” Scaffold:
1^{st} polypeptide chain (SEQ ID NO:78)
   Domain A=Antigen 1 B-Body Domain A/H Scaffold (SEQ ID NO:76)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:79):
   Domain F=Antigen 1 B-Body Domain F/L Scaffold (SEQ ID NO:77)
   Domain G=CH3 (L351D; 445G, 446E, 447C tripeptide insertion)
3^{rd} polypeptide chain (SEQ ID NO:80):
   Domain H=Antigen 2 B-Body Domain A/H Scaffold (SEQ ID NO:76)
   Domain I=CL (Kappa)
      Domain J=CH2
      Domain K=CH3 (Y349C, D356E, L358M, T366S, L368A, Y407V)
4^{th} polypeptide chain (SEQ ID NO:81):
   Domain L=Antigen 2 B-Body Domain F/L Scaffold (SEQ ID NO:77)
   Domain M=CH1.

For ROR ABS candidates formatted into a bivalent bispecific 1×1 format with anti-CD3 SP34-89, domain H has the amino acid sequence of SEQ ID NO:69 and domain L has the amino acid sequence of SEQ ID NO:68, while domain A has the candidate ROR VL sequence and domain F had the candidate ROR VH sequence.

For BC1 1×2 formats, the variable domains were formatted into the 1(A)×2(B-A) format described in Section 6.3.17.4, which refers to FIG. 26. FIG. 26 presents a schematic of five polypeptide chains and their domains, with respective naming conventions, for the trivalent 1×2 antibody constructs described herein, wherein according to the naming convention, chain 5 is named “6^{th} polypeptide chain” in the schematic. Unless otherwise specified, the ROR antigen binding site (ABS) is the bivalent binder (the “A” specificity) and CD3 the monovalent binder (the “B” specificity). The SP34-89 1×2 chain 3 scaffold has the sequence of SEQ ID NO:82, where the junction between domain S and domain H is a 10 amino acid linker having the sequence TASSGGSSSG (SEQ ID NO:83), unless otherwise stated. Polypeptide chain 2 and chain 5 are identical in the 1(A)×2(B-A) format (see, e.g., FIG. 26, wherein according to the naming convention chain 5 is named “6^{th} polypeptide chain” in the schematic). 6.13.1.8. NFκB GFP Jurkat T cell Stimulation Assay

The NFκB/Jurkat/GFP transcriptional reporter cell line was purchased from System Biosciences (Cat #TR850-1). The anti-CD28 antibody used for co-stimulation was purchased from BD Pharmingen (Cat 555725). The Solution C background suppression dye was purchased from Life Technologies (K1037). Briefly, the Jurkat cells (effector cells, E) were mixed with the tumor cells (T) at an E:T ratio of 2:1 to 4:1 in the presence of a dilution series of B-body™ antibodies and an anti-CD28 antibody at 1 μg/mL in a 96 well black walled clear bottom plate. The plate was incubated at 37° C./5% CO₂ for 6 hours, following which a 6× solution of Solution C background suppressor was added to the plate and GFP fluorescence was read out on a plate reader. EC50 values, referring to the concentration of antibody that gives the half-maximal response, were determined from the dilution series.

### 6.13.1.9. Primary T Cell Cytotoxicity Assay

Cells expressing the target tumor antigen (T) and effector cells (E) were mixed at an E:T ratio ranging from 3:1 to 10:1. Effector cells used include PBMCs or isolated cytotoxic CD8+ T cells. The candidate redirecting T cell antibody was added in a dilution series to the cells. Controls included media only controls, tumor cell only controls, and untreated E:T cell controls. The mixed cells and control conditions were incubated at 37° C./5% CO₂ for 40-50 hours. The Cytotoxicity Detection Kit Plus (LDH) was purchased from Sigma (Cat 4744934001) and the manufacturer's directions were followed. Briefly, lysis solution added to tumor cells served as the 100% cytotoxicity control and untreated E:T cells served as the 0% cytotoxicity control. The level of lactate dehydrogenase (LDH) in each sample was determined via absorbance at 490 nm and normalize to the 100% and 0% controls. EC50 values, referring to the concentration of antibody that gives the half-maximal response, were determined from the dilution series.

### 6.13.2. Example 1: Bivalent monospecific construct and bivalent bispecific construct

A bivalent monospecific B-Body recognizing TNFα was constructed with the following architecture (VL(Certolizumab)—CH3(Knob)—CH2—CH3/VH(Certolizumab)-CH3(Hole)) using standard molecular biology procedures. In this construct,
1^{st} polypeptide chain (SEQ ID NO:1)
   Domain A=VL (certolizumab)
   Domain B=CH3 (IgG1) (knob: S354C+ T366W)
   Domain D=CH2 (IgG1)
   Domain E=CH3 (IgG1)
2^{nd} polypeptide chain (SEQ ID NO:2)
   Domain F=VH (certolizumab)
   Domain G=CH3 (IgG1) (hole:Y349C, T366S, L368A, Y407V)
3^{rd} polypeptide chain:
   identical to the 1^{st} polypeptide chain
4^{th} polypeptide chain:
   identical to the 2^{nd} polypeptide chain.

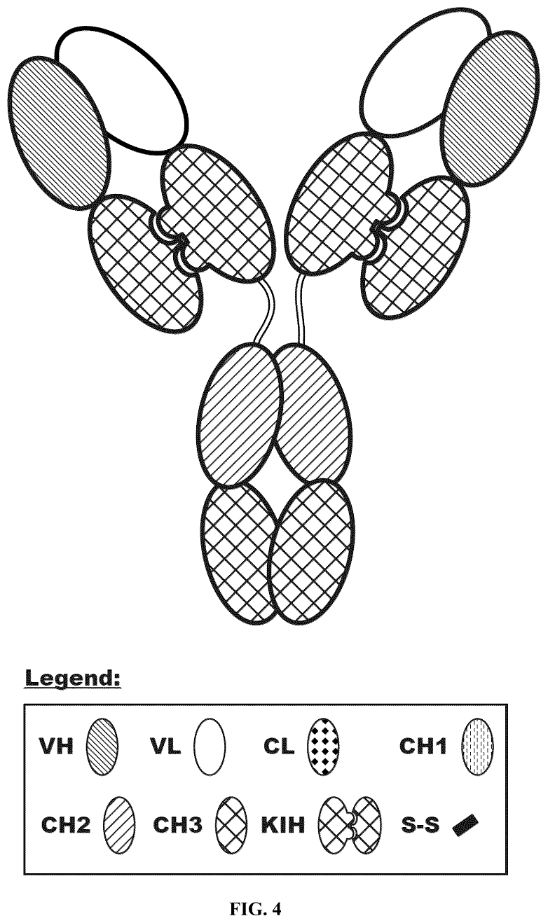

Domain and polypeptide chain references are in accordance with FIG. 3. The overall construct architecture is illustrated in FIG. 4. The sequence of the first polypeptide chain, with domain A identified in shorthand as “(VL)”, is provided in SEQ ID NO:1. The sequence of the second polypeptide chain, with domain F identified in shorthand as “(VH)”, is provided in SEQ ID NO:2.

The full-length construct was expressed in an E. coli cell free protein synthesis expression system for ˜18 hours at 26° C. with gentle agitation. Following expression, the cell-free extract was centrifuged to pellet insoluble material and the supernatant was diluted 2× with 10× Kinetic Buffer (Forte Bio) and used as analyte for biolayer interferometry.

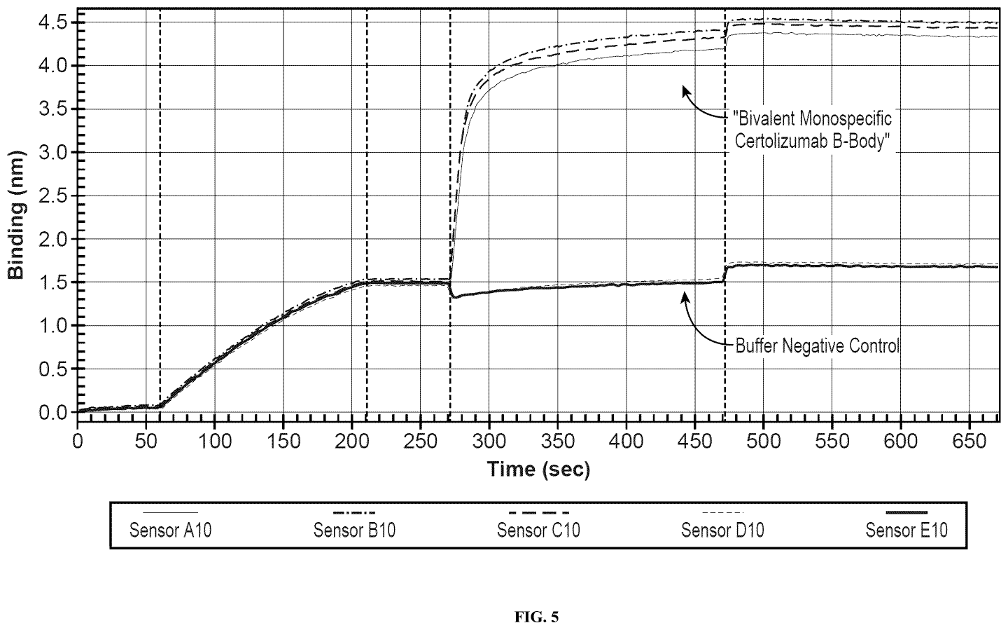

Biotinylated TNFα was immobilized on a streptavidin sensor to give a wave shift response of ˜1.5 nm. After establishing a baseline with 10× kinetic buffer, the sensor was dipped into the antibody construct analyte solution. The construct gave a response of ˜3 nm, comparable to the traditional IgG format of certolizumab, demonstrating the ability of the bivalent monospecific construct to assemble into a functional, full-length antibody. Results are shown in FIG. 5.

We also constructed a bivalent bispecific antibody with the following domain architecture:
1^{st} polypeptide chain: VL—CH3—CH2—CH3(Knob)
2^{nd} polypeptide chain: VH-CH3
3^{rd} polypeptide chain: VL-CL-CH2—CH3(Hole)
4^{th} polypeptide chain VH-CH1.

The sequences (except for the variable region sequences) are provided respectively in SEQ ID NO:3 (1^{st} polypeptide chain), SEQ ID NO:4 (2nd polypeptide chain), SEQ ID NO:5 (3rd polypeptide chain), SEQ ID NO:6 (4^{th} polypeptide chain).

6.13.3. Example 2: Bivalent bispecific B-Body “BC1”

We constructed a bivalent bispecific construct, termed “BC1”, specific for PD1 and a second antigen, “Antigen A”). Salient features of the “BC1” architecture are illustrated in FIG. 6.

In greater detail, with domain and polypeptide chain references in accordance with FIG. 3 and modifications from native sequence indicated in parentheses, the architecture was:
1^{st} polypeptide chain (SEQ ID NO:8)
   Domain A=VL (“Antigen A”)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:9):
   Domain F=VH (“Antigen A”)
   Domain G=CH3 (L351D; 445G, 446E, 447C tripeptide insertion)
3^{rd} polypeptide chain (SEQ ID NO:10):
   Domain H=VL (“Nivo”)
   Domain I=CL (Kappa)
   Domain J=CH2
   Domain K=CH3 (Y349C, D356E, L358M, T366S, L368A, Y407V)
4^{th} polypeptide chain (SEQ ID NO:11):
   Domain L=VH (“Nivo”)
   Domain M=CH1.

The A domain (SEQ ID NO: 12) and F domain (SEQ ID NO: 16) form an antigen binding site (A:F) specific for “Antigen A”. The H domain has the VH sequence from nivolumab and the L domain has the VL sequence from nivolumab; H and L associate to form an antigen binding site (H:L) specific for human PD1.

The B domain (SEQ ID NO:13) has the sequence of human IgG1 CH3 with several mutations: T366K, 445K, 446S, and 447C insertion. The T366K mutation is a charge pair cognate of the L351D residue in Domain G. The “447C” residue on domain B comes from the C-terminal KSC tripeptide insertion.

Domain D (SEQ ID NO: 14) has the sequence of human IgG1 CH2

Domain E (SEQ ID NO: 15) has the sequence of human IgG1 CH3 with the mutations T366W and S354C. The 366W is the “knob” mutation. The 354C introduces a cysteine that is able to form a disulfide bond with the cognate 349C mutation in Domain K.

Domain G (SEQ ID NO:17) has the sequence of human IgG1 CH3 with the following mutations: L351D, and 445G, 446E, 447C tripeptide insertion. The L351D mutation introduces a charge pair cognate to the Domain B T366K mutation. The “447C” residue on domain G comes from the C-terminal GEC tripeptide insertion.

Domain I (SEQ ID NO: 19) has the sequence of human C kappa light chain (Cx)

Domain J [SEQ ID NO: 20] has the sequence of human IgG1 CH2 domain, and is identical to the sequence of domain D.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in Domain E. The 356E and L358M introduce isoallotype amino acids that reduce immunogenicity. The 366S, 368A, and 407V are “hole” mutations.

Domain M [SEQ ID NO: 23] has the sequence of the human IgG1 CH1 region.

“BC1” could readily be expressed at high levels using mammalian expression at concentrations greater than 100 μg/ml.

We found that the bivalent bispecific “BC1” protein could easily be purified in a single step using a CH1-specific CaptureSelect™ affinity resin from ThermoFisher.

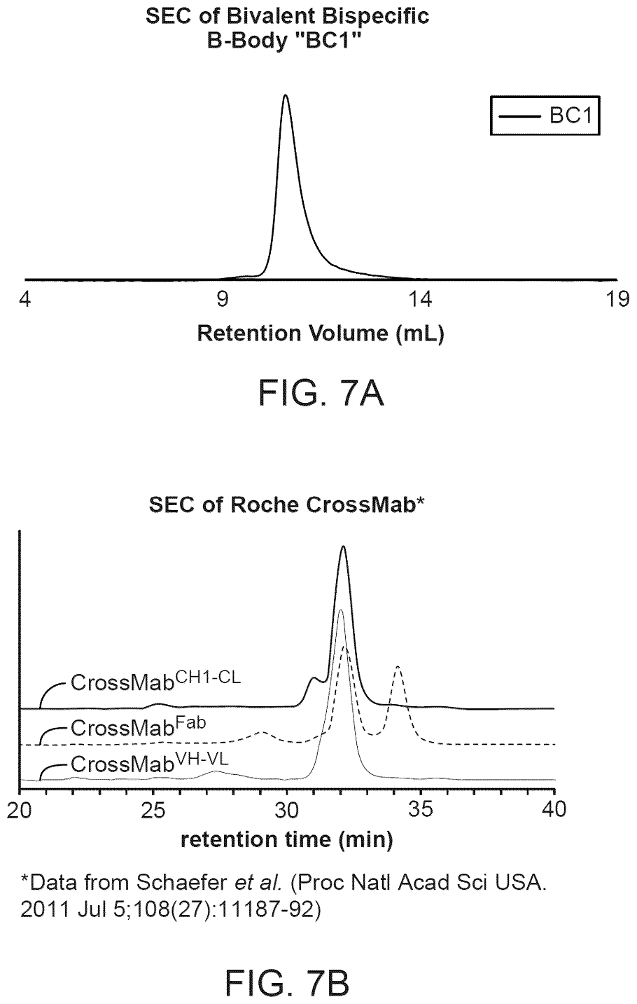

As shown in FIG. 7A, SEC analysis demonstrates that a single-step CH1 affinity purification step yields a single, monodisperse peak via gel filtration in which>98% is monomer. FIG. 7B shows comparative literature data of SEC analysis of a CrossMab bivalent antibody construct.

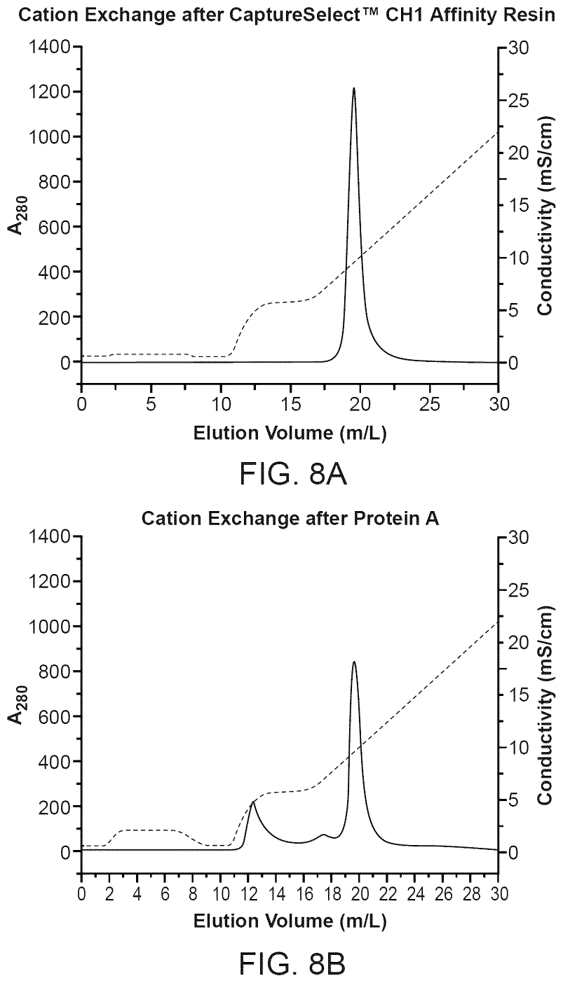

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification, showing additional elution peaks consistent with the co-purification of incomplete assembly products.

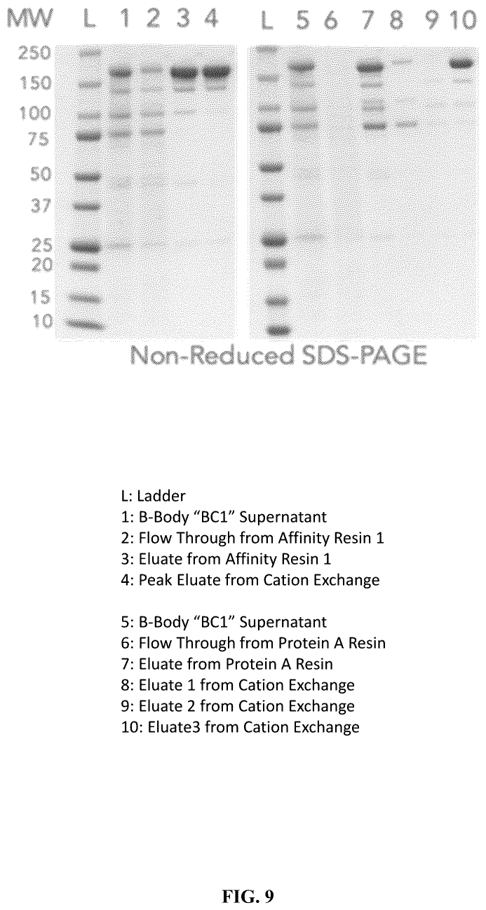

FIG. 9 shows SDS-PAGE gels under non-reducing conditions. As seen in lane 3, single-step purification of “BC1” with CH1 affinity resin provides a nearly homogeneous single band, with lane 4 showing minimal additional purification with a subsequent cation exchange polishing step. Lane 7, by comparison, shows less substantial purification using standard Protein A purification, with lanes 8-10 demonstrating further purification of the Protein A purified material using cation exchange chromatography.

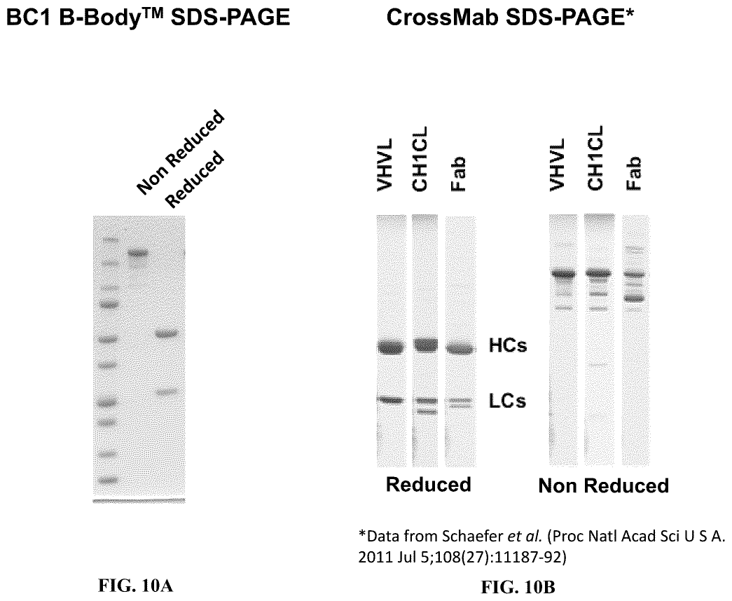

FIG. 10 compares SDS-PAGE gels of “BC1” after single-step CH1-affinity purification, under both non-reducing and reducing conditions (Panel A) with SDS-PAGE gels of a CrossMab bispecific antibody under non-reducing and reducing conditions as published in the referenced literature (Panel B).

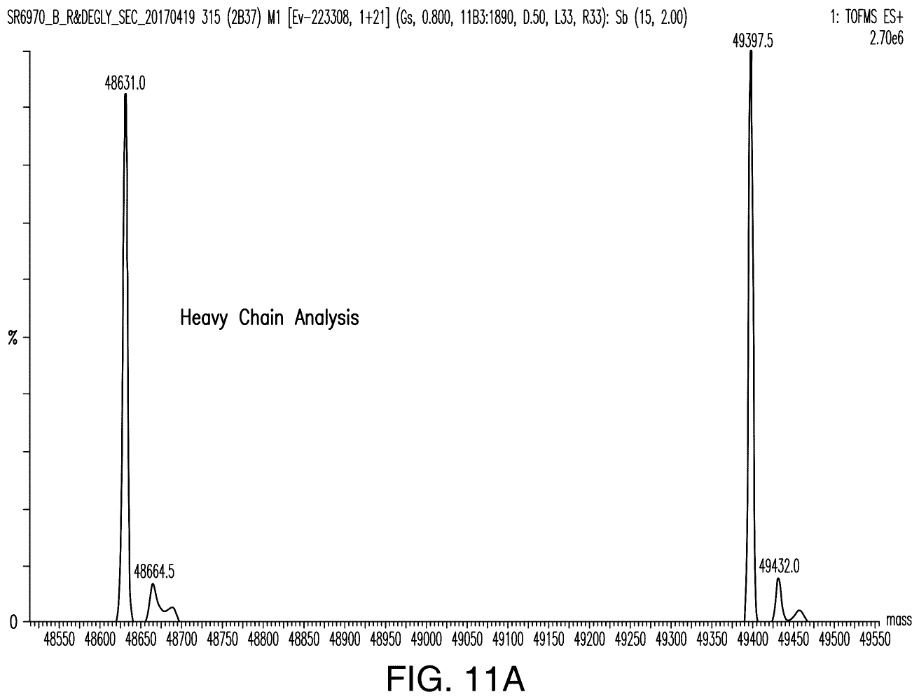

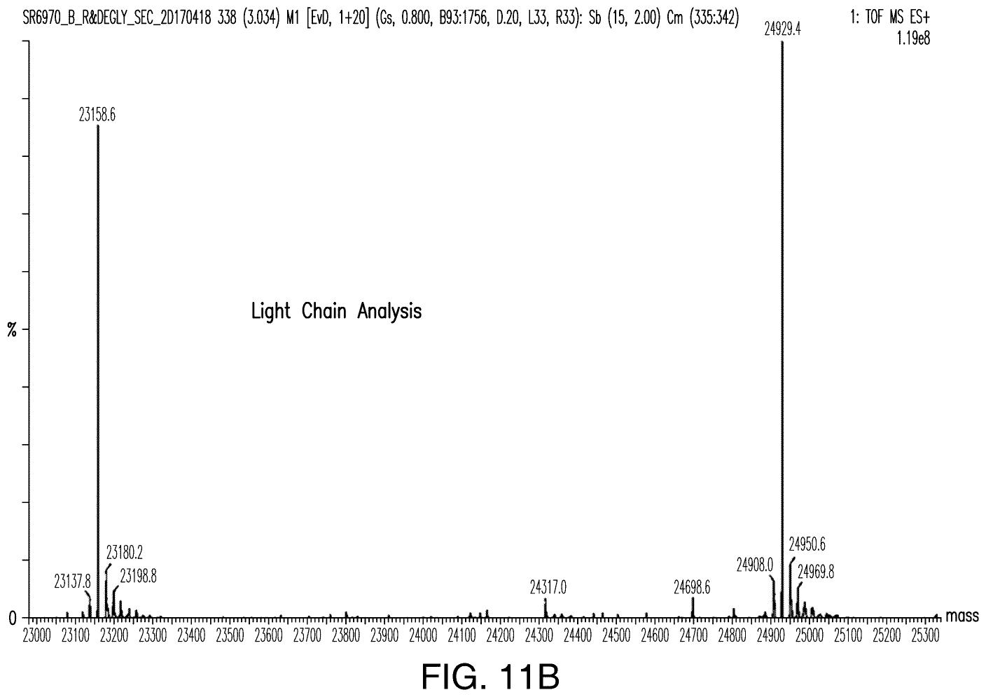

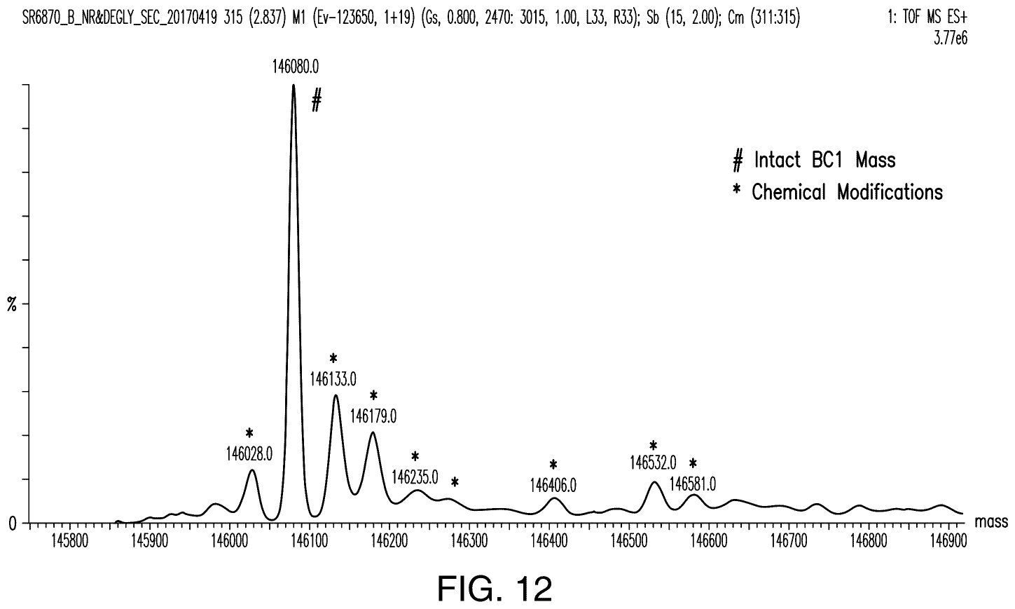

FIG. 11 shows mass spec analysis of “BC1”, demonstrating two distinct heavy chains (FIG. 11A) and two distinct light chains (FIG. 111B) under reducing conditions. The mass spectrometry data in FIG. 12 confirms the absence of incomplete pairing after purification.

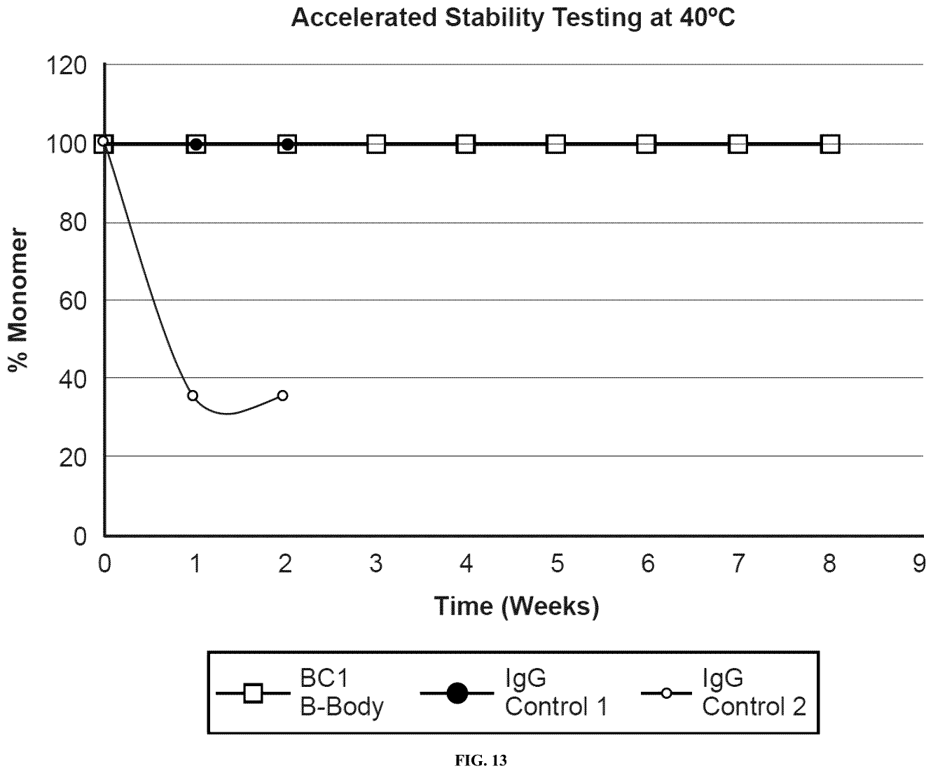

Accelerated stability testing was performed to evaluate the long-term stability of the “BC1” B-Body design. The purified B-Body was concentrated to 8.6 mg/ml in PBS buffer and incubated at 40° C. The structural integrity was measured weekly using analytical size exclusion chromatography (SEC) with a Shodex KW-803 column. The structural integrity was determined by measuring the percentage of intact monomer (% Monomer) in relation to the formation of aggregates. Data are shown in FIG. 13. The IgG Control 1 is a positive control with good stability properties. IgG Control 2 is a negative control that is known to aggregate under the incubation conditions. The “BC1” B-Body has been incubated for 8 weeks without any loss of structural integrity as determined by the analytical SEC.

We have also determined that “BC1” has high thermostability, with a TM of the bivalent construct of ˜72° C.

Table 1 compares “BC1” to CrossMab in key developability characteristics:

TABLE 1

| | | Roche | |
|---|---|---|---|
| Parameter | Unit | CrossMab* | “BC1” |
| Purification yield after protein | mg/L | 58.5 | 300 |
| A/SEC | | | |
| Homogeneity After purification | % SEC Area | 50-85 | 98 |
| Denaturation Temp (Tm) | degrees C. | 69.2 | 72 |

*Data from Schaefer et al. (Proc Natl Acad Sci USA. 2011 Jul. 5; 108(27): 11187-92)

### 6.13.4. Example 3: Bivalent bispecific B-Body “BC6”

We constructed a bivalent bispecific B-Body, termed “BC6”, that is identical to “BC1” but for retaining wild type residues in Domain B at residue 366 and Domain G at residue 351. “BC6” thus lacks the charge-pair cognates T366K and L351D that had been designed to facilitate correct pairing of domain B and domain G in “BC1”. Salient features of the “BC6” architecture are illustrated in FIG. 14.

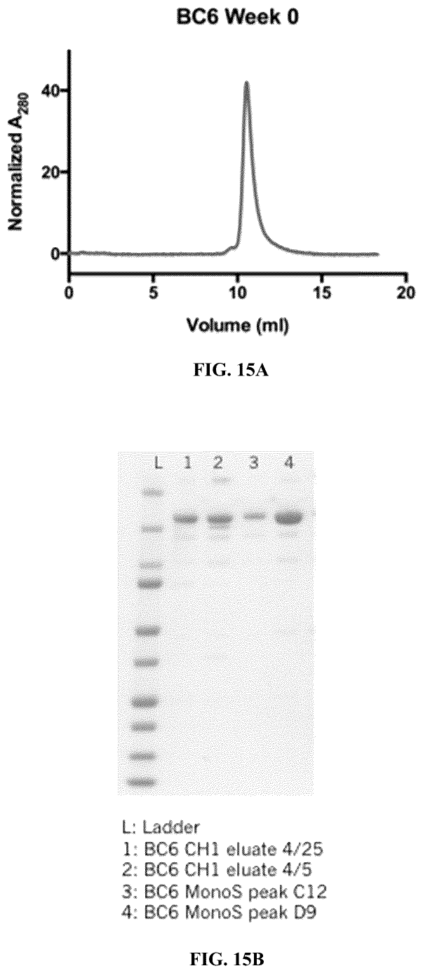

Notwithstanding the absence of the charge-pair residues present in “BC1”, we found that a single step purification of “BC6” using CH1 affinity resin resulted in a highly homogeneous sample. FIG. 15A shows SEC analysis of “BC6” following one-step purification using the CaptureSelect™ CH1 affinity resin. The data demonstrate that the single step CH1 affinity purification yields a single monodisperse peak, similar to what we observed with “BC1”, demonstrating that the disulfide bonds between polypeptide chains 1 and 2 and between polypeptide chains 3 and 4 are intact. The chromatogram also shows the absence of non-covalent aggregates.

FIG. 15B shows a SDS-PAGE gel under non-reducing conditions, with lane 1 loaded with a first lot of “BC6” after a single-step CH1 affinity purification, lane 2 loaded with a second lot of “BC6” after a single-step CH1 affinity purification. Lanes 3 and 4 demonstrate further purification can be achieved with ion exchange chromatography subsequent to CH1 affinity purification. 6.13.5. Example 4: Bivalent bispecific B-Bodies “BC28”, “BC29”, “BC30”, “BC31”

We constructed bivalent 1×1 bispecific B-Bodies “BC28”, “BC29”, “BC30” and “BC31” having an engineered disulfide within the CH3 interface in Domains B and G as an alternative S-S linkage to the C-terminal disulfide present in “BC1” and “BC6”. Literature indicates that CH3 interface disulfide bonding is insufficient to enforce orthogonality in the context of Fc CH3 domains. The general architecture of these B-Body constructs is schematized in FIG. 16 with salient features of “BC28” summarized below:
Polypeptide chain 1: “BC28” chain 1 (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
Polypeptide chain 2: “BC28” chain 2 (SEQ ID NO:25)
   Domain F=VH (Antigen “A”)
   Domain G=CH3 (S354C; 445P, 446G, 447K insertion)
Polypeptide chain 3: “BC1” chain 3 (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
Polypeptide chain 4: “BC1” chain 4 (SEQ ID NO:11)
   Domain L=VH (“Nivo”)
   Domain M=CH1.

The “BC28” A:F antigen binding site is specific for “Antigen A”. The “BC28” H:L antigen binding site is specific for PD1 (nivolumab sequences). “BC28” domain B has the following changes as compared to wild type CH3: Y349C; 445P, 446G, 447K insertion. “BC28” domain E has the following changes as compared to wild type CH3: S354C, T366W. “BC28” domain G has the following changes as compared to wild type: S354C; 445P, 446G, 447K insertion.

“BC28” thus has an engineered cysteine at residue 349C of Domain B and engineered cysteine at residue 354C of domain G (“349C-354C”).

“BC29” has engineered cysteines at residue 351C of Domain B and 351C of Domain G (“351C-351C”). “BC30” has an engineered cysteine at residue 354C of Domain B and 349C of Domain G (“354C-349C”). BC31 has an engineered cysteine at residue 394C and engineered cysteine at 394C of Domain G (“394C-394C”). BC32 has engineered cysteines at residue 407C of Domain B and 407C of Domain G (“407C-407C”).

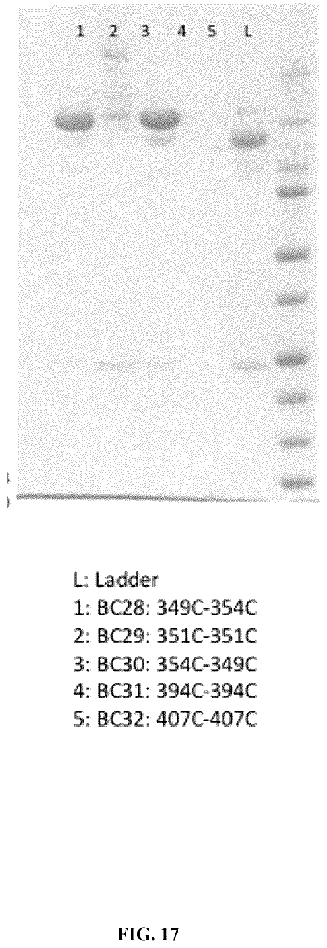

FIG. 17 shows SDS-PAGE analysis under non-reducing conditions following one-step purification using the CaptureSelect™ CH1 affinity resin. Lanes 1 and 3 show high levels of expression and substantial homogeneity of intact “BC28” (lane 1) and “BC30” (lane 3). Lane 2 shows oligomerization of BC29. Lanes 4 and 5 show poor expression of BC31 and BC32, respectively, and insufficient linkage in BC32. Another construct, BC9, which had cysteines introduced at residue 392 in domain B and 399 in Domain G (“392C-399C”), a disulfide pairing reported by Genentech, demonstrated oligomerization on SDS PAGE (data not shown).

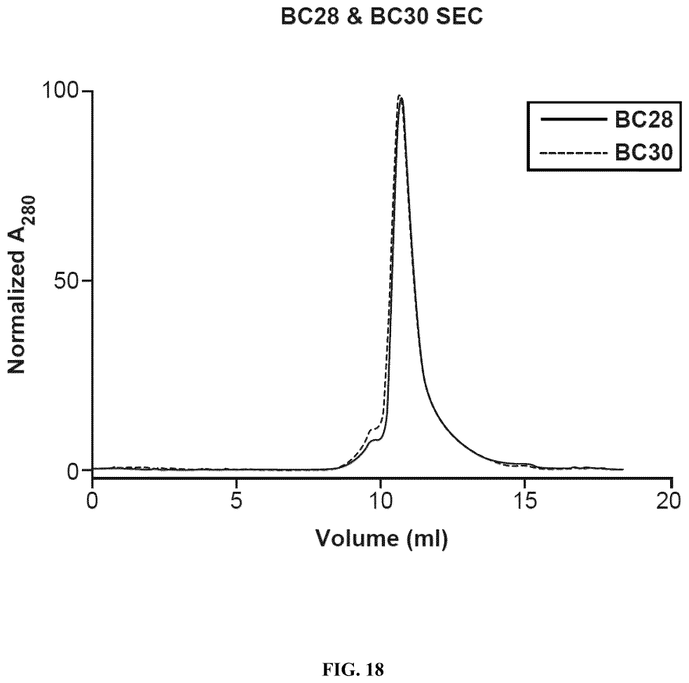

FIG. 18 shows SEC analysis of “BC28” and “BC30” following one-step purification using the CaptureSelect™ CH1 affinity resin. We have also demonstrated that “BC28” can readily be purified using a single step purification using Protein A resin (results not shown). 6.13.6. Example 5: Bivalent bispecific B-Body “BC44”

FIG. 19 shows the general architecture of the bivalent bispecific 1×1 B-Body “BC44”, our currently preferred bivalent bispecific 1×1 construct.
first polypeptide chain (“BC44” chain 1) (SEQ ID NO:32)
   Domain A=VL (Antigen “A”)
   Domain B=CH3 (P343V; Y349C; 445P, 446G, 447K insertion)
   Domain E=CH2
   Domain E=CH3 (S354C, T366W)
second polypeptide chain (=“BC28” polypeptide chain 2) (SEQ NO:25)
   Domain F=VH (Antigen “A”)
   Domain G=CH3 (S354C; 445P, 446G, 447K insertion)
third polypeptide chain (=“BC1” polypeptide chain 3) (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
fourth polypeptide chain (=“BC1” polypeptide chain 4) (SEQ ID NO:11)
   Domain L=VH (“Nivo”)
   Domain M=CH1.

### 6.13.7. Example 6: Variable-CH3 junction engineering

We produced a series of variants in which we mutated the VL—CH3 junction between Domains A and B and the VH-CH3 junction between domains F and G to assess the expression level, assembly and stability of bivalent 1×1 B-Body constructs. Although there are likely many solutions, to reduce introduction of T cell epitopes we chose to only use residues found naturally within the VL, VH and CH3 domains. Structural assessment of the domain architecture further limits desirable sequence combinations. Table 2 and Table 3 below show junctions for several junctional variants based on “BC1” and other bivalent constructs.

TABLE 2
Variants of Variable Domain/Constant Domain
Junctions for 1^{st} Polypeptide Chain

| | VL | | | | | | CH3 | | | | |
|---|---|---|---|---|---|---|---|---|---|---|---|
| Variant | 106 | 107 | 108 | 109 | 110 | 111 | 343 | 344 | 345 | 346 | Sequence |
| BC1 | I | K | R | T | | | P | R | E | P | IKRTPREP |
| | | | | | | | | | | | (SEQ ID |
| | | | | | | | | | | | NO: 57) |
| BC13 | I | K | R | T | | | P | R | E | P | IKRTPREP |
| | | | | | | | | | | | (SEQ ID |
| | | | | | | | | | | | NO: 57) |
| BC14 | I | K | R | T | | | P | R | E | P | IKRTPREP |
| | | | | | | | | | | | (SEQ ID |
| | | | | | | | | | | | NO: 57) |
| BC15 | I | K | R | T | V | | | R | E | P | IKRTVREP |
| | | | | | | | | | | | (SEQ ID |
| | | | | | | | | | | | NO: 58) |
| BC16 | I | K | R | T | | | | R | E | P | IKRTREP |
| | | | | | | | | | | | (SEQ ID |
| | | | | | | | | | | | NO: 59) |
| BC17 | I | K | R | T | V | | P | R | E | P | IKRTVPREP |
| | | | | | | | | | | | (SEQ ID |
| | | | | | | | | | | | NO: 60) |
| BC24 | I | K | R | T | | | P | R | E | P | IKRTPREP |
| | | | | | | | | | | | (SEQ ID |
| | | | | | | | | | | | NO: 57) |
| BC25 | I | K | R | T | | | P | R | E | P | IKRTPREP |
| | | | | | | | | | | | (SEQ ID |
| | | | | | | | | | | | NO: 57) |
| BC26 | I | K | R | T | V | A | | | E | P | IKRTVAEP |
| | | | | | | | | | | | (SEQ ID |
| | | | | | | | | | | | NO: 61) |
| BC27 | I | K | R | T | V | A | P | R | E | P | IKRTVAPREP |
| | | | | | | | | | | | (SEQ ID |
| | | | | | | | | | | | NO: 62) |
| BC44 | I | K | R | T | | | V | R | E | P | IKRTVREP |
| | | | | | | | | | | | (SEQ ID |
| | | | | | | | | | | | NO: 58) |
| BC45 | I | K | R | T | | | P | R | E | P | IKRTPREP |
| | | | | | | | | | | | (SEQ ID |
| | | | | | | | | | | | NO: 57) |
| BC5 | I | K | R | T | | | P | R | E | P | IKRTPREP |
| | | | | | | | | | | | (SEQ ID |
| | | | | | | | | | | | NO: 57) |
| BC6 | I | K | R | T | | | P | R | E | P | IKRTPREP |
| | | | | | | | | | | | (SEQ ID |
| | | | | | | | | | | | NO: 57) |
| BC28 | I | K | R | T | | | P | R | E | P | IKRTPREP |
| | | | | | | | | | | | (SEQ ID |
| | | | | | | | | | | | NO: 57) |
| BC30 | I | K | R | T | | | P | R | E | P | IKRTPREP |
| | | | | | | | | | | | (SEQ ID |
| | | | | | | | | | | | NO: 57) |

TABLE 3
Variants of Variable Domain/Constant Domain
Junctions for 2^{nd} Polypeptide Chain

| | VH | | | | | | | CH3 | | | | |
|---|---|---|---|---|---|---|---|---|---|---|---|---|
| Variant | 112 | 113 | 114 | 115 | 116 | 117 | 118 | 343 | 344 | 345 | 346 | Sequence |
| BC1 | S | S | A | S | | | | P | R | E | P | SSASPREP |
| | | | | | | | | | | | | (SEQ ID NO: 63) |
| BC13 | S | S | A | S | T | | | | R | E | P | SSASTREP |
| | | | | | | | | | | | | (SEQ ID NO: 64) |
| BC14 | S | S | A | S | T | | | P | R | E | P | SSASTPREP |
| | | | | | | | | | | | | (SEQ ID NO: 65) |
| BC15 | S | S | A | S | | | | P | R | E | P | SSASPREP |
| | | | | | | | | | | | | (SEQ ID NO: 63) |
| BC16 | S | S | A | S | | | | P | R | E | P | SSASPREP |
| | | | | | | | | | | | | (SEQ ID NO: 63) |
| BC17 | S | S | A | S | | | | P | R | E | P | SSASPREP |
| | | | | | | | | | | | | (SEQ ID NO: 63) |
| BC24 | S | S | A | S | T | K | G | | | E | P | SSASTKGEP |
| | | | | | | | | | | | | (SEQ ID NO: 66) |
| BC25 | S | S | A | S | T | K | G | | R | E | P | SSASTKGREP |
| | | | | | | | | | | | | (SEQ ID NO: 67) |
| BC26 | S | S | A | S | | | | P | R | E | P | SSASPREP |
| | | | | | | | | | | | | (SEQ ID NO: 63) |
| BC27 | S | S | A | S | | | | P | R | E | P | SSASPREP |
| | | | | | | | | | | | | (SEQ ID NO: 63) |
| BC44 | S | S | A | S | | | | P | R | E | P | SSASPREP |
| | | | | | | | | | | | | (SEQ ID NO: 63) |
| BC45 | S | S | A | S | | | | P | R | E | P | SSASPREP |
| | | | | | | | | | | | | (SEQ ID NO: 63) |
| BC5 | S | S | A | S | | | | P | R | E | P | SSASPREP |
| | | | | | | | | | | | | (SEQ ID NO: 63) |
| BC6 | S | S | A | S | | | | P | R | E | P | SSASPREP |
| | | | | | | | | | | | | (SEQ ID NO: 63) |
| BC28 | S | S | A | S | | | | P | R | E | P | SSASPREP |
| | | | | | | | | | | | | (SEQ ID NO: 63) |
| BC30 | S | S | A | S | | | | P | R | E | P | SSASPREP |
| | | | | | | | | | | | | (SEQ ID NO: 63) |

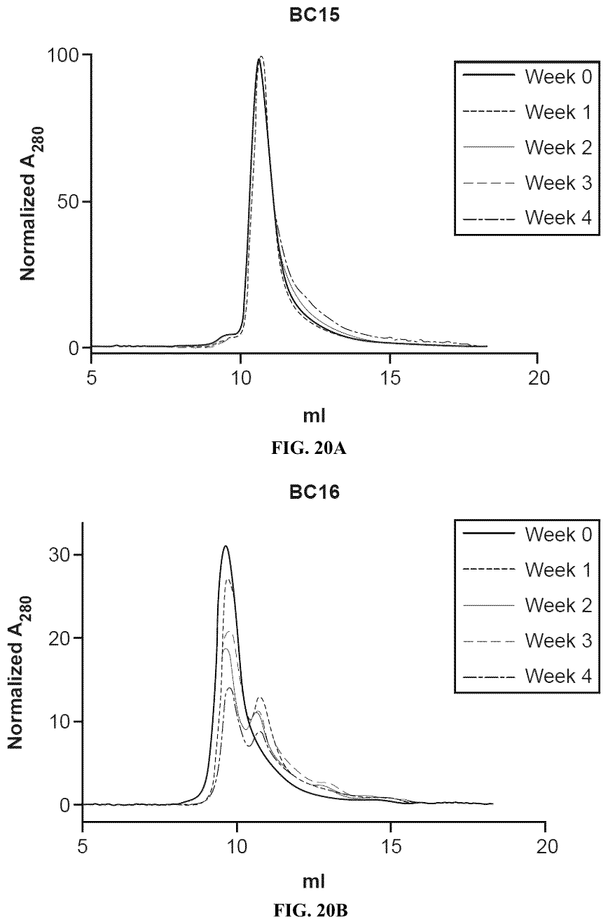

FIG. 20 shows size exclusion chromatography of “BC15” and “BC16” samples at the indicated week of an accelerated stability testing protocol at 40° C. “BC15” remained stable; “BC16” proved to be unstable over time.

### 6.13.8. Example 7: Trivalent 2×1 Bispecific B-Body construct (“BC1-2×1”)

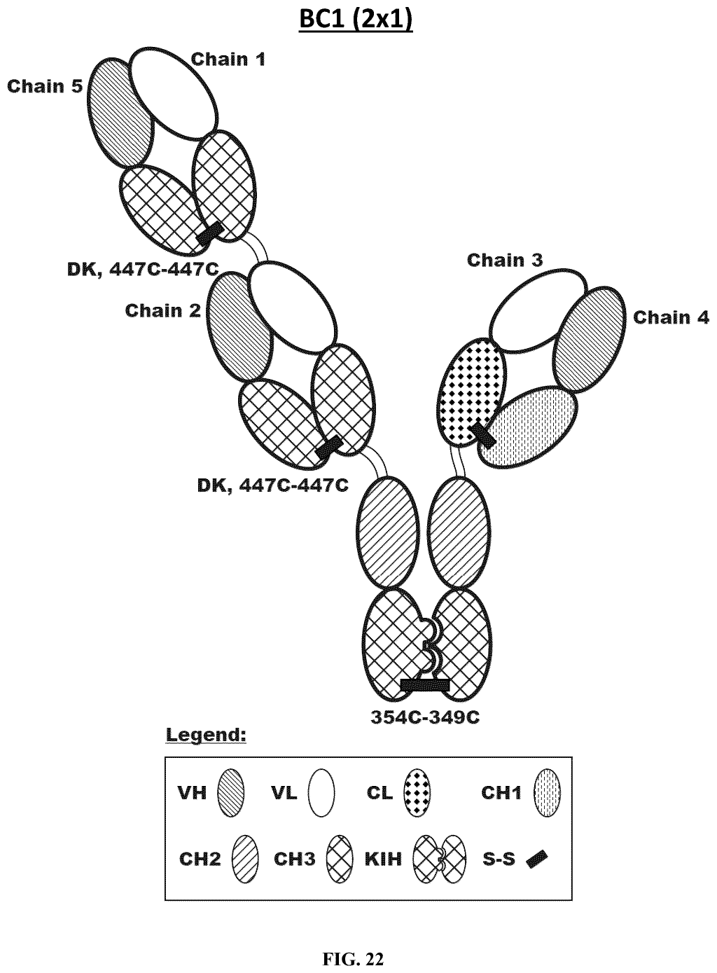

We constructed a trivalent 2×1 bispecific B-Body “BC1-2×1” based on “BC1”. Salient features of the architecture are illustrated in FIG. 22.

In greater detail, using the domain and polypeptide chain references summarized in FIG. 21,
1^{st} polypeptide chain
   Domain N=VL (“Antigen A”)
   Domain 0=CH3 (T366K, 447C)
   Domain A=VL (“Antigen A”)
   Domain B=CH3 (T366K, 447C)
   Domain D=CH2
   Domain E=CH3 (Knob, 354C)
5^{th} polypeptide chain (=“BC1” chain 2)
   Domain P=VH (“Antigen A”)
   Domain Q=CH3 (L351D, 447C)
2^{nd} polypeptide chain (=“BC1” chain 2)
   Domain F=VH (“Antigen A”)
   Domain G=CH3 (L351D, 447C)
3^{rd} polypeptide chain (=“BC1” chain 3)
   Domain H=VL (“Nivo”)
   Domain I=CL (Kappa)
   Domain J=CH2
   Domain K=CH3 (Hole, 349C)
4^{th} polypeptide chain (=“BC1” chain 4)
   Domain L=VH (“Nivo”)
   Domain M=CH1.

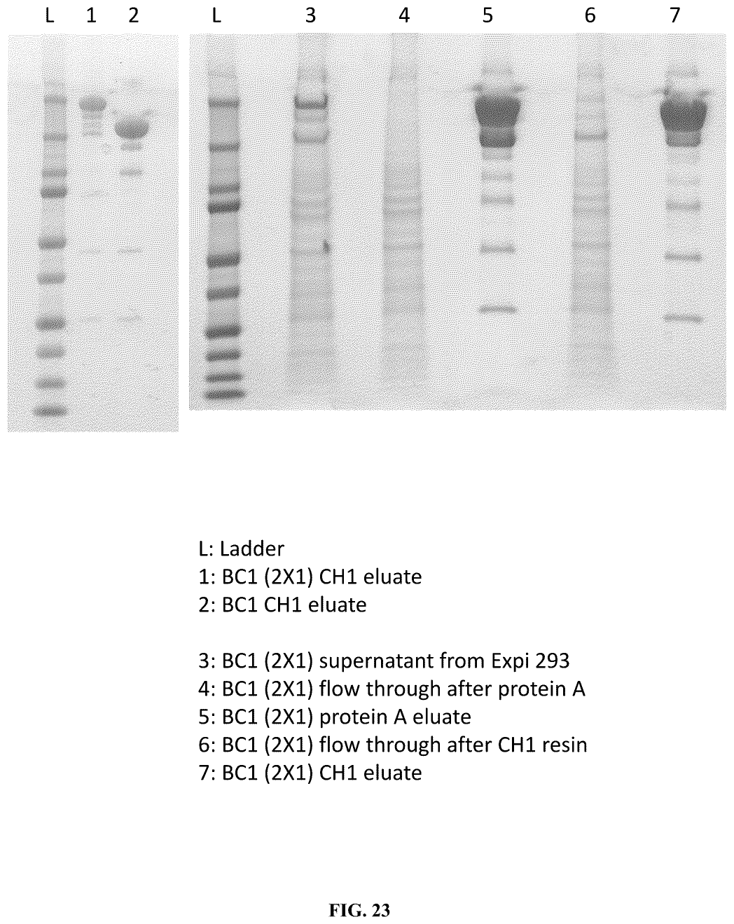

FIG. 23 shows non-reducing SDS-PAGE of protein expressed using the ThermoFisher Expi293 transient transfection system.

Lane 1 shows the eluate of the trivalent 2×1 “BC1-2X1” protein following one-step purification using the CaptureSelect™ CH1 affinity resin. Lane 2 shows the lower molecular weight, faster migrating, bivalent “BC1” protein following one-step purification using the CaptureSelect™ CH1 affinity resin. Lanes 3-5 demonstrate purification of “BC1-2×1” using protein A. Lanes 6 and 7 show purification of “BC1-2×1” using CH1 affinity resin.

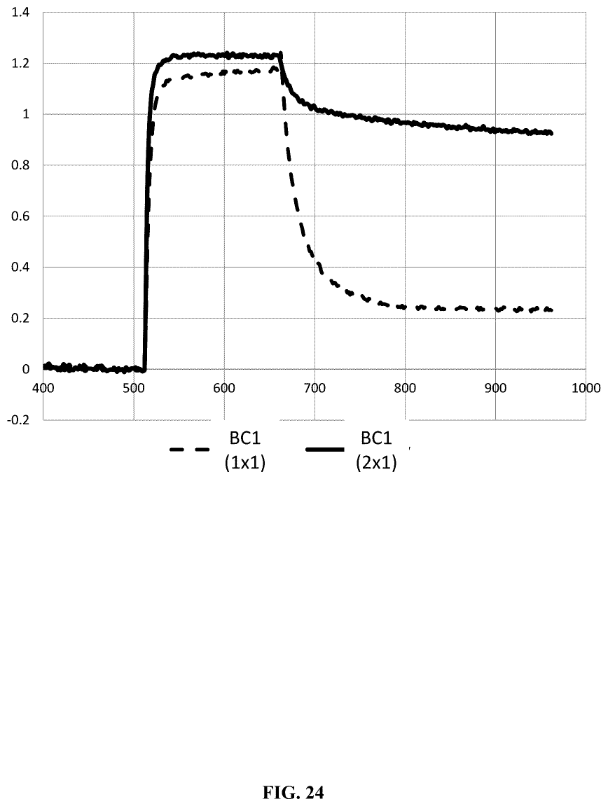

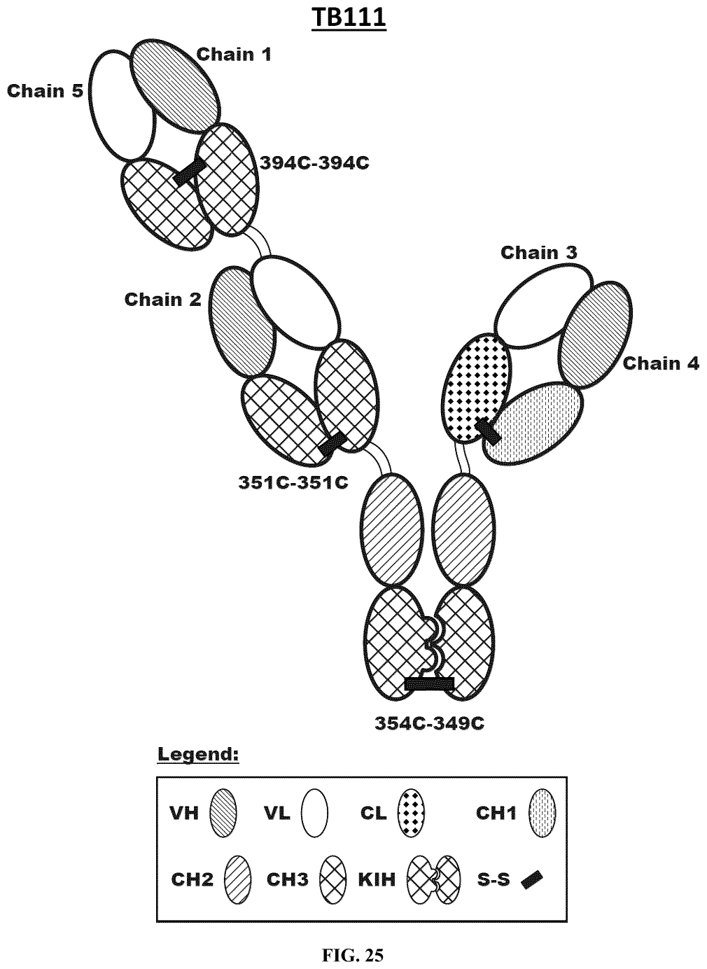

FIG. 24 compares the avidity of the bivalent “BC1” construct to the avidity of the trivalent 2×1 “BC1-2×1” construct using an Octet (Pall ForteBio) analysis. Biotinylated antigen “A” is immobilized on the surface, and the antibody constructs are passed over the surface for binding analysis.

### 6.13.9. Example 8: Trivalent 2×1 Trispecific B-Body construct (“TB111”)

We designed a trivalent 2×1 trispecific molecule, “TB111”, having the architecture schematized in FIG. 25. With reference to the domain naming conventions set forth in FIG. 21, TB 111 has the following architecture (“Ada” indicates a V region from adalimumab):
polypeptide chain 1
   Domain N: VH (“Ada”)
   Domain 0: CH3 (T366K, 394C)
   Domain A: VL (“Antigen A”)
   Domain B: CH3 (T366K, 349C)
   Domain D: CH2
   Domain E: CH3 (Knob, 354C)
polypeptide chain 5
   Domain P: VL (“Ada”)
   Domain Q: CH3 (L351D, 394C)
polypeptide chain 2
   Domain F: VH (“Antigen A”)
   Domain G: CH3 (L351D, 351C)
polypeptide chain 3
   Domain H: VL (“Nivo”)
   Domain I: CL (kappa)
   Domain J: CH2
   Domain K: CH3 (Hole, 349C)
polypeptide chain 4 (=“BC1” chain 4)
   Domain L: VH (“Nivo”)
   Domain M: CH1
      This construct did not express.

### 6.13.10. Example 9: Trivalent 1×2 Bispecific Construct (“BC28-1×2”)

We constructed a trivalent 1×2 bispecific B-Body having the following domain and chain structure with reference to the domain and chain nomenclature set forth in FIG. 26:
1^{st} polypeptide chain (=“BC28” chain 1) (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:37)
   Domain R=VL (Antigen “A”)
   Domain S=CH3 (Y349C; 445P, 446G, 447K insertion)
   Linker=GSGSGS (SEQ ID NO:541)
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11):
   Domain L=VH (“Nivo”)
   Domain M=CH1.
6^{th} polypeptide chain (=“BC28” chain 2) (SEQ ID NO:25)
   Domain T=VH (Antigen “A”)
   Domain U=CH3 (S354C; 445P, 446G, 447K insertion)

The A:F antigen binding site is specific for “Antigen A”, as is the H:L binding antigen binding site. The R:T antigen binding site is specific for PD. The specificity of this construct is thus Antigen “A” x (PD1-Antigen “A”).

### 6.13.11. Example 10: Trivalent 1×2 bispecific construct (“CTLA4-4 × Nivo x CTLA4-4”)

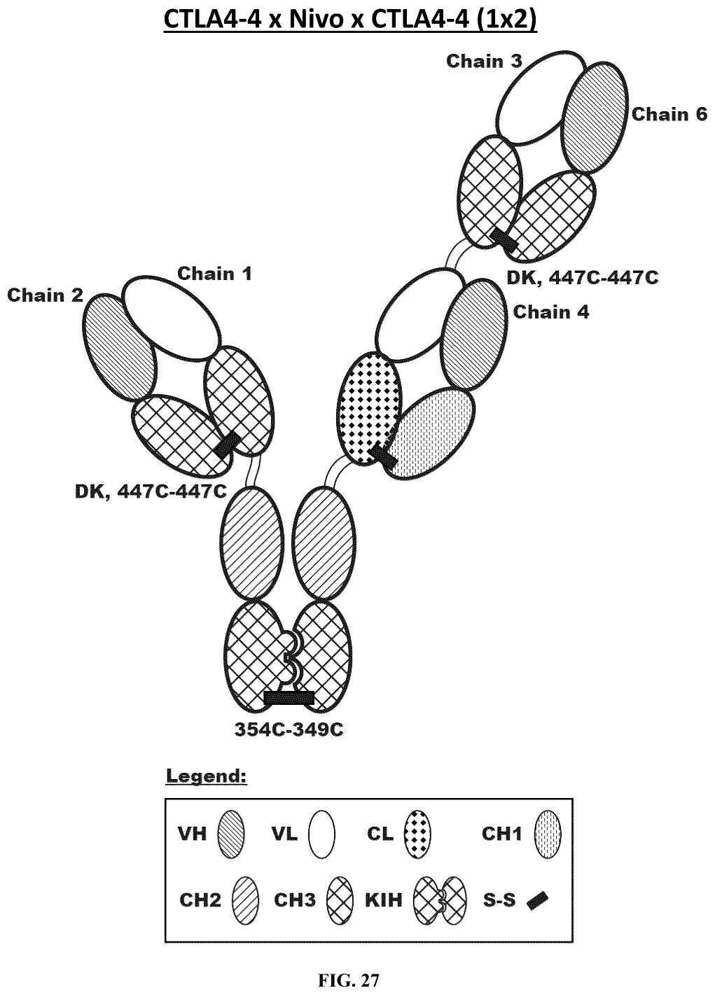

We constructed a trivalent 1×2 bispecific molecule having the general structure schematized in FIG. 27 (“CTLA4-4 × Nivo x CTLA4-4”). Domain nomenclature is set forth in FIG. 26.

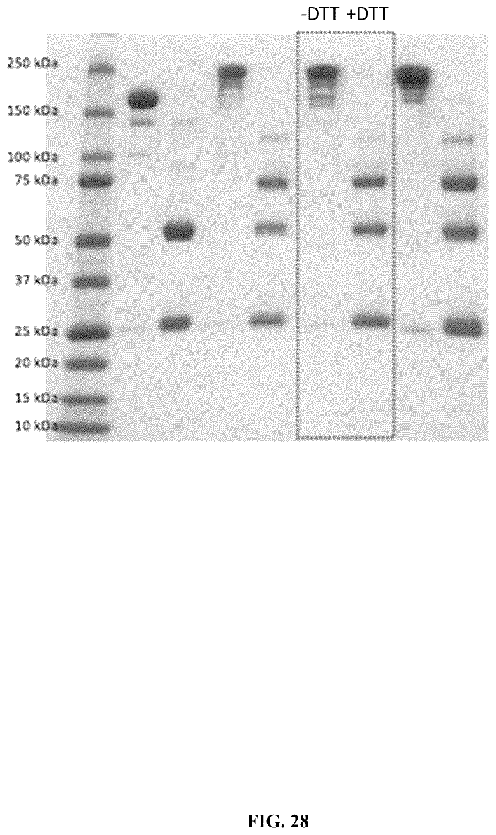

FIG. 28 is a SDS-PAGE gel in which the lanes showing the “CTLA4-4 × Nivo x CTLA4-4” construct under non-reducing and reducing conditions have been boxed.

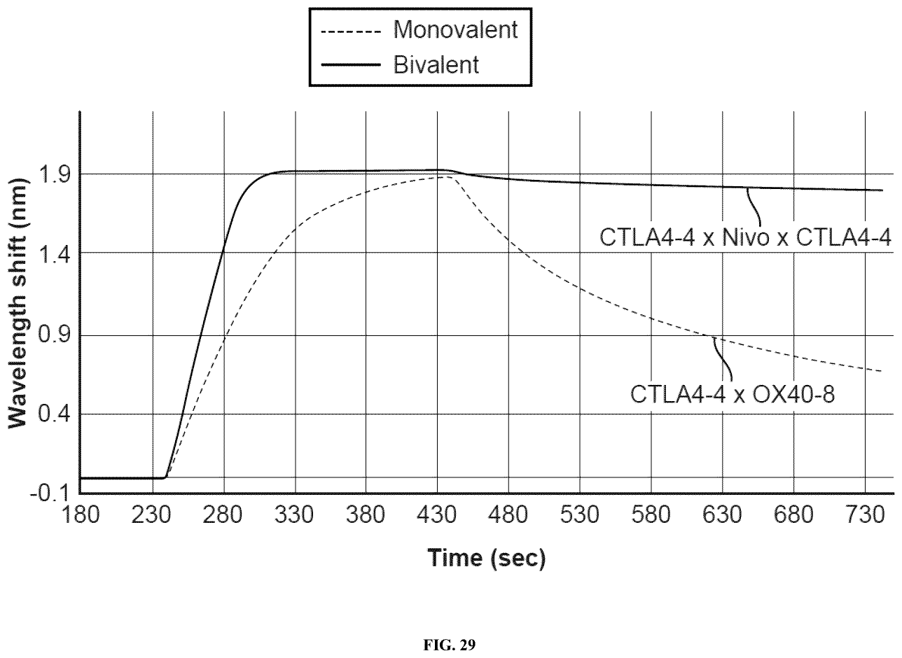

FIG. 29 compares antigen binding of two antibodies: “CTLA4-4 × OX40-8” and “CTLA4-4 × Nivo x CTLA4-4”. “CTLA4-4 × OX40-8” binds to CTLA4 monovalently; while “CTLA4-4 × Nivo x CTLA4-4” bind to CTLA4 bivalently.

### 6.13.12. Example 11: Trivalent 1×2 trispecific construct “BC28-1×1×1a”

We constructed a trivalent 1×2 trispecific molecule having the general structure schematized in FIG. 30. With reference to the domain and chain nomenclature set forth in FIG. 26,
1^{st} polypeptide chain (=“BC28” chain1) [SEQ ID NO:24]
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:45)
   Domain R=VL (CTLA4-4)
   Domain S=CH3 (T366K; 445K, 446S, 447C insertion)
   Linker=GSGSGS (SEQ ID NO:541)
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11)
   Domain L=VH (“Nivo”)
   Domain M=CH1
6^{th} polypeptide chain (=hCTLA4-4 chain2) (SEQ ID NO:53)
   Domain T=VH (CTLA4)
   Domain U=CH3 (L351D, 445G, 446E, 447C insertion)

The antigen binding sites of this trispecific construct were: Antigen binding site A:F was specific for “Antigen A” Antigen binding site H:L was specific for PD1 (nivolumab sequence) Antigen binding site R:T was specific for CTLA4.

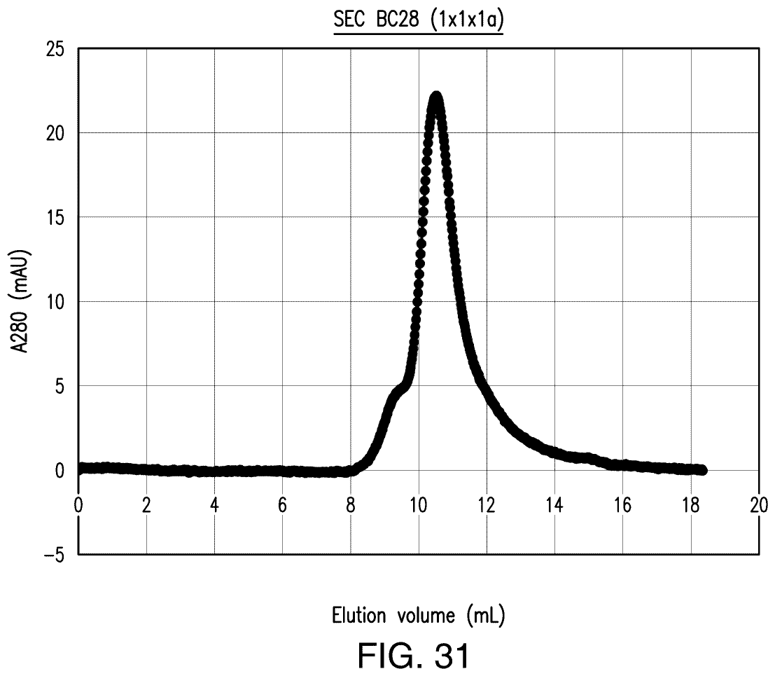

FIG. 31 shows size exclusion chromatography with “BC28-1x1x1a” following transient expression and one-step purification using the CaptureSelect™ CH1 affinity resin, demonstrating a single well-defined peak.

### 6.13.13. Example 12: SDS-PAGE Analysis of Bivalent and Trivalent Constructs

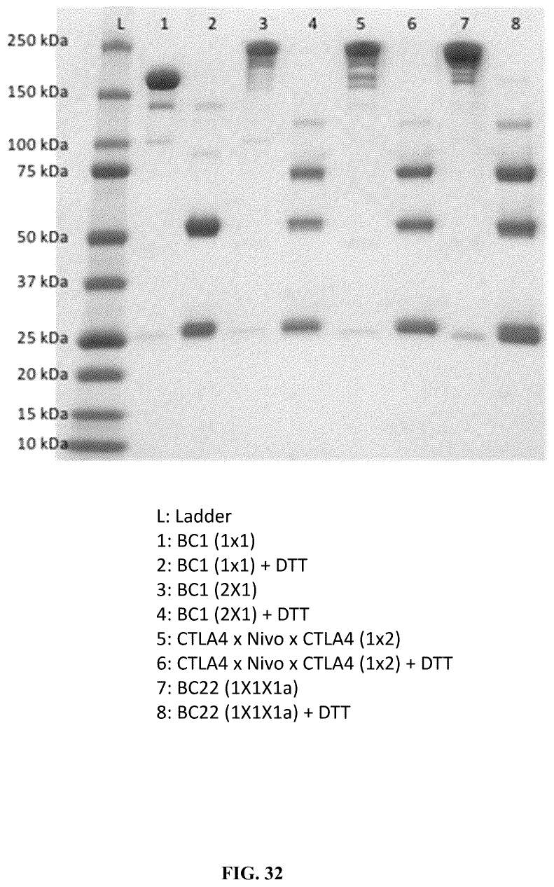

FIG. 32 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the trivalent bispecific 2×1 construct “BC1-2×1” (see Example 7). Lanes 5 (nonreducing) and 6 (reducing) are the trivalent 1×2 bispecific construct “CTLA4-4 × Nivo x CTLA4-4” (see Example 10). Lanes 7 (nonreducing) and 8 (reducing) are the trivalent 1×2 trispecific “BC28-1x1x1a” construct described in Example 11.

The SDS-PAGE gel demonstrates the complete assembly of each construct, with the predominant band in the non-reducing gel appearing at the expected molecular weight for each construct.

### 6.13.14. Example 13: Binding analysis

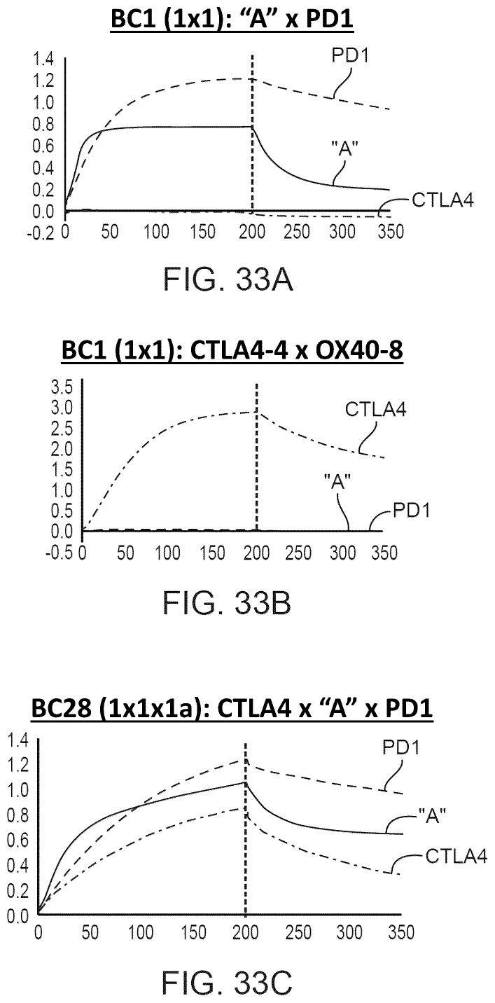

FIG. 33 shows Octet binding analyses to 3 antigens: PD1, Antigen “A”, and CTLA-4. In each instance, the antigen is immobilized and the B-Body is the analyte. For reference, 1×1 bispecifics “BC1” and “CTLA4-4 × OX40-8” were also compared to demonstrate 1×1 B-Bodies bind specifically only to antigens for which the antigen binding sites were selected.

FIG. 33A shows binding of “BC1” to PD1 and to Antigen “A”, but not CTLA4.

FIG. 33B shows binding of a bivalent bispecific 1×1 construct “CTLA4-4 × OX40-8” to CTLA4, but not to Antigen “A” or PD1. FIG. 33C shows the binding of the trivalent trispecific 1×2 construct, “BC28-1x1x1a” to PD1, Antigen “A”, and CTLA4.

### 6.13.15. Example 14: Tetravalent Constructs

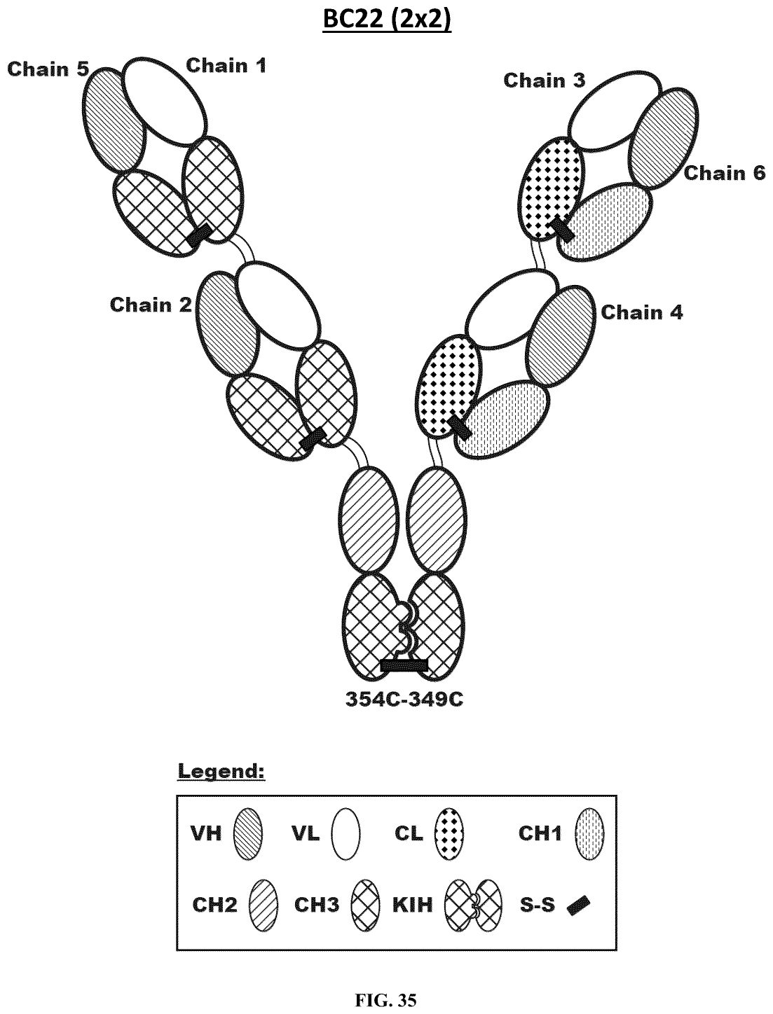

FIG. 35 shows the overall architecture of a 2×2 tetravalent bispecific construct “BC22-2×2”. The 2×2 tetravalent bispecific was constructed with “BC1” scaffold by duplicating each variable domain-constant domain segment. Domain nomenclature is schematized in FIG. 34.

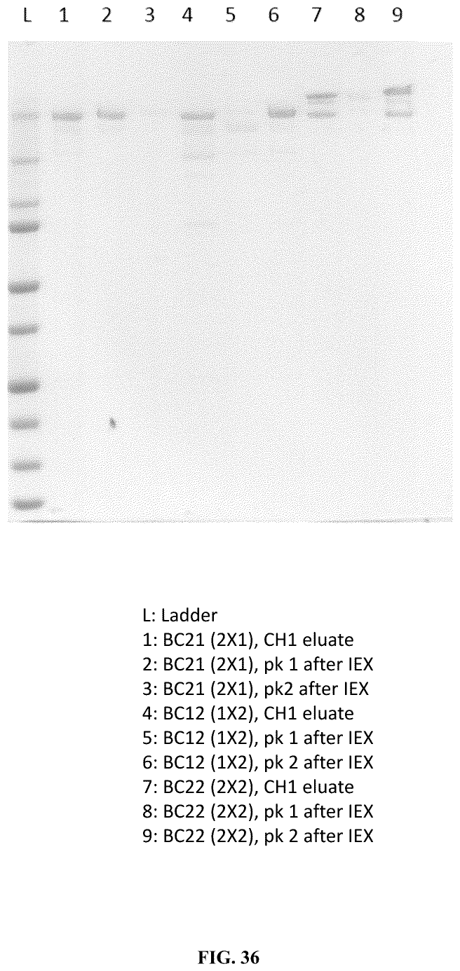

FIG. 36 is a SDS-PAGE gel. Lanes 7-9 show the “BC22-2×2” tetravalent construct respectively following one-step purification using the CaptureSelect™ CH1 affinity resin (“CH1 eluate”), and after an additional ion exchange chromatography purification (lane 8, “pk 1 after IEX”; lane 9, “pk 2 after IEX”). Lanes 1-3 are the trivalent 2×1 construct “BC21-2×1” after CH1 affinity purification (lane 1) and, in lanes 2 and 3, subsequent ion exchange chromatography. Lanes 4-6 are the 1×2 trivalent construct “BC12-1×2”.

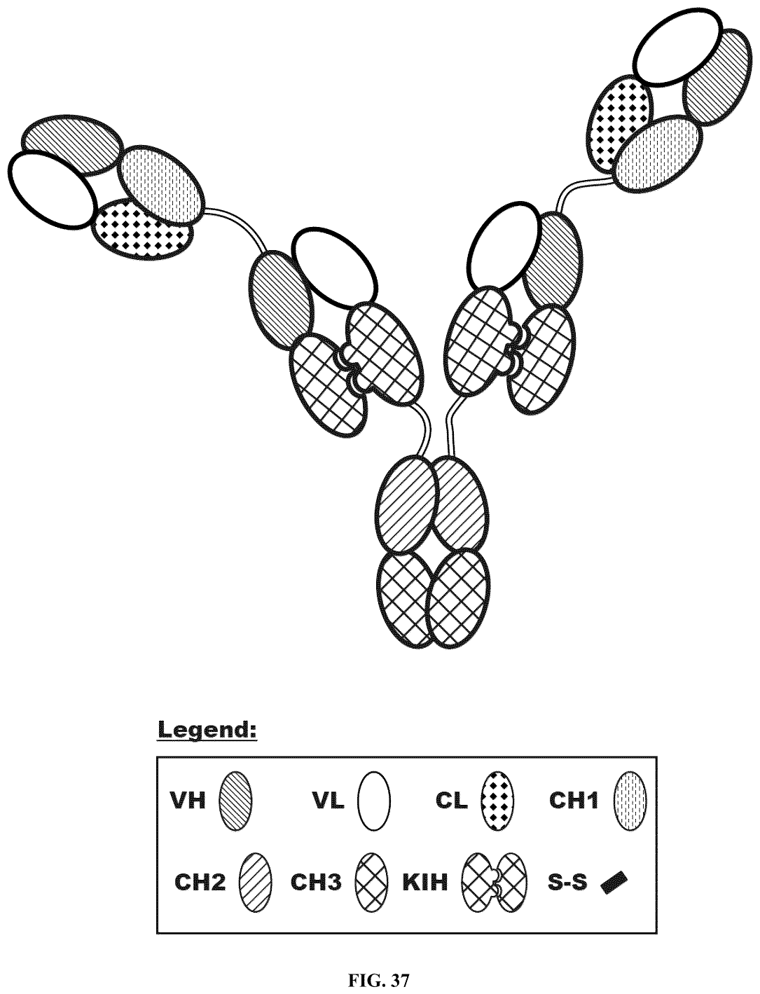

FIG. 37 shows the overall architecture of a 2×2 tetravalent construct.

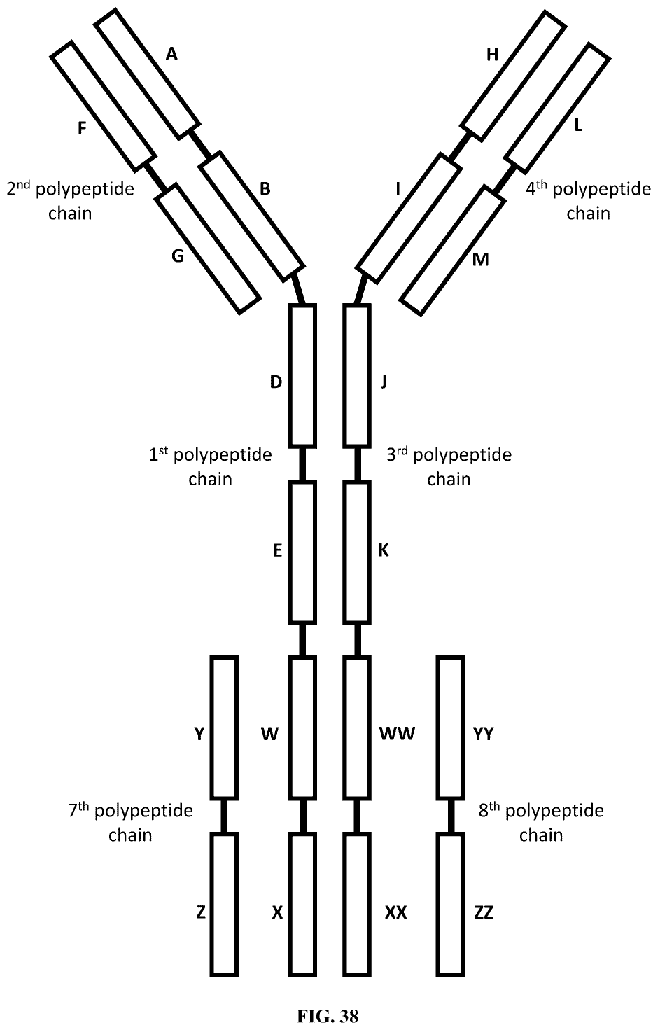

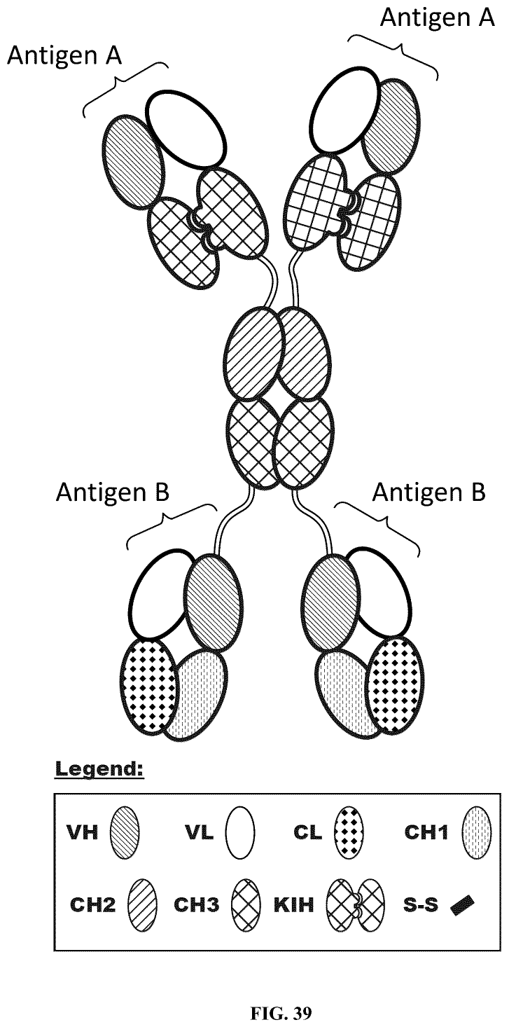

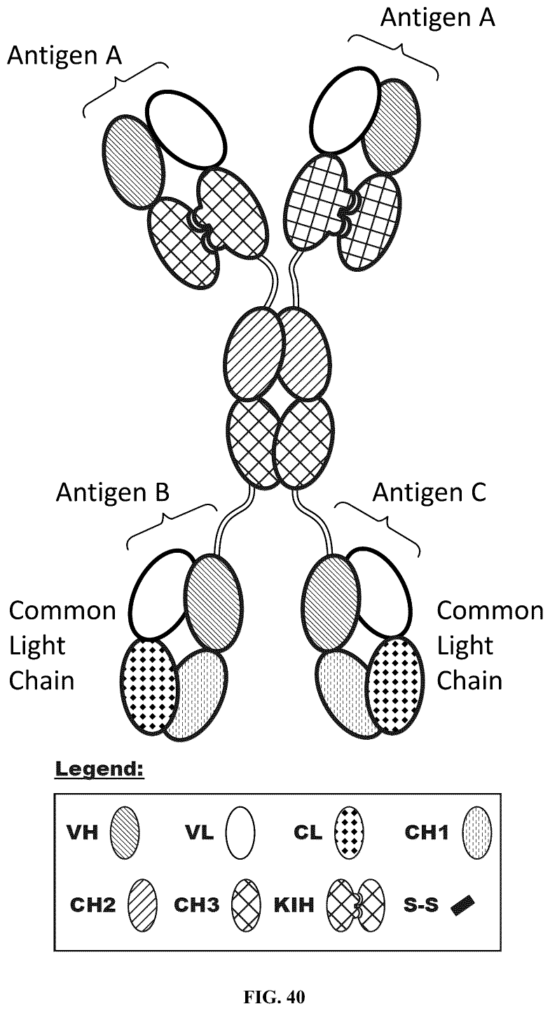

FIGS. 39 and 40 schematize tetravalent constructs having alternative architectures. Domain nomenclature is presented in FIG. 38.

### 6.13.16. Example 15: Bispecific Antigen Engagement by B-Body

A tetravalent bispecific 2×2 B-Body “B-Body-IgG 2×2” was constructed. In greater detail, using the domain and polypeptide chain nomenclature references summarized in FIG. 38,
1^{st} polypeptide chain
   Domain A=VL (Certolizumab)
   Domain B=CH3 (IgG1, knob)
   Domain D=CH2 (IgG1)
   Domain E=CH3 (IgG1)
   Domain W=VH (Antigen “A”)
   Domain X=CH1 (IgG1)
3^{rd} polypeptide chain (identical to first polypeptide chain)
   Domain H=VL (Certolizumab)
   Domain I=CH3 (IgG1, knob)
   Domain J=CH2 (IgG1)
   Domain K=CH3 (IgG1)
   Domain WW=VH (Antigen “A”)
   Domain XX=CH1 (IgG1)
2^{nd} polypeptide chain
   Domain F=VH (Certolizumab)
   Domain G=CH3 (IgG1, hole)
4^{th} polypeptide chain (identical to third polypeptide chain)
   Domain F=VH (Certolizumab)
   Domain G=CH3 (IgG1, hole)
7^{th} polypeptide chain
   Domain Y=VH (“Antigen A”)
   Domain Z=CL Kappa
8^{th} polypeptide chain (identical to seventh polypeptide chain)
   Domain YY=VH (“Antigen A”)
   Domain ZZ=CL Kappa.

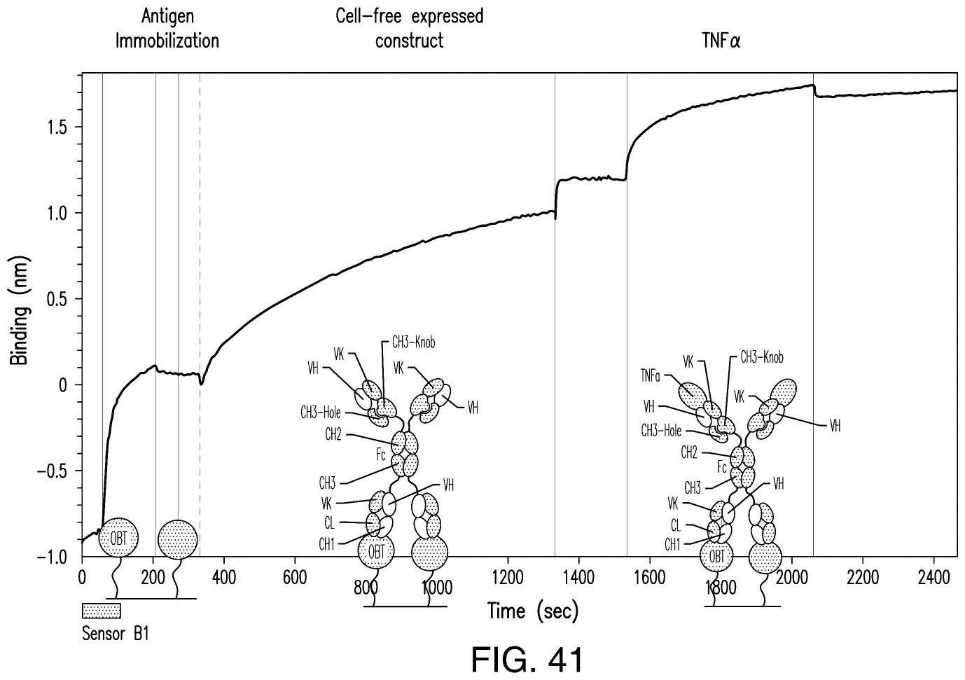

This was cloned and expressed as described in Example 1. Here, the BLI experiment consisted of immobilization of biotinylated antigen “A” on a streptavidin sensor, followed by establishing baseline with 10× kinetic buffer. The sensor was then dipped in cell-free expressed “B-Body-IgG 2×2” followed by establishment of a new baseline. Finally, the sensor was dipped in 100 nM TNFα where a second binding event was observed, confirming the bispecific binding of both antigens by a single “B-Body-IgG 2×2” construct. Results are shown in FIG. 41.

### 6.13.17. Example 16: Antigen-Specific Cell Binding of “BB-IgG 2×2”

Expi-293 cells were either mock transfected or transiently transfected with Antigen “B” using the Expi-293 Transfection Kit (Life Technologies). Forty-eight hours after transfection, the Expi-293 cells were harvested and fixed in 4% paraformaldehyde for 15 minutes at room temperature. The cells were washed twice in PBS. 200,000 Antigen B or Mock transfected Expi-293 cells were placed in a V-bottom 96 well plate in 100 μL of PBS.

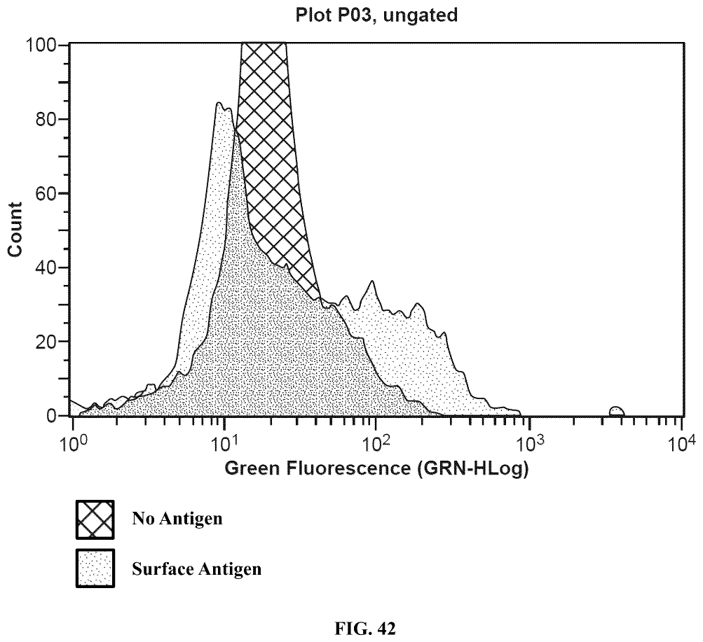

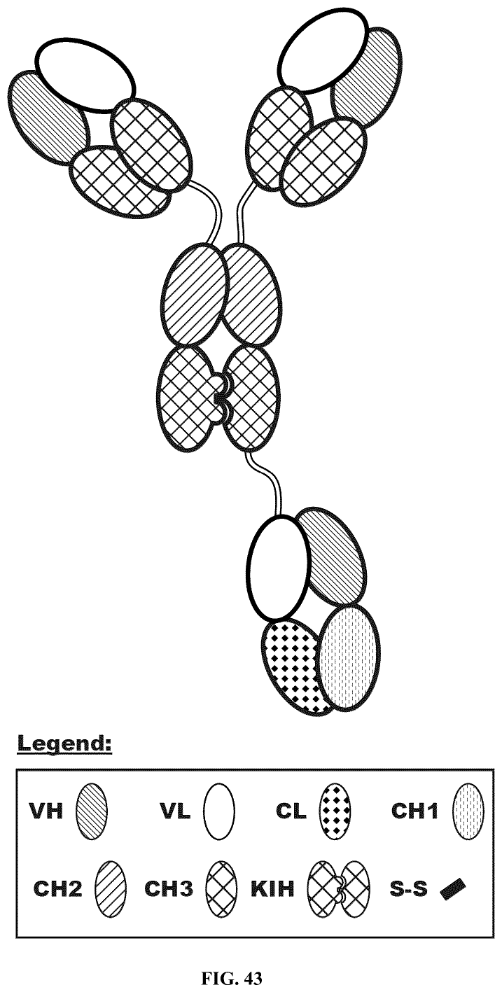

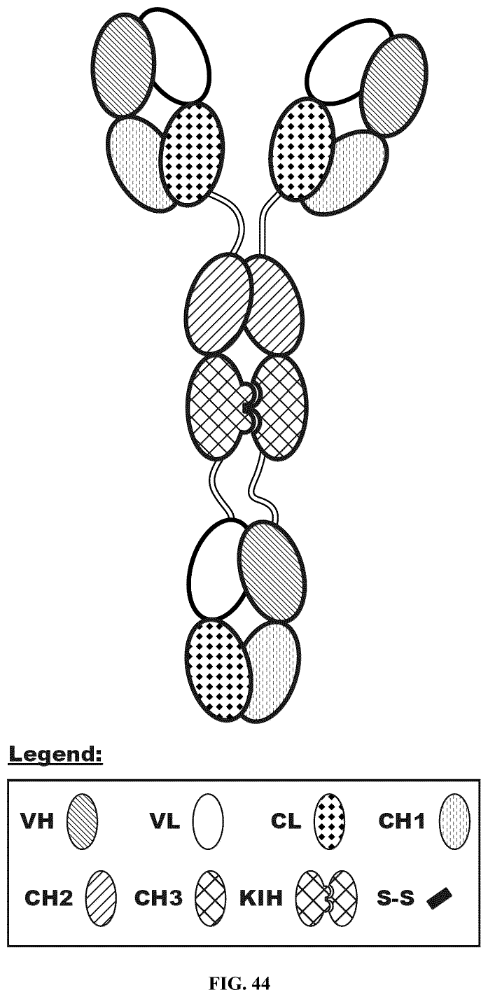

The cells were incubated with the “B-Body-IgG 2×2” at a concentration of 3 μg/mL for 1.5 hours at room temperature. The cells were centrifuged at 300×G for 7 minutes, washed in PBS, and incubated with 100 μL of FITC labeled goat-anti human secondary antibody at a concentration of 8 μg/mL for 1 hour at room temperature. The cells were centrifuged at 300×G for 7 minutes, washed in PBS, and cell binding was confirmed by flow cytometry using a Guava easyCyte. Results are shown in FIG. 42.

### 6.13.18. Example 17: SDS-PAGE analysis of bivalent and trivalent constructs

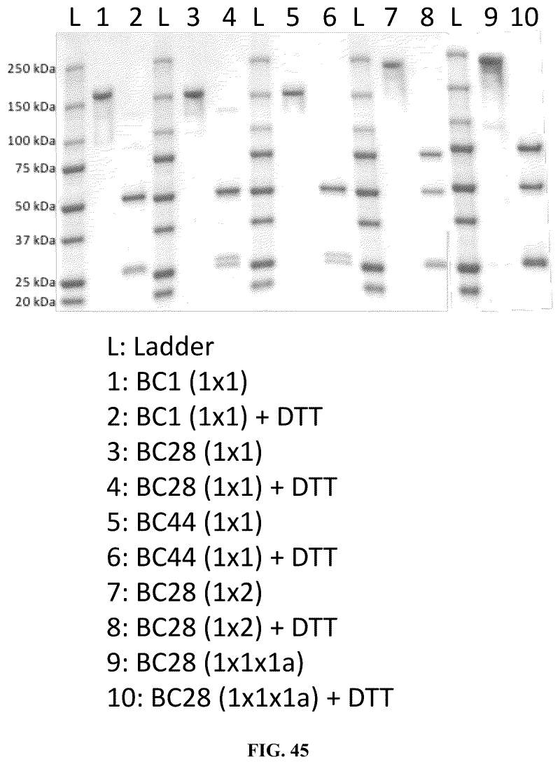

FIG. 45 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the bivalent 1×1 bispecific construct “BC28” (see Example 4). Lanes 5 (nonreducing) and 6 (reducing) are the bivalent 1×1 bispecific construct “BC44” (see Example 5). Lanes 7 (nonreducing) and 8 (reducing) are the trivalent 1×2 bispecific “BC28-1×2” construct (see

### Example 9). Lanes 9 (Nonreducing) and 10 (Reducing) are the Trivalent 1×2 Trispecific “BC28-1x1x1a” Construct Described in Example 11

The SDS-PAGE gel demonstrates the complete assembly of each construct, with the predominant band in the non-reducing gel appearing at the expected molecular weight for each construct.

### 6.13.19. Example 18: Stability Analysis of Variable-CH3 Junction Engineering

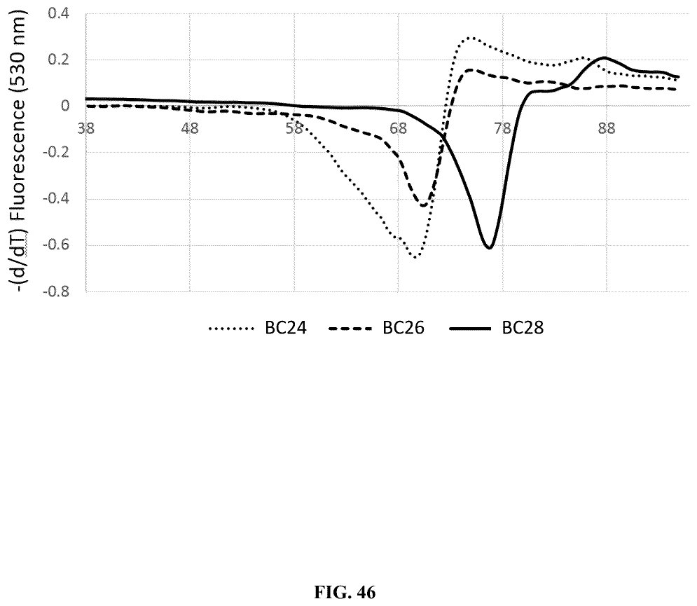

Pairing stability between various junctional variant combinations was assessed. Differential scanning fluorimetry was performed to determine the melting temperature of various junctional variant pairings between VL—CH3 polypeptides from Chain 1 (domains A and B) and VH-CH3 polypeptides from 2 (domains F and G). Junctional variants “BC6jv”, “BC28jv”, “BC30jv”, “BC44jv”, and “BC45jv”, each having the corresponding junctional sequences of “BC6”, “BC28”, “BC30”, “BC44”, and “BC45” found in Table 2 and Table 3 above, demonstrate increased pairing stability with Tm's in the 76-77 degree range (see Table 4). FIG. 46 shows differences in the thermal transitions for “BC24jv”, “BC26jv”, and “BC28jv”, with “BC28jv” demonstrating the greatest stability of the three. The x-axis of the figure is temperature and the y-axis is the change in fluorescence divided by the change in temperature (-dFluor/dTemp). Experiments were performed as described in Niesen et al. (Nature Protocols, (2007) 2, 2212-2221), which is hereby incorporated by reference for all it teaches.

TABLE 4
Melting Temperatures of Junctional Variant Pairs

| JUNCTIONAL | MELTING TEMP | MELTING TEMP |
|---|---|---|
| VARIANT PAIR | #1 (° C.) | #2 (° C.) |
| BC1jv | 69.7 | 55.6 |
| BC5jv | 71.6 | |
| BC6jv | 77 | |
| BC15jv | 68.2 | 54 |
| BC16jv | 65.9 | |
| BC17jv | 68 | |
| BC24jv | 69.7 | |
| BC26jv | 70.3 | |
| BC28jv | 76.7 | |
| BC30jv | 76.8 | |
| BC44jv | 76.2 | |
| BC45jv | 76 | |

### 6.13.20. Example 19: RORxCD3 Candidate Binding Molecules PR4T

Various RORxCD3 antibodies were constructed and tested as described below. 6.13.20.1. CD3 Binding Arm

A series of CD3 binding arm variants based on a humanized version of the SP34 anti-CD3 antibody (SP34-89, SEQ TD NOs:68 and 69) were engineered with point mutations in either the VH or VL amino acid sequences (SEQ ID Nos:70-73). The various VH and VL sequences were paired together as described in Table 5.

TABLE 5
Anti-CD3 SP34 Binding Arm Variants

| | SP34-89 VL-WT | SP34-89 VL-W57G |
|---|---|---|
| VL/VH Variants | (SEQ ID NO: 69) | (SEQ ID NO: 73) |
| SP34-89 VH-WT | SP34-89 VL-WT/ | SP34-89 VL-W57G/ |
| SEQ ID NO: 68 | SP34-89 VH-WT | SP34-89 VH-WT |
| SP34-89 VH-N30S | SP34-89 VL-WT/ | SP34-89 VL-W57G/ |
| SEQ ID NO: 70 | SP34-89 VH-N30S | SP34-89 VH-N30S |
| SP34-89 VH-G65D | SP34-89 VL-WT/ | SP34-89 VL-W57G/ |
| SEQ ID NO: 71 | SP34-89 VH-G65D | SP34-89 VH-G65D |
| SP34-89 VH-S68T | SP34-89 VL-WT/ | SP34-89 VL-W57G/ |
| SEQ ID NO: 72 | SP34-89 VH-S68T | SP34-89 VH-S68T |

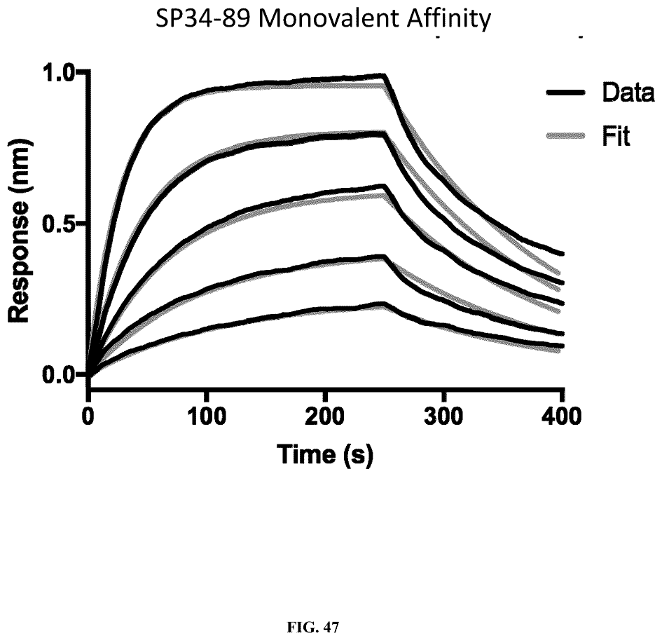

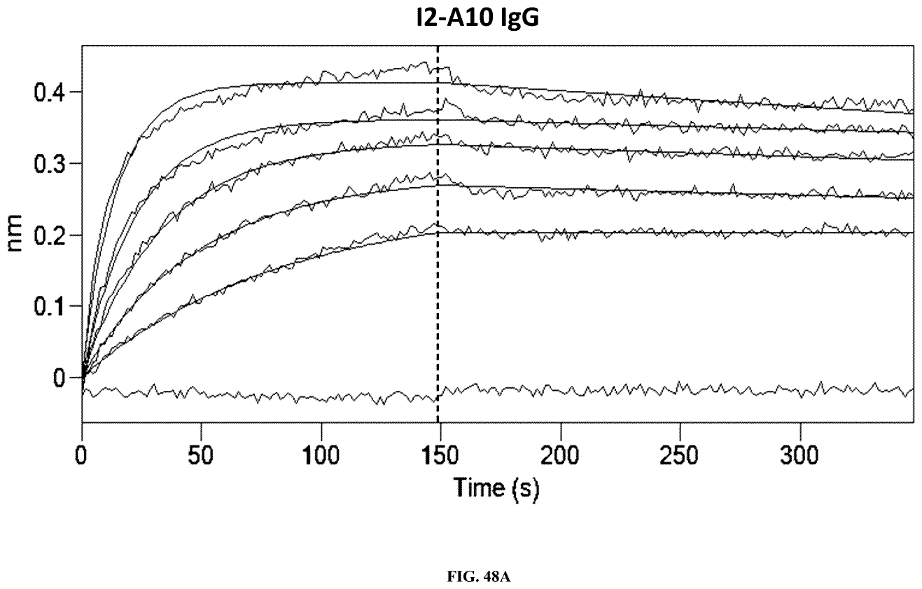

The VL and VH variants were cloned into one arm of a 1×1 BC1 B-Body, while the other arm contained an irrelevant antigen binding site. FIG. 47 demonstrates binding affinity of the non-mutagenized SP34-89 monovalent B-Body as determined by Octet (Pall ForteBio) biolayer interferometry analysis. A two-fold serial dilution (200-12.5 nM) of the construct was used to determine a binding affinity of 23 nM for SP34-89 (kₒₙ=3×10⁵M⁻¹s⁻¹, k_{off}=7.1×10⁻³ s⁻¹), matching the affinity for other SP34 variants in the literature. The kinetic affinity also matched the equilibrium binding affinity.

### 6.13.20.2. ROR Binding Arm

A chemically synthetic Fab phage library with diversity introduced into the Fab CDRs was screened against ROR antigens using a monoclonal phage ELISA format where plate-immobilized ROR variants were assessed for binding to phage, as described above. Phage clones expressing Fabs that recognized antigens were sequenced. A first screening campaign for binding to ROR1 identified the antigen binding site (ABS) clones designated “12A” in Table 6, and a second screening campaign for binding to ROR2 identified the ABS clones designated “I2C” in Table 6.

TABLE 6
ROR Antigen Binding Site Candidates

| ABS | CDR1 VH | CDR2 VH | CDR3 VH | CDR1 VL | CDR2 VL | CDR3 VL |
|---|---|---|---|---|---|---|
| I2A-1 | FSSYFI | AIYPEGGYTY | DYKYVGAL | RASQSVSSAVA | SASSLYS | YYYFPG |
| | (SEQ ID NO: 153) | (SEQ ID NO: 154) | (SEQ ID NO: 155) | (SEQ ID NO: 156) | (SEQ ID NO: 157) | (SEQ ID |
| | | | | | | NO: 158) |
| I2A-2 | FSYYGI | FIYSRGGYTI | YIGAGL | RASQSVSSAVA | SASSLYS | YYWDPI |
| | (SEQ ID NO: 159) | (SEQ ID NO: 160) | (SEQ ID NO: 161) | (SEQ ID NO: 162) | (SEQ ID NO: 163) | (SEQ ID |
| | | | | | | NO: 164) |
| I2A-3 | FTSYEI | HIDPYGGYTQ | RGVAVF | RASQSVSSAVA | SASSLYS | WAYAPV |
| | (SEQ ID NO: 165) | (SEQ ID NO: 166) | (SEQ ID NO: 167) | (SEQ ID NO: 168) | (SEQ ID NO: 169) | (SEQ ID |
| | | | | | | NO: 170) |
| I2A-4 | FYSYDI | YISPYWGITT | YIGSSYWDAL | RASQSVSSAVA | SASSLYS | SDSSLV |
| | (SEQ ID NO: 171) | (SEQ ID NO: 172) | (SEQ ID NO: 173) | (SEQ ID NO: 174) | (SEQ ID NO: 175) | (SEQ ID |
| | | | | | | NO: 176) |
| I2A-5 | FSSYGI | WISPTGSITI | SYMIYGGL | RASQSVSSAVA | SASSLYS | RVSSPW |
| | (SEQ ID NO: 177) | (SEQ ID NO: 178) | (SEQ ID NO: 179) | (SEQ ID NO: 180) | (SEQ ID NO: 181) | (SEQ ID |
| | | | | | | NO: 182) |
| I2A-6 | FSLYAI | EIDSWLGYTY | RPVTEVYYSAL | RASQSVSSAVA | SASSLYS | YDRSLH |
| | (SEQ ID NO: 183) | (SEQ ID NO: 184) | (SEQ ID NO: 185) | (SEQ ID NO: 186) | (SEQ ID NO: 187) | (SEQ ID |
| | | | | | | NO: 188) |
| I2A-7 | FSRYYI | DIDSYGGFTY | AHRFLQGGYVL | RASQSVSSAVA | SASSLYS | YSWGLW |
| | (SEQ ID NO: 189) | (SEQ ID NO: 190) | (SEQ ID NO: 191) | (SEQ ID NO: 192) | (SEQ ID NO: 193) | (SEQ ID |
| | | | | | | NO: 194) |
| I2A-8 | FYGYYI | GIRPGGTYTY | YRYPAF | RASQSVSSAVA | SASSLYS | RRQHLW |
| | (SEQ ID NO: 195) | (SEQ ID NO: l96) | (SEQ ID NO: 197) | (SEQ ID NO: 198) | (SEQ ID NO: 199) | (SEQ ID |
| | | | | | | NO: 200) |
| I2A-9 | FSSYTI | AIDSGWSYTD | AYGGVM | RASQSVSSAVA | SASSLYS | YWWPG |
| | (SEQ ID NO: 20l) | (SEQ ID NO: 202) | (SEQ ID NO: 203) | (SEQ ID NO: 204) | (SEQ ID NO: 205) | (SEQ ID |
| | | | | | | NO: 206) |
| I2A-10 | FSSYFI | GIYPSDGYTS | YYVSGM | RASQSVSSAVA | SASSLYS | YYYYPG |
| | (SEQ ID NO: 207) | (SEQ ID NO: 208) | (SEQ ID NO: 209) | (SEQ ID NO: 210) | (SEQ ID NO: 211) | (SEQ ID |
| | | | | | | NO: 212) |
| I2A-10 | FSSYFI | GIYPSEGYTS | YYVSGM | RASQSVSSAVA | SASSLQS | YYYYPG |
| D54E | (SEQ ID NO: 213) | (SEQ ID NO: 214) | (SEQ ID NO: 215) | (SEQ ID NO: 216) | (SEQ ID NO: 217) | (SEQ ID |
| Y55Q | | | | | | NO: 218) |
| I2A-11 | FSSYVI | AIYPYTSSTQ | SYGTGGF | RASQSVSSAVA | SASSLYS | WYSYPL |
| | (SEQ ID NO: 219) | (SEQ ID NO: 220) | (SEQ ID NO: 221) | (SEQ ID NO: 222) | (SEQ ID NO: 223) | (SEQ ID |
| | | | | | | NO: 224) |
| I2A-12 | FTTYYI | YISPEDGYTS | AYYSAVM | RASQSVSSAVA | SASSLYS | SWSPAT |
| | (SEQ ID NO: 225) | (SEQ ID NO: 226) | (SEQ ID NO: 227) | (SEQ ID NO: 228) | (SEQ ID NO: 229) | (SEQ ID |
| | | | | | | NO: 230) |
| I2A-13 | FSYYFI | VIYPDGGYTL | IYYPSGAM | RASQSVSSAVA | SASSLYS | TYWYPG |
| | (SEQ ID NO: 231) | (SEQ ID NO: 232) | (SEQ ID NO: 233) | (SEQ ID NO: 234) | (SEQ ID NO: 235) | (SEQ ID |
| | | | | | | NO: 236) |
| I2A-14 | FDSYVI | YIFSFGGYTY | SPYGTFAL | RASQSVSSAVA | SASSLYS | YYYTPG |
| | (SEQ ID NO: 237) | (SEQ ID NO: 238) | (SEQ ID NO: 239) | (SEQ ID NO: 240) | (SEQ ID NO: 24l) | (SEQ ID |
| | | | | | | NO: 242) |
| I2A-15 | FWGYVI | AIDSWDGDTD | SFYYIYVM | RASQSVSSAVA | SASSLYS | LYSTLV |
| | (SEQ ID NO: 243) | (SEQ ID NO: 244) | (SEQ ID NO: 245) | (SEQ ID NO: 246) | (SEQ ID NO: 247) | (SEQ ID |
| | | | | | | NO: 248) |
| I2A-16 | FSGYFI | AIFPYRGGTS | GGVSPGGF | RASQSVSSAVA | SASSLYS | YYLYPG |
| | (SEQ ID NO: 249) | (SEQ ID NO: 250) | (SEQ ID NO: 251) | (SEQ ID NO: 252) | (SEQ ID NO: 253) | (SEQ ID |
| | | | | | | NO: 254) |
| I2A-17 | FESYDI | AIFSYGGYTT | GSYGDGRGM | RASQSVSSAVA | SASSLYS | YYYWPG |
| | (SEQ ID NO: 255) | (SEQ ID NO: 256) | (SEQ ID NO: 257) | (SEQ ID NO: 258) | (SEQ ID NO: 259) | (SEQ ID |
| | | | | | | NO: 260) |
| I2A-18 | FSSYFI | AIHPAFSFTY | PRLSSAVVL | RASQSVSSAVA | SASSLYS | * |
| | (SEQ ID NO: 261) | (SEQ ID NO: 262) | (SEQ ID NO: 263) | (SEQ ID NO: 264) | (SEQ ID NO: 265) | |
| I2A-19 | FSSYFI | WIYPSGSYTY | EMDRVGYSGM | RASQSVSSAVA | SASSLYS | YRTPLG |
| | (SEQ ID NO: 266) | (SEQ ID NO: 267) | (SEQ ID NO: 268) | (SEQ ID NO: 269) | (SEQ ID NO: 270) | (SEQ ID |
| | | | | | | NO: 271) |
| I2A-20 | FSDYGI | EIDSWLGYTY | SPYHYLYYGL | RASQSVSSAVA | SASSLYS | LSSSLG |
| | (SEQ ID NO: 272) | (SEQ ID NO: 273) | (SEQ ID NO: 274) | (SEQ ID NO: 275) | (SEQ ID NO: 276) | (SEQ ID |
| | | | | | | NO: 277) |
| I2A-21 | FSGYFI | GISPWAGYTS | GGGRAF | RASQSVSSAVA | SASSLYS | YYWYPG |
| | (SEQ ID NO: 278) | (SEQ ID NO: 279) | (SEQ ID NO: 280) | (SEQ ID NO: 281) | (SEQ ID NO: 282) | (SEQ ID |
| | | | | | | NO: 283) |
| I2A-22 | FSSYFI | AIYPSGWYTS | VQAGVF | RASQSVSSAVA | SASSLYS | YYYYPG |
| | (SEQ ID NO: 284) | (SEQ ID NO: 285) | (SEQ ID NO: 286) | (SEQ ID NO: 287) | (SEQ ID NO: 288) | (SEQ ID |
| | | | | | | NO: 289) |
| I2A-23 | FDDYFI | AISSEGGYTD | AYRGVF | RASQSVSSAVA | SASSLYS | YYYFPG |
| | (SEQ ID NO: 290) | (SEQ ID NO: 291) | (SEQ ID NO: 292) | (SEQ ID NO: 293) | (SEQ ID NO: 294) | (SEQ ID |
| | | | | | | NO: 295) |
| I2A-24 | FSTYGI | AIYPGTSYTG | EYFMGM | RASQSVSSAVA | SASSLYS | YYYWPG |
| | (SEQ ID NO: 296) | (SEQ ID NO: 297) | (SEQ ID NO: 298) | (SEQ ID NO: 299) | (SEQ ID NO: 300) | (SEQ ID |
| | | | | | | NO: 301) |
| I2A-25 | FYGYTI | AIYPYTDSTR | DYRRAL | RASQSVSSAVA | SASSLYS | YTDFPW |
| | (SEQ ID NO: 302) | (SEQ ID NO: 303) | (SEQ ID NO: 304) | (SEQ ID NO: 305) | (SEQ ID NO: 306) | (SEQ ID |
| | | | | | | NO: 307) |
| I2A-26 | FQSYDI | AIDPTGRSTA | DYGVF | RASQSVSSAVA | SASSLYS | FYRSPA |
| | (SEQ ID NO: 308) | (SEQ ID NO: 309) | (SEQ ID NO: 310) | (SEQ ID NO: 311) | (SEQ ID NO: 312) | (SEQ ID |
| | | | | | | NO: 313) |
| I2A-27 | FKGYYI | AIYPYGGSTD | VYIYGVF | RASQSVSSAVA | SASSLYS | YYSSPR |
| | (SEQ ID NO: 314) | (SEQ ID NO: 315) | (SEQ ID NO: 316) | (SEQ ID NO: 317) | (SEQ ID NO: 318) | (SEQ ID |
| | | | | | | NO: 3l9) |
| I2A-28 | FSSYWI | WIYPGTRYTE | DYVWPYGF | RASQSVSSAVA | SASSLYS | ASWSPV |
| | (SEQ ID NO: 320) | (SEQ ID NO: 321) | (SEQ ID NO: 322) | (SEQ ID NO: 323) | (SEQ ID NO: 324) | (SEQ ID |
| | | | | | | NO: 325) |
| I2A-29 | FSSYWI | WIYSSGGYTF | EYFLYTGF | RASQSVSSAVA | SASSLYS | YSSGPV |
| | (SEQ ID NO: 326) | (SEQ ID NO: 327) | (SEQ ID NO: 328) | (SEQ ID NO: 329) | (SEQ ID NO: 330) | (SEQ ID |
| | | | | | | NO: 331) |
| I2A-30 | FDSYFI | YIYSWGSYTH | GHRRYFAL | RASQSVSSAVA | SASSLYS | VYFTPG |
| | (SEQ ID NO: 332) | (SEQ ID NO: 333) | (SEQ ID NO: 334) | (SEQ ID NO: 335) | (SEQ ID NO: 336) | (SEQ ID |
| | | | | | | NO: 337) |
| I2A-31 | FSSYWI | FIGPSGGYTY | ETDSYTGF | RASQSVSSAVA | SASSLYS | YYSWLG |
| | (SEQ ID NO: 338) | (SEQ ID NO: 339) | (SEQ ID NO: 340) | (SEQ ID NO: 341) | (SEQ ID NO: 342) | (SEQ ID |
| | | | | | | NO: 343) |
| I2A-32 | FQSYVI | AIYPYSSSTI | SWSVYLGM | RASQSVSSAVA | SASSLYS | SYDSPR |
| | (SEQ ID NO: 344) | (SEQ ID NO: 345) | (SEQ ID NO: 346) | (SEQ ID NO: 347) | (SEQ ID NO: 348) | (SEQ ID |
| | | | | | | NO: 349) |
| I2A-33 | FDDYYI | WIDSYGGYTS | SSYYYPGGF | RASQSVSSAVA | SASSLYS | WDSTLY |
| | (SEQ ID NO: 350) | (SEQ ID NO: 351) | (SEQ ID NO: 352) | (SEQ ID NO: 353) | (SEQ ID NO: 354) | (SEQ ID |
| | | | | | | NO: 355) |
| I2A-34 | FSWYVI | YIAPYTGSTY | AFFGIRLGL | RASQSVSSAVA | SASSLYS | AISSPY |
| | (SEQ ID NO: 356) | (SEQ ID NO: 357) | (SEQ ID NO: 358) | (SEQ ID NO: 359) | (SEQ ID NO: 360) | (SEQ ID |
| | | | | | | NO: 361) |
| I2A-35 | FSAYDI | WIDPYGGDTD | SPSYMQYGGL | RASQSVSSAVA | SASSLYS | YYSSLL |
| | (SEQ ID NO: 362) | (SEQ ID NO: 363) | (SEQ ID NO: 364) | (SEQ ID NO: 365) | (SEQ ID NO: 366) | (SEQ ID |
| | | | | | | NO: 367) |
| I2A-36 | FSQYWI | AIYSSTKYTI | ESMYFYSYGL | RASQSVSSAVA | SASSLYS | LPSTPL |
| | (SEQ ID NO: 368) | (SEQ ID NO: 369) | (SEQ ID NO: 370) | (SEQ ID NO: 371) | (SEQ ID NO: 372) | (SEQ ID |
| | | | | | | NO: 373) |
| I2A-37 | FSWYGI | YIDSYTSSTY | SHFGHYDYVM | RASQSVSSAVA | SASSLYS | AYDQLY |
| | (SEQ ID NO: 374) | (SEQ ID NO: 375) | (SEQ ID NO: 376) | (SEQ ID NO: 377) | (SEQ ID NO: 378) | (SEQ ID |
| | | | | | | NO: 379) |
| I2A-38 | FDWYRI | WIDSYGSWTG | SYFGPYGYVL | RASQSVSSAVA | SASSLYS | * |
| | (SEQ ID NO: 380) | (SEQ ID NO: 381) | (SEQ ID NO: 382) | (SEQ ID NO: 383) | (SEQ ID NO: 384) | |
| I2C-1 | FTSYGI | AIYPHSGFTS | TSYRGF | RASQSVSSAVA | SASSLYS | YYWYPG |
| | (SEQ ID NO: 385) | (SEQ ID NO: 386) | (SEQ ID NO: 387) | (SEQ ID NO: 388) | (SEQ ID NO: 389) | (SEQ ID |
| | | | | | | NO: 390) |
| I2C-2 | FSDYFI | GIYPYSGYTK | DHSPVL | RASQSVSSAVA | SASSLYS | WYYWPG |
| | (SEQ ID NO: 391) | (SEQ ID NO: 392) | (SEQ ID NO: 393) | (SEQ ID NO: 394) | (SEQ ID NO: 395) | (SEQ ID |
| | | | | | | NO: 396) |
| I2C-3 | FSHYWI | LIAPGGDYTS | SGLPGF | RASQSVSSAVA | SASSLYS | YKSSPL |
| | (SEQ ID NO: 397) | (SEQ ID NO: 398) | (SEQ ID NO: 399) | (SEQ ID NO: 400) | (SEQ ID NO: 401) | (SEQ ID |
| | | | | | | NO: 402) |
| I2C-4 | FWSYFI | YIHPSSSYTD | TSRDGAM | RASQSVSSAVA | SASSLYS | WYSPPE |
| | (SEQ ID NO: 403) | (SEQ ID NO: 404) | (SEQ ID NO: 405) | (SEQ ID NO: 406) | (SEQ ID NO: 407) | (SEQ ID |
| | | | | | | NO: 408) |
| I2C-5 | FSSYDI | WIYPYWGYTI | GTYAPAL | RASQSVSSAVA | SASSLYS | FYSYLS |
| | (SEQ ID NO: 409) | (SEQ ID NO: 410) | (SEQ ID NO: 411) | (SEQ ID NO: 412) | (SEQ ID NO: 413) | (SEQ ID |
| | | | | | | NO: 414) |
| I2C-6 | FSWYFI | RIYSTGGYTE | SAFFGAL | RASQSVSSAVA | SASSLYS | YPSGPE |
| | (SEQ ID NO: 415) | (SEQ ID NO: 416) | (SEQ ID NO: 417) | (SEQ ID NO: 418) | (SEQ ID NO: 419) | (SEQ ID |
| | | | | | | NO: 420) |
| I2C-7 | FDSYYI | WIDPYGLDTK | EPGDYGM | RASQSVSSAVA | SASSLYS | AYGSLL |
| | (SEQ ID NO: 421) | (SEQ ID NO: 422) | (SEQ ID NO: 423) | (SEQ ID NO: 424) | (SEQ ID NO: 425) | (SEQ ID |
| | | | | | | NO: 426) |
| 12C-8 | FSGYFI | AIFPYRGGTS | GGVSPGGF | RASQSVSSAVA | SASSLYS | YYLYPG |
| | (SEQ ID NO: 427) | (SEQ ID NO: 428) | (SEQ ID NO: 429) | (SEQ ID NO: 430) | (SEQ ID NO: 431) | (SEQ ID |
| | | | | | | NO: 432) |
| I2C-9 | FTDYDI | RIWPHGSYTF | SLTHSYGF | RASQSVSSAVA | SASSLYS | YYTWLI |
| | (SEQ ID NO: 433) | (SEQ ID NO: 434) | (SEQ ID NO: 435) | (SEQ ID NO: 436) | (SEQ ID NO: 437) | (SEQ ID |
| | | | | | | NO: 438) |
| I2C-10 | FSSYFI | TIHSYFDGTS | TRPTGGAF | RASQSVSSAVA | SASSLYS | AYWSPA |
| | (SEQ ID NO: 439) | (SEQ ID NO: 440) | (SEQ ID NO: 441) | (SEQ ID NO: 442) | (SEQ ID NO: 443) | (SEQ ID |
| | | | | | | NO: 444) |
| I2C-11 | FGSYFI | AIFPAGGYTY | YGSMGGAF | RASQSVSSAVA | SASSLYS | YYWFPG |
| | (SEQ ID NO: 445) | (SEQ ID NO: 446) | (SEQ ID NO: 447) | (SEQ ID NO: 448) | (SEQ ID NO: 449) | (SEQ ID |
| | | | | | | NO: 450) |
| I2C-12 | FSSYFI | AIHPAFSFTY | PRLSSAVVL | RASQSVSSAVA | SASSLYS | GYYFPG |
| | (SEQ ID NO: 451) | (SEQ ID NO: 452) | (SEQ ID NO: 453) | (SEQ ID NO: 454) | (SEQ ID NO: 455) | (SEQ ID |
| | | | | | | NO: 456) |
| I2C-13 | FSSYGI | YIHSGIGYTI | TSSTSTSVM | RASQSVSSAVA | SASSLYS | TYWSPG |
| | (SEQ ID NO: 457) | (SEQ ID NO: 458) | (SEQ ID NO: 459) | (SEQ ID NO: 460) | (SEQ ID NO: 461) | (SEQ ID |
| | | | | | | NO: 462) |
| I2C-14 | FESYDI | AIFSYGGYTT | GSYGDGRGM | RASQSVSSAVA | SASSLYS | YYYWPG |
| | (SEQ ID NO: 463) | (SEQ ID NO: 464) | (SEQ ID NO: 465) | (SEQ ID NO: 466) | (SEQ ID NO: 467) | (SEQ ID |
| | | | | | | NO: 468) |
| I2C-15 | FSSYFI | LIYPDTDDTY | YGYLGVGAF | RASQSVSSAVA | SASSLYS | LYWTPG |
| | (SEQ ID NO: 469) | (SEQ ID NO: 470) | (SEQ ID NO: 471) | (SEQ ID NO: 472) | (SEQ ID NO: 473) | (SEQ ID |
| | | | | | | NO: 474) |
| I2C-16 | FSSYWI | AIHPSGSYTY | VGLLVTSVM | RASQSVSSAVA | SASSLYS | YYYFPG |
| | (SEQ ID NO: 475) | (SEQ ID NO: 476) | (SEQ ID NO: 477) | (SEQ ID NO: 478) | (SEQ ID NO: 479) | (SEQ ID |
| | | | | | | NO: 480) |
| I2C-17 | FSDYFI | YIYPASGGTC | GYIPHMAAL | RASQSVSSAVA | SASSLYS | YYWWPG |
| | (SEQ ID NO: 48l) | (SEQ ID NO: 482) | (SEQ ID NO: 483) | (SEQ ID NO: 484) | (SEQ ID NO: 485) | (SEQ ID |
| | | | | | | NO: 486) |
| I2C-18 | FAGYPI | AIDPDGGYTY | HTGFHRYRGM | RASQSVSSAVA | SASSLYS | YYWFPP |
| | (SEQ ID NO: 487) | (SEQ ID NO: 488) | (SEQ ID NO: 489) | (SEQ ID NO: 490) | (SEQ ID NO: 49l) | (SEQ ID |
| | | | | | | NO: 492) |
| I2C-19 | FTSYDI | WIDPGLSYTS | ASIGGGVPVM | RASQSVSSAVA | SASSLYS | YVTGPY |
| | (SEQ ID NO: 493) | (SEQ ID NO: 494) | (SEQ ID NO: 495) | (SEQ ID NO: 496) | (SEQ ID NO: 497) | (SEQ ID |
| | | | | | | NO: 498) |
| I2C-20 | FSSYYI | IIIPYSGYTY | RGYFSLGTAM | RASQSVSSAVA | SASSLYS | VYWWPG |
| | (SEQ ID NO: 499) | (SEQ ID NO: 500) | (SEQ ID NO: 501) | (SEQ ID NO: 502) | (SEQ ID NO: 503) | (SEQ ID |
| | | | | | | NO: 504) |
| I2C-21 | FSSYYI | WIDPYISYTY | DSELVSGYAM | RASQSVSSAVA | SASSLYS | GDSSLV |
| | (SEQ ID NO: 505) | (SEQ ID NO: 506) | (SEQ ID NO: 507) | (SEQ ID NO: 508) | (SEQ ID NO: 509) | (SEQ ID |
| | | | | | | NO: 510) |
| I2C-22 | FSSYSI | AIYPYWGTTE | PSGVTYGYAL | RASQSVSSAVA | SASSLYS | YYYSPW |
| | (SEQ ID NO: 511) | (SEQ ID NO: 5l2) | (SEQ ID NO: 513) | (SEQ ID NO: 514) | (SEQ ID NO: 515) | (SEQ ID |
| | | | | | | NO: 516) |
| I2C-23 | FSSYEI | SIYPFSGDTY | PGRAIYYAVM | RASQSVSSAVA | SASSLYS | AGSHLF |
| | (SEQ ID NO: 517) | (SEQ ID NO: 518) | (SEQ ID NO: 519) | (SEQ ID NO: 520) | (SEQ ID NO: 52l) | (SEQ ID |
| | | | | | | NO: 522) |
| I2C-24 | FSRYYI | DIDSYGGFTY | AHRFLQGGYVL | RASQSVSSAVA | SASSLYS | YSWGLW |
| | (SEQ ID NO: 523) | (SEQ ID NO: 524) | (SEQ ID NO: 525) | (SEQ ID NO: 526) | (SEQ ID NO: 527) | (SEQ ID |
| | | | | | | NO: 528) |
| I2C-25 | FSDYYI | YIAPYGGFTY | DSYRRGYVSGF | RASQSVSSAVA | SASSLYS | RYSSPS |
| | (SEQ ID NO: 529) | (SEQ ID NO: 530) | (SEQ ID NO: 531) | (SEQ ID NO: 532) | (SEQ ID NO: 533) | (SEQ ID |
| | | | | | | NO: 534) |
| I2C-26 | FSDYYI | DIDSYGGFTY | DPHFLDDVYVL | RASQSVSSAVA | SASSLYS | YSWGLW |
| | (SEQ ID NO: 535) | (SEQ ID NO: 536) | (SEQ ID NO: 537) | (SEQ ID NO: 538) | (SEQ ID NO: 539) | (SEQ ID |
| | | | | | | NO: 540) |

“I2A” screened against ROR1;
“I2C” screened against ROR2;
* denotes no sequence determined;
bold denotes potential isomerization sites (see below)

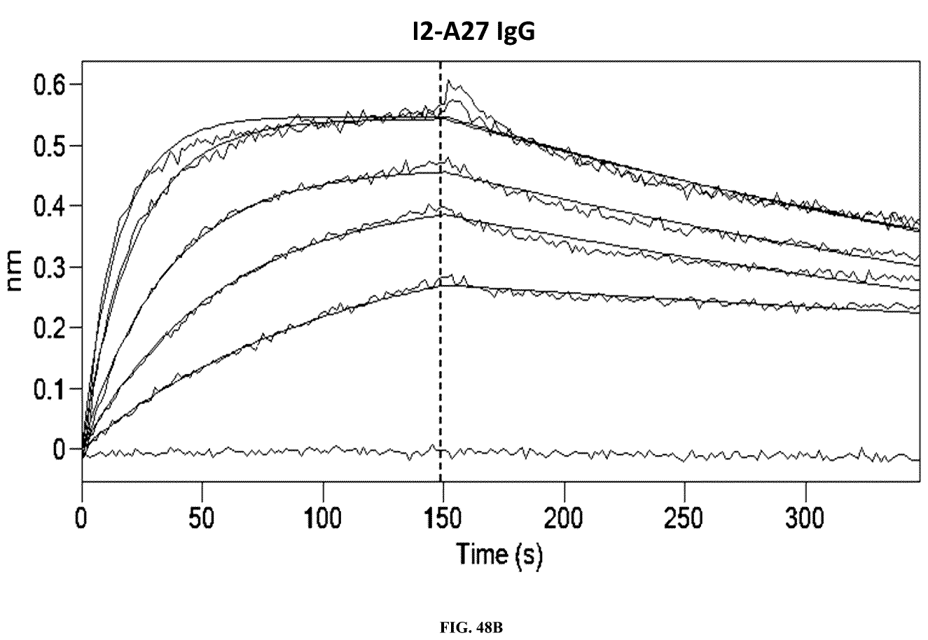

The VH and VL sequences above were formatted into a bivalent monospecific native human full-length IgG1 architecture. FIGS. 48A-48B demonstrate Octet (Pall ForteBlio) biolayer interferometry analysis of a two-fold serial dilution (200-12.5 nM) for two ROR binding candidates (FIG. 48A clone I2-A10; FIG. 48B clone I2-A27). Binding affinities and kinetics were determined from the serial dilutions and are shown in Table 7.

TABLE 7
Binding Kinetics of ROR ABS Candidates

| Candidate | KD (nM) | Kₒₙ (1/Ms) | K_{dis} (1/s) | RMax |
|---|---|---|---|---|
| I2-A10 | 0.82 | 5.38 × 10⁵ | 3.8 × 10⁻⁴ | 0.4159 |
| I2-A27 | 4.1 | 5.11 × 10⁵ | 1.85 × 10⁻³ | 0.5668 |

ROR binding candidates, formatted in the bivalent monospecific native human full-length IgG1 architecture, were further characterized for binding to ROR1 and/or ROR2. Table 8 presents candidates that bound specifically to RORM only, bound specifically to ROR2 only, and were cross-reactive for both ROR1 and ROR2. ROR binding candidates were also characterized for their binding to specific ROR1 domains. Table 9 presents candidates that bound specifically to Frizzled, Ig-like, and Kringle domains.

TABLE 8
ROR Binding Candidate Specificity

| ROR1 Specific | ROR2 Specific | ROR1/ROR2 Cross Reactive |
|---|---|---|
| I2A-3 | I2C-3 | I2A-1 |
| I2A-4 | I2C-5 | I2A-10 |
| I2A-6 | I2C-7 | I2A-10 |
| | | D54E Y55Q |
| I2-A8 | I2C-19 | I2A-11 |
| I2-A12 | I2C-21 | I2-A13 |
| I2A-20 | I2C-25 | I2A-14 |
| I2-A25 | I2C-26 | I2A-16 |
| I2A-26 | | I2-A18 |
| I2A-27 | | I2A-19 |
| I2-A30 | | I2A-22 |
| I2-A32 | | I2A-24 |
| I2-A33 | | I2-A26 |
| I2-A37 | | I2-A34 |
| | | I2-A36 |
| | | I2C-1 |
| | | I2C-2 |
| | | I2C-4 |
| | | I2C-6 |
| | | I2C-8 |
| | | I2C-9 |
| | | I2C-10 |
| | | I2C-11 |
| | | I2C-12 |
| | | I2C-13 |
| | | I2C-14 |
| | | I2C-15 |
| | | I2C-16 |
| | | I2C-17 |
| | | I2C-18 |
| | | I2C-20 |
| | | I2C-22 |
| | | I2C-23 |
| | | I2C-24 |

TABLE 9
Domain Mapping

| Clone | Domain of ROR1 |
|---|---|
| I2A-11 | Frizzled |
| I2A-19 | Frizzled |
| I2C-23 | Frizzled |
| I2C-6 | Frizzled |
| I2A-32 | Frizzled |
| I2A-34 | Frizzled |
| I2A-26 | Frizzled |
| I2A-25 | Frizzled |
| I2A-26 | Frizzled |
| I2-A12 | Frizzled |
| I2A-1 | Ig-like |
| I2A-10 | Ig-like |
| I2A-10 | Ig-like |
| D54E Y55Q | |
| I2A-14 | Ig-like |
| I2A-16 | Ig-like |
| I2A-20 | Ig-like |
| I2A-22 | Ig-like |
| I2A-3 | Ig-like |
| I2A-35 | Ig-like |
| I2A-4 | Ig-like |
| I2-A6 | Ig-like |
| I2-A13 | Ig-like |
| I2-A18 | Ig-like |
| I2-A30 | Ig-like |
| I2-A33 | Ig-like |
| I2-A37 | Ig-like |
| I2C-1 | Ig-like |
| I2C-10 | Ig-like |
| I2C-11 | Ig-like |
| I2C-12 | Ig-like |
| I2C-13 | Ig-like |
| I2C-14 | Ig-like |
| I2C-15 | Ig-like |
| I2C-16 | Ig-like |
| I2C-17 | Ig-like |
| I2C-2 | Ig-like |
| I2C-20 | Ig-like |
| I2C-24 | Ig-like |
| I2C-4 | Ig-like |
| I2C-8 | Ig-like |
| I2C-9 | Ig-like |
| I2A-27 | Kringle |

Select ROR binding candidates were further analyzed for sequence motifs that could adversely affect antibody properties that are relevant to clinical development, such as stability, mutability, and immunogenicity. Computational analysis was performed according to Kumar and Singh (Developability of biotherapeutics: computational approaches. Boca Raton: CRC Press, Taylor & Francis Group, 2016). Analysis results are presented in Table 10, and demonstrate a limited number of adverse sequence motifs are present in the listed clones illustrating the potential for further clinical development.

TABLE 10
Number of Potential Adverse Sequence Motifs

| | | | | | Other Sites | |
|---|---|---|---|---|---|---|
| | Deamidation | | N-linked | | (LLQG, HPQ, | |
| | Sites | Isomerization | Glycosylation | Cys | FHENSP, | |
| | (NG, NS, NA, | Sites | Sites | in | LPRWG, | # T-cell |
| ABS | NH, ND) | (DG, DP, DS) | (NXS/T) | CDR | HHH) | Epitopes* |
| I2A-27 | 0 | 1 | 0 | 0 | 0 | 2 |
| I2A-3 | 0 | 1 | 0 | 0 | 0 | 0 |
| I2A-10 | 0 | 2 | 0 | 0 | 0 | 0 |
| I2A-11 | 0 | 1 | 0 | 0 | 0 | 0 |
| I2A-19 | 0 | 1 | 0 | 0 | 0 | 0 |

*Predicted T-cell epitope found in herceptin is present in these molecules

### 6.13.21. Example 20: RORxCD3 Bispecific B-Body Efficacy in vitro

Candidate ROR and CD3 antigen binding sites were formatted into B-Body BC1 1×1 and 1×2 formats and tested in a series of tumor efficacy models.

### 6.13.21.1. Bispecific B-body Format Comparison

ROR antigen binding site (ABS) candidate I2A-3 and CD3 ABS candidate SP34-89 were formatted into bispecific B-Body “BC1” 1×1 and 1×2 formats. With reference to FIG. 3 and FIG. 26, the ROR ABS candidate forms the A:F and the R:T binding sites, while the CD3 ABS candidate forms the H:L binding site. Two 1×2 formats were constructed with either a 10 amino acid junction or 16 amino acid junction between the S domain and the H domain, with reference to FIG. 26. The different constructs were tested in the NFκB GFP Jurkat T cell stimulation assay described herein. Briefly, reporter T cells (effector cells) were mixed with either the non-small cell lung cancer target tumor cell line HOP-92, which expresses the ROR1 antigen, or the B16 melanoma target tumor cell line, which does not express ROR1. A dilution series of the different B-body constructs were then incubated with the effector:target mixture.

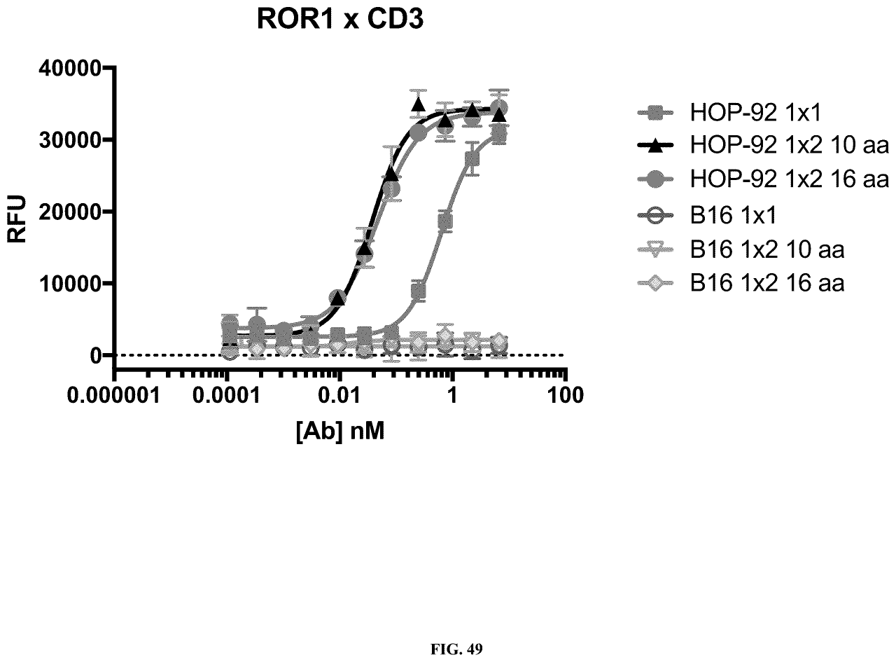

As shown in FIG. 49 and presented in Table 11, the RORxCD3 bispecific 1×1 and 1×2 B-bodies resulted in activation of the reporter T cells when mixed with ROR1 expressing tumor lines (HOP-92), but no activation was observed when the RORxCD3 bispecific 1×1 and 1×2 B-bodies were mixed with tumor lines that do not express ROR1 (B16). In addition, the 1×2 B-body format possessing bivalent specificity for ROR1 was more potent than the 1×1 B-body format, and varying the junction length resulted in minimal differences in potency.

TABLE 11
B-body bispecific format comparison

| Cell Line/Antibody | EC50(pM) |
|---|---|
| HOP-92 1 × 1 | 634 |
| HOP-92 1 × 2 16 | 47 |
| aa linker | |
| HOP-92 1 × 2 10 | 37 |
| aa linker | |

6.13.21.2. CD3 Binding Alone does not Activate T Cells

ROR ABS candidate I2A-10 and CD3 ABS candidate SP34-89 was formatted into a bispecific B-Body “BC1” 1×2 format (“I2-A10 1X2”). In a separate construct, a control arm to a tumor antigen other than ROR1 was also formatted with CD3 ABS candidate SP34-89 into a bispecific B-Body “BC1” 1×2 format (“Neg Ctrl”). With reference to FIG. 3 and FIG. 26, the ROR candidate ABS and “Neg Ctrl” arm ABS forms the A:F and the R:T binding sites, while the CD3 ABS candidate forms the H:L binding site. The different constructs were tested in a T cell cytotoxicity assay. Briefly, isolated CD8+ T cells (effector cells) were mixed with the triple negative breast cancer tumor cell line MDA-MD-231, which expresses the ROR1 antigen (target cells). A dilution series of the different B-body constructs were then incubated with the effector:target mixture.

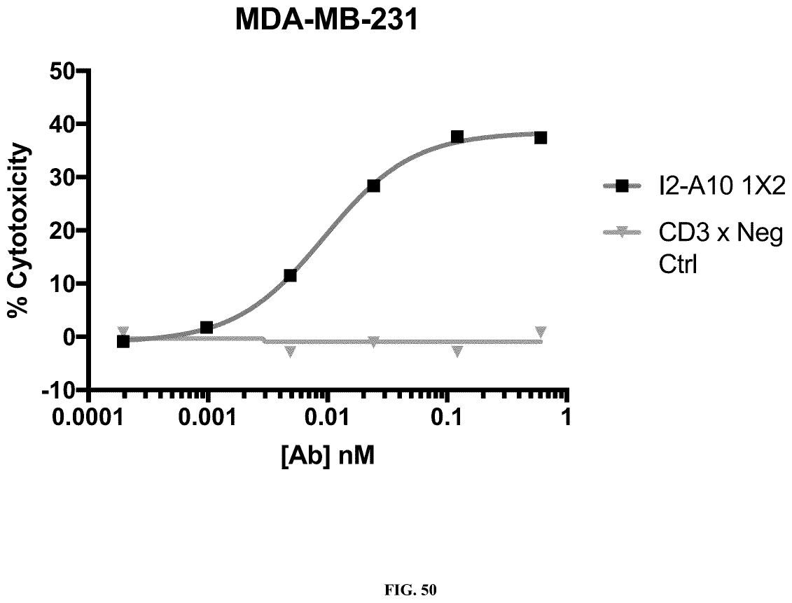

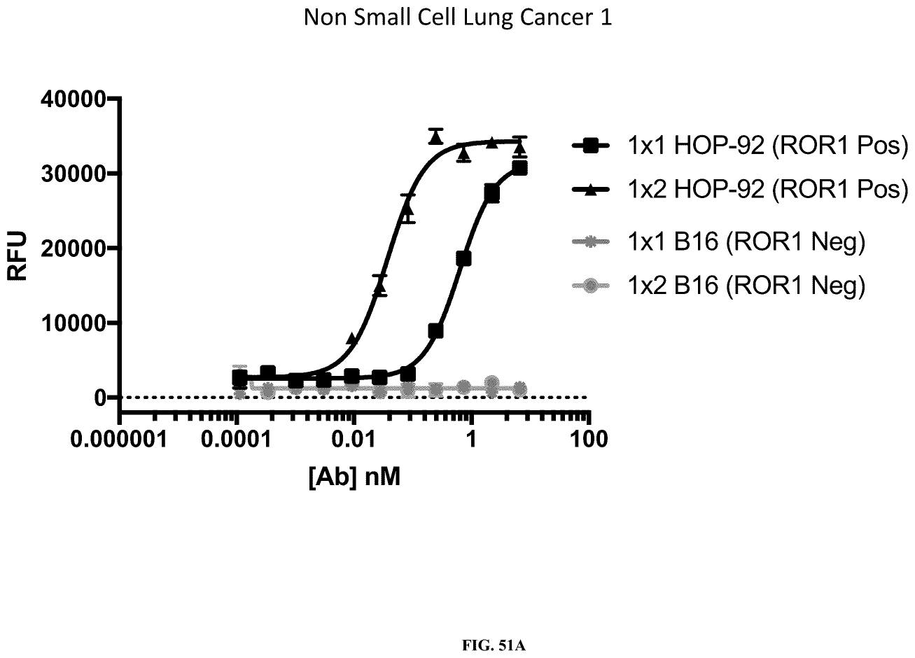

As shown in FIG. 50, the RORxCD3 trivalent bispecific 1×2 B-body resulted in cytotoxic T cell mediated killing when mixed with ROR1 expressing tumor lines (MDA-MD-231), but did not result in cytotoxicity when a CD3 bispecific B-body having an irrelevant tumor ABS (e.g., a tumor antigen not expressed in MDA-MD-231) was added to the mixture.

### 6.13.21.3. RORxCD3 Bispecific B-body Efficacy in Multiple Tumor Models

ROR ABS candidate I2A-3 and CD3 ABS candidate SP34-89 were formatted into bispecific B-Body “BC1” 1×1 and 1×2 formats, as described above, with the 1×2 format having a 10 amino acid junction between the S and the H domains. The different constructs were tested in the NFκB GFP Jurkat T cell stimulation assay described herein. Briefly, reporter T cells (effector cells) were mixed with ROR1 expressing tumor cell lines HOP-92 (non-small cell lung cancer 1), A549 (non-small cell lung cancer 2), MDA-MD-231 (triple negative breast cancer), JeKo-1 (mantle cell lymphoma), and RPMI-8226 (multiple myeloma). The constructs were also mixed with the B16 melanoma tumor cell line that does not express ROR1 (target cells). A dilution series of the different B-body constructs were then incubated with the effector:target mixture.

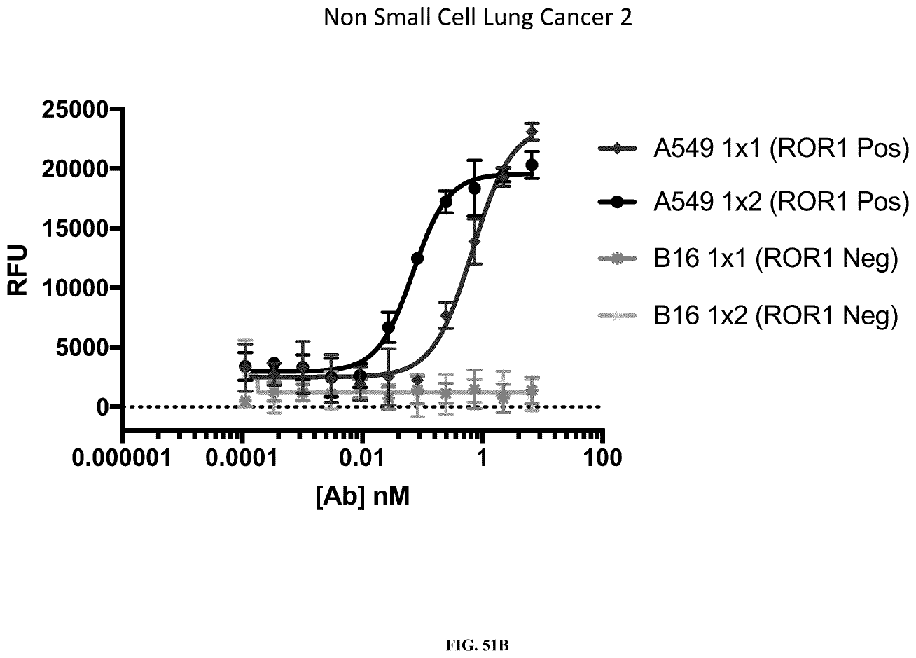

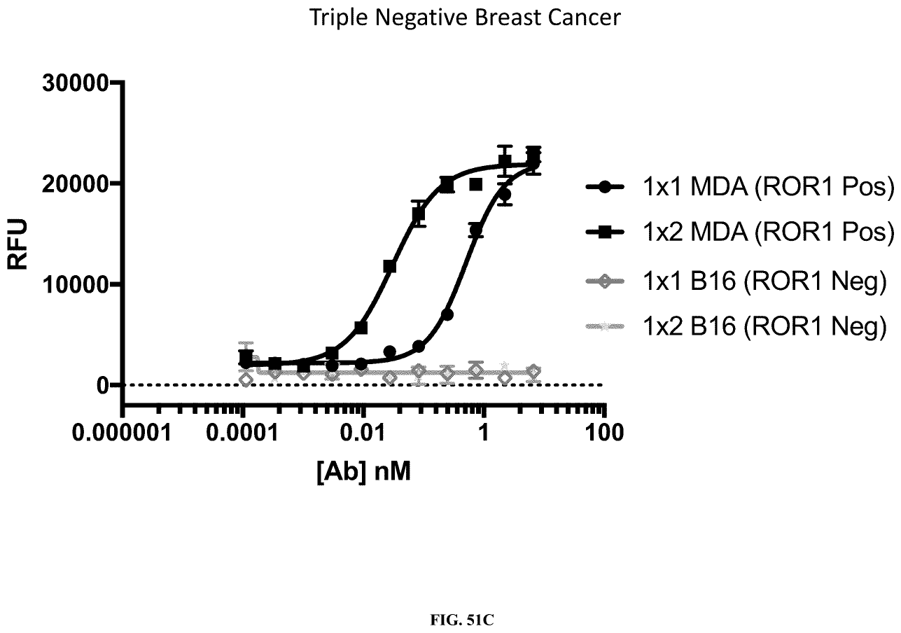

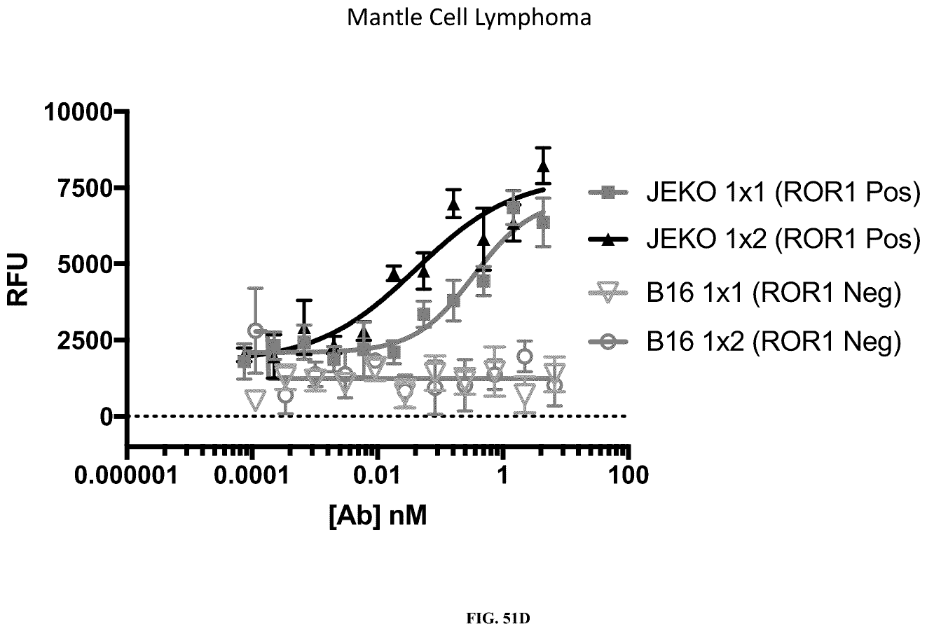

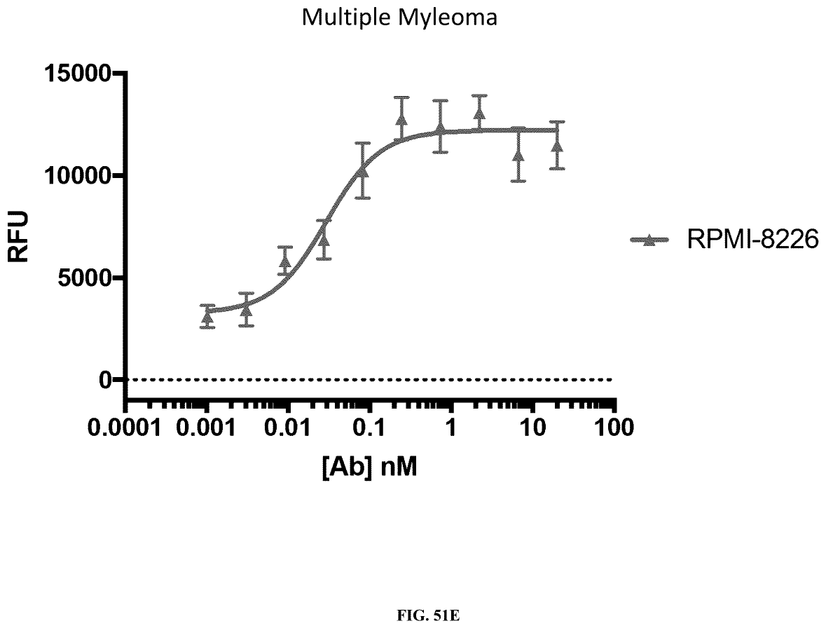

As shown in FIGS. 51A-51E and presented in Table 12, the RORxCD3 bispecific 1×1 and 1×2 B-bodies resulted in activation of the reporter T cells when mixed with ROR1 expressing tumor lines HOP-92 (FIG. 51A), A549 (FIG. 51B), MDA-MD-231 (FIG. 51C), JeKo-1 (FIG. 51D), and RPMI-8226 (FIG. 51E), but there was no activation when these constructs were mixed with tumor lines that do not express ROR1 (B16). The 1×2 B-body format possessing bivalent specificity for ROR1 was more potent than the 1×1 B-body format in all tumor lines tested. Thus, the RORxCD3 bispecific 1×1 and 1×2 B-bodies demonstrated efficacy across a range of tumor models.

TABLE 12
B-body bispecific efficacy in multiple tumor models

| Cell Line/Antibody | EC50 (pM) |
|---|---|
| HOP-92 1 × 1 | 634 |
| HOP-92 1 × 2 | 37 |
| MDA-MB-231 1 × 1 | 510 |
| MDA-MB-231 1 × 2 | 31 |
| A549 1 × 1 | 678 |
| A549 1 × 2 | 70 |
| JeKo-1 1 × 1 | 342 |
| JeKo-1 1 × 2 | 41 |
| RPMI-8226 | 29 |

6.13.21.4. ROR ABS Candidate Screening for T cell Activation

ROR ABS candidates 12A-1, 12A-3, 12A-10, 12A-14, 12A-16, 12A-20, 12A-22, and I2A-27 were formatted with CD3 ABS candidate SP34-89 into a bispecific B-Body “BC1” 1×2 format, as described above, with the 1×2 format having a 10 amino acid junction between the S and the H domains. The different constructs were tested in the T cell cytotoxicity assay described above. Briefly, isolated CD8+ T cells (effector cells) were mixed with the triple negative breast cancer tumor cell line MDA-MD-231, which expresses the ROR1 antigen (target cells) at an E:T ratio of 6:1. A dilution series of the different B-body constructs were then incubated with the effector:target mixture.

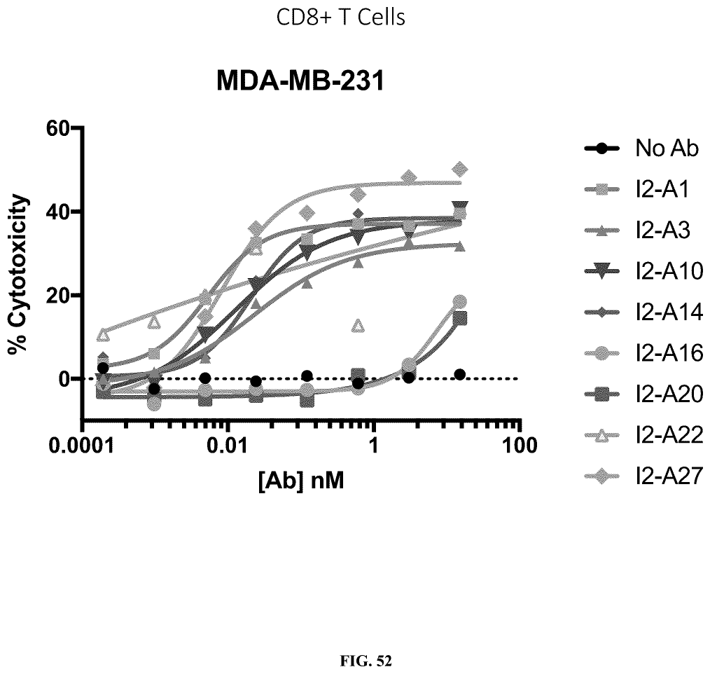

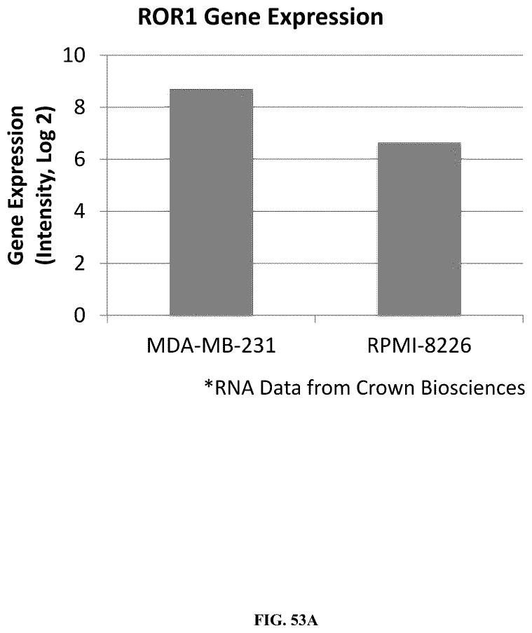

As shown in FIG. 52 and presented in Table 13, the RORxCD3 bispecific 1×2 B-bodies I2A-1, 12A-3, 12A-10, 12A-14, 12A-22, and I2A-27 resulted in cytotoxic T cell mediated killing when mixed with ROR1 expressing tumor lines (MDA-MD-231), but 1×2 B-bodies I2A-16 and I2A-20 did not result in potent cytotoxicity. In addition, I2A-22 resulted in an irregular dose response curve. Thus, ROR ABS candidates 12A-1, I2A-3, 12A-10, I2A-14, and I2A-17 resulted in effective T cell mediated killing.

TABLE 13
ROR ABS Candidate T cell Mediated Killing

| ABS | EC50 (pM) |
|---|---|
| I2-A1 | 5.3 |
| I3-A3 | 23 |
| I2-A10 | 13 |
| I2-A14 | 21 |
| I2-A16 | ND |
| I2-A20 | ND |
| I2-A22 | ND |
| I2-A27 | 8.2 |

6.13.21.5. Cytotoxic Killing Correlates with ROR1 Expression

ROR ABS candidate I2A-10 and CD3 ABS candidate SP34-89 were formatted into a bispecific B-Body “BC1” 1×2 format, as described above, with the 1×2 format having a 10 amino acid junction between the S and the H domains. The I2A-10 candidate was tested in the T cell cytotoxicity assay described. Briefly, isolated CD8+ T cells (effector cells) were mixed with ROR1 expressing tumor cell lines MDA-MD-231 (triple negative breast cancer) and RPMI-8226 (multiple myeloma) at an E:T ratio of 4:1. A dilution series of the candidate incubated with the effector:target mixture.

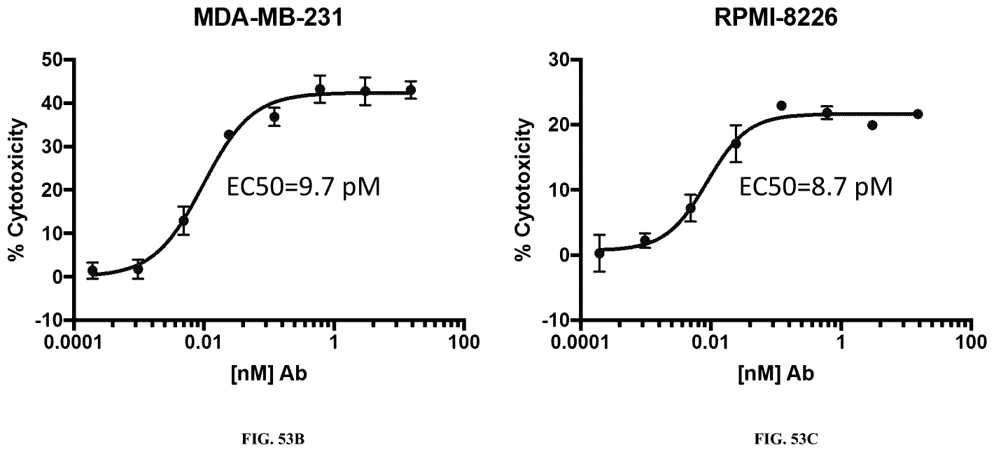

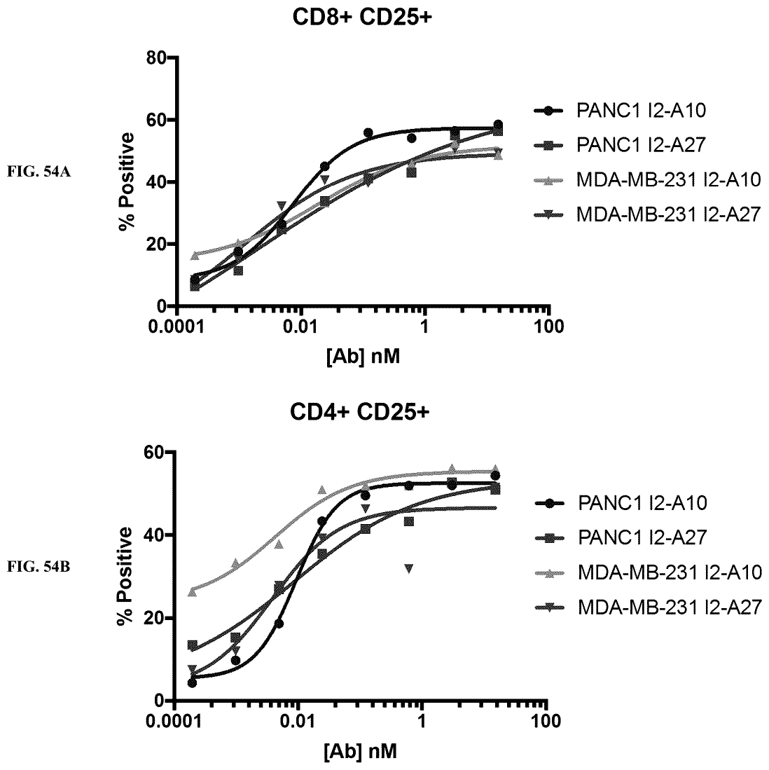

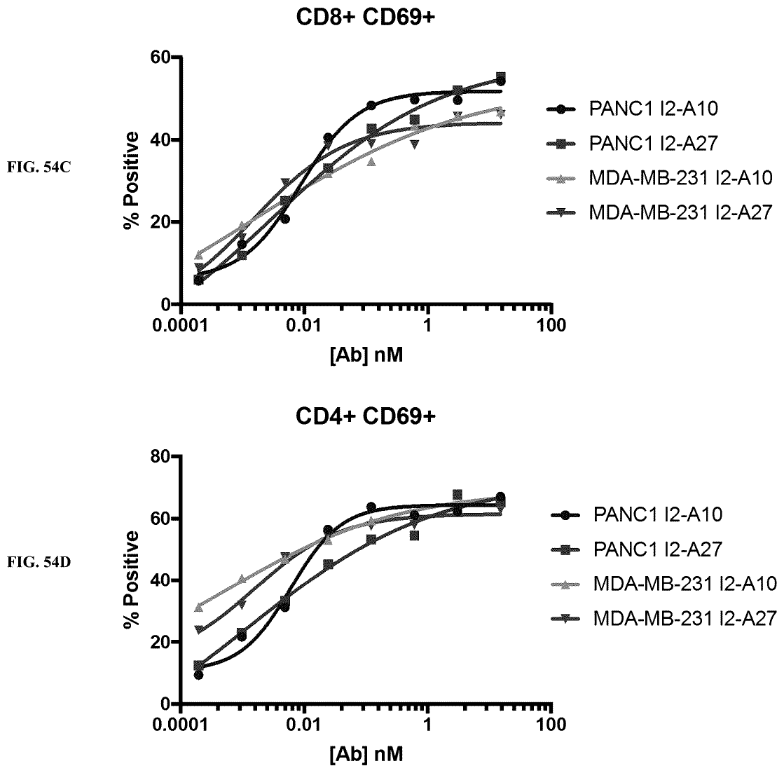

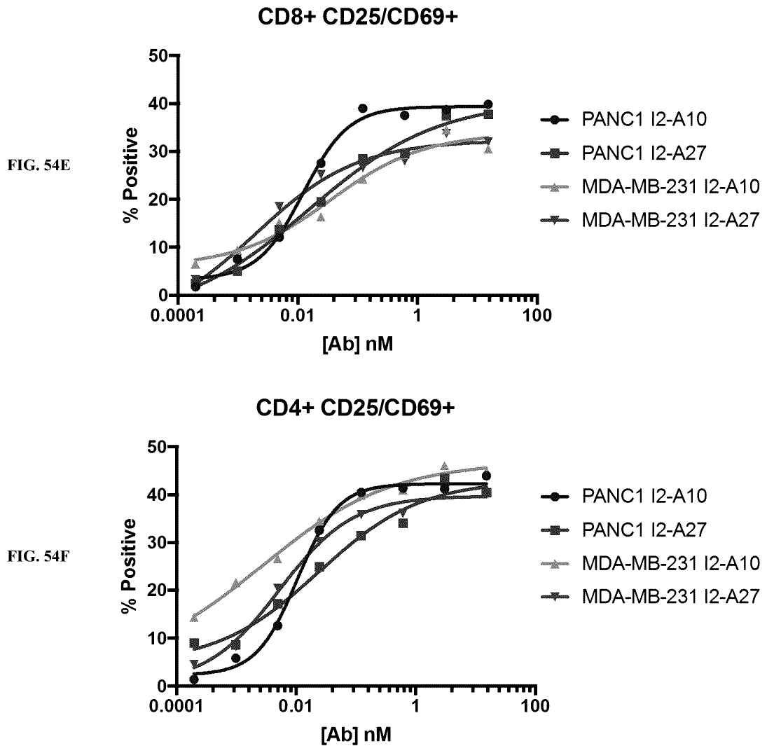

FIG. 53A illustrates published ROR1 expression data for the MDA-MD-231 and RPMI-8226 tumor lines. FIG. 53B and FIG. 53C demonstrate that the cytotoxicity efficacy observed in our experiments correlates with ROR1 in MDA-MD-231 and RPMI-8226 tumor cell lines.

6.13.21.6. Primary T cell Activation with ROR ABS Candidates I2A-10 and I2A-27

ROR ABS candidates 12A-10 and I2A-27 were formatted with CD3 ABS candidate SP34-89 into a bispecific B-body “BC1” 1×2 format, as described above, with the 1×2 format having a 10 amino acid junction between the S and the H domains. The amino acid sequences for the four polypeptide chains that form the I2A-10 BC1 1×2 B-body are listed in SEQ ID NOs:84, 85, 87, and 88. The amino acid sequences for the four polypeptide chains that form the I2A-27 BC1 1×2 are listed in SEQ ID NOs:89, 90, 92, and 93.

The different constructs were tested in the T cell activation assay, as quantified by flow cytometry. Briefly, isolated peripheral blood mononuclear cells (PBMCs, effector cells) were mixed with the triple negative breast cancer tumor cell line MDA-MD-231 and the pancreatic carcinoma tumor cell line PANC1 (target cells), each expresses the ROR1 antigen, at an E:T ratio of 7:1. A dilution series of the different B-body constructs were then incubated with the effector:target mixture and incubated together for 44 hours. Cells were then stained for T cell markers CD3, CD4, and CD8, as well as activation markers CD25 and CD69 and analyzed by flow cytometry.

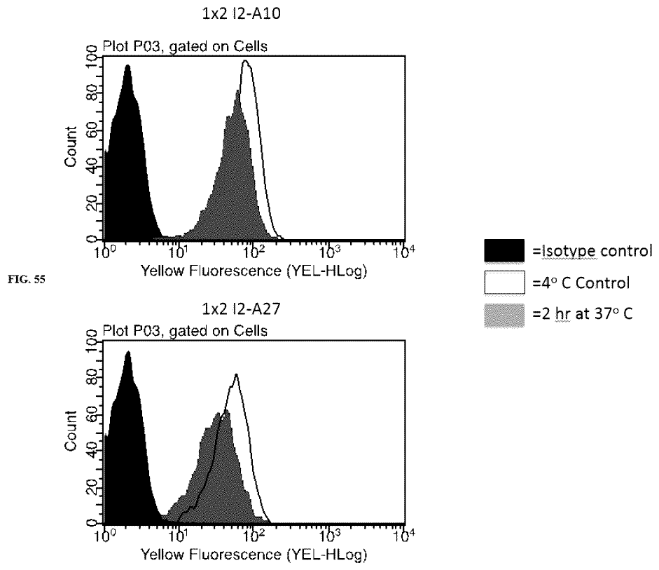

As shown in FIGS. 54A-54F, I2A-10 and I2A-27 B-bodies activated CD8+ T cells in the PBMC population as determined by expression of CD25 (FIG. 54A), CD69 (FIG. 54C), both CD25 and CD69 (FIG. 54E), and activated CD4+ T cells in the PBMC population as determined by CD25 (FIG. 54B), CD69 (FIG. 54D), both CD25 and CD69 (FIG. 54F). Thus, ROR ABS candidates are capable of activating primary T cells.

### 6.13.21.7. Internalization of ROR ABS Candidates 12A-10 and I2A-27

ROR ABS candidates 12A-10 and I2A-27 were formatted with CD3 ABS candidate SP34-89 into a bispecific B-body “BC1” 1×2 format, as described above, with the 1×2 format having a 10 amino acid junction between the S and the H domains. The different constructs were tested for internalization by tumor cell lines, as quantified by flow cytometry. Briefly, MDA-MB-231 cells were incubated for 2 hours at 370 C or 4° C. with I2-A10, I2-A27, or an isotype control. After 2 hours, a labeled secondary antibody was added at 4° C. for 30 min and then analyzed by flow cytometry. Percent internalization was calculated by normalizing between the isotype control (0%) and the 4° C. control (100%) based upon Mean Fluorescence Intensity (MFI).

As shown in FIG. 55, 26% and 36% of candidates I2A-10 (top panel) and I2A-27 (bottom panel) were internalized following a 2 hour incubation with MDA-MB-231 cells, respectively. Internalization by tumor cells makes possible various antibody-drug conjugate strategies for killing tumor cells expression a ROR antigen.

### 6.13.22. Example 21: RORxCD3 Bispecific B-Body Single Step Purification

ROR ABS candidates 12A-10 and I2A-27 were formatted with CD3 ABS candidate SP34-89 into a bispecific B-body “BC1” trivalent 1×2 format, as described above, with the 1×2 format having a 10 amino acid junction between the S and the H domains. The constructs were purified using a one-step purification using the CaptureSelect™ CH1 affinity resin.

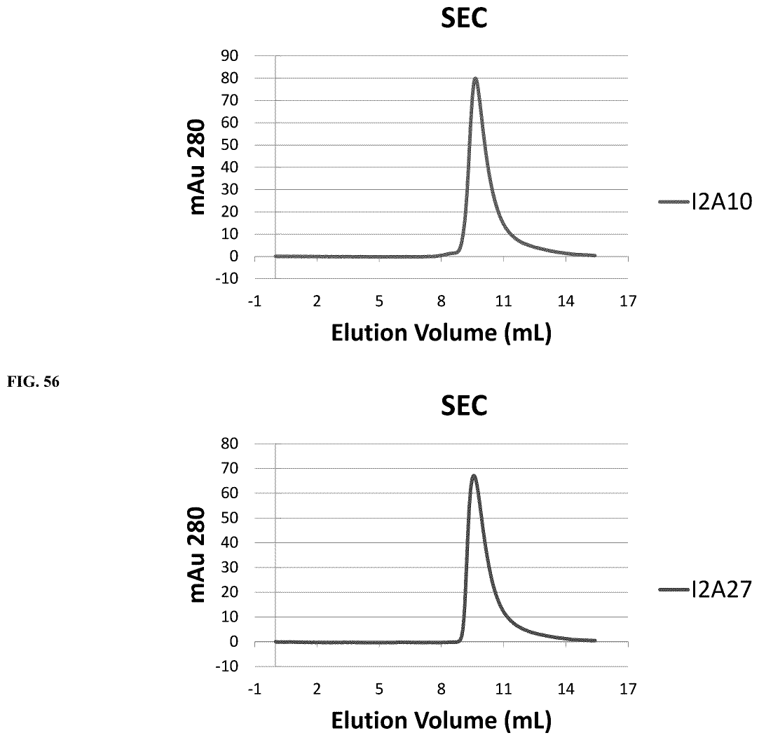

FIG. 56 shows size exclusion chromatography (SEC) analysis, demonstrating that a single-step CH1 affinity purification step yields single, monodisperse peaks via gel filtration in which>98% is unaggregated protein for 1×2 B-body candidates I2A-10 (top panel) and I2A-27 (bottom panel).

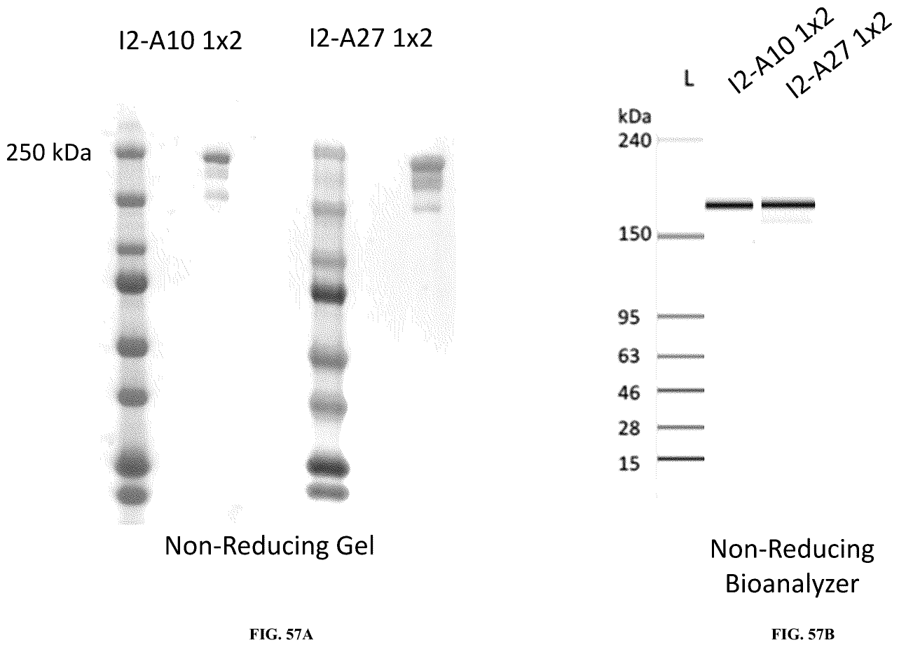

FIG. 57A shows non-reducing SDS-PAGE gels of 1×2 B-body candidates I2A-10 (left panel) and I2A-27 (right panel), demonstrating a major band of fully assembled constructs (high-migrating 250 kDa band).FIG. 57B shows Bioanalyzer (Agilent) analysis of non-reduced samples for 1×2 B-body candidates I2A-10 and I2A-27 demonstrating a major band of fully assembled constructs.

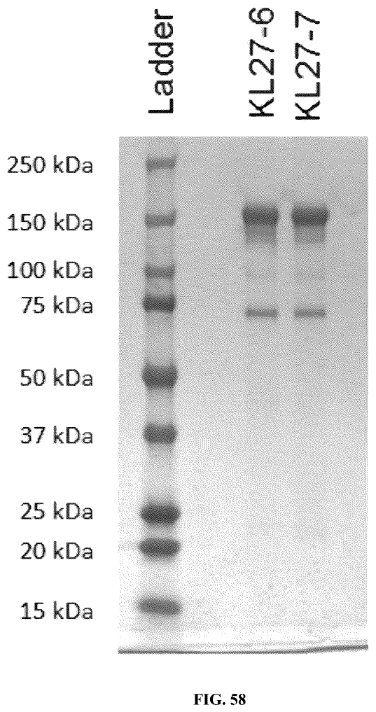

Anti-CH1 purification efficiency of bispecific antibodies was also tested for ROR binding molecules having only standard knob-hole orthogonal mutations introduced into CH3 domains found in their native positions within the Fc portion of the bispecific antibody, with no other domain modifications. Therefore, the two antibodies tested, KL27-6 and KL27-7, each contained two CH1 domains, one on each arm of the antibody. As described in more detail in Section 6.13.1, each bispecific antibody was expressed, purified from undesired protein products on an anti-CH1 column, and run on an SDS-PAGE gel. As shown in FIG. 58, a significant band at 75 kDa representing an incomplete bispecific antibody was present, interpreted as a complex containing only (i) a first and second or (ii) third and fourth polypeptide chains with reference to FIG. 3. Thus, methods using anti-CH1 to purify complete bispecific molecules that have a CH1 domain in each arm resulted in background contamination due to incomplete antibody complexes.

### 6.13.23. Example 22: Fc Mutations Reducing Effector Function

A series of engineered Fc variants were generated in the monoclonal IgG1 antibody trastuzumab (Herceptin, “WT-IgG1”) with mutations at positions L234, L235, and P329 of the CH2 domain. The specific mutations for the variants tested are described in Table 14 below and include sFc1 (PALALA), sFc7 (PGLALA), and sFc10 (PKLALA). All variants displayed similar stability as determined by melting temperatures (Table 14 TM1 and TM2).

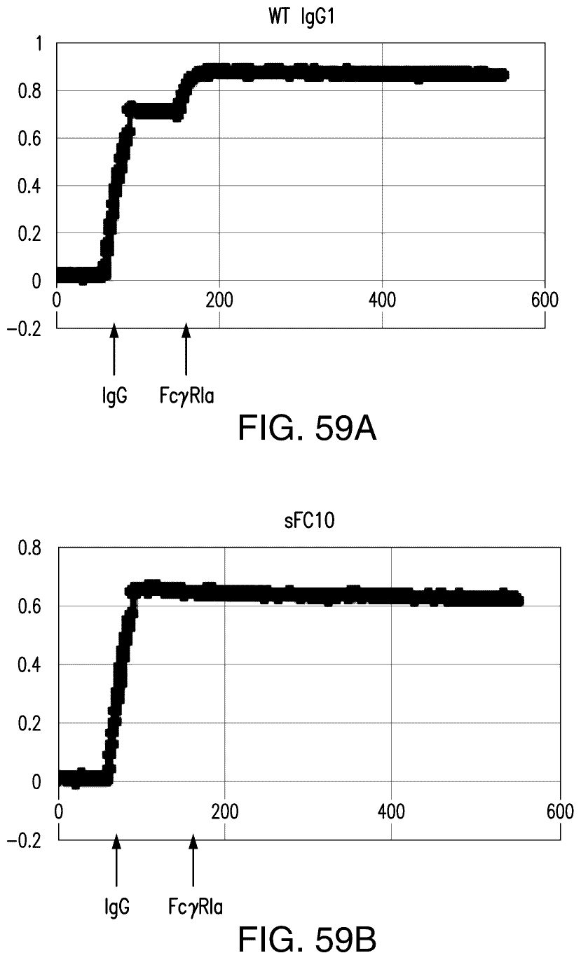

WT-IgG1 and the Fc variants were immobilized to the Octet biosensor and soluble FcγRIa was added to the system to determine binding. FIGS. 59A-59B shows Octet (Pall ForteBio) biolayer interferometry analysis demonstrating FcγRIa binding to trastuzumab (FIG. 59A “WT IgG1”), but not sFc10 (FIG. 59B). Upon addition of FcγRIa, an increase in signal was seen for trastuzumab, but no observable signal increase was detected for sFc10 demonstrating FcγRIa no longer binds the antibody with the engineered mutations. Binding summaries for the variants tested presented in Table 14. In addition, all variants retained strong binding to HER2 (not shown).

TABLE 14
Fc Variant Comparison

| | | | | TM1 | TM2 | FcγRIa | |
|---|---|---|---|---|---|---|---|
| Variant | L234 | L235 | P329 | (° C.) | (° C.) | Binding | |
| sFc1 | A | A | P | 68.1 | 81.8 | Yes | (weak) |
| sFc7 | A | A | G | 65.7 | 81.8 | No | |
| sFc10 | A | A | K | 65.3 | 81.1 | No | |
| WT IgG1 | L | L | P | 66.2 | 81.1 | Yes | (strong) |

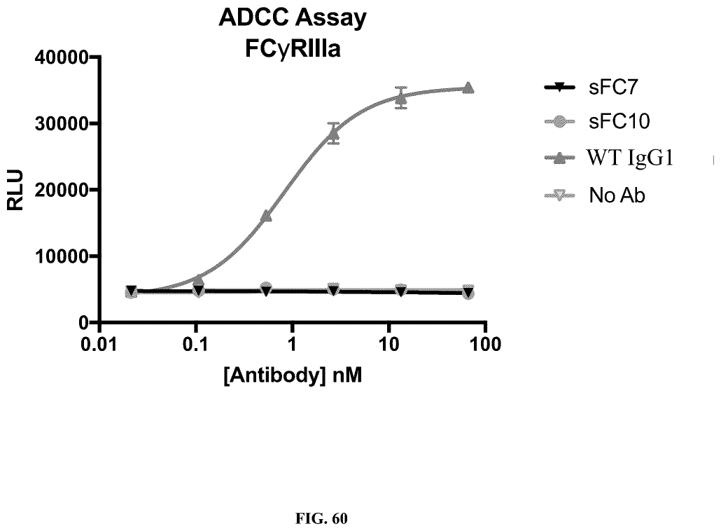

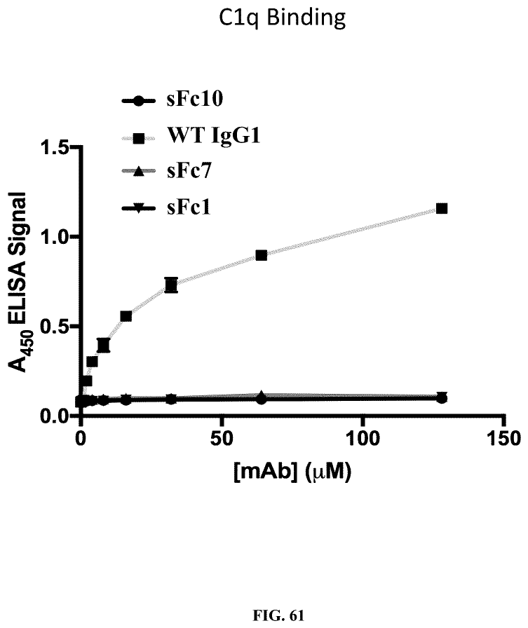

WT-IgG1 and the Fc variants were tested in an antibody dependent cellular cytotoxicity (ADCC) assay as another measure of FcγR binding, specifically FcγRIIIa. As shown in FIG. 60, trastuzumab (Herceptin, “WT-IgG1”) demonstrated killing, while neither sFc7 nor sFc10 resulted in detectable levels of killing. WT-IgG1 and the Fc variants were also tested for complement component C1q binding by ELISA. As shown in FIG. 61, trastuzumab (Herceptin, “WT-IgG1”) demonstrated C1q binding, while neither sFc1, sFc7, nor sFc10 resulted in detectable C1q binding. Thus, the results demonstrate that the Fc variants tested have reduced levels of Fc effector function. 6.13.24. Example 23: RORxCD3 Bispecific B-Body Efficacy in vivo

In vivo efficacy was measured using xenograft studies in humanized mice. Briefly, 5×10⁶ MDA-MB-231 tumor cells were engrafted subcutaneously into the hind flank of NOD Scid Gamma (NSG) mice (Jackson Labs) and grown to approximately 120-150 mm3. The mice were then humanized through the intravenous (IV) injection of 1×107 human PBMCs from a single donor. The humanized NSG mice were randomized into three groups of eight and dosed IV with PBS, 0.5 mg/Kg ROR ABS candidate I2-A10 formatted with CD3 ABS candidate SP34-89 into a 1×2 B-body architecture (“I2-A10”), or 0.5 mg/Kg ROR ABS candidate I2-A27 formatted with CD3 ABS candidate SP34-89 into a 1×2 B-body architecture (“12-A27”) three days following PBMC engraftment. Dosing continued twice a week for three weeks. Animals were monitored for tumor growth, body weight, and general health. Upon completion of the study, the animals were sacrificed for analysis. Flow cytometric analysis was performed using standard techniques to determine the humanization status of the NSG mice.

Tumors are collected and analyzed using standard immunohistochemistry techniques to monitor the infiltration of human T-cells into the tumors in response to treatment. IHC is carried out using standard techniques. Briefly, FFPE samples are deparaffinized and rehydrated by baking at 60° C., placing in 100% xylene solution, and then rehydrated through a series of ethanol washes (100% ethanol, 95% ethanol, 70% ethanol, 50% ethanol, PBS). Antigen retrieval is carried out by incubating the slides for 10 minutes in a 10 mM NaCitrate +0.05% Tween 20, pH 6.0 buffer at 95° C. The AbCam Mouse on Mouse IHC kit (Cat. Ab127055) is used according to manufacturer's directions for staining. Briefly, endogenous peroxidase activity is blocked with a hydrogen peroxide solution and endogenous non-specific interactions are blocked with a Rodent Block solution. The slides are incubated with the primary antibodies according to the manufacturer's recommendations (generally 2 hr. at room temperature or overnight at 4° C.) and then stained with the mouse on mouse HRP polymer from the AbCam kit. DAB chromagen staining with hemotoxylin counterstaining are used for visualization.

### 6.13.24.1. RORxCD3 Trivalent Bispecific B-Body Results in Tumor Growth Reduction

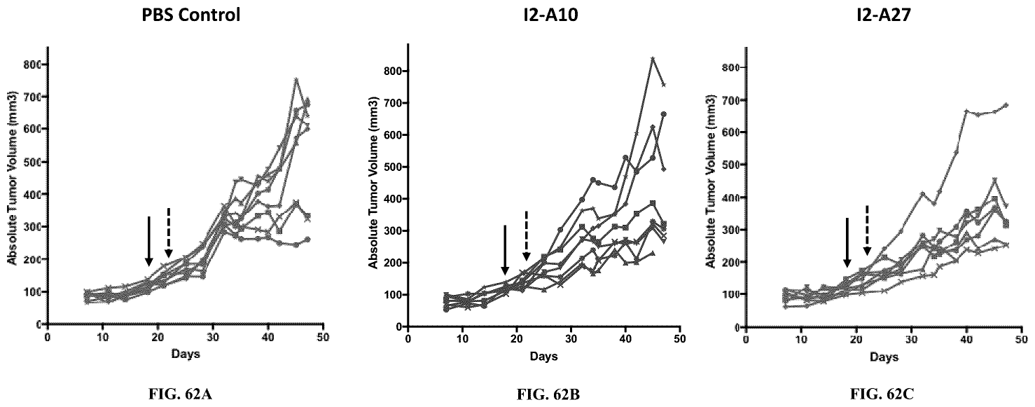

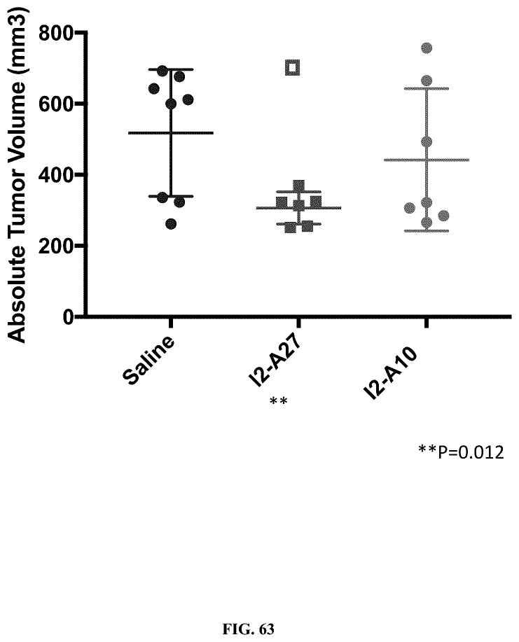

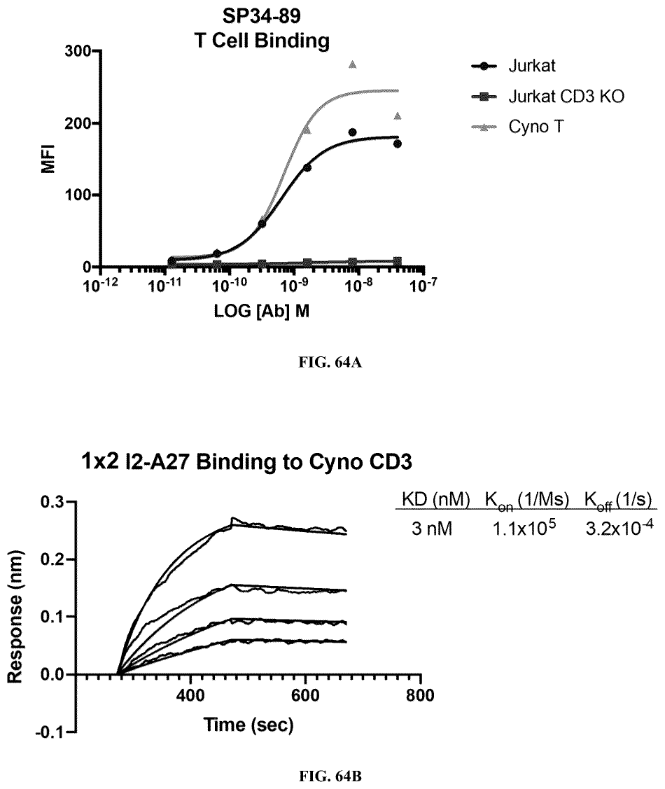

As shown in FIGS. 62A-62C, tumor volume was monitored for mice engrafted with tumor cells, humanized with PBMCs (left solid arrow), then subsequently treated IV (right dashed arrow) with PBS (FIG. 62A), 1×2 B-body candidate I2-A10 (FIG. 62B), or 1×2 B-body candidate I2-A27 (FIG. 62C). FIG. 63 shows tumor volume at the conclusion of the study for each of the mice, with mean and standard deviation for each group shown. The open square for the I2-A27 group was removed from the analysis due to probable non-humanization by PMBCs. The results demonstrate that the I2-A27 treatment resulted in a significant reduction in tumor growth of approximately 40%.

Humanization status of the NSG mice in the study was confirmed through flow cytometric analysis. As shown in Table 15, all mice analyzed were successfully humanized with human lymphocytes, as determined by staining for human CD45 (% hu CD45+). Sample 03_B10 (b) was not analyzed. The humanized lymphocyte populations in all mice analyzed also had human T cells, including CD4 and CD8 positive T cells, as determined by staining for human CD3 (% CD3 gated on huCD45+ lymphocytes, and % CD4+/CD8+gated on human CD3+ T cells).

Immunohistochemistry is performed on the tumor samples and confirms infiltration of human T-cells into the tumors in response to treatment.

TABLE 15
Humanization of NSG Mice

| | | Non-doublet gate | | huCD45+ gate | human CD3+ gate | |
|---|---|---|---|---|---|---|
| Sample ID | Condition | % ms CD45+ | % hu CD45+ | % CD3+ | % CD4+ | % CD8+ |
| 01_A01 | PBS | 48.1 | 50.8 | 99.6 | 44.5 | 46.1 |
| 01_A02 | PBS | 60.1 | 39.0 | 98.5 | 76.6 | 13.7 |
| 01_A07 | PBS | 56.0 | 43.8 | 99.1 | 23.5 | 64.1 |
| 01_B06 | PBS | 42.6 | 56.4 | 99.8 | 80.6 | 9.8 |
| 01_C04 | PBS | 51.0 | 47.7 | 99.2 | 64.6 | 23.8 |
| 01_C05 | PBS | 22.1 | 77.0 | 98.5 | 77.9 | 14.1 |
| 01_C09 | PBS | 67.8 | 31.8 | 99.4 | 27.7 | 64.6 |
| 01_D05 | PBS | 37.0 | 62.0 | 98.7 | 77.2 | 18.0 |
| 02_B01 | I2-A10 1 × 2 | 44.8 | 53.5 | 99.0 | 83.3 | 10.4 |
| 02_B07 | I2-A10 1 × 2 | 50.4 | 47.8 | 98.3 | 78.6 | 16.5 |
| 02_C02 | I2-A10 1 × 2 | 6.9 | 92.8 | 98.5 | 81.5 | 11.9 |
| 02_C03 | I2-A10 1 × 2 | 32.6 | 65.2 | 99.5 | 81.6 | 9.2 |
| 02_C06 (a) | I2-A10 1 × 2 | 2.9 | 94.4 | 95.0 | 60.2 | 30.9 |
| 02_C08 (a) | I2-A10 1 × 2 | 31.3 | 65.2 | 98.7 | 53.0 | 39.6 |
| 02_D04 (a) | I2-A10 1 × 2 | 29.9 | 66.5 | 99.3 | 33.9 | 61.1 |
| 03_A03 | I2-A27 1 × 2 | 32.4 | 66.4 | 93.0 | 84.7 | 3.8 |
| 03_A04 | I2-A27 1 × 2 | 30.5 | 66.9 | 98.9 | 84.9 | 6.2 |
| 03_A10 | I2-A27 1 × 2 | 58.6 | 40.2 | 98.9 | 92.5 | 4.2 |
| 03_B02 | I2-A27 1 × 2 | 20.5 | 78.2 | 99.2 | 86.6 | 7.6 |
| 03_B10 (b) | I2-A27 1 × 2 | — | — | — | — | — |
| 03_C10 | I2-A27 1 × 2 | 34.2 | 64.2 | 98.8 | 91.7 | 4.8 |
| 03_D03 | I2-A27 1 × 2 | 60.9 | 38.5 | 99.4 | 49.8 | 43.2 |

### 6.13.25. Example 24: ROR1xCD3 Bispecific B-body™ Efficacy

Additional ROR antigen binding molecules comprising the six CDRs of 12A-27 as shown in Table 6 were prepared and experiments were performed to test their efficacy as trivalent bispecific constructs in a ROR1xCD3 bispecific B-body™ format.

6.13.25.1. Generation of additional ROR1xCD3 1×2 B-body™ bispecific antibody based on I2-A27

ROR ABS candidate I2A-27 was formatted with CD3 ABS candidate SP34-89 into a bispecific B-body “BC1” 1×2 format, as described above, with the 1×2 format having a 10 amino acid junction between the S and the H domains (see, e.g., FIG. 26 for schematic of domains).. The amino acid sequences for the five polypeptide chains that form an exemplary I2A-27 BC1 1×2 trivalent bispecific ROR antigen binding molecule are listed as SEQ ID NO:96 (Chain 1), SEQ ID NO:97 (Chain 2), SEQ ID NO:98 (Chain 3), SEQ ID NO:99 (Chain 4), and SEQ ID NO:97 (Chain 5).

In the following description in this Example, the term “12-A27 1×2 B-body™” or “12-A27” or “A27” refers to this ROR1xCD3 1×2 B-body™ having a Chain 1 of SEQ ID NO:96, a Chain 2 of SEQ ID NO:97, a Chain 3 of SEQ ID NO:98, a Chain 4 of SEQ ID NO:99, and a Chain 5 of SEQ ID NO: 97.

6.13.25.2. Binding of ROR1xCD3 1×2 B-body™ bispecific Antibody to CD3 and ROR1

As described in Example 19 above, SP34-89 showed a monovalent Kd of 23 nM to human CD3 delta and epsilon heterodimer (see FIG. 47).

In this Example, the binding to human and cynomolgus CD3 was further characterized by assessing cell binding to Jurkat T cells, a Jurkat CD3 knock-out cell line, or a cynomolgus T cell line. Specifically, binding of the SP34-89 humanized antibody to human Jurkat and cynomolgus T cells was determined by flow cytometry. SP34-89 was incubated with the indicated cell lines at the indicated concentrations followed by labeling with a fluorescently labeled secondary antibody. The mean fluorescence intensity was plotted versus the concentration of SP34-89. As shown in FIG. 64A, SP34-89 exhibited strong binding to both the Jurkat and cynomolgus T cells but did not show significant binding to the Jurkat CD3 knock-out cell line, verifying that the human and cynomolgus cross reactivity and cell binding was maintained.

In addition, the binding affinity of the resulting I2-A27 1×2 B-body™ bispecific antibody as described in section 6.13.25.1 to cynomolgus CD3 delta and epsilon heterodimer was determined by bio-layer interferometry (BLI) binding assessment.

More specifically, monovalent binding affinity analysis was performed using the Octet^{QK} 384 (ForteBio) using Fc biosensors. Unlabeled B-body™ is first bound to the Fc biosensor at a concentration to yield approximately 1 nm response (typically 10 to 50 nM protein). The biosensors are then equilibrated in 10× Kinetic Buffer (ForteBio) to establish a baseline. The untagged monomer of each antigen is contacted with the antibody-coated sensors at concentrations ranging from 50 nm to 0.75 nM. The association response and dissociation were monitored and recorded. The resulting Kₒₙ and K_{off} was fit using the Octet analysis software. As shown in FIG. 64B, the bispecific antibody exhibited a monovalent Kd of 3 nM to cynomolgus CD3 delta and epsilon heterodimer.

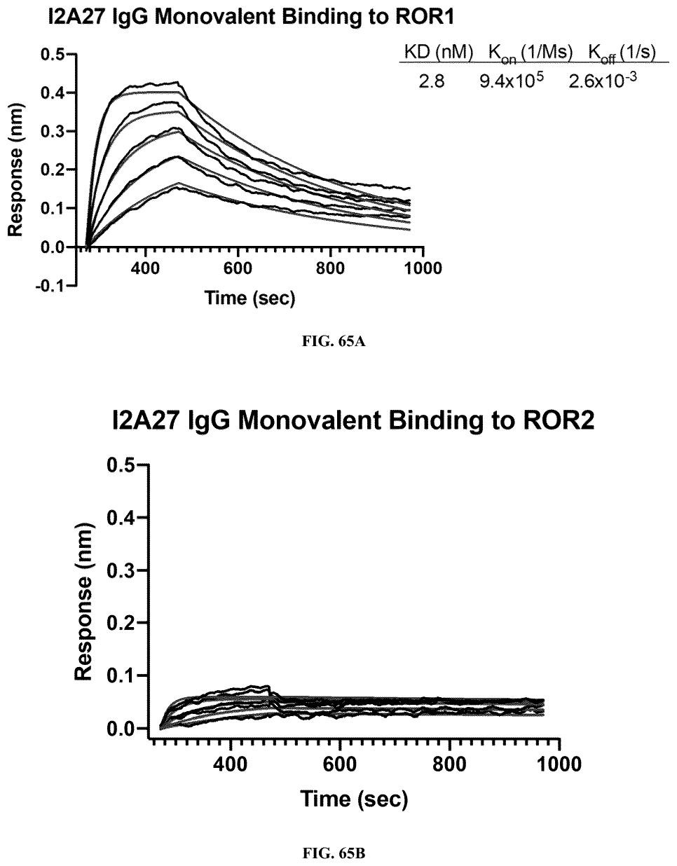

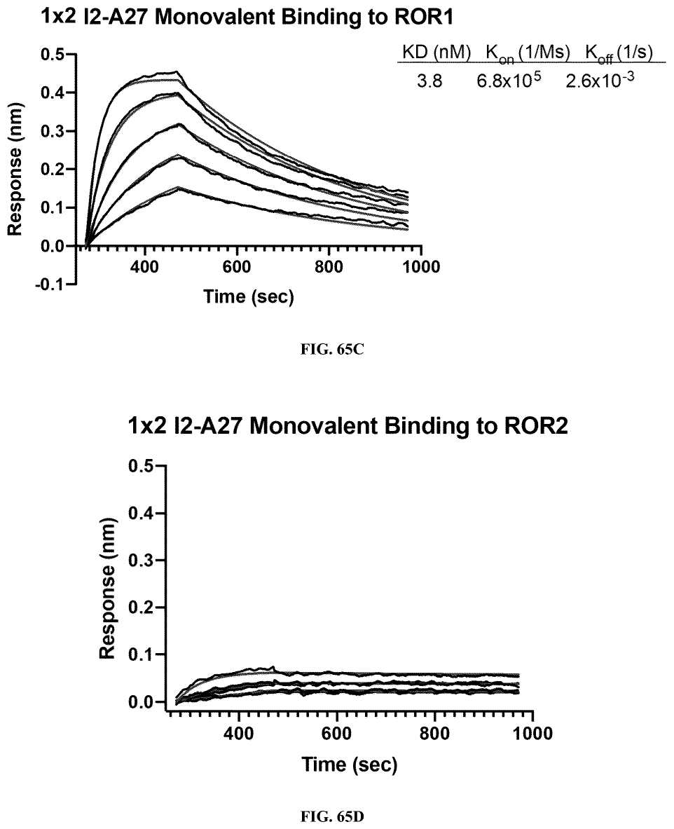

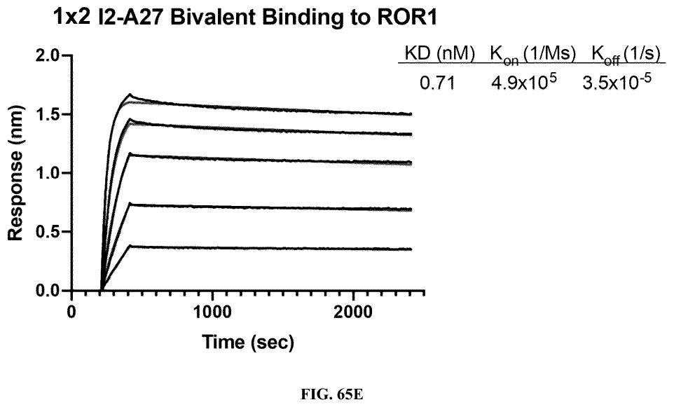

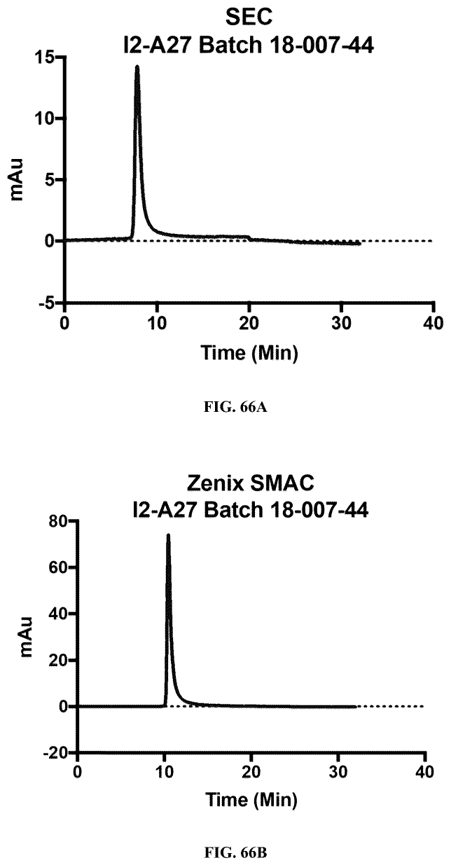

Next, the binding to ROR1 was assessed. Consistent with the data described in 6.13.20.2 above (see, e.g., FIG. 48B), when the VH and VL sequences were formatted into a bivalent monospecific IgG architecture, the I2-A27 IgG antibody showed monovalent binding of 2.8 nM to ROR1 and minimal binding to ROR2 (FIG. 65A and FIG. 65B).

Then, the monovalent and bivalent binding of the I2-A27 1×2 B-body™ bispecific antibody to ROR1 and ROR2 was analyzed using BLI.

Monovalent binding analysis was performed as described above and the antibody was captured with an Fc sensor and contacted with the indicated antigen for the association phase followed by a dissociation phase in buffer only.

Bivalent binding analysis was performed using the Octet^{QK} 384 (ForteBio) using streptavidin biosensors. Biotinylated antigen was first bound to the streptavidin biosensor at a concentration to yield approximately 1 nm response (typically 10 to 50 nM biotinylated protein). The biosensors were then equilibrated in 10× Kinetic Buffer (ForteBio) to establish a baseline. The 1×2 B-body™ was contacted with the antigen-coated sensors at concentrations ranging from 50 nm to 0.75 nM. The association response and dissociation were monitored and recorded. The resulting Kₒₙ and K_{off} was fit using the Octet analysis software.

As shown in FIGS. 65C, 65D, and 65E, the I2-A27 1×2 B-body™ format showed monovalent binding of 3.8 nM and bivalent binding of 0.71 nM to ROR1 and minimal binding to ROR2.

6.13.25.3. Further Characterization of ROR1xCD3 1×2 B-body™ bispecific antibody

An I2-A27 1×2 B-body™ bispecific antibody as described in section 6.13.25.1 was produced through transient transfection of DNA encoding polypeptide chains 1-4 (polypeptide chain 5 is the same as polypeptide chain 2) in Expi-CHO cells. The five chain antibody was purified with a CH1 affinity resin and buffer exchanged into PBS, pH 7.4.

The assembly and purity of the resulting B-body™ was assessed through non-reducing and reducing SDS-PAGE.

Non-reducing SDS-PAGE was performed using 2 μg of antibody ranging from −0.2 to 1 mg/mL. Sample, up to 10 μL, was mixed with 4 μL 2× Laemmli Sample Buffer (Bio-Rad) in 200 μL PCR tubes. To a separate tube was added 4 μL molecular weight ladder (Precision Plus Protein Dual Color Standards, Bio-Rad). PCR tubes were incubated at 55° C. for 10 min and cooled to 4° C. using a thermocycler. Tubes were centrifuged and the entire volume of each tube was loaded into individual wells of a 4-15% Bis-Tris Gel (Mini-PROTEAN TGX, Bio-Rad). Samples were electrophoresed over 30 min using a Bio-Rad Mini-PROTEAN Tetra System with Tris/Glycine/SDS running buffer (diluted from 10× to 1X) operated at constant voltage (220 V) using an external power supply. Gels were washed with deionized water and stained with coomassie dye (GelCode Blue Safe Protein Stain, ThermoFisher Scientific) for a minimum of 15 min. Gels were subsequently destained with deionized water for a minimum of 30 min and imaged.

Reducing SDS-PAGE was performed using 3 μg of antibody ranging from −0.3 to 1.5 mg/mL. Sample, up to 10 μL, was mixed with 4 μL 2× Laemmli Sample Buffer (Bio-Rad) in 200 μL PCR tubes and 5 μL of 5 M dithiothreitol. To a separate tube was added 4 μL molecular weight ladder (Precision Plus Protein Dual Color Standards, Bio-Rad). PCR tubes were incubated at room temperature for 30 min. Samples were then incubated at 55° C. for 10 min and cooled to 4° C. using a thermocycler. Tubes were centrifuged and up to 14 μL of sample was loaded into individual wells of a 4-15% Bis-Tris Gel (Mini-PROTEAN TGX, Bio-Rad). Samples were electrophoresed over 30 min using a Bio-Rad Mini-PROTEAN Tetra System with Tris/Glycine/SDS running buffer (diluted from 10× to 1X) operated at constant voltage (220 V) using an external power supply. Gels were washed with deionized water and stained with coomassie dye (GelCode Blue Safe Protein Stain, ThermoFisher Scientific) for a minimum of 15 min. Gels were subsequently destained with deionized water for a minimum of 30 min and imaged.

The 1×2 B-body™ ran near the expected molecular weight of 200 kD in a non-reduced format (data not shown here) In a reduced format, the expected 3 bands were resolved (one chain at 75 kD, one chain at 50 kD, and 2 chains at 25 kD) (data not shown here).

The protein was subsequently analyzed by capillary electrophoresis in a reduced and non-reduced format.

Capillary electrophoresis was run on the Agilent 2100 Bioanalyzer following the manufacturer's instructions. Briefly, the samples were incubated with labeling dye in labeling buffer on ice in the dark for 30 min. Ethanolamine was then and the samples were incubated for 10 min on ice in the dark to quench unincorporated dye. For reduced samples, 1 M DTT was added to the sample buffer. Samples were incubated at 98° C. for 5 min to denature the samples prior to loading in the NanoFluidic chip. The percent purity was calculated from the three major bands in the reduced sample to be 95.54% (data not shown here).

The antibody protein was then analyzed via a set of columns to assess the homogeneity with size-exclusion chromatography (SEC), the propensity towards aggregation with standup monolayer adsorption chromatography (SMAC), and the hydrophobicity with hydrophobic interaction chromatography (HIC).

SEC analysis was performed using a 7.8 mm ID x 30 cm TSKgel G3000SWXL column (Tosoh Bioscience LLC, PN 08541) on an Agilent 1100 HPLC. Antibodies were normalized to 1 mg/mL concentration in dPBS (pH 7.4) and clarified via centrifugation to pellet particulates. The mobile phase buffer was dPBS (pH 7.4, without calcium and magnesium). For each sample, 10 μL was loaded and isocratically eluted at 1.0 mL/min over 20 min. Absorbance was monitored at 280 nm. Chromatographic peaks were integrated to determine % homogeneity and retention time.

SMAC analysis was performed using a 4.6 mm ID x 300 mm Zenix SEC 300 column (Sepax Technologies, PN 213300P-4630) on an Agilent 1100 HPLC. Antibodies were normalized to 1 mg/mL concentration in dPBS (pH 7.4) and clarified via centrifugation to pellet particulates. The mobile phase buffer was dPBS (pH 7.4, without calcium and magnesium). For each sample, 10 μL was loaded and isocratically eluted at 0.25 mL/min over 32 min. Absorbance was monitored at 280 nm. Sample retention time was calculated and compared to a set of standard controls to identify antibodies with increased retention time (increased propensity to form aggregates).

HIC analysis was performed using a 4.6 mm ID x 3.5 cm TSKgel Butyl-NPR column (Tosoh Bioscience LLC, PN 14947) on an Agilent 1100 HPLC. Antibodies were normalized to 2 mg/mL concentration in dPBS (pH 7.4) and then diluted with an equal volume of mobile phase buffer B to a final protein concentration of 1 mg/mL. The column was equilibrated with 100% mobile phase Buffer B (2 M ammonium sulfate/20 mM sodium phosphate, pH 7.0) at a flow rate of 1 mL/min. For each sample, 10 μL was loaded and eluted using a gradient from 100% mobile phase buffer B to 100% mobile phase buffer A (20 mM sodium phosphate, pH 7.0) at 1.0 mL/min over 15 min, held at 100% A for 3 min to wash the column, and returned 100% B for 2 min for equilibration. Absorbance was monitored at 280 nm. Sample retention time was calculated and compared to a set of standard controls to identify antibodies with increased retention time (increased hydrophobicity).

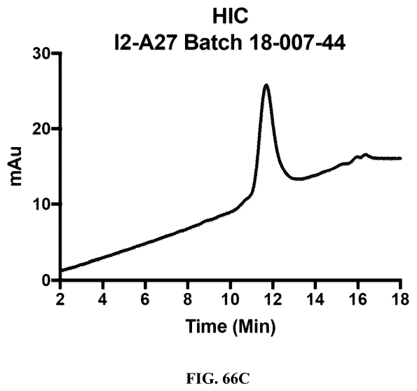

The results of these analysis are shown in FIGS. 66A, 66B and 66C. As shown, the protein appeared >99% homogeneous by SEC and resulted in a single peak with both SMAC and HIC that fell within the range observed for other developable antibodies suggesting an acceptable aggregation and hydrophobicity profile.

The protein was next analyzed with the UNcle analytic instrument which is capable of measuring the Tm, PD1, and hydrodynamic diameter using dynamic light scattering (DLS), static light scattering (SLS), and fluorescence. Antibodies were normalized to 1 mg/mL concentration in dPBS (pH 7.4) and clarified via centrifugation to pellet particulates. Samples were aliquoted into UNcle's 9 μL quartz capillary cuvette device (Uni) and sealed. PDI and hydrodynamic diameter were measured by DLS at 15° C. The temperature was ramped from 15° C. to 95° C. at 0.5° C./min during which Tm was measured by intrinsic fluorescence. Data were analyzed using UNcle Analysis Software v 3.1.

The protein showed melting temperatures of 67.4° C. and 73.7° C. The median PDI from three measurements was 0.19 which is indicative of a monodisperse sample (PDI<0.25 is considered monodisperse) with a hydrodynamic diameter of 14.4 nm (see Table 16 below).

TABLE 16
Results for melting temperature, polydispersity index, and
hydrodynamic diameter from the UNcle analytical instrument

| | Tm1 (° C.) | Tm2 (° C.) | PDI | Diameter (nm) |
|---|---|---|---|---|
| I2-A27 1 × 2 | 67.4 | 73.7 | 0.19 | 14.4 |
| B-body ™ | | | | |

### 6.13.25.4. In Vitro Functional Assessment of ROR1xCD3 1×2 B-Body™ Bispecific Antibody

The functional capability of an I2-A27 B-body™ in cellular co-culture assays of T cells and ROR1 expressing cancer cells was analyzed. Cancer cell lines that expressed ROR1 and/or ROR2 were assessed.

MDA-MB-231, RPMI-8226, and K562 were assessed for the number of copies of surface expressed ROR1 and the closely related ROR2 protein via flow cytometry using a ROR1 specific antibody (I2-A27) and a ROR2 specific antibody (I2-C21). Specificity of the antibodies used for copy number assessment was determined by BLI and it was confirmed that I2-A27 selectively binds to ROR1 and I2-C21 selectively binds to ROR2. The MDA-MB-231 cell line was determined to primarily express ROR1, the RPMI-8226 cell line was determined to express both ROR1 and ROR2, and the K562 cell line was determined to express primarily ROR2 as shown in Table 17.

TABLE 17
Surface protein copy number of ROR1 and ROR2
on MDA-MB-231, RPMI-8226, and K562 cells

| | ROR1 Copy Number | ROR2 Copy Number |
|---|---|---|
| MDA-MB-231 | 114,000 | <7,000 |
| RPMI-8226 | 110,000 | 72,000 |
| K562 | <7,000 | 68,000 |

To assess whether the I2-A27 B-body™ could activate T cells in the presence of ROR1 or ROR2 expressing cancer cells, an NFkB Jurkat co-culture reporter gene assay was utilized. Activation of the Jurkat T cells leads to activation of the NFkB response element and production of eGFP. Specifically, RPMI-8226 or K562 cells were plated the day of the assay (35,000 cells/well) or MDA-MB-231 cells were plated the day before the assay (25,000 cells/well) in a half area, black-walled, clear-bottom 96 well plate. The day of the assay, dilutions of the antibody at the concentrations indicated were added in the presence of 1 μg/mL anti-CD28 antibody. NFkB-GFP Jurkat reporter cells were added to the well at 75,000 cells/well. The plate was incubated for 6 hours at 37° C./5% C02. A background suppression dye was added and a Safire plate reader was used to determine the fluorescence at 520 nm.

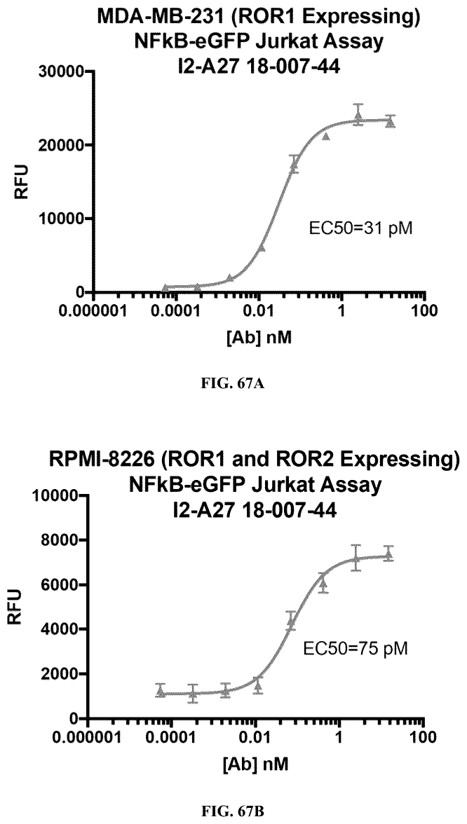

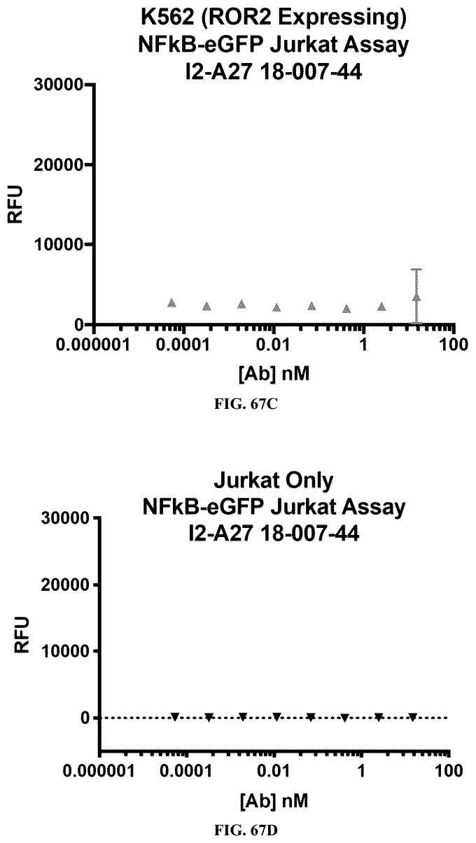

As shown in FIGS. 67A, 67B, 67C and 67D, the I2-A27 1×2 B-body™ bispecific antibody was shown to be able to activate the Jurkat reporter cell line in the pM range in the presence of MDA-MB-231 (ROR1 expressing) or RPMI-8226 (ROR1 and ROR2 expressing) cells but not in the presence of K562 (ROR2 expressing) cells or in the absence of a target cell line.

Next, the function of the I2-A27 1×2 B-body™ bispecific antibody to activate primary CD8+ T cells was studied in the presence of ROR1 expressing cell lines as assessed by increased expression levels of the early T cell activation marker, CD69, and the late T cell activation marker, CD25. Specifically, the I2-A27 1×2 B-body™ bispecific antibody, CD8+T cells, with or without MDA-MB-231 cells or RPMI-8226 cells at a 6:1 E:T ratio, were incubated for 48-hours. Then, cells were labeled with PE-Cy7 CD69 antibody (BD Biosciences 561928) and a BB515 CD25 antibody (BD Biosciences 564467) for 1 hr at 4° C. After labeling, samples were centrifuged at 300×g and resuspended in live cell imaging solution (Thermo Fisher) prior to analysis by flow cytometry on the IntelliCyt IQue screener.

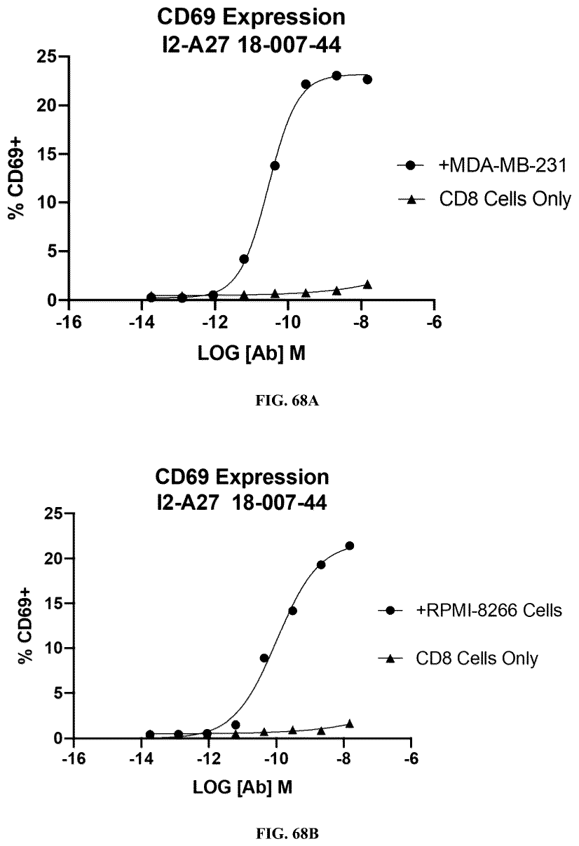

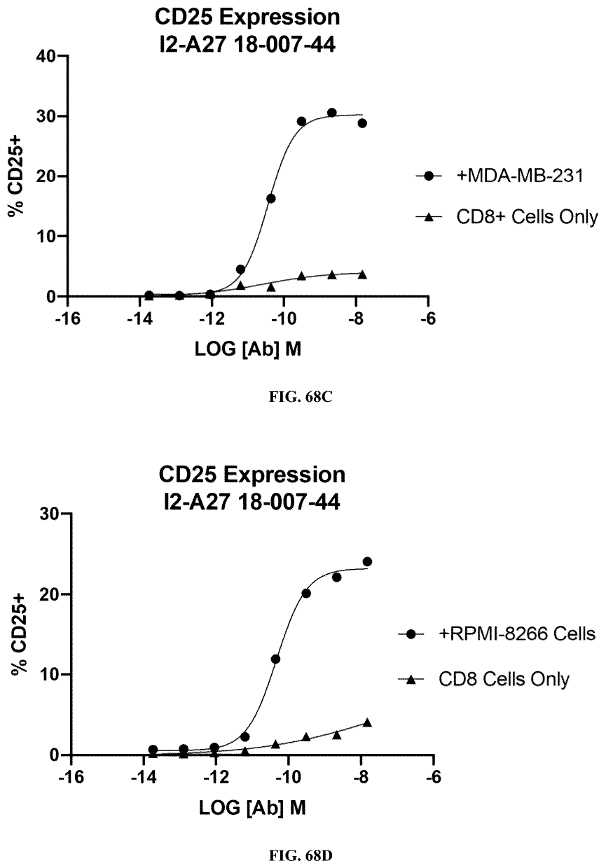

As shown in FIGS. 68A-68D, the I2-A27 1×2 B-body™ bispecific antibody led to a dose dependent increase in expression of CD69 and CD25 when in the presence of ROR1 expressing cells (MDA-MB-231 or RPMI-8226) but not in the absence of these cells.

Whether the activated T cells could subsequently kill the ROR1 expressing cell lines was assessed by LDH release from the target cells and secretion of Granzyme B, TNFα, and IFNγ by the CD8+effector cells.

LDH release from the target cells was analyzed as follows: RPMI-8226 cells were plated the day of the assay or MDA-MB-231 cells were plated the day before the assay in half area, black walled, clear-bottom 96-well plates. CD8+cells were added at an effector to target cell ratio of 6:1. Assay media was RPMI+2% heat inactivated FBS. Cells were incubated for two days at 37° C./5% C02. A lactate dehydrogenase (LDH) release assay (Roche) was performed following the manufacturer's protocol using the assay media from each well. Data was normalized to a no CD8+control (0% Cytotoxicity) and a detergent killed control (100% Cytotoxicity).

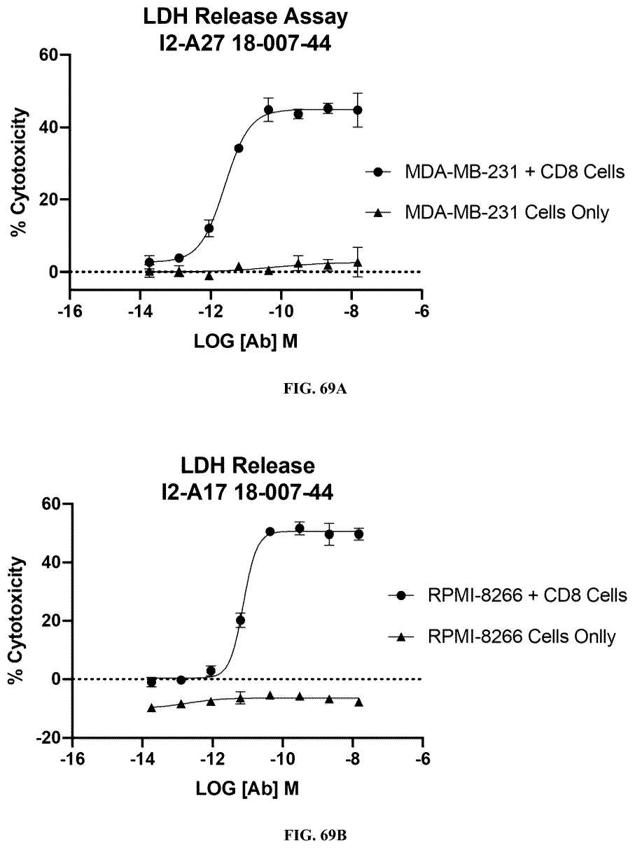

As shown in FIGS. 69A and 69B, the I2-A27 1×2 B-body™ bispecific antibody led to dose dependent cytotoxicity of ROR1 expressing cells in the presence of CD8+ T cells, but not in the absence of CD8+ T cells.

Secretion of Granzyme B, TNFα, and IFNγ by the CD8+effector cells was analyzed as follows: MultiCyt QBeads PlexScreen Secreted Protein Assay Kit (IntelliCyt) was used to determine the level of secreted TNFα, IFNγ, and Granzyme B following the Manufacturer's protocol. Briefly, capture beads for each analyte are provided as a 50× concentrate. The beads are combined together and diluted to a 2× concentrate prior to use. 10 μL of supernatant is transferred to a V bottom plate, 10 μL of prepared beads are added to the samples, the plate is mixed on a plate shaker, and the plate is incubated at room temperature for 1 hr. Following the incubation, 10 μL of detection reagent is added to each well, the plate is mixed on a plate shaker, and the plate is incubated at room temperature for 2 hr.

Following the incubation, 50 μL of wash buffer is added to each well, the plate is centrifuged for 5 min at 1100xg, the supernatants are aspirated and the samples are resuspended by adding 10 μL of wash buffer to each well. The data is acquired on the IntelliCyt IQue Screener flow cytometer.

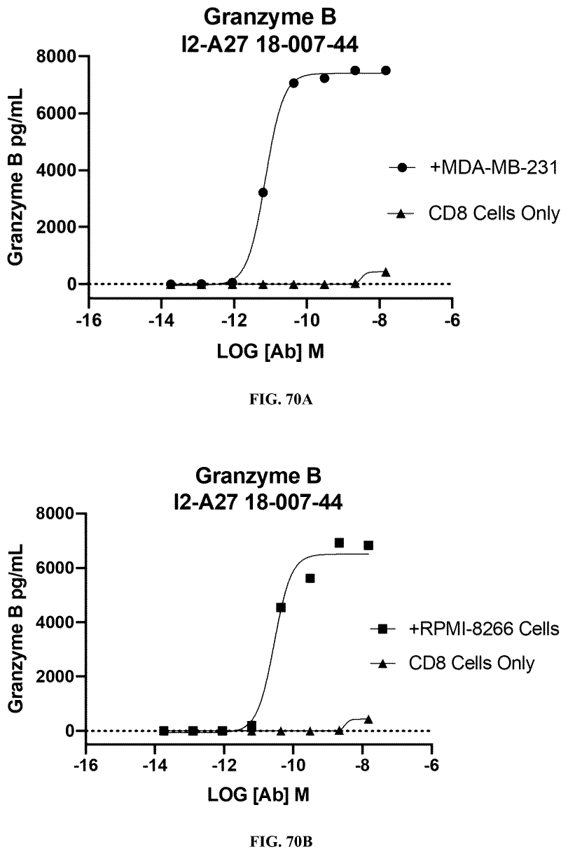

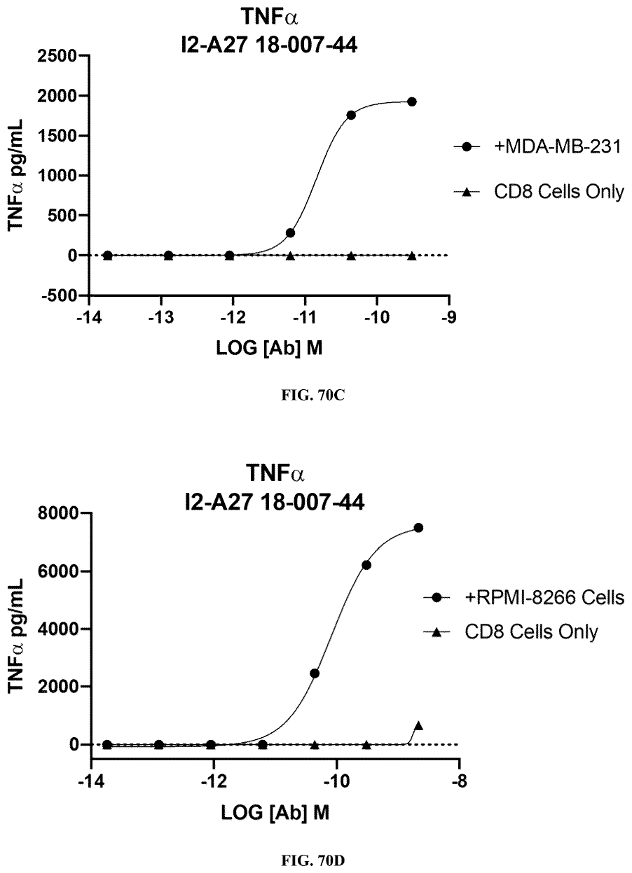

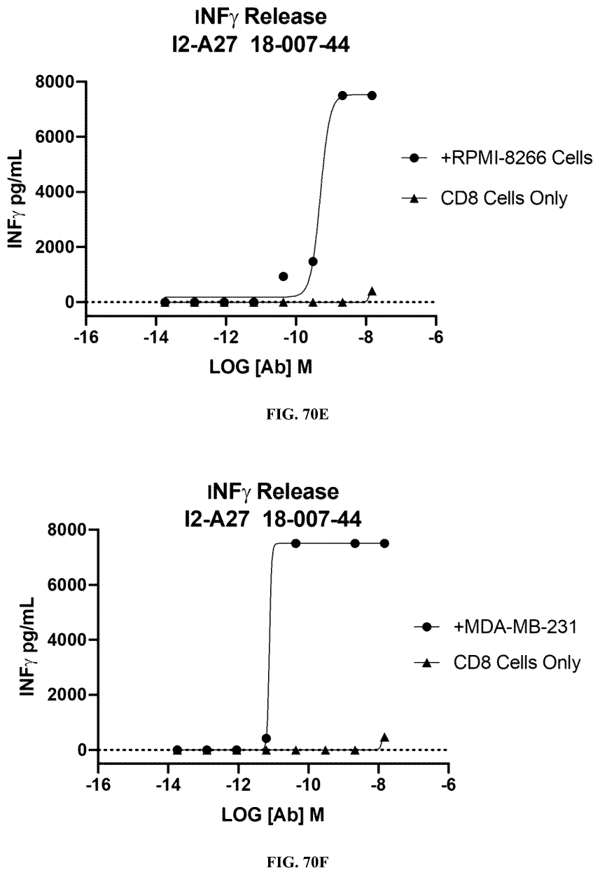

A dose dependent increase in Granzyme B, TNFα, and IFNγ released by the CD8+ T cells was observed in the presence of I2-A27 and ROR1 expressing cells; no such increase was observed in the absence of ROR1 expressing cells, as shown in FIGS. 70A-70F.

### 6.13.25.5. Serum Stability Assessment of ROR1xCD3 1×2 B-Body™ Bispecific Antibody

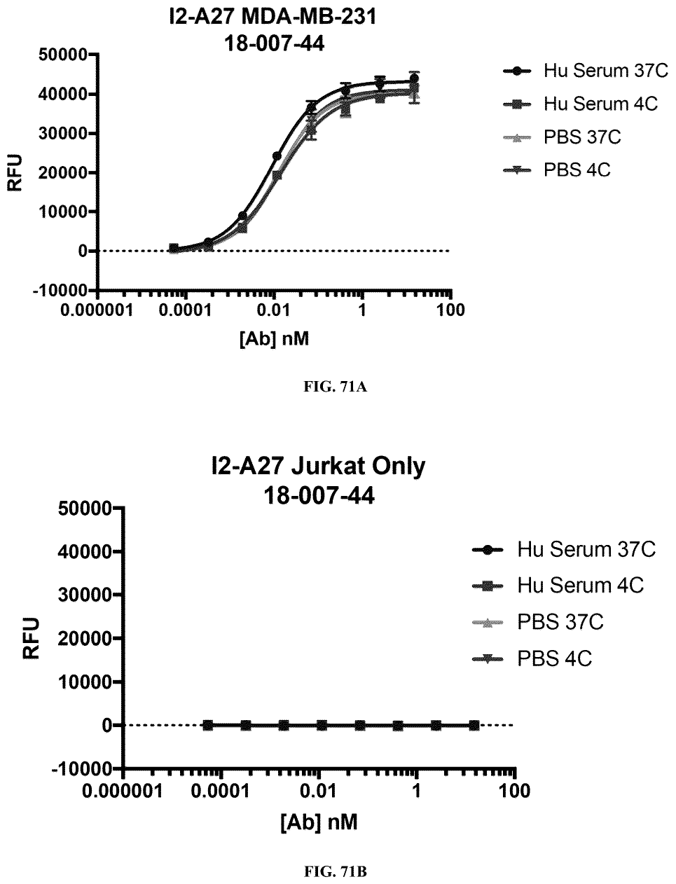

Serum stability of an I2-A27 1×2 B-body™ bispecific antibody was analyzed. The I2-A27 1×2 B-body™ bispecific antibody was diluted from 2.5 mg/mL in PBS to 170 μg/mL in human serum (-93% final serum concentration) or PBS. Samples were then incubated at 37° C. or 4° C. for 1 week. Following the week-long incubation, the Jurkat activity assay was carried out on the samples stored in human serum or the PBS control. It was found that there was no loss in activity for samples that were stored in human serum at 4° C. or 37° C. for 1 week (see FIG. 71A). If material had aggregated during the week-long incubation in serum, the B-body™ may be expected to lead to an increase in signal in the absence of ROR1 expressing cells due to direct activation of the CD3 receptor. No increase in activity was detected when the assay was run in the absence of ROR1 expressing cells (see FIG. 71B).

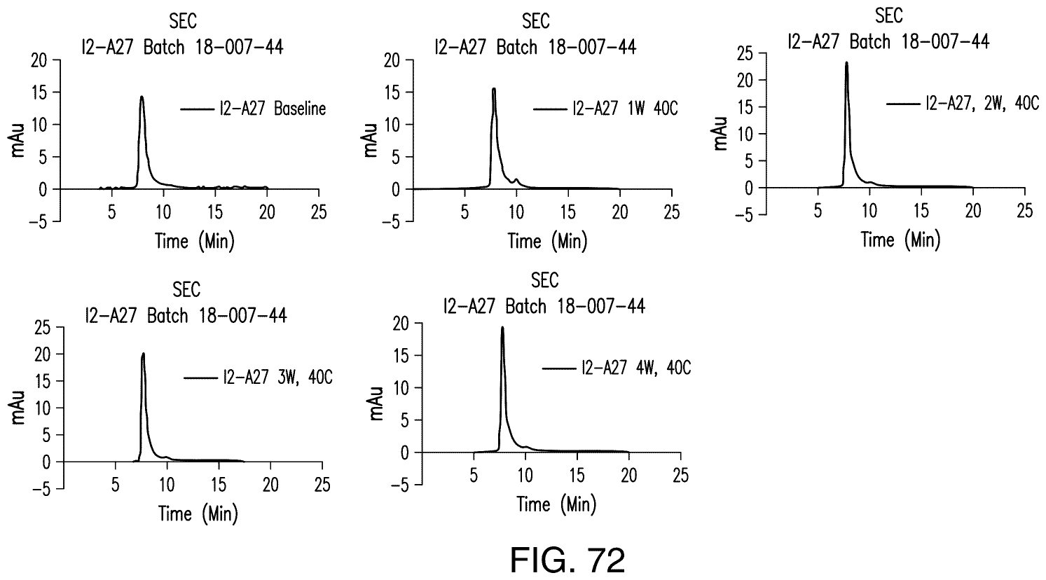

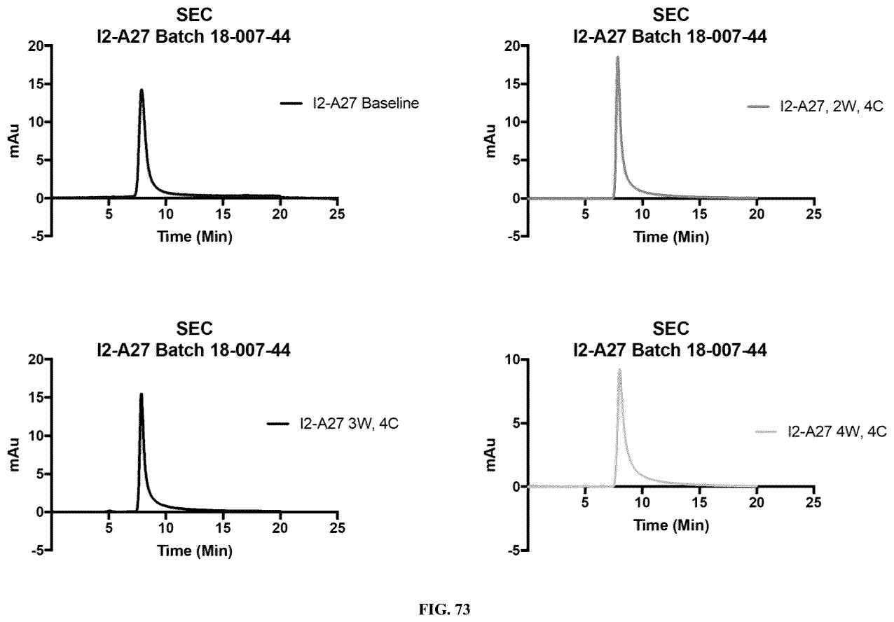

The stability of the I2-A27 1×2 B-body™ bispecific antibody was then assessed by storing the antibody at 4° C. or 40° C. at 2.5 mg/mL in PBS and assessing the homogeneity of the samples by SEC and PDI weekly. For accelerated stability assays, the antibody was stored at 2.5 mg/mL in PBS at 40° C. for up to four weeks. The antibody appeared to be stable under the accelerated stability conditions (see FIG. 72). For real time assays, the antibody was stored at 2. 5 mg/mL in PBS at 4° C. for up to twelve weeks. The antibody also appeared to be stable under the real time conditions (see FIG. 73).

The PDI and Z-ave Diameter for each accelerated and real time stability sample was determined using the UNcle analytical instrument, and results are shown in Table 18 below.

TABLE 18
The PDI and Z-ave Diameter for each accelerated and real time stability
sample as determined using the UNcle analytical instrument

| I2-A27 | | |
|---|---|---|
| Batch 18-007-44 | PDI | Z-Ave Diameter (nm) |
| 1 W, 4 C. | 0.10 | 12.16 |
| 2 W, 4 C. | 0.10 | 11.82 |
| 3 W, 4 C. | N/D | N/D |
| 4 W, 4 C. | 0.16 | 11.72 |
| 5 W, 4 C. | 0.17 | 11.57 |
| 6 W, 4 C. | | |
| 8 W, 4 C. | | |
| 10 W, 4 C. | | |
| 12 W, 4 C. | | |
| 1 W, 40 C. | 0.22 | 12.41 |
| 2 W, 40 C. | 0.14 | 12.41 |
| 3 W, 40 C. | 0.10 | 12.28 |

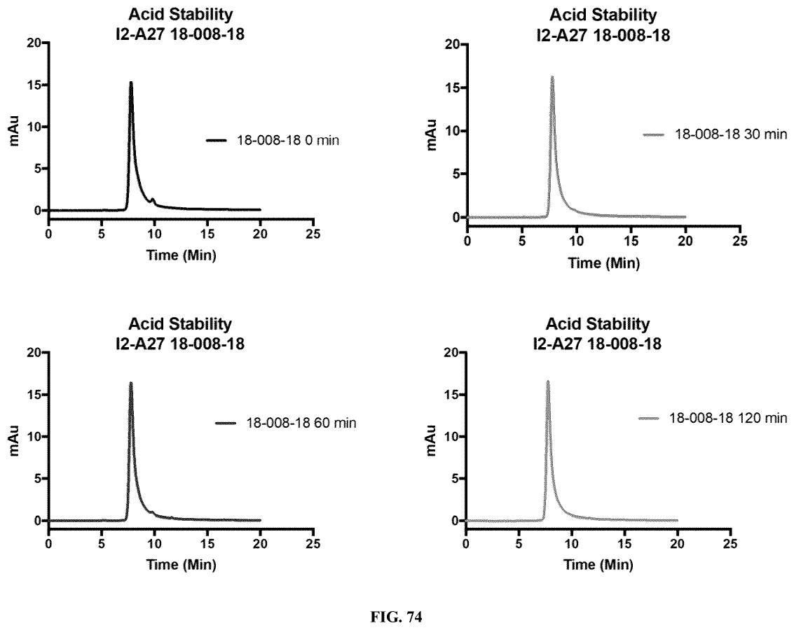

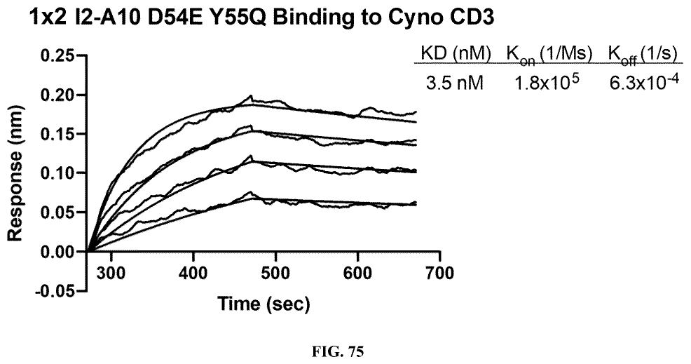

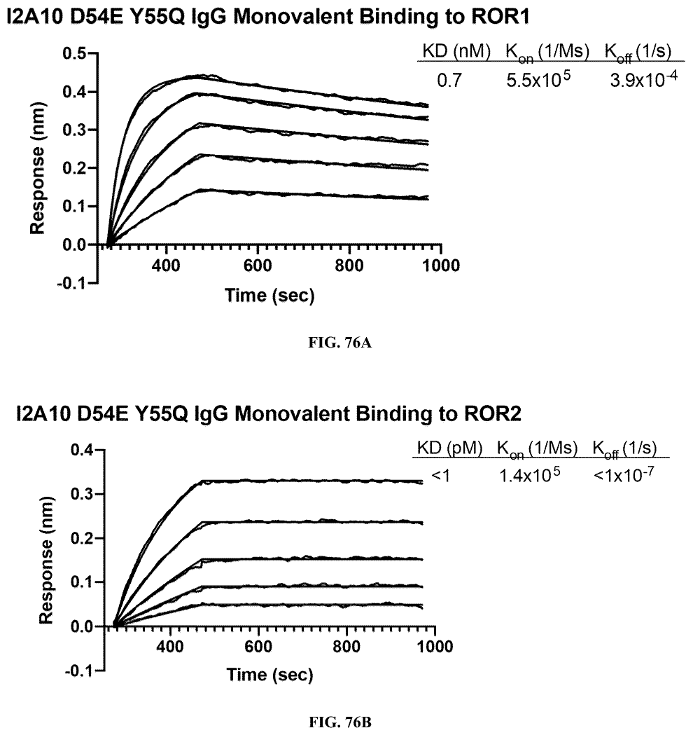

Next the stability of the antibody in acid was assessed. The B-body™ was subjected to the purification procedure as outlined above by binding to a CH1 affinity resin with elution using 100 mM sodium acetate, pH 3.5. The B-body™ was left in the acid for 0, 30, 60, or 120 min prior to neutralization with Tris-HCl buffer. The protein was then buffer exchanged into PBS prior to being analyzed by SEC. As shown in FIG. 74, the I2-A27 1×2 B-body™ bispecific antibody was stable in acid for up to 2 hours.

### 6.13.25.6. In Vivo Efficacy Study of ROR1xCD3 1×2 B-body™ Bispecific Antibody

The in vivo efficacy of an exemplary ROR1xCD3 bispecific B-body™, the I2-A27 1×2 B-body™, was studied in a PBMC-humanized NSG™ mouse model of breast cancer.

More specifically, NSM™ (Jackson Laboratory, Stock No. 005557, also known as NOD-scid IL2Rgamma^{null}, NOD-scid IL2Rg^{null}, NSG, NOD scid gamma) female mice aged six- to eight-weeks old were inoculated orthotopically in the mammary fat pad with 5×10⁶ MDA-MB-231 re-suspended in 1:1 mixture of Matrigel with PBS or serum free media. Body weights and clinical observations were recorded once to twice weekly. Digital caliper measurements were initiated to determine tumor volume once to twice weekly when tumors become palpable. Mice were randomized based on tumor volumes when the tumor volumes reach ˜60-80 mm³ (Study Day-1 or Study Day 0). Mice were injected with PBMCs on Study Day 0. Following injection of PBMCs, dosing began based on the results of the phase 1 validation. Mice were dosed at 0.5 mg/kg, 2.5 mg/kg, and 10 mg/kg. Body weights, clinical observations and digital caliper measurements were recorded twice weekly post dose initiation. Animals that reached a body condition score of <2, a body weight loss of≥20% or a tumor volume >2000 mm³ were euthanized before study terminus. Animals with ulcerated tumors were euthanized before study terminus. Tissues were not collected from animals that were found dead. On Study Day 30, all animals were euthanized by C02 asphyxiation and tissues collected. Tumors were then collected and separated into fragments. One fragment was placed in media for flow cytometry analysis. The following markers were examined: CD45, CD3, CD8, CD4, and 7AAD. One fragment was fixed in 10% neutral buffered formalin (NBF) for paraffin embedding (FFPE). One fragment was flash frozen. Whole blood was collected at the end of study. About 50 μL whole blood was obtained for flow cytometry analysis. The following markers were examined: CD45, CD3, CD8, CD4, and 7AAD.

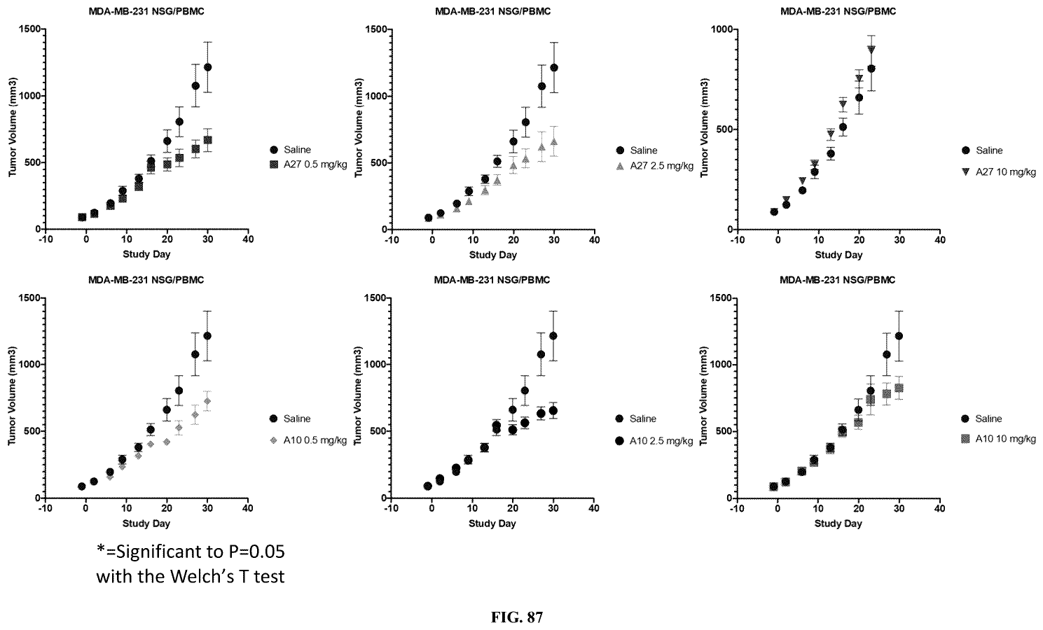

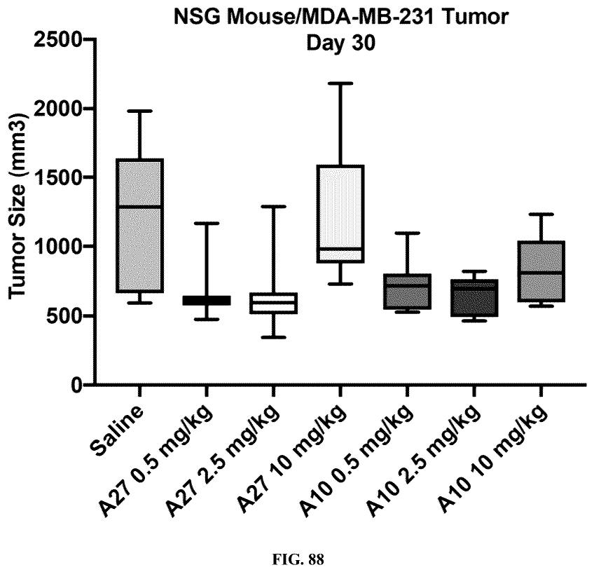

The results are shown in FIG. 87 (upper panel) and FIG. 88. As shown, the I2-A27 1×2 B-body™ demonstrated in vivo efficacy in reducing tumor growth at dose 0.5 mg/kg and 2.5 mg/kg.

### 6.13.26. Example 25: ROR1/ROR2xCD3 Bispecific B-body™ Efficacy

Additional ROR antigen binding molecules comprising the six CDRs of 12A-10 as shown in Table 6 and various CDR mutants were prepared and experiments were performed to test their efficacy as trivalent bispecific constructs in a ROR1xCD3 bispecific B-body™ format.

6.13.26.1. Generation of additional ROR1/ROR2xCD3 1×2 B-body™ bispecific antibodies based on I2-A10

Selective mutations in the CDR regions of an I2-A10 antibody and antibody constructs in the IgG, 1×1 B-body™, and 1×2 B-body™ formats were made and tested.

For example, ROR ABS candidate designated 12A-10 was formatted with CD3 ABS candidate SP34-89 into a bispecific B-body “BC1” 1×2 format, as described above, with the 1×2 format having a 10 amino acid junction between the S and the H domains (see, e.g., FIG. 26 for schematic of domains). The amino acid sequences for the five polypeptide chains that form an exemplary I2A-10 BC1 1×2 trivalent bispecific ROR antigen binding molecule are listed as SEQ ID NO:114 (Chain 1), SEQ ID NO:115 (Chain 2), SEQ ID NO:116 (Chain 3), SEQ ID NO:117 (Chain 4), and SEQ ID NO:115 (Chain 5).

In the following description in this Example, the term “12-A10 1×2 B-body™” or “12-A10” or “A10” refers to this ROR1/ROR2xCD3 1×2 B-body™ having a Chain 1 of SEQ ID NO:114, a Chain 2 of SEQ ID NO:115, a Chain 3 of SEQ ID NO:116, a Chain 4 of SEQ ID NO:117, and a Chain 5 of SEQ ID NO:115.

The I2-A1I antibody constructs with mutations included 12-Ax2 R66G, B2-A10R66K, I2-A10D54G, I2-A10D54E, I2-A10Y55E, I2-A10Y55Q, I2-A10Y56S, I2-A10Y93SY94S, and I2-A10A32Y.

Expression titer and yield of these antibody constructs were analyzed as shown in Table 19.

TABLE 19
E × pression Titer and Yield

| | IgG | | 1 × 1 B-Body | | 1 × 2 B-Body | |
|---|---|---|---|---|---|---|
| | Pre Yield | Post Yield | Pre Yield | Post Yield | Pre Yield | Post Yield |
| | (μg/mL) | (μg/mL) | (μg/mL) | (μg/mL) | (μg/mL) | (μg/mL) |
| I2-A10 | 140 | 114 | 450 | 144 | 125 | 22 (47) |
| I2-A27 | 140 | 98 | 525 | 174 | 135 | 20 (62) |
| I2-A10 R66G | N/D | N/D | 215 | 48 | 224 | 12 (63) |
| I2-A10 R66K | N/D | N/D | 245 | 68 | 225 | 45 (84) |
| I2-A10 D54G | N/D | N/D | 235 | 72 | 226 | 11 (67) |
| I2-A10 D54E | N/D | N/D | 61 | 24 | 213 | 47 |
| I2-A10 Y55E | N/D | N/D | 122 | 52 | 252 | 84 |
| I2-A10 Y55Q | N/D | N/D | 119 | 58 | 233 | 64 |
| I2-A10 Y56S | N/D | N/D | 148 | 38 | 230 | 64 |
| I2-A10 | N/D | N/D | 98 | 66 | 217 | 62 |
| Y93SY94S | | | | | | |
| I2-A10 A32Y | N/D | N/D | 116 | 54 | 245 | 68 |

The binding to ROR1 of these antibody constructs was then tested and each showed positive binding, except the A32Y and Y93/94S constructs which showed poor binding.

These antibody constructs were then tested in the Jurkat functional assay described herein (see, e.g., 6.13.1.8). The results are shown in Table 20.

TABLE 20
Jurkat Functional Assay for I2-A10 Constructs with Mutations

| | | | K562 | K562 | RPMI | RPMI |
|---|---|---|---|---|---|---|
| | MDAEC50 | MDAEC50 | EC50 | EC50 | EC50 | EC50 |
| | (pM) | (pM) | (pM) | (pM) | (pM) | (pM) |
| Ab | 1 × 1 | 1 × 2 | 1 × 1 | 1 × 2 | 1 × 1 | 1 × 2 |
| A27 | 378 | 58 | N/A | N/A | 351 | 19 |
| A10 | 102 | 33 | 33 | 3.2 | 36 | 12 |
| R66G | 314 | 30 | 91 | 3.5 | N/D | 218 |
| R66K | 121 | 20 | 24 | 1.2 | 49 | 7.7 |
| D54G | 51 | 19 | 20 | 0.2 | 23 | 5.9 |
| D54E | 43 | 9 | 26 | 0.3 | 16 | 6.7 |
| Y55E | 552 | 36 | 322 | 4.7 | 512 | 16 |
| Y55Q | 256 | 49 | 68 | 0.8 | 192 | 8.8 |
| Y56S | 480 | 49 | 85 | 13 | 235 | 12 |

These variants were also tested in the cell killing assay. The results are shown in Table 21.

TABLE 21
Cell Killing Assay for I2-A10 Variants

| Ab | LDH pM | Max % Act |
|---|---|---|
| I2-A10 | 5.4 | 36 |
| I2-A27 | 0.3 | 46 |
| I2-A10 R66G | 47.3 | 35 |
| I2-A10 R66K | 5.8 | 31 |
| I2-A10 D54G | 3.6 | 58 |
| I2-A10 Y55E | 10.2 | 29 |
| I2-A10 Y55Q | 6.1 | 39 |
| I2-A10 Y56S | 15.1 | 35 |
| I2-A10 D54E | 2.5 | 48 |

Then, the antibody constructs were analyzed to assess the homogeneity with SEC, the propensity towards aggregation with SMAC, and the hydrophobicity with HIC. SEC, SMAC and HIC assays were preformed as described in Example 24. D54G was not acceptable according to the SEC assay. Y55Q (and Y55E) showed improved HIC values over the I2-A10 parent. Differential scanning fluorimetry (DSF) was also used to test the antibody constructs.

Based on the above described assays and analyses, the D54E and Y55Q mutations were selected for the preparation of a novel A10-related antibody designated I2-A10 D54E Y55Q. The D54E mutation which corrects a potential isomerization site unexpectedly resulted in an antibody with better activity (in vitro functional assays) than the activity of the I2-A10 parent antibody. The Y55Q mutation which reverts a rare residue to germline unexpectedly resulted in an antibody with improved HIC values as compared to the I2-A10 parent antibody.

An additional ROR1/ROR2xCD3 1×2 B-body™ was then generated incorporating the D54E and Y55Q mutations. Y55Q is a mutation in the CDR2 region of the VL of the antigen binding site for ROR1 and ROR2 as shown in Table 6, and was introduced into chain 1 and chain 3 as shown below. D54E is a mutation in the CDR2 region of the VH of the antigen binding site for ROR1 and ROR2 as shown in Table 6, and was introduced into chain 2 and chain 5 as shown below. In addition, mutations to generate a knob-and-hole configuration were included in the second CH3 domains in chain 1 (hole) and chain 3 (knob).

ROR ABS candidate designated I2A-10 D54E Y55Q was formatted with CD3 ABS candidate SP34-89 into a bispecific B-body “BC1” 1×2 format, as described above, with the 1×2 format having a 10 amino acid junction between the S and the H domains (see, e.g., FIG. 26 for schematic of domains). The amino acid sequences for the five polypeptide chains that form an exemplary I2A-10 D54E Y55Q BC1 1×2 trivalent bispecific ROR antigen binding molecule are listed as SEQ ID NO:132 (Chain 1), SEQ ID NO:133 (Chain 2), SEQ ID NO:134 (Chain 3), SEQ ID NO:135 (Chain 4), and SEQ ID NO:133 (Chain 5).

In the following description in this Example, the term “12-A10 D54E Y55Q 1×2 B-body™” or “12-A10 D54E Y55Q” refers to this ROR1/ROR2xCD3 1×2 B-body™ having a Chain 1 of SEQ ID NO:132, a Chain 2 of SEQ ID NO:133, a Chain 3 of SEQ ID NO:134, a Chain 4 of SEQ ID NO:135, and a Chain 5 of SEQ ID NO:133.

### 6.13.26.2. Binding of ROR1/ROR2xCD3 1×2 B-body™ Bispecific Antibody to CD3 and ROR1

The binding affinity of an I2-A10 D54E Y55Q 1×2 B-body™ to cynomolgus CD3 delta and epsilon heterodimer was determined by the monovalent BLI binding assessment described in Example 24 above. As shown in FIG. 75, the bispecific antibody exhibited a monovalent Kd of 3.5 nM to cynomolgus CD3 delta and epsilon heterodimer.

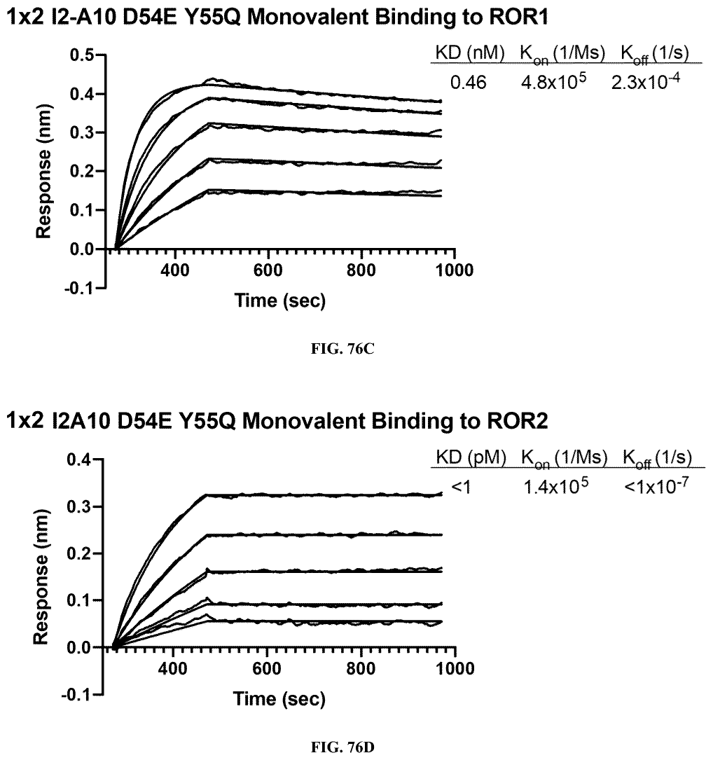

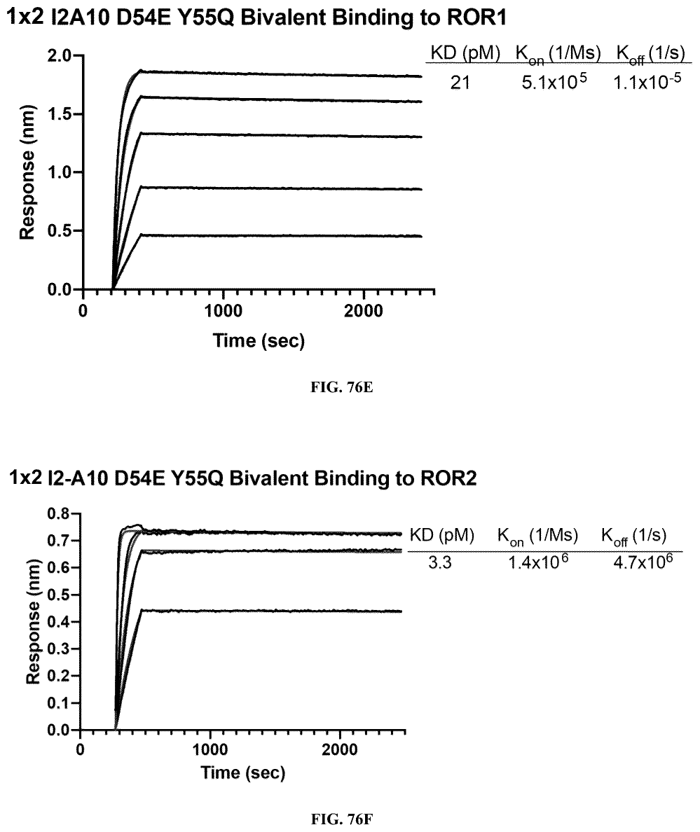

Next, the binding of the antibody to ROR1 and ROR2 was assessed using the monovalent and bivalent BLI binding assessments as described in Example 24. As shown in FIGS. 76A-76B, I2-A10 D54E Y55Q IgG antibody showed monovalent binding of 0.7 nM to ROR1 and less than 1 pM to ROR2. The I2-A10D54E Y55Q 1×2 B-body™ showed a monovalent binding of 0.46 nM to ROR1 and less than 1 pM to ROR2 and bivalent binding of 21 pM to ROR1 and 3 pM to ROR2 (see FIGS. 76C-76F).

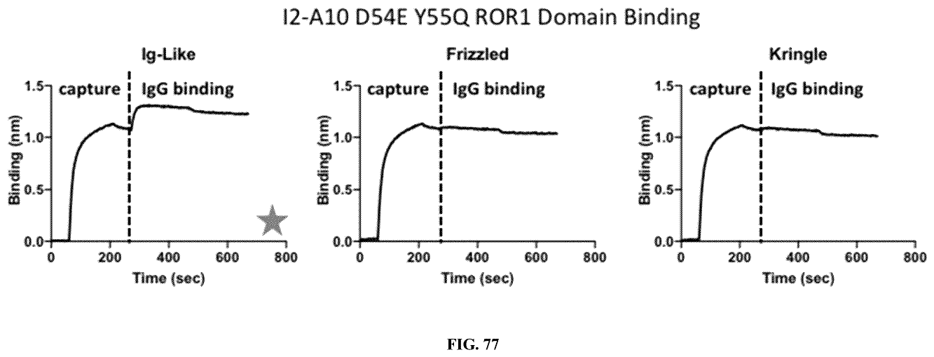

Binding of I2-A10 D54E Y55Q IgG to Ig-like, Frizzled, and Kringle domains was then determined by BLI. As shown in FIG. 77, I2-A10 D54E Y55Q IgG binds to the Ig-like domain of ROR1.

### 6.13.26.3. Further Characterization of ROR1/ROR2xCD3 1×2 B-body™

An I2-A10 D54E Y55Q 1×2 B-body™ was produced through transient transfection of DNA encoding polypeptide chains 1-4 (polypeptide chain 5 is the same as polypeptide chain 2) in Expi-CHO cells. The five chain antibody was purified with a CH1 affinity resin and buffer exchanged into PBS, pH 7.4.

The assembly and purity of the resulting B-body™ was assessed through non-reducing and reducing SDS-PAGE as described in Example 24. The 2×1 B-body™ ran near the expected molecular weight of 200 kD in a non-reduced format. In a reduced format, the expected 3 bands were resolved (one chain at 75 kD, one chain at 50 kD, and 2 chains at 25 kD) (data not shown here). The protein was subsequently analyzed by capillary electrophoresis in a reduced and non-reduced format as described in Example 24 and the percent purity was calculated from the three major bands in the reduced sample to be 96.25%.

The antibody protein was then analyzed via a set of columns to assess the homogeneity with SEC, the propensity towards aggregation with SMAC, and the hydrophobicity with HIC. SEC, SMAC and HIC assays were performed as described in Example 24.

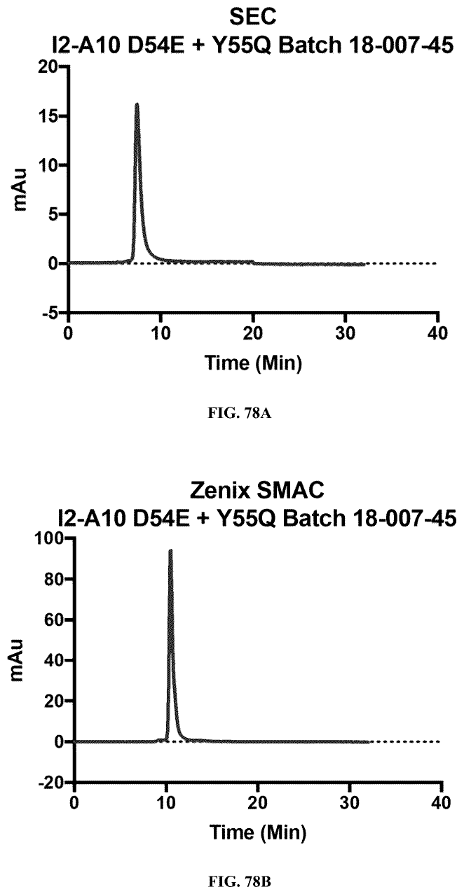

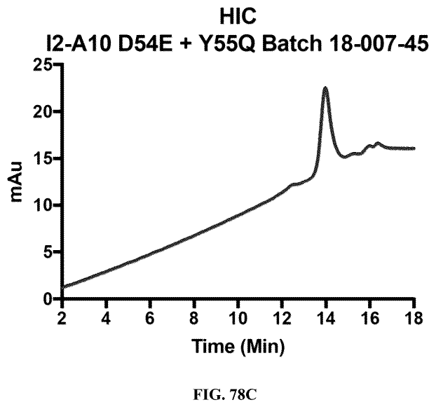

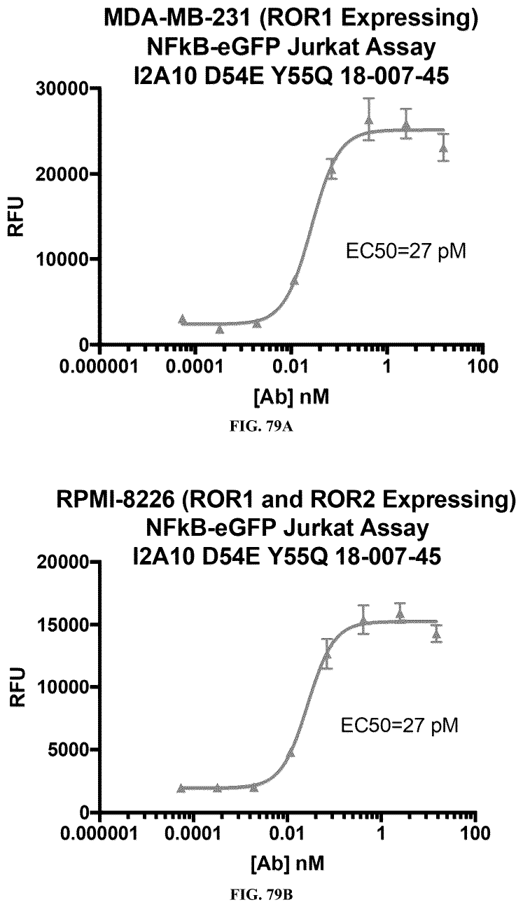

The results of these analyses are shown in FIGS. 78A-78C. As shown, the protein appeared>99% homogeneous by SEC and resulted in a single peak with both SMAC and HIC that fell within the range observed for other developable antibodies suggesting an acceptable aggregation and hydrophobicity profile.

The protein was next analyzed with the UNcle analytic instrument as described in Example 24. The antibody showed melting temperatures of 68.5° C. and 77.4° C. The median PDI from three measurements was 0.21 which is indicative of a monodisperse sample (PDI<0.25 is considered monodisperse) with a hydrodynamic diameter of 12.6 nm (see Table 22 below).

TABLE 22
Results for melting temperature, polydispersity index, and
hydrodynamic diameter from the UNcle analytical instrument

| | Tm1 (° C.) | Tm2 (° C.) | PDI | Diameter (nm) |
|---|---|---|---|---|
| I2-A10 D54E Y55Q | 68.5 | 77.4 | 0.21 | 12.6 |
| 1 × 2 B-body ™ | | | | |

### 6.13.26.4. In Vitro Functional Assessment of ROR1/ROR2xCD3 1×2 B-body™

The functional capability of an I2-A10 D54E Y55Q 1×2 B-body™ in cellular co-culture assays of T cells and ROR1 expressing cancer cells was analyzed. Cancer cell lines that expressed ROR1 and/or ROR2 were assessed.

MDA-MB-231, RPMI-8226, and K562 cells were used in this study, and as shown in Example 24, the MDA-MB-231 cell line was determined to primarily express ROR1, the RPMI-8226 cell line was determined to express both ROR1 and ROR2, and the K562 cell line was determined to express primarily ROR2.

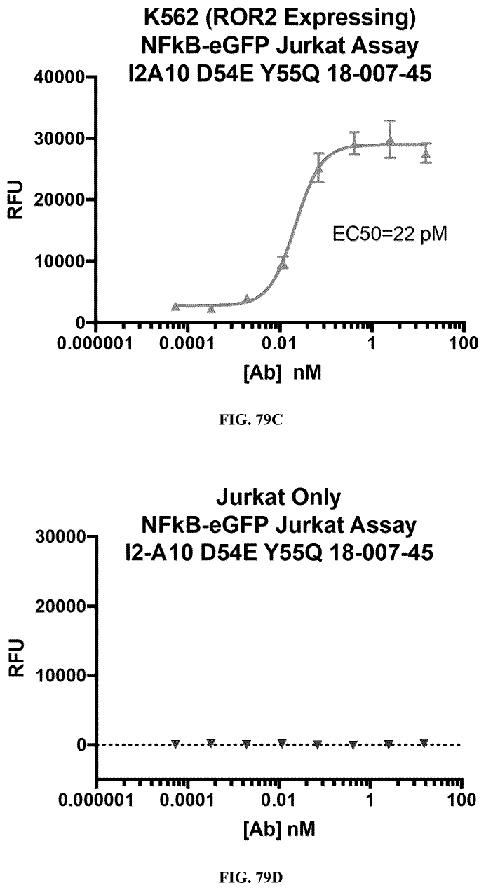

To assess whether the I2-A10 D54E Y55Q 1×2 B-body™ could activate T cells in the presence of ROR1 or ROR2 expressing cancer cells, an NFkB Jurkat co-culture reporter gene assay as described in Example 24 was utilized. Activation of the Jurkat T cells leads to activation of the NFkB response element and production of eGFP. As shown in FIGS. 79A-79D, the I2-A10 D54E Y55Q 1×2 B-body™ was able to activate the Jurkat reporter cell line in the pM range in the presence of MDA-MB-231 (ROR1 expressing), RPMI-8226 (ROR1 and ROR2 expressing), and K562 (ROR2 expressing) cells but not in the absence of a ROR1 or ROR2 expressing cell line.

Next, the function of the I2-A10 D54E Y55Q 1×2 B-body™ to activate primary CD8+ T cells was studied. This functional assay is as described in Example 24 above, in the presence of ROR1 expressing cell lines as assessed by increased expression levels of the early T cell activation marker, CD69, and the late T cell activation marker, CD25.

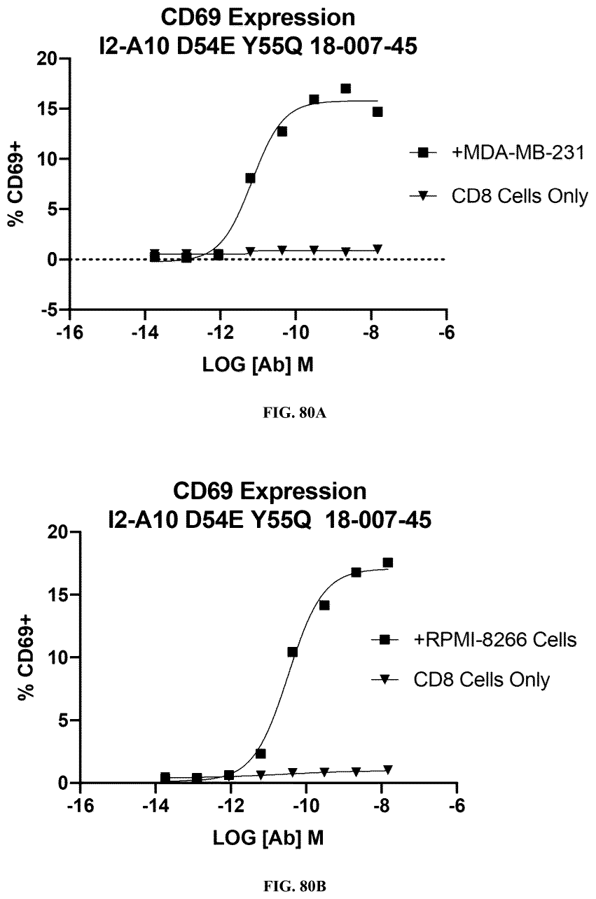

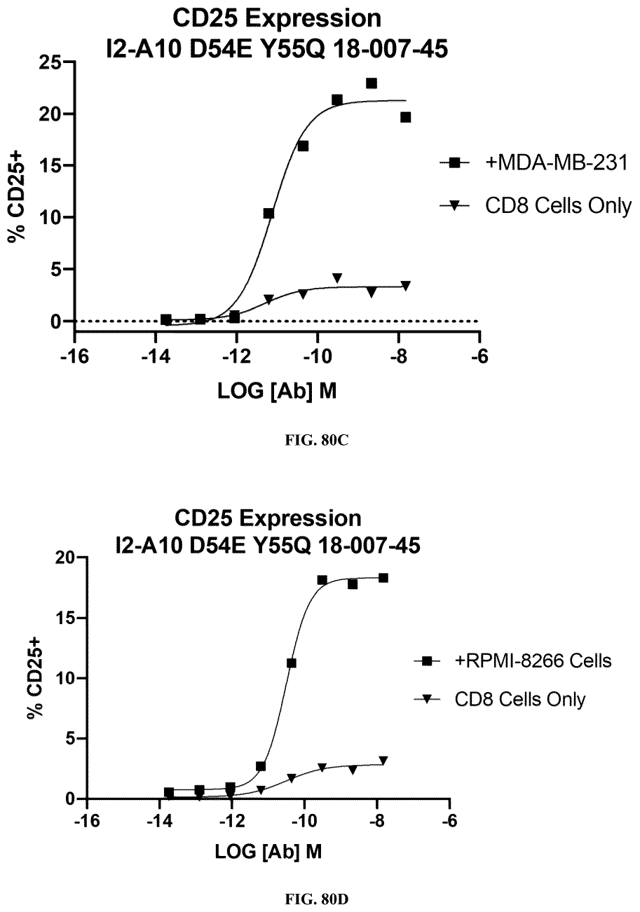

As shown in FIGS. 80A-80D, the I2-A10 D54E Y55Q 1×2 B-body™ led to a dose dependent increase in expression of CD69 and CD25 when in the presence of ROR1 expressing cells (MDA-MB-231 or RPMI-8226) but not in the absence of these cells.

Whether the activated T cells could subsequently kill the ROR1 expressing cell lines was assessed by LDH release from the target cells and secretion of Granzyme B, TNFα, and IFNγ by the CD8+effector cells.

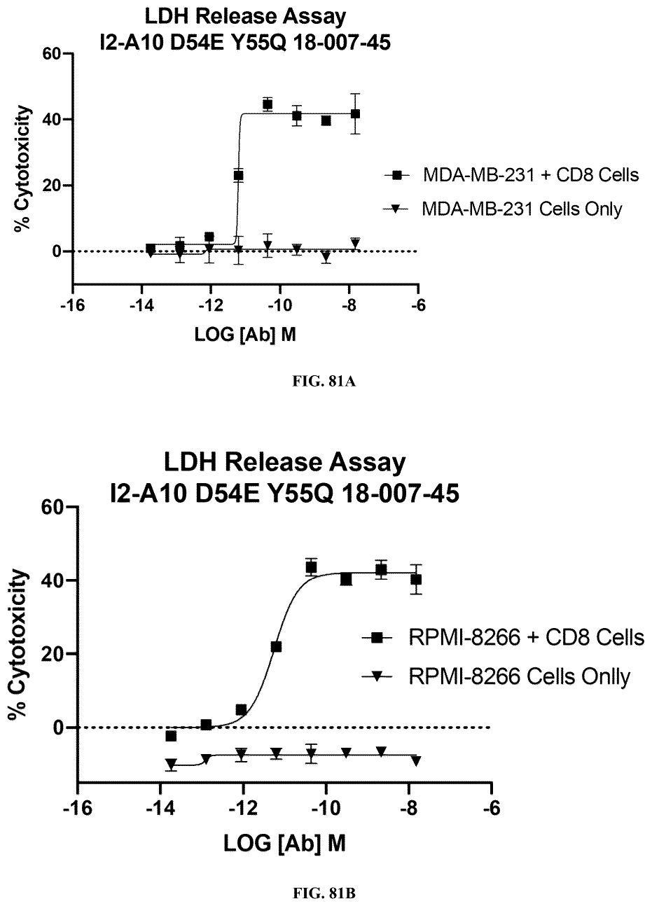

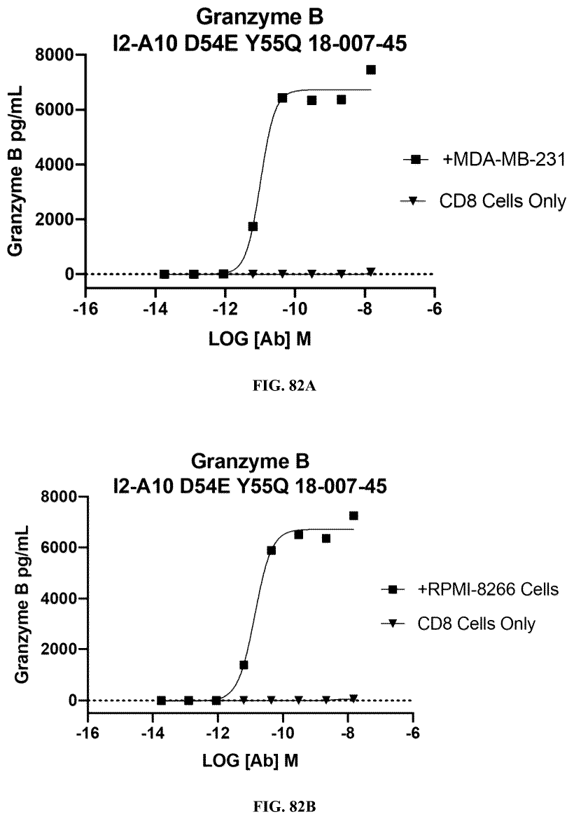

LDH release from the target cells was analyzed as described in Example 24. As shown in FIGS. 81A and 81B, the I2-A10 D54E Y55Q 1×2 B-body™ led to dose dependent cytotoxicity of ROR1 expressing cells in the presence of CD8+ T cells, but not in the absence of CD8+ T cells.

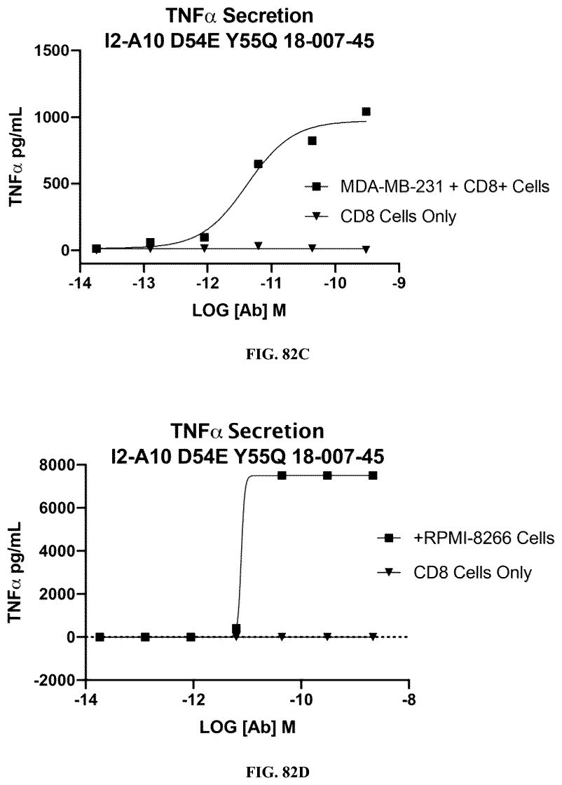

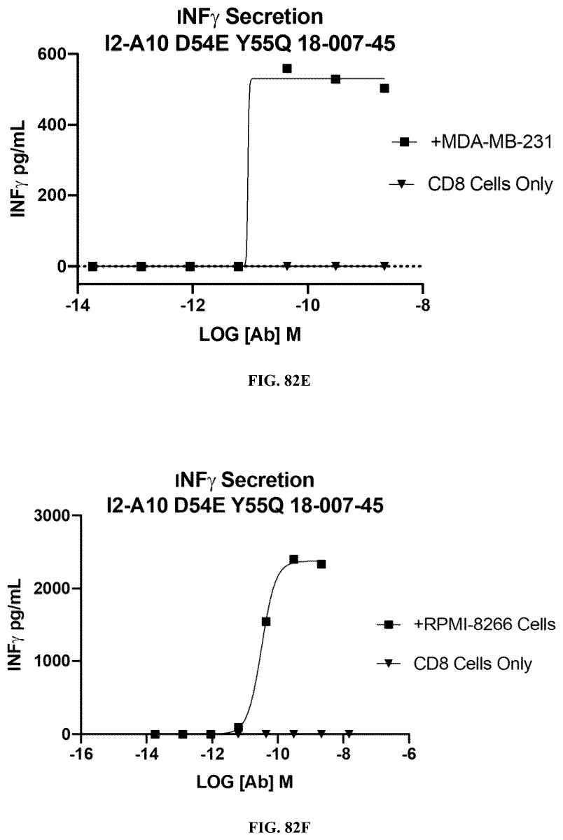

Secretion of Granzyme B, TNFα, and IFNγ by the CD8+effector cells was analyzed as described in Example 24. A dose dependent increase in Granzyme B, TNFα, and IFNγ released by the CD8+ T cells was observed in the presence of the I2-A10 D54E Y55Q 1×2 B-body™ and ROR1 expressing cells; no such increase was observed in the absence of ROR1 expressing cells, as shown in FIGS. 82A-82F.

6.13.26.5. Serum Stability Assessment of ROR1xCD3 1×2 B-body™ bispecific antibody

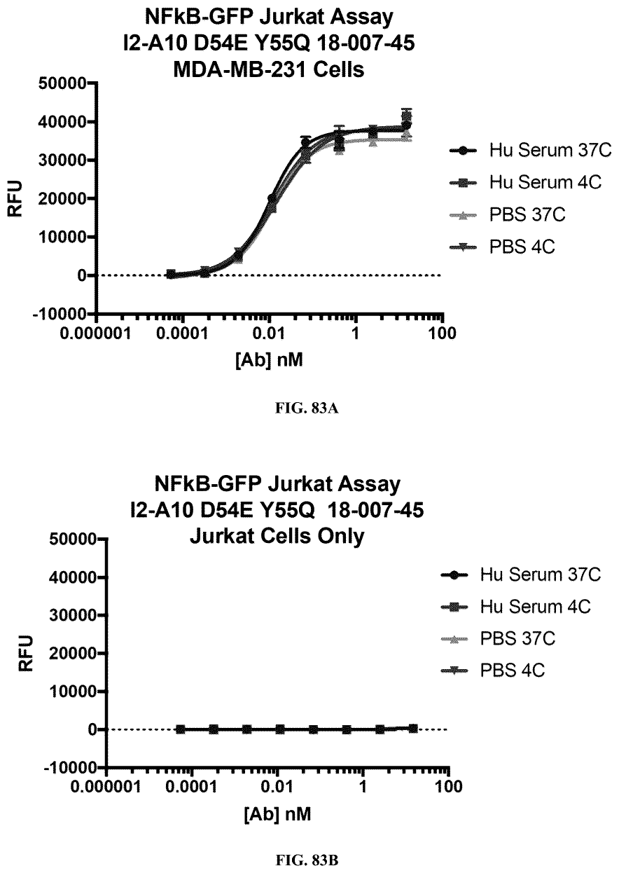

Serum stability of the I2-A10 D54E Y55Q 1×2 B-body™ was analyzed. The bispecific antibody was diluted from 2.5 mg/mL in PBS to 170 μg/mL in human serum (˜93% final serum concentration) or PBS. Samples were then incubated at 37° C. or 4° C. for 1 week. Following the week-long incubation, the Jurkat activity assay was carried out on the samples stored in human serum or the PBS control. It was found that there was no loss in activity for samples that were stored in human serum at 4° C. or 37° C. for 1 week (see FIG. 83A). If material had aggregated during the week-long incubation in serum, the B-body™ may be expected to lead to an increase in signal in the absence of ROR1 expressing cells due to direct activation of the CD3 receptor. No increase in activity was detected when the assay was run in the absence of ROR1 expressing cells (see FIG. 83B).

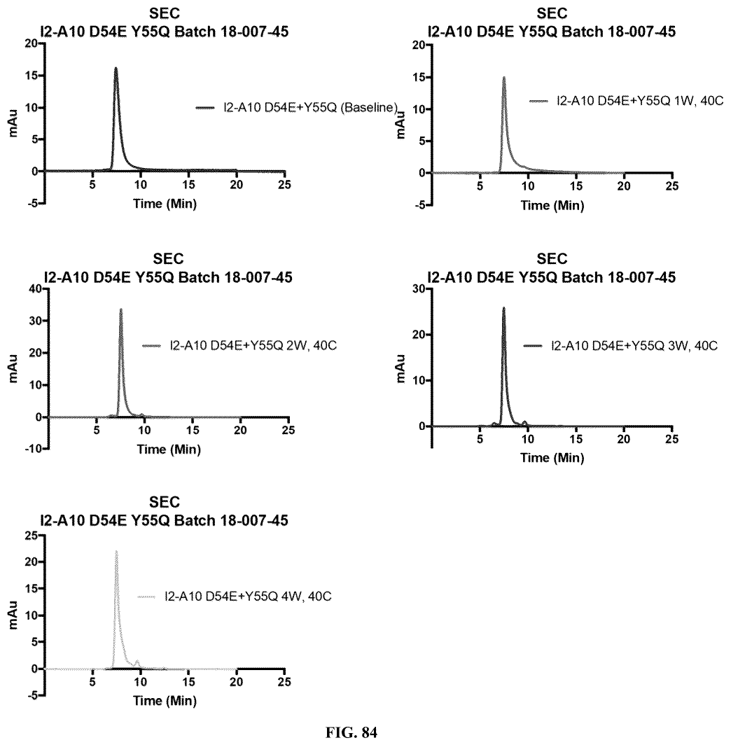

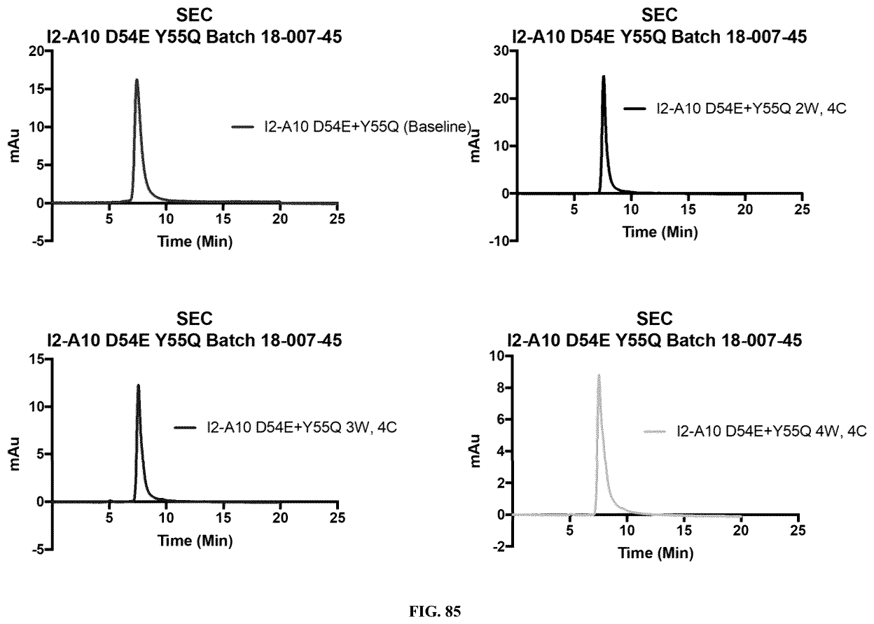

The stability of the I2-A10 D54E Y55Q 1×2 B-body™ was then assessed by storing the antibody at 4° C. or 40° C. at 2.5 mg/mL in PBS and assessing the homogeneity of the samples by SEC and PDI weekly. For accelerated stability assays, the antibody was stored at 2.5 mg/mL in PBS at 40° C. for up to four weeks. The antibody appeared to be stable under the accelerated stability conditions (see FIG. 84). For real time assays, the antibody was stored at 2. 5 mg/mL in PBS at 4° C. for up to twelve weeks. The antibody also appeared to be stable under the real time conditions (see FIG. 85).

The PDI and Z-ave Diameter for each accelerated and real time stability sample was determined using the UNcle analytical instrument, and results are shown in Table 23.

TABLE 23
The PDI and Z-ave Diameter for each accelerated and real time stability
sample as determined using the UNcle analytical instrument

| I2-A10 D54E Y55Q | | |
|---|---|---|
| Batch 18-007-45 | PDI | Z-Ave Diameter (nm) |
| 1 W, 4 C. | 0.13 | 12.24 |
| 2 W, 4 C. | 0.14 | 11.68 |
| 3 W, 4 C. | 0.21 | 12.17 |
| 4 W, 4 C. | 0.12 | 11.68 |
| 5 W, 4 C. | 0.25 | 11.33 |
| 6 W, 4 C. | | |
| 8 W, 4 C. | | |
| 10 W, 4 C. | | |
| 12 W, 4 C. | | |
| 1 W, 40 C. | 0.184 | 12.15 |
| 2 W, 40 C. | 0.139 | 12.01 |
| 3 W, 40 C. | 0.058 | 14.72 |

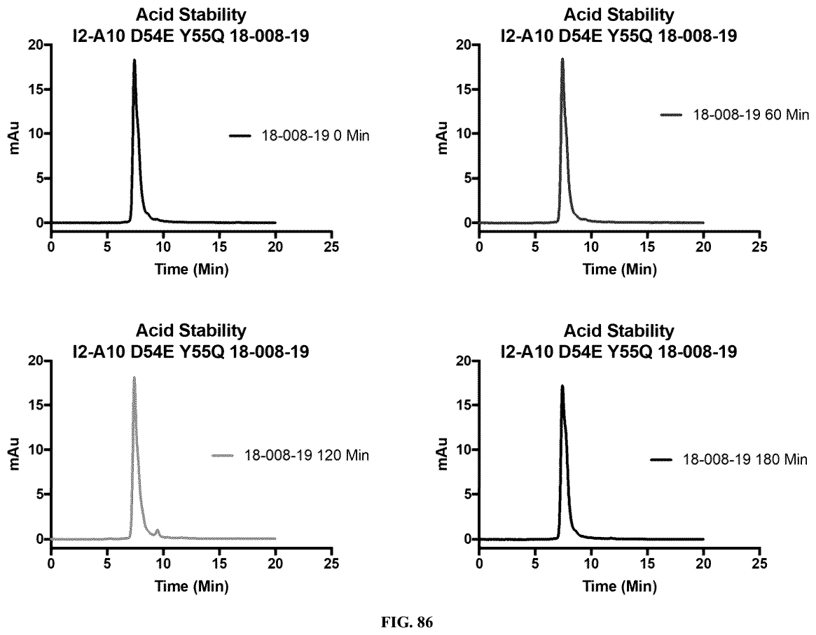

Next the stability of the antibody in acid was assessed. The I2-A10 D54E Y55Q 1×2 B-body™ was subjected to the purification procedure as outlined above by binding to a CH1 affinity resin with elution using 100 mM sodium acetate, pH 3.5. The bispecific antibody was left in the acid for 0, 30, 60, or 120 min prior to neutralization with Tris-HCl buffer. The protein was then buffer exchanged into PBS prior to being analyzed by SEC. As shown in FIG. 86, the I2-A10 D54E Y55Q 1×2 B-body™ was stable in acid for up to 2 hours. 6.13.26.6. In Vivo Efficacy Study of ROR1/ROR2xCD3 1×2 B-body™ bispecific antibody

The in vivo efficacy of an exemplary ROR1/ROR2xCD3 bispecific B-body™, 12-A10 D54E Y55Q 1×2 B-body™, was studied in a PBMC-humanized NSG™ mouse model of breast cancer. The study was performed as described in Example 24 above.

The results are shown in FIG. 87 (lower panel) and FIG. 88. As shown, the I2-A10 D54E Y55Q 1×2 B-body™ demonstrated in vivo efficacy in reducing tumor growth at dose 0.5 mg/kg, 2.5 mg/kg, and 10 mg/kg.

6.14. Sequences
>Example 1, bivalent monospecific construct
CHAIN 1 [SEQ ID NO: 1]
(VL)~VEIKRTPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQKSLSLSPGK
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTI
SKAKGQPREPQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSLSPGK
>Example 1, bivalent monospecific construct
CHAIN 2 [SEQ ID NO: 2]
(VH)~VTVSSASPREPQVYTLPPCRDELTKNQVSLWCLVKGFYPSDIAVEWESNGQPE
NNYKTTPPVLDSDGSFFLYSKLTVDKSRWQQGNVFSCSVMHEALHNHYTQKSLSLSPG
K
>Example 1, bivalent, bispecific construct
CHAIN 1 [SEQ ID NO: 3]
(VL)~VEIKRTPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQKSLSLSPGK
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTI
SKAKGQPREPQVYTLPPCRDELTKNQVSLWCLVKGFYPSDIAVEWESNGQPENNYKT
TPPVLDSDGSFFLYSKLTVDKSRWQQGNVFSCSVMHEALHNHYTQKSLSLSPGK
VL- CH3- Hinge- CH2- CH3(knob)
>Example 1, bivalent, bispecific construct
CHAIN 2 [SEQ ID NO: 4]
(VH)~VTVSSASPREPQVYTLPPCRDELTKNQVSLWCLVKGFYPSDIAVEWESNGQPE
NNYKTTPPVLDSDGSFFLYSKLTVDKSRWQQGNVFSCSVMHEALHNHYTQKSLSLSPG
K
VH- CH3
>Example 1, bivalent, bispecific construct
CHAIN 3_ [SEQ ID NO: 5]
(VL)~VEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTI
SKAKGQPREPQVCTLPPSREEMTKNQVSLSCAVKGFYPSDIAVEWESNGQPENNYKTT
PPVLDSDGSFFLVSKLTVDKSRWQQGNVFSCSVMHEALHNHYTQKSLSLSPGK
VL- CL- Hinge- CH2- CH3(hole)
>Example 1, bivalent, bispecific construct
CHAIN 4 [SEQ ID NO: 6]
(VH)~VTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPPKSC
VH- CH1
>Fc Fragment of Human IgG1 [SEQ ID NO: 7]
GQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSP
>BC1 chain 1 [SEQ ID NO: 8]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPC
RDELTKNQVSLWCLVKGFYPSDIAVEWESNGQPENNYKTTPPVLDSDGSFFLYSKLTV
DKSRWQQGNVFSCSVMHEALHNHYTQKSLSLSPGK
Domain arrangement:
A- B- Hinge- D- E
VL- CH3- Hinge- CH2- CH3(knob)
Mutations in first CH3 (Domain B):
T366K; 445K, 446S, 447C insertion
Mutations in second CH3 (Domain E):
S354C, T366W
>BC1 chain 2 [SEQ ID NO: 9]
EVQLVESGGGLVQPGGSLRLSCAASGFTFSDYDIHWVRQAPGKGLEWVGDITPYDGTT
NYADSVKGRFTISADTSKNTAYLQMNSLRAEDTAVYYCARLVGEIATGFDYWGQGTLV
TVSSASPREPQVYTDPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSGEC
Domain arrangement:
F- G
VH- CH3
Mutations in CH3 (Domain G):
L351D; 445G, 446E, 447C insertion
>BC1 chain 3 [SEQ ID NO: 10]
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIKRTVAAPSVF
IFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGECDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVCTLPPS
REEMTKNQVSLSCAVKGFYPSDIAVEWESNGQPENNYKTTPPVLDSDGSFFLVSKLTV
DKSRWQQGNFFSCSVMHEALHNHYTQKSLSLSPGK
Domain arrangement:
H- I- Hinge- J- K
VL- CL- Hinge- CH2- CH3(hole)
Mutations in CH3 (domain K):
Y349C, D356E, L358M, T366S, L368A, Y407V
>BC1 chain 4 [SEQ ID NO: 11]
QVQLVESGGGVVQPGRSLRLDCKASGITFSNSGMHWVRQAPGKGLEWVAVIWYDGSKR
YYADSVKGRFTISRDNSKNTLFLQMNSLRAEDTAVYYCATNDDYWGQGTLVTVSSAST
KGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPPKSC
Domain arrangement:
L- M
VH- CH1
>BC1 Domain A [SEQ ID NO: 12]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRT
>BC1 Domain B [SEQ ID NO: 13]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>BC1 Domain D [SEQ ID NO: 14]
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAK
>BC1 Domain E [SEQ ID NO: 15]
GQPREPQVYTLPPCRDELTKNQVSLWCLVKGFYPSDIAVEWESNGQPENNYKTTPPVL
DSDGSFFLYSKLTVDKSRWQQGNVFSCSVMHEALHNHYTQKSLSLSPGK
>BC1 Domain F [SEQ ID NO: 16]
EVQLVESGGGLVQPGGSLRLSCAASGFTFSDYDIHWVRQAPGKGLEWVGDITPYDGTT
NYADSVKGRFTISADTSKNTAYLQMNSLRAEDTAVYYCARLVGEIATGFDYWGQGTLV
TVSSAS
>BC1 Domain G [SEQ ID NO: 17]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
>BC1 Domain H [SEQ ID NO: 18]
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIK
>BC1 Domain I [SEQ ID NO: 19]
RTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
>BC1 Domain J [SEQ ID NO: 20]
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAK
>BC1 Domain K [SEQ ID NO: 21]
GQPREPQVCTLPPSREEMTKNQVSLSCAVKGFYPSDIAVEWESNGQPENNYKTTPPVL
DSDGSFFLVSKLTVDKSRWQQGNVFSCSVMHEALHNHYTQKSLSLSPGK
>BC1 Domain L [SEQ ID NO: 22]
QVQLVESGGGVVQPGRSLRLDCKASGITFSNSGMHWVRQAPGKGLEWVAVIWYDGSKR
YYADSVKGRFTISRDNSKNTLFLQMNSLRAEDTAVYYCATNDDYWGQGTLVTVSS
>BC1 Domain M [SEQ ID NO: 23]
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPPKSC
>BC28 chain 1 [SEQ ID NO: 24]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRTPREPQVC
TLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGKDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPC
RDELTKNQVSLWCLVKGFYPSDIAVEWESNGQPENNYKTTPPVLDSDGSFFLYSKLTV
DKSRWQQGNVFSCSVMHEALHNHYTQKSLSLSPGK
Domain arrangement:
A- B- Hinge- D- E
VL- CH3- Hinge- CH2- CH3(knob)
Mutations in domain B:
Y349C; 445P, 446G, 447K insertion
Mutations in domain E:
S354C, T366W
>BC28 chain 2 [SEQ ID NO: 25]
EVQLVESGGGLVQPGGSLRLSCAASGFTFSDYDIHWVRQAPGKGLEWVGDITPYDGTT
NYADSVKGRFTISADTSKNTAYLQMNSLRAEDTAVYYCARLVGEIATGFDYWGQGTLV
TVSSASPREPQVYTLPPCRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK
Domain arrangement:
F- G
VH- CH3
Mutations in domain G:
S354C; 445P, 446G, 447K insertion
>BC28 domain A [SEQ ID NO: 26]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRT
>BC28 domain B [SEQ ID NO: 27]
PREPQVCTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
>BC28 domain D [SEQ ID NO: 28]
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAK
>BC28 domain E [SEQ ID NO: 29]
GQPREPQVYTLPPCRDELTKNQVSLWCLVKGFYPSDIAVEWESNGQPENNYKTTPPVL
DSDGSFFLYSKLTVDKSRWQQGNVFSCSVMHEALHNHYTQKSLSLSPGK
>BC28 domain F [SEQ ID NO: 30]
EVQLVESGGGLVQPGGSLRLSCAASGFTFSDYDIHWVRQAPGKGLEWVGDITPYDGTT
NYADSVKGRFTISADTSKNTAYLQMNSLRAEDTAVYYCARLVGEIATGFDYWGQGTLV
TVSSAS
>BC28 domain G [SEQ ID NO: 31]
PREPQVYTLPPCRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
>BC44 chain 1 [SEQ ID NO: 32]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRTVREPQVC
TLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGKDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPC
RDELTKNQVSLWCLVKGFYPSDIAVEWESNGQPENNYKTTPPVLDSDGSFFLYSKLTV
DKSRWQQGNVFSCSVMHEALHNHYTQKSLSLSPGK
Domain arrangement:
A- B- Hinge- D- E
VL- CH3- Hinge- CH2- CH3(knob)
Mutations in domain B:
P343V; Y349C; 445P, 446G, 447K insertion
Mutations in domain E:
S354C, T366W
>BC44 Domain A [SEQ ID NO: 33]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRT
>BC44 Domain B [SEQ ID NO: 34]
VREPQVCTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
>BC44 Domain D [SEQ ID NO: 35]
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAK
>BC44 Domain E [SEQ ID NO: 36]
GQPREPQVYTLPPCRDELTKNQVSLWCLVKGFYPSDIAVEWESNGQPENNYKTTPPVL
DSDGSFFLYSKLTVDKSRWQQGNVFSCSVMHEALHNHYTQKSLSLSPGK
>BC28 bivalent chain 3 equivalent to SEQ ID NO: 10
>BC28 bivalent chain 4 equivalent to SEQ ID NO: 11
>BC28 1x2 chain 3 [SEQ ID NO: 37]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEI
KRTPREPQVCTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSP
GKGSGSGSEIVLTQSPATLSLSPGERATLSCRASQSVSSYLAWYQQKPGQAPRL
LIYDASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSP
VTKSFNRGECDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNG
KEYKCKVSNKALPAPIEKTISKAKGQPREPQVCTLPPSREEMTKNQVSLSCAV
KGFYPSDIAVEWESNGQPENNYKTTPPVLDSDGSFFLVSKLTVDKSRWQQGNV
FSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
Y349C; 445P, 446G, 447K insertion
Six amino acids linker insertion: GSGSGS (SEQ ID NO: 541)
Mutations in domain K:
Y349C, D356E, L358M, T366S, L368A, Y407V
>BC28 1x2 domain R [SEQ ID NO: 38]
DIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRT
>BC28 1x2 domain S [SEQ ID NO: 39]
PREPQVCTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
>BC28 1x2 linker [SEQ ID NO: 40]
GSGSGS
>BC28 1x2 domain H [SEQ ID NO: 41]
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIK
>BC28 1x2 domain I [SEQ ID NO: 42]
RTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
>BC28 1x2 domain J [SEQ ID NO: 43]
APELLGGPSVFLEPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAK
>BC28 1x2 domain K [SEQ ID NO: 44]
GQPREPQVCTLPPSREEMTKNQVSLSCAVKGFYPSDIAVEWESNGQPENNYKTTPPVL
DSDGSFFLVSKLTVDKSRWQQGNVFSCSVMHEALHNHYTQKSLSLSPGK
>BC28-1x1x1a chain 3 [SEQ ID NO: 45]
DIQMTQSPSSLSASVGDRVTITCRASQSVSSAVAWYQQKPGKAPKLLIYSASS
LYSGVPSRFSGSRSGTDFTLTISSLQPEDFATYYCQQRDSYLWTFGQGTKVEI
KRTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLSK
SCGSGSGSEIVLTQSPATLSLSPGERATLSCRASQSVSSYLAWYQQKPGQAPRL
LIYDASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSP
VTKSFNRGECDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNG
KEYKCKVSNKALPAPIEKTISKAKGQPREPQVCTLPPSREEMTKNQVSLSCAV
KGFYPSDIAVEWESNGQPENNYKTTPPVLDSDGSFFLVSKLTVDKSRWQQGN
VFSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
T366K; 445K, 446S, 447C insertion
Six amino acids linker insertion: GSGSGS (SEQ ID NO: 541)
Mutations in domain K:
Y349C, D356E, L358M, T366S, L368A, Y407V
>BC28-1x1x1a domain R [SEQ ID NO: 46]
DIQMTQSPSSLSASVGDRVTITCRASQSVSSAVAWYQQKPGKAPKLLIYSASSLYSGV
PSRFSGSRSGTDFTLTISSLQPEDFATYYCQQRDSYLWTFGQGTKVEIKRT
>BC28-1x1x1a domain S [SEQ ID NO: 47]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>BC28-1x1x1a linker [SEQ ID NO: 48]
GSGSGS
>BC28-1x1x1a domain H [SEQ ID NO: 49]
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIK
>BC28-1x1x1a domain I [SEQ ID NO: 50]
RTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
>BC28-1x1x1a domain J [SEQ ID NO: 51]
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAK
>BC28-1x1x1a domain K [SEQ ID NO: 52]
GQPREPQVCTLPPSREEMTKNQVSLSCAVKGFYPSDIAVEWESNGQPENNYKTTPPVL
DSDGSFFLVSKLTVDKSRWQQGNVFSCSVMHEALHNHYTQKSLSLSPGK
>hCTLA4-4.chain 2 [SEQ ID NO: 53]
EVQLVESGGGLVQPGGSLRLSCAASGFTFSTYYIHWVRQAPGKGLEWVAVIYPYTGFT
YYADSVKGRFTISADTSKNTAYLQMNSLRAEDTAVYYCARGEYTVLDYWGQGTLVTVS
SASPREPQVYTDPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSGEC
Domain arrangement:
F- G
VH- CH3
Mutations in domain G
L351D, 445G, 446E, 447C insertion
>hCTLA4-4 domain F [SEQ ID NO: 54]
EVQLVESGGGLVQPGGSLRLSCAASGFTFSTYYIHWVRQAPGKGLEWVAVIYPYTGFT
YYADSVKGRFTISADTSKNTAYLQMNSLRAEDTAVYYCARGEYTVLDYWGQGTLVTVS
SAS
>hCTLA4-4 domain G [SEQ ID NO: 55]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
Other Sequences:
>Hinge: DKTHTCPPCP [SEQ ID NO: 56]
>BC1-Polypeptide 1 Domain Junction: IKRTPREP [SEQ ID NO: 57]
>BC15-Polypeptide 1 Domain Junction: IKRTVREP [SEQ ID NO: 58]
>BC16-Polypeptide 1 Domain Junction: IKRTREP [SEQ ID NO: 59]
>BC17-Polypeptide 1 Domain Junction: IKRTVPREP [SEQ ID NO: 60]
>BC26-Polypeptide 1 Domain Junction: IKRTVAEP [SEQ ID NO: 61]
>BC27-Polypeptide 1 Domain Junction: IKRTVAPREP [SEQ ID NO: 62]
>BC1-Polypeptide 2 Domain Junction: SSASPREP [SEQ ID NO: 63]
>BC13-Polypeptide 2 Domain Junction: SSASTREP [SEQ ID NO: 64]
>BC14-Polypeptide 2 Domain Junction: SSASTPREP [SEQ ID NO: 65]
>BC24-Polypeptide 2 Domain Junction: SSASTKGEP [SEQ ID NO: 66]
>BC25-Polypeptide 2 Domain Junction: SSASTKGREP [SEQ ID NO: 67]
>SP34-89 VH [SEQ ID NO: 68]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTV
>SP34-89 VL [SEQ ID NO: 69]
QAVVTQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVL
>SP34-89 VH-N30S VH [SEQ ID NO: 70] lower case
denotes mutation
EVQLVESGGGLVQPGGSLRLSCAASGFTFsTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTV
>SP34-89 VH-G65D VH [SEQ ID NO: 71] lower case
denotes mutation
EVQLVESGGGLVQPGGSLRLSCAASGFTFNTYAMNWVRQAPGKGLEWVARIRSKYNNY
ATYYADSVKdRFSISRDDSKNTAYLQMNSLRAEDTAVYYCVRHGNFGNSYVSWFAYWG
QGTLVTV
>SP34-89 VH-S68T VH [SEQ ID NO: 72] lower case
denotes mutation
EVQLVESGGGLVQPGGSLRLSCAASGFTFNTYAMNWVRQAPGKGLEWVARIRSKYNNY
ATYYADSVKGRFtISRDDSKNTAYLQMNSLRAEDTAVYYCVRHGNFGNSYVSWFAYWG
QGTLVTV
>SP34-89 VL-W57G VL [SEQ ID NO: 73] lower case
denotes mutation
QAVVTQEPSLTVSPGGTVTLTCRSSTGAVTTSNYANWVQQKPGQAPRGLIGGTNKRAP
gTPARFSGSLLGGKAALTITGAQAEDEADYYCALWYSNLWVFGGGTKLTVL
>Phage display heavy chain [SEQ ID NO: 74]:
EVQLVESGGGLVQPGGSLRLSCAASGFTExxxxIHWVRQAPGKGLEWVAxxxx
xxxxxxxYADSVKGRFTISADTSKNTAYLQMNSLRAEDTAVYYCARxxxxxxxxxx
xxxDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQ
GNVFSCSVMHEALHNHYTQKSLSLSPGK
>Phage display light chain [SEQ ID NO: 75]:
DIQMTQSPSSLSASVGDRVTITCRASQSVSSAVAWYQQKPGKAPKLLIYSASSL
YSGVPSRFSGSRSGTDFTLTISSLQPEDFATYYCQQxxxxxxTFGQGTKVEIKRT
VAAPSVFIFPPSDSQLKSGTASVVCLLNNFYPREAKVQWKVDNALQSGNSQES
VTEQDSKDSTYSLSSTLTLSKADYEKHKVYACEVTHQGLSSPVTKSFNRGEC
>B-Body Domain A/H Scaffold [SEQ ID NO: 76]:
DIQMTQSPSSLSASVGDRVTITCRASQSVSSAVAWYQQKPGKAPKLLIYSASSL
YSGVPSRFSGSRSGTDFTLTISSLQPEDFATYYCQQxxxxxxTFGQGTKVEIKRT
>B-Body Domain F/L Scaffold [SEQ ID NO: 77]:
EVQLVESGGGLVQPGGSLRLSCAASGFTFxxxxIHWVRQAPGKGLEWVAxxxx
xxxxxxxYADSVKGRFTISADTSKNTAYLQMNSLRAEDTAVYYCARxxxxxxxxxx
xxxDYWGQGTLVTVSSAS
>BC1 Chain 1 Scaffold [SEQ ID NO: 78]
DIQMTQSPSSLSASVGDRVTITCRASQSVSSAVAWYQQKPGKAPKLLIYSASSL
YSGVPSRFSGSRSGTDFTLTISSLQPEDFATYYCQQxxxxxxTFGQGTKVEIKRTP
REPQVYTLPPSRDELTKNQVSLKCLVKGFYPSDIAVEWESNGQPENNYKTTPP
VLDSDGSFFLYSKLTVDKSRWQQGNVFSCSVMHEALHNHYTQKSLSLSKSCDK
THTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKAL
PAPIEKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVMHEALHN
HYTQKSLSLSPGK
“x” represents CDR amino acids that were varied to create the
library, and bold italic represents the CDR sequences that
were constant
Domain arrangement:
A- B- Hinge- D- E
VL- CH3- Hinge- CH2- CH3(knob)
Mutations in first CH3 (Domain B):
T366K; 445K, 446S, 447C insertion
Mutations in second CH3 (Domain E):
S354C, T366W
>BC1 Chain 2 Scaffold [SEQ ID NO: 79]
EVQLVESGGGLVQPGGSLRLSCAASGFTFxxxxIHWVRQAPGKGLEWVAxxxx
xxxxxxxYADSVKGRFTISADTSKNTAYLQMNSLRAEDTAVYYCARxxxxxxxxxx
xxxDYWGQGTLVTVSSASPREPQVYTDPPSRDELTKNQVSLTCLVKGFYPSDIA
VEWESNGQPENNYKTTPPVLDSDGSFFLYSKLTVDKSRWQQGNVFSCSVMHEAL
HNHYTQKSLSLSGEC
“x” represents CDR amino acids that were varied to create the
library, and bold italic represents the CDR sequences that
were constant
Domain arrangement:
F- G
VH- CH3
Mutations in CH3 (Domain G):
L351D; 445G, 446E, 447C insertion
>BC1 Chain 3 Scaffold [SEQ ID NO: 80]
DIQMTQSPSSLSASVGDRVTITCRASQSVSSAVAWYQQKPGKAPKLLIYSASSL
YSGVPSRFSGSRSGTDFTLTISSLQPEDFATYYCQQxxxxxxTFGQGTKVEIKRT
VAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGECD
KTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKA
LPAPIEKTISKAKGQPREPQVCTLPPSREEMTKNQVSLSCAVKGFYPSDIAVE
WESNGQPENNYKTTPPVLDSDGSFFLVSKLTVDKSRWQQGNVFSCSVMHEALH
NHYTQKSLSLSPGK
“x” represents CDR amino acids that were varied to create the
library, and bold italic represents the CDR sequences that
were constant
Domain arrangement:
H- I- Hinge- J- K
VL- CL- Hinge- CH2- CH3(hole)
Mutations in CH3 (domain K):
Y349C, D356E, L358M, T366S, L368A, Y407V
>BC1 Chain 4 Scaffold [SEQ ID NO: 81]
EVQLVESGGGLVQPGGSLRLSCAASGFTFxxxxIHWVRQAPGKGLEWVAxxxx
xxxxxxxYADSVKGRFTISADTSKNTAYLQMNSLRAEDTAVYYCARxxxxxxxxxx
xxxDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPS
NTKVDKKVEPPKSC
“x” represents CDR amino acids that were varied to create the
library, and bold italic represents the CDR sequences that
were constant
Domain arrangement:
L- M
VH- CH1
>BC1 Chain 3 1(A)x2(B-A) SP34-89 Scaffold [SEQ ID NO: 82]
DIQMTQSPSSLSASVGDRVTITCRASQSVSSAVAWYQQKPGKAPKLLIYSASSL
YSGVPSRFSGSRSGTDFTLTISSLQPEDFATYYCQQxxxxxxTFGQGTKVEIKRTP
REPQVYTLPPSRDELTKNQVSLKCLVKGFYPSDIAVEWESNGQPENNYKTTPP
VLDSDGSFFLYSKLTVDKSRWQQGNVFSCSVMHEALHNHYTQKSLSLSKSCT
ASSGGSSSGQAVVTQEPSLTVSPGGTVTLTCRSSTGAVTTSNYANWVQQKPGQ
APRGLIGGTNKRAPWTPARFSGSLLGGKAALTITGAQAEDEADYYCALWYSNL
WVFGGGTKLTVLGRTVAAPSVFIFPPSDEQLKSGTASVVCLLNNFYPREAKVQ
WKVDNALQSGNSQESVTEQDSKDSTYSLSSTLTLSKADYEKHKVYACEVTHQG
LSSPVTKSFNRGECDKTHTCPPCPAPEAAGGPSVFLFPPKPKDTLMISRTPEV
TCVVVDVSHEDPEVKFNWYVDGVEVHNAKTKPREEQYNSTYRVVSVLTVLHQD
WLNGKEYKCKVSNKALKAPIEKTISKAKGQPREPQVCTLPPSREEMTKNQVSL
SCAVKGFYPSDIAVEWESNGQPENNYKTTPPVLDSDGSFFLVSKLTVDKSRWQ
QGNVFSCSVMHEALHNHYTQKSLSLSPGK
“x” represents CDR amino acids that were varied to create the
library, and bold italic represents the CDR sequences that
were constant
Domain arrangement:
R- S- linker- H- I- Hinge- J- K
VL- CH3- linker- SP34- CL- Hinge- CH2- CH3(hole)
Mutations in domain S:
T366K; 445K, 446S, 447C insertion
Ten amino acids linker insertion: TASSGGSSSG (SEQ ID NO: 83)
Mutations in Domain J:
L234A, L235A, and P329K
Mutations in domain K:
Y349C, D356E, L358M, T366S, L368A, Y407V
>BC1 Chain 3 1(A)x2(B-A) SP34-89 S-H Junction [SEQ ID NO: 83]
TASSGGSSSG
>I2A-10 Chain 1 BC1 1x1 [SEQ ID NO: 84]
DIQMTQSPSSLSASVGDRVTITCRASQSVSSAVAWYQQKPGKAPKLLIYSASSLYSGV
PSRFSGSRSGTDFTLTISSLQPEDFATYYCQQYYYYPGTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCDKTHTCPPCPAPEAAGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALKAPIEKTISKAKGQPREPQVYTLPPC
RDELTKNQVSLWCLVKGFYPSDIAVEWESNGQPENNYKTTPPVLDSDGSFFLYSKLTV
DKSRWQQGNVFSCSVMHEALHNHYTQKSLSLSPGK
>I2A-10 Chain 2 BC1 1x1 [SEQ ID NO: 85]
EVQLVESGGGLVQPGGSLRLSCAASGFTFSSYFIHWVRQAPGKGLEWVAGIYPSDGYT
SYADSVKGRFTISADTSKNTAYLQMNSLRAEDTAVYYCARYYSGMDYWGQGTLVTVSS
ASPREPQVYTDPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSGEC
>I2A-10 Chain 3 BC1 1x1 [SEQ ID NO: 86]
QAVVTQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLGRTVAAP
SVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECDKTHTCPPCPAPEAA
GGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALKAPIEKTISKAKGQPREPQVCTL
PPSREEMTKNQVSLSCAVKGFYPSDIAVEWESNGQPENNYKTTPPVLDSDGSFFLVSK
LTVDKSRWQQGNVFSCSVMHEALHNHYTQKSLSLSPGK
>I2A-10 Chain 4 BC1 1x1 [SEQ ID NO: 87]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
>I2A-10 Chain 3 BC1 1x2 [SEQ ID NO: 88]
DIQMTQSPSSLSASVGDRVTITCRASQSVSSAVAWYQQKPGKAPKLLIYSASSLYSGV
PSRFSGSRSGTDFTLTISSLQPEDFATYYCQQYYYYPGTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCTASSGGSSSGQAVVTQEP
SLTVSPGGTVTLTCRSSTGAVTTSNYANWVQQKPGQAPRGLIGGTNKRAPWTPARFSG
SLLGGKAALTITGAQAEDEADYYCALWYSNLWVFGGGTKLTVLGRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEAAGGPSVFLF
PPKPKDTLMISRTPEVTCVVVDVSHEDPEVKFNWYVDGVEVHNAKTKPREEQYNSTYR
VVSVLTVLHQDWLNGKEYKCKVSNKALKAPIEKTISKAKGQPREPQVCTLPPSREEMT
KNQVSLSCAVKGFYPSDIAVEWESNGQPENNYKTTPPVLDSDGSFFLVSKLTVDKSRW
QQGNVFSCSVMHEALHNHYTQKSLSLSPGK
>I2A-27 Chain 1 BC1 1x1 [SEQ ID NO: 89]
DIQMTQSPSSLSASVGDRVTITCRASQSVSSAVAWYQQKPGKAPKLLIYSASSLYSGV
PSRFSGSRSGTDFTLTISSLQPEDFATYYCQQYYSSPRTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCDKTHTCPPCPAPEAAGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALKAPIEKTISKAKGQPREPQVYTLPPC
RDELTKNQVSLWCLVKGFYPSDIAVEWESNGQPENNYKTTPPVLDSDGSFFLYSKLTV
DKSRWQQGNVFSCSVMHEALHNHYTQKSLSLSPGK
>I2A-27 Chain 2 BC1 1x1 [SEQ ID NO: 90]
EVQLVESGGGLVQPGGSLRLSCAASGFTFKGYYIHWVRQAPGKGLEWVAAIYPYGGST
DYADSVKGRFTISADTSKNTAYLQMNSLRAEDTAVYYCARVYIYGVFDYWGQGTLVTV
SSASPREPQVYTDPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSGEC
>I2A-27 Chain 3 BC1 1x1 [SEQ ID NO: 91]
QAVVTQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLGRTVAAP
SVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECDKTHTCPPCPAPEAA
GGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALKAPIEKTISKAKGQPREPQVCTL
PPSREEMTKNQVSLSCAVKGFYPSDIAVEWESNGQPENNYKTTPPVLDSDGSFFLVSK
LTVDKSRWQQGNVFSCSVMHEALHNHYTQKSLSLSPGK
>I2A-27 Chain 4 BC1 1x1 [SEQ ID NO: 92]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
>I2A-27 Chain 3 BC1 1x2 [SEQ ID NO: 93]
DIQMTQSPSSLSASVGDRVTITCRASQSVSSAVAWYQQKPGKAPKLLIYSASSLYSGV
PSRFSGSRSGTDFTLTISSLQPEDFATYYCQQYYSSPRTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCTASSGGSSSGQAVVTQEP
SLTVSPGGTVTLTCRSSTGAVTTSNYANWVQQKPGQAPRGLIGGTNKRAPWTPARFSG
SLLGGKAALTITGAQAEDEADYYCALWYSNLWVFGGGTKLTVLGRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPEAAGGPSVFLF
PPKPKDTLMISRTPEVTCVVVDVSHEDPEVKFNWYVDGVEVHNAKTKPREEQYNSTYR
VVSVLTVLHQDWLNGKEYKCKVSNKALKAPIEKTISKAKGQPREPQVCTLPPSREEMT
KNQVSLSCAVKGFYPSDIAVEWESNGQPENNYKTTPPVLDSDGSFFLVSKLTVDKSRW
QQGNVFSCSVMHEALHNHYTQKSLSLSPGK
>Human ROR1 (UniProt accession #Q01973) [SEQ ID NO: 94]
MHRPRRRGTRPPLLALLAALLLAARGAAAQETELSVSAELVPTSSWNISSELNKDSYL
TLDEPMNNITTSLGQTAELHCKVSGNPPPTIRWFKNDAPVVQEPRRLSFRSTIYGSRL
RIRNLDTTDTGYFQCVATNGKEVVSSTGVLFVKFGPPPTASPGYSDEYEEDGFCQPYR
GIACARFIGNRTVYMESLHMQGEIENQITAAFTMIGTSSHLSDKCSQFAIPSLCHYAF
PYCDETSSVPKPRDLCRDECEILENVLCQTEYIFARSNPMILMRLKLPNCEDLPQPES
PEAANCIRIGIPMADPINKNHKCYNSTGVDYRGTVSVTKSGRQCQPWNSQYPHTHTFT
ALRFPELNGGHSYCRNPGNQKEAPWCFTLDENFKSDLCDIPACDSKDSKEKNKMEILY
ILVPSVAIPLAIALLFFFICVCRNNQKSSSAPVQRQPKHVRGQNVEMSMLNAYKPKSK
AKELPLSAVRFMEELGECAFGKIYKGHLYLPGMDHAQLVAIKTLKDYNNPQQWTEFQQ
EASLMAELHHPNIVCLLGAVTQEQPVCMLFEYINQGDLHEFLIMRSPHSDVGCSSDED
GTVKSSLDHGDFLHIAIQIAAGMEYLSSHFFVHKDLAARNILIGEQLHVKISDLGLSR
EIYSADYYRVQSKSLLPIRWMPPEAIMYGKFSSDSDIWSFGVVLWEIFSFGLQPYYGF
SNQEVIEMVRKRQLLPCSEDCPPRMYSLMTECWNEIPSRRPRFKDIHVRLRSWEGLSS
HTSSTTPSGGNATTQTTSLSASPVSNLSNPRYPNYMFPSQGITPQGQIAGFIGPPIPQ
NQRFIPINGYPIPPGYAAFPAAHYQPTGPPRVIQHCPPPKSRSPSSASGSTSTGHVTS
LPSSGSNQEANIPLLPHMSIPNHPGGMGITVFGNKSQKPYKIDSKQASLLGDANIHGH
TESMISAEL
>Human ROR2 (UniProt accession #Q01974) [SEQ ID NO: 95]
MARGSALPRRPLLCIPAVWAAAALLLSVSRTSGEVEVLDPNDPLGPLDGQDGPIPTLK
GYFLNFLEPVNNITIVQGQTAILHCKVAGNPPPNVRWLKNDAPVVQEPRRIIIRKTEY
GSRLRIQDLDTTDTGYYQCVATNGMKTITATGVLFVRLGPTHSPNHNFQDDYHEDGFC
QPYRGIACARFIGNRTIYVDSLQMQGEIENRITAAFTMIGTSTHLSDQCSQFAIPSFC
HFVFPLCDARSRTPKPRELCRDECEVLESDLCRQEYTIARSNPLILMRLQLPKCEALP
MPESPDAANCMRIGIPAERLGRYHQCYNGSGMDYRGTASTTKSGHQCQPWALQHPHSH
HLSSTDFPELGGGHAYCRNPGGQMEGPWCFTQNKNVRMELCDVPSCSPRDSSKMGILY
ILVPSIAIPLVIACLFFLVCMCRNKQKASASTPQRRQLMASPSQDMEMPLINQHKQAK
LKEISLSAVRFMEELGEDRFGKVYKGHLFGPAPGEQTQAVAIKTLKDKAEGPLREEFR
HEAMLRARLQHPNVVCLLGVVTKDQPLSMIFSYCSHGDLHEFLVMRSPHSDVGSTDDD
RTVKSALEPPDFVHLVAQIAAGMEYLSSHHVVHKDLATRNVLVYDKLNVKISDLGLFR
EVYAADYYKLLGNSLLPIRWMAPEAIMYGKFSIDSDIWSYGVVLWEVFSYGLQPYCGY
SNQDVVEMIRNRQVLPCPDDCPAWVYALMIECWNEFPSRRPRFKDIHSRLRAWGNLSN
YNSSAQTSGASNTTQTSSLSTSPVSNVSNARYVGPKQKAPPFPQPQFIPMKGQIRPMV
PPPQLYVPVNGYQPVPAYGAYLPNFYPVQIPMQMAPQQVPPQMVPKPSSHHSGSGSTS
TGYVTTAPSNTSMADRAALLSEGADDTQNAPEDGAQSTVQEAEEEEEGSVPETELLGD
CDTLQVDEAQVQLEA
I2A-27 Chain 1 [SEQ ID NO: 96]
DIQMTQSPSSLSASVGDRVTITCRASQSVSSAVAWYQQKPGKAPKLLIYSASSLYSGV
PSRFSGSRSGTDFTLTISSLQPEDFATYYCQQYYSSPRTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCDKTHTCPPCPAPEAAGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALKAPIEKTISKAKGQPREPQVCTLPPS
REEMTKNQVSLSCAVKGFYPSDIAVEWESNGQPENNYKTTPPVLDSDGSFFLVSKLTV
DKSRWQQGNVFSCSVMHEALHNHYTQKSLSLSPGK
Domain arrangement:
A- B- Hinge- D- E
VL- CH3- Hinge- CH2- CH3(hole)
Mutations in first CH3 (Domain B):
T366K; 445K, 446S, 447C insertion
Hinge sequence: DKTHTCPPCP (SEQ ID NO: 56)
Mutations in second CH3 (Domain E):
Y349C, D356E, L358M, T366S, L368A, Y407V
Exemplary signal sequence for Chain 1:
MGWSLILLFLVAVATRVLS (SEQ ID NO: 150)
>I2A-27 Chain 2 [SEQ ID NO: 97]
EVQLVESGGGLVQPGGSLRLSCAASGFTFKGYYIHWVRQAPGKGLEWVAAIYPYGGST
DYADSVKGRFTISADTSKNTAYLQMNSLRAEDTAVYYCARVYIYGVFDYWGQGTLVTV
SSASPREPQVYTDPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSGEC
Domain arrangement:
F- G
VH- CH3
Mutations in CH3 (Domain G):
L351D; 445G, 446E, 447C insertion
Exemplary signal sequence for Chain 2:
MDFQVQIISFLLISASVIMSRG (SEQ ID NO: 151)
>I2A-27 Chain 3 [SEQ ID NO: 98]
DIQMTQSPSSLSASVGDRVTITCRASQSVSSAVAWYQQKPGKAPKLLIYSASS
LYSGVPSRFSGSRSGTDFTLTISSLQPEDFATYYCQQYYSSPRTFGQGTKVEI
KRTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLSK
SCTASSGGSSSGQAVVTQEPSLTVSPGGTVTLTCRSSTGAVTTSNYANWVQQKP
GQAPRGLIGGTNKRAPWTPARFSGSLLGGKAALTITGAQAEDEADYYCALWY
SNLWVFGGGTKLTVLGRTVAAPSVFIFPPSDEQLKSGTASVVCLLNNFYPRE
AKVQWKVDNALQSGNSQESVTEQDSKDSTYSLSSTLTLSKADYEKHKVYACEV
THQGLSSPVTKSFNRGECDKTHTCPPCPAPEAAGGPSVFLFPPKPKDTLMISR
TPEVTCVVVDVSHEDPEVKFNWYVDGVEVHNAKTKPREEQYNSTYRVVSVLTV
LHQDWLNGKEYKCKVSNKALKAPIEKTISKAKGQPREPQVYTLPPCRDELTKN
QVSLWCLVKGFYPSDIAVEWESNGQPENNYKTTPPVLDSDGSFFLYSKLTVDK
SRWQQGNVFSCSVMHEALHNHYTQKSLSLSPGK
Domain arrangement:
R- S- linker- H- I- Hinge- J- K
VL- CH3- linker- VL- CL- Hinge- CH2- CH3(knob)
Mutations in first CH3 (Domain S):
T366K; 445K, 446S, 447C insertion
Ten amino acid linker insertion: TASSGGSSSG (SEQ ID NO: 83)
Hinge sequence: DKTHTCPPCP (SEQ ID NO: 56)
Mutations in second CH3 (Domain K):
S354C, K366W
Exemplary signal sequence for Chain 3:
MDFQVQIISFLLISASVIMSRGS (SEQ ID NO: 152)
>I2A-27 Chain 4 [SEQ ID NO: 99]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement:
L- M
VH- CH1
Exemplary signal sequence for Chain 4:
MDFQVQIISFLLISASVIMSRG (SEQ ID NO: 151)
>I2A-27 Chain 5 [SEQ ID NO: 97]
EVQLVESGGGLVQPGGSLRLSCAASGFTFKGYYIHWVRQAPGKGLEWVAAIYPYGGST
DYADSVKGRFTISADTSKNTAYLQMNSLRAEDTAVYYCARVYIYGVFDYWGQGTLVTV
SSASPREPQVYTDPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSGEC
Domain arrangement:
T- U
VH- CH3
Mutations in CH3 (Domain U):
L351D; 445G, 446E, 447C insertion
Exemplary signal sequence for Chain 5:
MDFQVQIISFLLISASVIMSRG (SEQ ID NO: 151)
>I2A-27 Domain A [SEQ ID NO: 100]
DIQMTQSPSSLSASVGDRVTITCRASQSVSSAVAWYQQKPGKAPKLLIYSASSLYSGV
PSRFSGSRSGTDFTLTISSLQPEDFATYYCQQYYSSPRTFGQGTKVEIKRT
>I2A-27 Domain B [SEQ ID NO: 101]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>I2A-27 Domain D [SEQ ID NO: 102]
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALKAPIEKTISKAK
>I2A-27 Domain E [SEQ ID NO: 103]
GQPREPQVCTLPPSREEMTKNQVSLSCAVKGFYPSDIAVEWESNGQPENNYKTTPPVL
DSDGSFFLVSKLTVDKSRWQQGNVFSCSVMHEALHNHYTQKSLSLSPGK
>I2A-27 Domain F [SEQ ID NO: 104]
EVQLVESGGGLVQPGGSLRLSCAASGFTFKGYYIHWVRQAPGKGLEWVAAIYPYGGST
DYADSVKGRFTISADTSKNTAYLQMNSLRAEDTAVYYCARVYIYGVFDYWGQGTLVTV
SSAS
>I2A-27 Domain G [SEQ ID NO: 105]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
>I2A-27 Domain R [SEQ ID NO: 106]
DIQMTQSPSSLSASVGDRVTITCRASQSVSSAVAWYQQKPGKAPKLLIYSASSLYSGV
PSRFSGSRSGTDFTLTISSLQPEDFATYYCQQYYSSPRTFGQGTKVEIKRT
>I2A-27 Domain S [SEQ ID NO: 107]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>I2A-27 Domain H [SEQ ID NO: 108]
QAVVTQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLGRTV
>I2A-27 Domain I [SEQ ID NO: 109]
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
>I2A-27 Domain J [SEQ ID NO: 110]
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALKAPIEKTISKAK
>I2A-27 Domain K [SEQ ID NO: 111]
GQPREPQVYTLPPCRDELTKNQVSLWCLVKGFYPSDIAVEWESNGQPENNYKTTPPVL
DSDGSFFLYSKLTVDKSRWQQGNVFSCSVMHEALHNHYTQKSLSLSPGK
>I2A-27 Domain L [SEQ ID NO: 112]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSAS
>I2A-27 Domain M [SEQ ID NO: 113]
TKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPPKSC
>I2A-27 Domain T [SEQ ID NO: 104]
EVQLVESGGGLVQPGGSLRLSCAASGFTFKGYYIHWVRQAPGKGLEWVAAIYPYGGST
DYADSVKGRFTISADTSKNTAYLQMNSLRAEDTAVYYCARVYIYGVFDYWGQGTLVTV
SSAS
>I2A-27 Domain U [SEQ ID NO: 105]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
>I2A-10 Chain 1 [SEQ ID NO: 114]
DIQMTQSPSSLSASVGDRVTITCRASQSVSSAVAWYQQKPGKAPKLLIYSASSLYSGV
PSRFSGSRSGTDFTLTISSLQPEDFATYYCQQYYYYPGTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCDKTHTCPPCPAPEAAGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALKAPIEKTISKAKGQPREPQVCTLPPS
REEMTKNQVSLSCAVKGFYPSDIAVEWESNGQPENNYKTTPPVLDSDGSFFLVSKLTV
DKSRWQQGNVFSCSVMHEALHNHYTQKSLSLSPGK
Domain arrangement:
A- B- Hinge- D- E
VL- CH3- Hinge- CH2- CH3(hole)
Mutations in first CH3 (Domain B):
T366K; 445K, 446S, 447C insertion
Hinge sequence: DKTHTCPPCP (SEQ ID NO: 56)
Mutations in second CH3 (Domain E):
Y349C, D356E, L358M, T366S, L368A, Y407V
Exemplary signal sequence for Chain 1:
MGWSLILLFLVAVATRVLS (SEQ ID NO: 150)
>I2A-10 Chain 2 [SEQ ID NO: 115]
EVQLVESGGGLVQPGGSLRLSCAASGFTFSSYFIHWVRQAPGKGLEWVAGIYPSDGYT
SYADSVKGRFTISADTSKNTAYLQMNSLRAEDTAVYYCARYYVSGMDYWGQGTLVTVS
SASPREPQVYTDPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSGEC
Domain arrangement:
F- G
VH- CH3
Mutations in CH3 (Domain G):
L351D; 445G, 446E, 447C insertion
Exemplary signal sequence for Chain 2:
MDFQVQIISFLLISASVIMSRG (SEQ ID NO: 151)
>I2A-10 Chain 3 [SEQ ID NO: 116]
DIQMTQSPSSLSASVGDRVTITCRASQSVSSAVAWYQQKPGKAPKLLIYSASS
LYSGVPSRFSGSRSGTDFTLTISSLQPEDFATYYCQQYYYYPGTFGQGTKVEI
KRTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLSK
SCTASSGGSSSGQAVVTQEPSLTVSPGGTVTLTCRSSTGAVTTSNYANWVQQKP
GQAPRGLIGGTNKRAPWTPARFSGSLLGGKAALTITGAQAEDEADYYCALWY
SNLWVFGGGTKLTVLGRTVAAPSVFIFPPSDEQLKSGTASVVCLLNNFYPRE
AKVQWKVDNALQSGNSQESVTEQDSKDSTYSLSSTLTLSKADYEKHKVYACEV
THQGLSSPVTKSFNRGECDKTHTCPPCPAPEAAGGPSVFLFPPKPKDTLMISR
TPEVTCVVVDVSHEDPEVKFNWYVDGVEVHNAKTKPREEQYNSTYRVVSVLTV
LHQDWLNGKEYKCKVSNKALKAPIEKTISKAKGQPREPQVYTLPPCRDELTKN
QVSLWCLVKGFYPSDIAVEWESNGQPENNYKTTPPVLDSDGSFFLYSKLTVDK
SRWQQGNVFSCSVMHEALHNHYTQKSLSLSPGK
Domain arrangement:
R- S- linker- H- I- Hinge- J- K
VL- CH3- linker- VL- CL- Hinge- CH2- CH3(knob)
Mutations in first CH3 (Domain S):
T366K; 445K, 446S, 447C insertion
Ten amino acid linker insertion: TASSGGSSSG (SEQ ID NO: 83)
Hinge sequence: DKTHTCPPCP (SEQ ID NO: 56)
Mutations in second CH3 (Domain K):
S354C, K366W
Exemplary signal sequence for Chain 3:
MDFQVQIISFLLISASVIMSRGS (SEQ ID NO: 152)
>I2A-10 Chain 4 [SEQ ID NO: 117]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement:
L- M
VH- CH1
Exemplary signal sequence for Chain 4:
MDFQVQIISFLLISASVIMSRG (SEQ ID NO: 151)
>I2A-10 Chain 5 [SEQ ID NO: 115]
EVQLVESGGGLVQPGGSLRLSCAASGFTFSSYFIHWVRQAPGKGLEWVAGIYPSDGYT
SYADSVKGRFTISADTSKNTAYLQMNSLRAEDTAVYYCARYYVSGMDYWGQGTLVTVS
SASPREPQVYTDPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSGEC
Domain arrangement:
T- U
VH- CH3
Mutations in CH3 (Domain U):
L351D; 445G, 446E, 447C insertion
Exemplary signal sequence for Chain 5:
MDFQVQIISFLLISASVIMSRG (SEQ ID NO: 151)
>I2A-10 Domain A [SEQ ID NO: 118]
DIQMTQSPSSLSASVGDRVTITCRASQSVSSAVAWYQQKPGKAPKLLIYSASSLYSGV
PSRFSGSRSGTDFTLTISSLQPEDFATYYCQQYYYYPGTFGQGTKVEIKRT
>I2A-10 Domain B [SEQ ID NO: 119]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>I2A-10 Domain D [SEQ ID NO: 120]
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALKAPIEKTISKAK
>I2A-10 Domain E [SEQ ID NO: 121]
GQPREPQVCTLPPSREEMTKNQVSLSCAVKGFYPSDIAVEWESNGQPENNYKTTPPVL
DSDGSFFLVSKLTVDKSRWQQGNVFSCSVMHEALHNHYTQKSLSLSPGK
>I2A-10 Domain F [SEQ ID NO: 122]
EVQLVESGGGLVQPGGSLRLSCAASGFTFSSYFIHWVRQAPGKGLEWVAGIYPSDGYT
SYADSVKGRFTISADTSKNTAYLQMNSLRAEDTAVYYCARYYVSGMDYWGQGTLVTVS
SAS
>I2A-10 Domain G [SEQ ID NO: 123]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
>I2A-10 Domain R [SEQ ID NO: 124]
DIQMTQSPSSLSASVGDRVTITCRASQSVSSAVAWYQQKPGKAPKLLIYSASSLYSGV
PSRFSGSRSGTDFTLTISSLQPEDFATYYCQQYYYYPGTFGQGTKVEIKRT
>I2A-10 Domain S [SEQ ID NO: 125]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>I2A-10 Domain H [SEQ ID NO: 126]
QAVVTQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLGRTV
>I2A-10 Domain I [SEQ ID NO: 127]
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
>I2A-10 Domain J [SEQ ID NO: 128]
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALKAPIEKTISKAK
>I2A-10 Domain K [SEQ ID NO: 129]
GQPREPQVYTLPPCRDELTKNQVSLWCLVKGFYPSDIAVEWESNGQPENNYKTTPPVL
DSDGSFFLYSKLTVDKSRWQQGNVFSCSVMHEALHNHYTQKSLSLSPGK
>I2A-10 Domain L [SEQ ID NO: 130]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSAS
>I2A-10 Domain M [SEQ ID NO: 131]
TKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPPKSC
>I2A-10 Domain T [SEQ ID NO: 122]
EVQLVESGGGLVQPGGSLRLSCAASGFTFSSYFIHWVRQAPGKGLEWVAGIYPSDGYT
SYADSVKGRFTISADTSKNTAYLQMNSLRAEDTAVYYCARYYVSGMDYWGQGTLVTVS
SAS
>I2A-10 Domain U [SEQ ID NO: 123]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
>I2A-10 D54E Y55Q Chain 1 [SEQ ID NO: 132]
DIQMTQSPSSLSASVGDRVTITCRASQSVSSAVAWYQQKPGKAPKLLIYSASSLQSGV
PSRFSGSRSGTDFTLTISSLQPEDFATYYCQQYYYYPGTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCDKTHTCPPCPAPEAAGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALKAPIEKTISKAKGQPREPQVCTLPPS
REEMTKNQVSLSCAVKGFYPSDIAVEWESNGQPENNYKTTPPVLDSDGSFFLVSKLTV
DKSRWQQGNVFSCSVMHEALHNHYTQKSLSLSPGK
Domain arrangement:
A- B- Hinge- D- E
VL- CH3- Hinge- CH2- CH3(hole)
Mutations in first CH3 (Domain B):
T366K; 445K, 446S, 447C insertion
Hinge sequence: DKTHTCPPCP (SEQ ID NO: 56)
Mutations in second CH3 (Domain E):
Y349C, D356E, L358M, T366S, L368A, Y407V
Exemplary signal sequence for Chain 1:
MGWSLILLFLVAVATRVLS (SEQ ID NO: 150)
>I2A-10 D54E Y55Q Chain 2 [SEQ ID NO: 133]
EVQLVESGGGLVQPGGSLRLSCAASGFTFSSYFIHWVRQAPGKGLEWVAGIYPSEGYT
SYADSVKGRFTISADTSKNTAYLQMNSLRAEDTAVYYCARYYVSGMDYWGQGTLVTVS
SASPREPQVYTDPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSGEC
Domain arrangement:
F- G
VH- CH3
Mutations in CH3 (Domain G):
L351D; 445G, 446E, 447C insertion
Exemplary signal sequence for Chain 2:
MDFQVQIISFLLISASVIMSRG (SEQ ID NO: 151)
>I2A-10 D54E Y55Q Chain 3 [SEQ ID NO: 134]
DIQMTQSPSSLSASVGDRVTITCRASQSVSSAVAWYQQKPGKAPKLLIYSASS
LQSGVPSRFSGSRSGTDFTLTISSLQPEDFATYYCQQYYYYPGTFGQGTKVEI
KRTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLSK
SCTASSGGSSSGQAVVTQEPSLTVSPGGTVTLTCRSSTGAVTTSNYANWVQQKP
GQAPRGLIGGTNKRAPWTPARFSGSLLGGKAALTITGAQAEDEADYYCALWY
SNLWVFGGGTKLTVLGRTVAAPSVFIFPPSDEQLKSGTASVVCLLNNFYPRE
AKVQWKVDNALQSGNSQESVTEQDSKDSTYSLSSTLTLSKADYEKHKVYACEV
THQGLSSPVTKSFNRGECDKTHTCPPCPAPEAAGGPSVFLFPPKPKDTLMISR
TPEVTCVVVDVSHEDPEVKFNWYVDGVEVHNAKTKPREEQYNSTYRVVSVLTV
LHQDWLNGKEYKCKVSNKALKAPIEKTISKAKGQPREPQVYTLPPCRDELTKN
QVSLWCLVKGFYPSDIAVEWESNGQPENNYKTTPPVLDSDGSFFLYSKLTVDK
SRWQQGNVFSCSVMHEALHNHYTQKSLSLSPGK
Domain arrangement:
R- S- linker- H- I- Hinge- J- K
VL- CH3- linker- VL- CL- Hinge- CH2- CH3(knob)
Mutations in first CH3 (Domain S):
T366K; 445K, 446S, 447C insertion
Ten amino acid linker insertion: TASSGGSSSG (SEQ ID NO: 83)
Hinge sequence: DKTHTCPPCP (SEQ ID NO: 56)
Mutations in second CH3 (Domain K):
S354C, K366W
Exemplary signal sequence for Chain 3:
MDFQVQIISFLLISASVIMSRGS (SEQ ID NO: 152)
>I2A-10 D54E Y55Q Chain 4 [SEQ ID NO: 135]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement:
L- M
VH- CH1
Exemplary signal sequence for Chain 4:
MDFQVQIISFLLISASVIMSRG (SEQ ID NO: 151)
>I2A-10 D54E Y55Q Chain 5 [SEQ ID NO: 133]
EVQLVESGGGLVQPGGSLRLSCAASGFTFSSYFIHWVRQAPGKGLEWVAGIYPSEGYT
SYADSVKGRFTISADTSKNTAYLQMNSLRAEDTAVYYCARYYVSGMDYWGQGTLVTVS
SASPREPQVYTDPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSGEC
Domain arrangement:
T- U
VH- CH3
Mutations in CH3 (Domain U):
L351D; 445G, 446E, 447C insertion
Exemplary signal sequence for Chain 5:
MDFQVQIISFLLISASVIMSRG (SEQ ID NO: 151)
>I2A-10 D54E Y55Q Domain A [SEQ ID NO: 136]
DIQMTQSPSSLSASVGDRVTITCRASQSVSSAVAWYQQKPGKAPKLLIYSASSLQSGV
PSRFSGSRSGTDFTLTISSLQPEDFATYYCQQYYYYPGTFGQGTKVEIKRT
>I2A-10 D54E Y55Q Domain B [SEQ ID NO: 137]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>I2A-10 D54E Y55Q Domain D [SEQ ID NO: 138]
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALKAPIEKTISKAK
>I2A-10 D54E Y55Q Domain E [SEQ ID NO: 139]
GQPREPQVCTLPPSREEMTKNQVSLSCAVKGFYPSDIAVEWESNGQPENNYKTTPPVL
DSDGSFFLVSKLTVDKSRWQQGNVFSCSVMHEALHNHYTQKSLSLSPGK
>I2A-10 D54E Y55Q Domain F [SEQ ID NO: 140]
EVQLVESGGGLVQPGGSLRLSCAASGFTFSSYFIHWVRQAPGKGLEWVAGIYPSEGYT
SYADSVKGRFTISADTSKNTAYLQMNSLRAEDTAVYYCARYYVSGMDYWGQGTLVTVS
SAS
>I2A-10 D54E Y55Q Domain G [SEQ ID NO: 141]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC
>I2A-10 D54E Y55Q Domain R [SEQ ID NO: 142]
DIQMTQSPSSLSASVGDRVTITCRASQSVSSAVAWYQQKPGKAPKLLIYSASSLQSGV
PSRFSGSRSGTDFTLTISSLQPEDFATYYCQQYYYYPGTFGQGTKVEIKRT
>I2A-10 D54E Y55Q Domain S [SEQ ID NO: 143]
PREPQVYTLPPSRDELTKNQVSLKCLVKGFYPSDIAVEWESNGQPENNYKTTPPVLDS
DGSFFLYSKLTVDKSRWQQGNVFSCSVMHEALHNHYTQKSLSLSKSC
>I2A-10 D54E Y55Q Domain H [SEQ ID NO: 144]
QAVVTQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLGRTV
>I2A-10 D54E Y55Q Domain I [SEQ ID NO: 145]
AAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
>I2A-10 D54E Y55Q Domain J [SEQ ID NO: 146]
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALKAPIEKTISKAK
>I2A-10 D54E Y55Q Domain K [SEQ ID NO: 147]
GQPREPQVYTLPPCRDELTKNQVSLWCLVKGFYPSDIAVEWESNGQPENNYKTTPPVL
DSDGSFFLYSKLTVDKSRWQQGNVFSCSVMHEALHNHYTQKSLSLSPGK
>I2A-10 D54E Y55Q Domain L [SEQ ID NO: 148]
EVQLVESGGGLVQPGGSLRLSCAASGFTFNTYAMNWVRQAPGKGLEWVARIRSKYNNY
ATYYADSVKGRFSISRDDSKNTAYLQMNSLRAEDTAVYYCVRHGNFGNSYVSWFAYWG
QGTLVTVSSAS
>I2A-10 D54E Y55Q Domain M [SEQ ID NO: 149]
TKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPPKSC
>I2A-10 D54E Y55Q Domain T [SEQ ID NO: 140]
EVQLVESGGGLVQPGGSLRLSCAASGFTFSSYFIHWVRQAPGKGLEWVAGIYPSEGYT
SYADSVKGRFTISADTSKNTAYLQMNSLRAEDTAVYYCARYYVSGMDYWGQGTLVTVS
SAS
>I2A-10 D54E Y55Q Domain U [SEQ ID NO: 141]
PREPQVYTDPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSGEC

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. (canceled)

2. A tyrosine-protein kinase transmembrane receptor (ROR) binding molecule, the ROR antigen binding molecule comprising
a first antigen binding site specific for a ROR antigen, wherein the first antigen binding site comprises an antibody heavy chain variable (VH) region, and an antibody light chain variable (VL) region, wherein the VH region comprises a VH CDR1, a VH CDR2, and a VH CDR3 as set forth in the amino acid sequence of SEQ ID NO: 104, and the VL region comprises a VL CDR1, a VL CDR2, and a VL CDR3 as set forth in the amino acid sequence of SEQ ID NO: 100.

3. (canceled)

4. The ROR antigen binding molecule of claim 2, wherein the ROR antigen is a ROR1 Kringle domain.

5. The ROR antigen binding molecule of claim 2, wherein the ROR antigen comprises a human ROR antigen.

6-8. (canceled)

9. The ROR antigen binding molecule of claim 2, wherein the ROR antigen binding molecule further comprises a second antigen binding site.

10. The ROR antigen binding molecule of claim 9, wherein the second antigen binding site is the same as the first antigen binding site.

11. The ROR antigen binding molecule of claim 9, wherein the second antigen binding site comprises
(1) a heavy chain CDR1 comprising the amino acid sequence of SEQ ID NO: 213,
a heavy chain CDR2 comprising the amino acid sequence of SEQ ID NO: 214,
a heavy chain CDR3 comprising the amino acid sequence of SEQ ID NO: 215,
a light chain CDR1 comprising the amino acid sequence of SEQ ID NO: 216,
a light chain CDR2 comprising the amino acid sequence of SEQ ID NO: 217, and
a light chain CDR3 comprising the amino acid sequence of SEQ ID NO: 218,
(2) a heavy chain CDR1 comprising the amino acid sequence of SEQ ID NO: 207,
a heavy chain CDR2 comprising the amino acid sequence of SEQ ID NO: 208,
a heavy chain CDR3 comprising the amino acid sequence of SEQ ID NO: 209
a light chain CDR1 comprising the amino acid sequence of SEQ ID NO: 210,
a light chain CDR2 comprising the amino acid sequence of SEQ ID NO: 211, and
a light chain CDR3 comprising the amino acid sequence of SEQ ID NO: 212;
(3) a heavy chain CDR1 comprising the amino acid sequence of SEQ ID NO: 153,
a heavy chain CDR2 comprising the amino acid sequence of SEQ ID NO: 154,
a heavy chain CDR3 comprising the amino acid sequence of SEQ ID NO: 155,
a light chain CDR1 comprising the amino acid sequence of SEQ ID NO: 156,
a light chain CDR2 comprising the amino acid sequence of SEQ ID NO: 157, and
a light chain CDR3 comprising the amino acid sequence of SEQ ID NO: 158,
(4) a heavy chain CDR1 comprising the amino acid sequence of SEQ ID NO: 219,
a heavy chain CDR2 comprising the amino acid sequence of SEQ ID NO: 220,
a heavy chain CDR3 comprising the amino acid sequence of SEQ ID NO: 221,
a light chain CDR1 comprising the amino acid sequence of SEQ ID NO: 222,
a light chain CDR2 comprising the amino acid sequence of SEQ ID NO: 223, and
a light chain CDR3 comprising the amino acid sequence of SEQ ID NO: 224,
(5) a heavy chain CDR1 comprising the amino acid sequence of SEQ ID NO: 231,
a heavy chain CDR2 comprising the amino acid sequence of SEQ ID NO: 232,
a heavy chain CDR3 comprising the amino acid sequence of SEQ ID NO: 233,
a light chain CDR1 comprising the amino acid sequence of SEQ ID NO: 234,
a light chain CDR2 comprising the amino acid sequence of SEQ ID NO: 235, and
a light chain CDR3 comprising the amino acid sequence of SEQ ID NO: 236,
(6) a heavy chain CDR1 comprising the amino acid sequence of SEQ ID NO: 237,
a heavy chain CDR2 comprising the amino acid sequence of SEQ ID NO: 238,
a heavy chain CDR3 comprising the amino acid sequence of SEQ ID NO: 239,
a light chain CDR1 comprising the amino acid sequence of SEQ ID NO: 240,
a light chain CDR2 comprising the amino acid sequence of SEQ ID NO: 241, and
a light chain CDR3 comprising the amino acid sequence of SEQ ID NO: 242,
(7) a heavy chain CDR1 comprising the amino acid sequence of SEQ ID NO: 249,
a heavy chain CDR2 comprising the amino acid sequence of SEQ ID NO: 250,
a heavy chain CDR3 comprising the amino acid sequence of SEQ ID NO: 251,
a light chain CDR1 comprising the amino acid sequence of SEQ ID NO: 252,
a light chain CDR2 comprising the amino acid sequence of SEQ ID NO: 253, and
a light chain CDR3 comprising the amino acid sequence of SEQ ID NO: 254,
(8) a heavy chain CDR1 comprising the amino acid sequence of SEQ ID NO: 261,
a heavy chain CDR2 comprising the amino acid sequence of SEQ ID NO: 262,
a heavy chain CDR3 comprising the amino acid sequence of SEQ ID NO: 263,
a light chain CDR1 comprising the amino acid sequence of SEQ ID NO: 264, and
a light chain CDR2 comprising the amino acid sequence of SEQ ID NO: 265,
(9) a heavy chain CDR1 comprising the amino acid sequence of SEQ ID NO: 266,
a heavy chain CDR2 comprising the amino acid sequence of SEQ ID NO: 267,
a heavy chain CDR3 comprising the amino acid sequence of SEQ ID NO: 268,
a light chain CDR1 comprising the amino acid sequence of SEQ ID NO: 269,
a light chain CDR2 comprising the amino acid sequence of SEQ ID NO: 270, and
a light chain CDR3 comprising the amino acid sequence of SEQ ID NO: 271,
(10) a heavy chain CDR1 comprising the amino acid sequence of SEQ ID NO: 284,
a heavy chain CDR2 comprising the amino acid sequence of SEQ ID NO: 285,
a heavy chain CDR3 comprising the amino acid sequence of SEQ ID NO: 286,
a light chain CDR1 comprising the amino acid sequence of SEQ ID NO: 287,
a light chain CDR2 comprising the amino acid sequence of SEQ ID NO: 288, and
a light chain CDR3 comprising the amino acid sequence of SEQ ID NO: 289,
(11) a heavy chain CDR1 comprising the amino acid sequence of SEQ ID NO: 296,
a heavy chain CDR2 comprising the amino acid sequence of SEQ ID NO: 297,
a heavy chain CDR3 comprising the amino acid sequence of SEQ ID NO: 298,
a light chain CDR1 comprising the amino acid sequence of SEQ ID NO: 299,
a light chain CDR2 comprising the amino acid sequence of SEQ ID NO: 300, and
a light chain CDR3 comprising the amino acid sequence of SEQ ID NO: 301,
(12) a heavy chain CDR1 comprising the amino acid sequence of SEQ ID NO: 308,
a heavy chain CDR2 comprising the amino acid sequence of SEQ ID NO: 309,
a heavy chain CDR3 comprising the amino acid sequence of SEQ ID NO: 310,
a light chain CDR1 comprising the amino acid sequence of SEQ ID NO: 311,
a light chain CDR2 comprising the amino acid sequence of SEQ ID NO: 312, and
a light chain CDR3 comprising the amino acid sequence of SEQ ID NO: 313,
(13) a heavy chain CDR1 comprising the amino acid sequence of SEQ ID NO: 356,
a heavy chain CDR2 comprising the amino acid sequence of SEQ ID NO: 357,
a heavy chain CDR3 comprising the amino acid sequence of SEQ ID NO: 358,
a light chain CDR1 comprising the amino acid sequence of SEQ ID NO: 359,
a light chain CDR2 comprising the amino acid sequence of SEQ ID NO: 360, and
a light chain CDR3 comprising the amino acid sequence of SEQ ID NO: 361,
(14) a heavy chain CDR1 comprising the amino acid sequence of SEQ ID NO: 368,
a heavy chain CDR2 comprising the amino acid sequence of SEQ ID NO: 369,
a heavy chain CDR3 comprising the amino acid sequence of SEQ ID NO: 370,
a light chain CDR1 comprising the amino acid sequence of SEQ ID NO: 371,
a light chain CDR2 comprising the amino acid sequence of SEQ ID NO: 372, and
a light chain CDR3 comprising the amino acid sequence of SEQ ID NO: 373,
(15) a heavy chain CDR1 comprising the amino acid sequence of SEQ ID NO: 385,
a heavy chain CDR2 comprising the amino acid sequence of SEQ ID NO: 386,
a heavy chain CDR3 comprising the amino acid sequence of SEQ ID NO: 387,
a light chain CDR1 comprising the amino acid sequence of SEQ ID NO: 388,
a light chain CDR2 comprising the amino acid sequence of SEQ ID NO: 389, and
a light chain CDR3 comprising the amino acid sequence of SEQ ID NO: 390,
(16) a heavy chain CDR1 comprising the amino acid sequence of SEQ ID NO: 391,
a heavy chain CDR2 comprising the amino acid sequence of SEQ ID NO: 392,
a heavy chain CDR3 comprising the amino acid sequence of SEQ ID NO: 393,
a light chain CDR1 comprising the amino acid sequence of SEQ ID NO: 394,
a light chain CDR2 comprising the amino acid sequence of SEQ ID NO: 395, and
a light chain CDR3 comprising the amino acid sequence of SEQ ID NO: 396,
(17) a heavy chain CDR1 comprising the amino acid sequence of SEQ ID NO: 403,
a heavy chain CDR2 comprising the amino acid sequence of SEQ ID NO: 404,
a heavy chain CDR3 comprising the amino acid sequence of SEQ ID NO: 405,
a light chain CDR1 comprising the amino acid sequence of SEQ ID NO: 406,
a light chain CDR2 comprising the amino acid sequence of SEQ ID NO: 407, and
a light chain CDR3 comprising the amino acid sequence of SEQ ID NO: 408,
(18) a heavy chain CDR1 comprising the amino acid sequence of SEQ ID NO: 415,
a heavy chain CDR2 comprising the amino acid sequence of SEQ ID NO: 416,
a heavy chain CDR3 comprising the amino acid sequence of SEQ ID NO: 417,
a light chain CDR1 comprising the amino acid sequence of SEQ ID NO: 418,
a light chain CDR2 comprising the amino acid sequence of SEQ ID NO: 419, and
a light chain CDR3 comprising the amino acid sequence of SEQ ID NO: 420,
(19) a heavy chain CDR1 comprising the amino acid sequence of SEQ ID NO: 427,
a heavy chain CDR2 comprising the amino acid sequence of SEQ ID NO: 428,
a heavy chain CDR3 comprising the amino acid sequence of SEQ ID NO: 429,
a light chain CDR1 comprising the amino acid sequence of SEQ ID NO: 430,
a light chain CDR2 comprising the amino acid sequence of SEQ ID NO: 431, and
a light chain CDR3 comprising the amino acid sequence of SEQ ID NO: 432,
(20) a heavy chain CDR1 comprising the amino acid sequence of SEQ ID NO: 433,
a heavy chain CDR2 comprising the amino acid sequence of SEQ ID NO: 434,
a heavy chain CDR3 comprising the amino acid sequence of SEQ ID NO: 435,
a light chain CDR1 comprising the amino acid sequence of SEQ ID NO: 436,
a light chain CDR2 comprising the amino acid sequence of SEQ ID NO: 437, and
a light chain CDR3 comprising the amino acid sequence of SEQ ID NO: 438,
(21) a heavy chain CDR1 comprising the amino acid sequence of SEQ ID NO: 439,
a heavy chain CDR2 comprising the amino acid sequence of SEQ ID NO: 440,
a heavy chain CDR3 comprising the amino acid sequence of SEQ ID NO: 441,
a light chain CDR1 comprising the amino acid sequence of SEQ ID NO: 442,
a light chain CDR2 comprising the amino acid sequence of SEQ ID NO: 443, and
a light chain CDR3 comprising the amino acid sequence of SEQ ID NO: 444,
(22) a heavy chain CDR1 comprising the amino acid sequence of SEQ ID NO: 445,
a heavy chain CDR2 comprising the amino acid sequence of SEQ ID NO: 446,
a heavy chain CDR3 comprising the amino acid sequence of SEQ ID NO: 447,
a light chain CDR1 comprising the amino acid sequence of SEQ ID NO: 448,
a light chain CDR2 comprising the amino acid sequence of SEQ ID NO: 449, and
a light chain CDR3 comprising the amino acid sequence of SEQ ID NO: 450,
(23) a heavy chain CDR1 comprising the amino acid sequence of SEQ ID NO: 451,
a heavy chain CDR2 comprising the amino acid sequence of SEQ ID NO: 452,
a heavy chain CDR3 comprising the amino acid sequence of SEQ ID NO: 453,
a light chain CDR1 comprising the amino acid sequence of SEQ ID NO: 454,
a light chain CDR2 comprising the amino acid sequence of SEQ ID NO: 455, and
a light chain CDR3 comprising the amino acid sequence of SEQ ID NO: 456,
(24) a heavy chain CDR1 comprising the amino acid sequence of SEQ ID NO: 457,
a heavy chain CDR2 comprising the amino acid sequence of SEQ ID NO: 458,
a heavy chain CDR3 comprising the amino acid sequence of SEQ ID NO: 459,
a light chain CDR1 comprising the amino acid sequence of SEQ ID NO: 460,
a light chain CDR2 comprising the amino acid sequence of SEQ ID NO: 461, and
a light chain CDR3 comprising the amino acid sequence of SEQ ID NO: 462,
(25) a heavy chain CDR1 comprising the amino acid sequence of SEQ ID NO: 463,
a heavy chain CDR2 comprising the amino acid sequence of SEQ ID NO: 464,
a heavy chain CDR3 comprising the amino acid sequence of SEQ ID NO: 465,
a light chain CDR1 comprising the amino acid sequence of SEQ ID NO: 466,
a light chain CDR2 comprising the amino acid sequence of SEQ ID NO: 467, and
a light chain CDR3 comprising the amino acid sequence of SEQ ID NO: 468,
(26) a heavy chain CDR1 comprising the amino acid sequence of SEQ ID NO: 469,
a heavy chain CDR2 comprising the amino acid sequence of SEQ ID NO: 470,
a heavy chain CDR3 comprising the amino acid sequence of SEQ ID NO: 471,
a light chain CDR1 comprising the amino acid sequence of SEQ ID NO: 472,
a light chain CDR2 comprising the amino acid sequence of SEQ ID NO: 473, and
a light chain CDR3 comprising the amino acid sequence of SEQ ID NO: 474,
(27) a heavy chain CDR1 comprising the amino acid sequence of SEQ ID NO: 475,
a heavy chain CDR2 comprising the amino acid sequence of SEQ ID NO: 476,
a heavy chain CDR3 comprising the amino acid sequence of SEQ ID NO: 477,
a light chain CDR1 comprising the amino acid sequence of SEQ ID NO: 478,
a light chain CDR2 comprising the amino acid sequence of SEQ ID NO: 479, and
a light chain CDR3 comprising the amino acid sequence of SEQ ID NO: 480,
(28) a heavy chain CDR1 comprising the amino acid sequence of SEQ ID NO: 481,
a heavy chain CDR2 comprising the amino acid sequence of SEQ ID NO: 482,
a heavy chain CDR3 comprising the amino acid sequence of SEQ ID NO: 483,
a light chain CDR1 comprising the amino acid sequence of SEQ ID NO: 484,
a light chain CDR2 comprising the amino acid sequence of SEQ ID NO: 485, and
a light chain CDR3 comprising the amino acid sequence of SEQ ID NO: 486,
(29) a heavy chain CDR1 comprising the amino acid sequence of SEQ ID NO: 487,
a heavy chain CDR2 comprising the amino acid sequence of SEQ ID NO: 488,
a heavy chain CDR3 comprising the amino acid sequence of SEQ ID NO: 489,
a light chain CDR1 comprising the amino acid sequence of SEQ ID NO: 490,
a light chain CDR2 comprising the amino acid sequence of SEQ ID NO: 491, and
a light chain CDR3 comprising the amino acid sequence of SEQ ID NO: 492,
(30) a heavy chain CDR1 comprising the amino acid sequence of SEQ ID NO: 499,
a heavy chain CDR2 comprising the amino acid sequence of SEQ ID NO: 500,
a heavy chain CDR3 comprising the amino acid sequence of SEQ ID NO: 501,
a light chain CDR1 comprising the amino acid sequence of SEQ ID NO: 502,
a light chain CDR2 comprising the amino acid sequence of SEQ ID NO: 503, and
a light chain CDR3 comprising the amino acid sequence of SEQ ID NO: 504,
(31) a heavy chain CDR1 comprising the amino acid sequence of SEQ ID NO: 511,
a heavy chain CDR2 comprising the amino acid sequence of SEQ ID NO: 512,
a heavy chain CDR3 comprising the amino acid sequence of SEQ ID NO: 513,
a light chain CDR1 comprising the amino acid sequence of SEQ ID NO: 514,
a light chain CDR2 comprising the amino acid sequence of SEQ ID NO: 515, and
a light chain CDR3 comprising the amino acid sequence of SEQ ID NO: 516,
(32) a heavy chain CDR1 comprising the amino acid sequence of SEQ ID NO: 517,
a heavy chain CDR2 comprising the amino acid sequence of SEQ ID NO: 518,
a heavy chain CDR3 comprising the amino acid sequence of SEQ ID NO: 519,
a light chain CDR1 comprising the amino acid sequence of SEQ ID NO: 520,
a light chain CDR2 comprising the amino acid sequence of SEQ ID NO: 521, and
a light chain CDR3 comprising the amino acid sequence of SEQ ID NO: 522, or
(33) a heavy chain CDR1 comprising the amino acid sequence of SEQ ID NO: 523,
a heavy chain CDR2 comprising the amino acid sequence of SEQ ID NO: 524,
a heavy chain CDR3 comprising the amino acid sequence of SEQ ID NO: 525,
a light chain CDR1 comprising the amino acid sequence of SEQ ID NO: 526,
a light chain CDR2 comprising the amino acid sequence of SEQ ID NO: 527, and
a light chain CDR3 comprising the amino acid sequence of SEQ ID NO: 528.

12. The ROR antigen binding molecule of claim 9, wherein the second antigen binding site comprises:
(1) a heavy chain CDR1 comprising the amino acid sequence of SEQ ID NO: 314,
a heavy chain CDR2 comprising the amino acid sequence of SEQ ID NO: 315,
a heavy chain CDR3 comprising the amino acid sequence of SEQ ID NO: 316,
a light chain CDR1 comprising the amino acid sequence of SEQ ID NO: 317,
a light chain CDR2 comprising the amino acid sequence of SEQ ID NO: 318, and
a light chain CDR3 comprising the amino acid sequence of SEQ ID NO: 319,
(2) a heavy chain CDR1 comprising the amino acid sequence of SEQ ID NO: 165.
a heavy chain CDR2 comprising the amino acid sequence of SEQ ID NO: 166,
a heavy chain CDR3 comprising the amino acid sequence of SEQ ID NO: 167,
a light chain CDR1 comprising the amino acid sequence of SEQ ID NO: 168,
a light chain CDR2 comprising the amino acid sequence of SEQ ID NO: 169, and
a light chain CDR3 comprising the amino acid sequence of SEQ ID NO: 170,
(3) a heavy chain CDR1 comprising the amino acid sequence of SEQ ID NO: 171.
a heavy chain CDR2 comprising the amino acid sequence of SEQ ID NO: 172,
a heavy chain CDR3 comprising the amino acid sequence of SEQ ID NO: 173,
a light chain CDR1 comprising the amino acid sequence of SEQ ID NO: 174,
a light chain CDR2 comprising the amino acid sequence of SEQ ID NO: 175, and
a light chain CDR3 comprising the amino acid sequence of SEQ ID NO: 176,
(4′) a heavy chain CDR1 comprising the amino acid sequence of SEQ ID NO: 183.
a heavy chain CDR2 comprising the amino acid sequence of SEQ ID NO: 184,
a heavy chain CDR3 comprising the amino acid sequence of SEQ ID NO: 185,
a light chain CDR1 comprising the amino acid sequence of SEQ ID NO: 186,
a light chain CDR2 comprising the amino acid sequence of SEQ ID NO: 187, and
a light chain CDR3 comprising the amino acid sequence of SEQ ID NO: 188;
(5) a heavy chain CDR1 comprising the amino acid sequence of SEQ ID NO: 195,
a heavy chain CDR2 comprising the amino acid sequence of SEQ ID NO: 196,
a heavy chain CDR3 comprising the amino acid sequence of SEQ ID NO: 197,
a light chain CDR1 comprising the amino acid sequence of SEQ ID NO: 198,
a light chain CDR2 comprising the amino acid sequence of SEQ ID NO: 199, and
a light chain CDR3 comprising the amino acid sequence of SEQ ID NO: 200;
(6) a heavy chain CDR1 comprising the amino acid sequence of SEQ ID NO: 225,
a heavy chain CDR2 comprising the amino acid sequence of SEQ ID NO: 226,
a heavy chain CDR3 comprising the amino acid sequence of SEQ ID NO: 227,
a light chain CDR1 comprising the amino acid sequence of SEQ ID NO: 228,
a light chain CDR2 comprising the amino acid sequence of SEQ ID NO: 229, and
a light chain CDR3 comprising the amino acid sequence of SEQ ID NO: 230;
(7) a heavy chain CDR1 comprising the amino acid sequence of SEQ ID NO: 272,
a heavy chain CDR2 comprising the amino acid sequence of SEQ ID NO: 273,
a heavy chain CDR3 comprising the amino acid sequence of SEQ ID NO: 274,
a light chain CDR1 comprising the amino acid sequence of SEQ ID NO: 275,
a light chain CDR2 comprising the amino acid sequence of SEQ ID NO: 276, and
a light chain CDR3 comprising the amino acid sequence of SEQ ID NO: 277;
(8) a heavy chain CDR1 comprising the amino acid sequence of SEQ ID NO: 302,
a heavy chain CDR2 comprising the amino acid sequence of SEQ ID NO: 303,
a heavy chain CDR3 comprising the amino acid sequence of SEQ ID NO: 304,
a light chain CDR1 comprising the amino acid sequence of SEQ ID NO: 305,
a light chain CDR2 comprising the amino acid sequence of SEQ ID NO: 306, and
a light chain CDR3 comprising the amino acid sequence of SEQ ID NO: 307;
(9) a heavy chain CDR1 comprising the amino acid sequence of SEQ ID NO: 308,
a heavy chain CDR2 comprising the amino acid sequence of SEQ ID NO: 309,
a heavy chain CDR3 comprising the amino acid sequence of SEQ ID NO: 310,
a light chain CDR1 comprising the amino acid sequence of SEQ ID NO: 311,
a light chain CDR2 comprising the amino acid sequence of SEQ ID NO: 312, and
a light chain CDR3 comprising the amino acid sequence of SEQ ID NO: 313:
(10) a heavy chain CDR1 comprising the amino acid sequence of SEQ ID NO: 332,
a heavy chain CDR2 comprising the amino acid sequence of SEQ ID NO: 333,
a heavy chain CDR3 comprising the amino acid sequence of SEQ ID NO: 334,
a light chain CDR1 comprising the amino acid sequence of SEQ ID NO: 335,
a light chain CDR2 comprising the amino acid sequence of SEQ ID NO: 336, and
a light chain CDR3 comprising the amino acid sequence of SEQ ID NO: 337:
(11) a heavy chain CDR1 comprising the amino acid sequence of SEQ ID NO: 344,
a heavy chain CDR2 comprising the amino acid sequence of SEQ ID NO: 345,
a heavy chain CDR3 comprising the amino acid sequence of SEQ ID NO: 346,
a light chain CDR1 comprising the amino acid sequence of SEQ ID NO: 347,
a light chain CDR2 comprising the amino acid sequence of SEQ ID NO: 348, and
a light chain CDR3 comprising the amino acid sequence of SEQ ID NO: 349:
(12) a heavy chain CDR1 comprising the amino acid sequence of SEQ ID NO: 350,
a heavy chain CDR2 comprising the amino acid sequence of SEQ ID NO: 351,
a heavy chain CDR3 comprising the amino acid sequence of SEQ ID NO: 352,
a light chain CDR1 comprising the amino acid sequence of SEQ ID NO: 353,
a light chain CDR2 comprising the amino acid sequence of SEQ ID NO: 354, and
a light chain CDR3 comprising the amino acid sequence of SEQ ID NO: 355: or (13) a heavy chain CDR1 comprising the amino acid sequence of SEQ ID NO: 374,
a heavy chain CDR2 comprising the amino acid sequence of SEQ ID NO: 375,
a heavy chain CDR3 comprising the amino acid sequence of SEQ ID NO: 376,
a light chain CDR1 comprising the amino acid sequence of SEQ ID NO: 377,
a light chain CDR2 comprising the amino acid sequence of SEQ ID NO: 378, and
a light chain CDR3 comprising the amino acid sequence of SEQ ID NO: 379.

13. The ROR antigen binding molecule of claim 9, wherein the second antigen binding site comprises:
(1) a heavy chain CDR1 comprising the amino acid sequence of SEQ ID NO: 505,
a heavy chain CDR2 comprising the amino acid sequence of SEQ ID NO: 506,
a heavy chain CDR3 comprising the amino acid sequence of SEQ ID NO: 507,
a light chain CDR1 comprising the amino acid sequence of SEQ ID NO: 508,
a light chain CDR2 comprising the amino acid sequence of SEQ ID NO: 509, and
a light chain CDR3 comprising the amino acid sequence of SEQ ID NO: 510;
(2) a heavy chain CDR1 comprising the amino acid sequence of SEQ ID NO: 397,
a heavy chain CDR2 comprising the amino acid sequence of SEQ ID NO: 398,
a heavy chain CDR3 comprising the amino acid sequence of SEQ ID NO: 399,
a light chain CDR1 comprising the amino acid sequence of SEQ ID NO: 400,
a light chain CDR2 comprising the amino acid sequence of SEQ ID NO: 401, and
a light chain CDR3 comprising the amino acid sequence of SEQ ID NO: 402;
(3) a heavy chain CDR1 comprising the amino acid sequence of SEQ ID NO: 409,
a heavy chain CDR2 comprising the amino acid sequence of SEQ ID NO: 410,
a heavy chain CDR3 comprising the amino acid sequence of SEQ ID NO: 411,
a light chain CDR1 comprising the amino acid sequence of SEQ ID NO: 412,
a light chain CDR2 comprising the amino acid sequence of SEQ ID NO: 413, and
a light chain CDR3 comprising the amino acid sequence of SEQ ID NO: 414;
(4) a heavy chain CDR1 comprising the amino acid sequence of SEQ ID NO: 421,
a heavy chain CDR2 comprising the amino acid sequence of SEQ ID NO: 422,
a heavy chain CDR3 comprising the amino acid sequence of SEQ ID NO: 423,
a light chain CDR1 comprising the amino acid sequence of SEQ ID NO: 424,
a light chain CDR2 comprising the amino acid sequence of SEQ ID NO: 425, and
a light chain CDR3 comprising the amino acid sequence of SEQ ID NO: 426;
(5) a heavy chain CDR1 comprising the amino acid sequence of SEQ ID NO: 493,
a heavy chain CDR2 comprising the amino acid sequence of SEQ ID NO: 494,
a heavy chain CDR3 comprising the amino acid sequence of SEQ ID NO: 495,
a light chain CDR1 comprising the amino acid sequence of SEQ ID NO: 496,
a light chain CDR2 comprising the amino acid sequence of SEQ ID NO: 497, and
a light chain CDR3 comprising the amino acid sequence of SEQ ID NO: 498;
(6) a heavy chain CDR1 comprising the amino acid sequence of SEQ ID NO: 529,
a heavy chain CDR2 comprising the amino acid sequence of SEQ ID NO: 530,
a heavy chain CDR3 comprising the amino acid sequence of SEQ ID NO: 531,
a light chain CDR1 comprising the amino acid sequence of SEQ ID NO: 532,
a light chain CDR2 comprising the amino acid sequence of SEQ ID NO: 533, and
a light chain CDR3 comprising the amino acid sequence of SEQ ID NO: 534; or
(7) a heavy chain CDR1 comprising the amino acid sequence of SEQ ID NO: 535,
a heavy chain CDR2 comprising the amino acid sequence of SEQ ID NO: 536,
a heavy chain CDR3 comprising the amino acid sequence of SEQ ID NO: 537,
a light chain CDR1 comprising the amino acid sequence of SEQ ID NO: 538,
a light chain CDR2 comprising the amino acid sequence of SEQ ID NO: 539, and
a light chain CDR3 comprising the amino acid sequence of SEQ ID NO: 540.

14. The ROR antigen binding molecule of claim 9, wherein the second antigen binding site is specific for a second antigen different from the ROR antigen.

15. The ROR antigen binding molecule of claim 14, wherein the second antigen is a CD3 antigen.

16-18. (canceled)

19. The ROR antigen binding molecule of claim 2, wherein the ROR antigen binding molecule comprises an antibody format selected from the group consisting of: full-length antibodies, Fab fragments, Fvs, scFvs, tandem scFvs, Diabodies, scDiabodies, DARTs, tandAbs, and minibodies.

20. The ROR antigen binding molecule of claim 2, wherein the molecule comprises a sequence of one or more constant regions.

21. The ROR antigen binding molecule of claim 20, wherein the constant region is a CH1, CH2, CH3 and/or CL constant region.

22-65. (canceled)

66. The ROR antigen binding molecule of claim 2, wherein the ROR antigen binding molecule further comprises a third antigen binding site.

67. The ROR antigen binding molecule of claim 66, wherein the third antigen binding site is specific for ROR1 and ROR2, ROR1, or ROR2.

68. The ROR antigen binding molecule of claim 67, wherein the third antigen binding site is the same as the first antigen binding site.

69. The ROR antigen binding molecule of claim 67, wherein the third antigen binding site comprises:
(1) a heavy chain CDR1 comprising the amino acid sequence of SEQ ID NO: 213,
a heavy chain CDR2 comprising the amino acid sequence of SEQ ID NO: 214,
a heavy chain CDR3 comprising the amino acid sequence of SEQ ID NO: 215,
a light chain CDR1 comprising the amino acid sequence of SEQ ID NO: 216,
a light chain CDR2 comprising the amino acid sequence of SEQ ID NO: 217, and
a light chain CDR3 comprising the amino acid sequence of SEQ ID NO: 218,
(2) a heavy chain CDR1 comprising the amino acid sequence of SEQ ID NO: 207,
a heavy chain CDR2 comprising the amino acid sequence of SEQ ID NO: 208,
a heavy chain CDR3 comprising the amino acid sequence of SEQ ID NO: 209 a light chain CDR1 comprising the amino acid sequence of SEQ ID NO: 210,
a light chain CDR2 comprising the amino acid sequence of SEQ ID NO: 211, and
a light chain CDR3 comprising the amino acid sequence of SEQ ID NO: 212;
(3) a heavy chain CDR1 comprising the amino acid sequence of SEQ ID NO: 153,
a heavy chain CDR2 comprising the amino acid sequence of SEQ ID NO: 154,
a heavy chain CDR3 comprising the amino acid sequence of SEQ ID NO: 155,
a light chain CDR1 comprising the amino acid sequence of SEQ ID NO: 156,
a light chain CDR2 comprising the amino acid sequence of SEQ ID NO: 157, and
a light chain CDR3 comprising the amino acid sequence of SEQ ID NO: 158,
(4) a heavy chain CDR1 comprising the amino acid sequence of SEQ ID NO: 219,
a heavy chain CDR2 comprising the amino acid sequence of SEQ ID NO: 220,
a heavy chain CDR3 comprising the amino acid sequence of SEQ ID NO: 221,
a light chain CDR1 comprising the amino acid sequence of SEQ ID NO: 222,
a light chain CDR2 comprising the amino acid sequence of SEQ ID NO: 223, and
a light chain CDR3 comprising the amino acid sequence of SEQ ID NO: 224,
(5) a heavy chain CDR1 comprising the amino acid sequence of SEQ ID NO: 231,
a heavy chain CDR2 comprising the amino acid sequence of SEQ ID NO: 232,
a heavy chain CDR3 comprising the amino acid sequence of SEQ ID NO: 233,
a light chain CDR1 comprising the amino acid sequence of SEQ ID NO: 234,
a light chain CDR2 comprising the amino acid sequence of SEQ ID NO: 235, and
a light chain CDR3 comprising the amino acid sequence of SEQ ID NO: 236,
(6) a heavy chain CDR1 comprising the amino acid sequence of SEQ ID NO: 237,
a heavy chain CDR2 comprising the amino acid sequence of SEQ ID NO: 238,
a heavy chain CDR3 comprising the amino acid sequence of SEQ ID NO: 239,
a light chain CDR1 comprising the amino acid sequence of SEQ ID NO: 240,
a light chain CDR2 comprising the amino acid sequence of SEQ ID NO: 241, and
a light chain CDR3 comprising the amino acid sequence of SEQ ID NO: 242,
(7) a heavy chain CDR1 comprising the amino acid sequence of SEQ ID NO: 249,
a heavy chain CDR2 comprising the amino acid sequence of SEQ ID NO: 250,
a heavy chain CDR3 comprising the amino acid sequence of SEQ ID NO: 251,
a light chain CDR1 comprising the amino acid sequence of SEQ ID NO: 252,
a light chain CDR2 comprising the amino acid sequence of SEQ ID NO: 253, and
a light chain CDR3 comprising the amino acid sequence of SEQ ID NO: 254,
(8) a heavy chain CDR1 comprising the amino acid sequence of SEQ ID NO: 261,
a heavy chain CDR2 comprising the amino acid sequence of SEQ ID NO: 262,
a heavy chain CDR3 comprising the amino acid sequence of SEQ ID NO: 263,
a light chain CDR1 comprising the amino acid sequence of SEQ ID NO: 264, and
a light chain CDR2 comprising the amino acid sequence of SEQ ID NO: 265,
(9) a heavy chain CDR1 comprising the amino acid sequence of SEQ ID NO: 266,
a heavy chain CDR2 comprising the amino acid sequence of SEQ ID NO: 267,
a heavy chain CDR3 comprising the amino acid sequence of SEQ ID NO: 268,
a light chain CDR1 comprising the amino acid sequence of SEQ ID NO: 269,
a light chain CDR2 comprising the amino acid sequence of SEQ ID NO: 270, and
a light chain CDR3 comprising the amino acid sequence of SEQ ID NO: 271,
(10) a heavy chain CDR1 comprising the amino acid sequence of SEQ ID NO: 284,
a heavy chain CDR2 comprising the amino acid sequence of SEQ ID NO: 285,
a heavy chain CDR3 comprising the amino acid sequence of SEQ ID NO: 286,
a light chain CDR1 comprising the amino acid sequence of SEQ ID NO: 287,
a light chain CDR2 comprising the amino acid sequence of SEQ ID NO: 288, and
a light chain CDR3 comprising the amino acid sequence of SEQ ID NO: 289,
(11) a heavy chain CDR1 comprising the amino acid sequence of SEQ ID NO: 296,
a heavy chain CDR2 comprising the amino acid sequence of SEQ ID NO: 297,
a heavy chain CDR3 comprising the amino acid sequence of SEQ ID NO: 298,
a light chain CDR1 comprising the amino acid sequence of SEQ ID NO: 299,
a light chain CDR2 comprising the amino acid sequence of SEQ ID NO: 300, and
a light chain CDR3 comprising the amino acid sequence of SEQ ID NO: 301,
(12) a heavy chain CDR1 comprising the amino acid sequence of SEQ ID NO: 308,
a heavy chain CDR2 comprising the amino acid sequence of SEQ ID NO: 309,
a heavy chain CDR3 comprising the amino acid sequence of SEQ ID NO: 310,
a light chain CDR1 comprising the amino acid sequence of SEQ ID NO: 311,
a light chain CDR2 comprising the amino acid sequence of SEQ ID NO: 312, and
a light chain CDR3 comprising the amino acid sequence of SEQ ID NO: 313,
(13) a heavy chain CDR1 comprising the amino acid sequence of SEQ ID NO: 356,
a heavy chain CDR2 comprising the amino acid sequence of SEQ ID NO: 357,
a heavy chain CDR3 comprising the amino acid sequence of SEQ ID NO: 358,
a light chain CDR1 comprising the amino acid sequence of SEQ ID NO: 359,
a light chain CDR2 comprising the amino acid sequence of SEQ ID NO: 360, and
a light chain CDR3 comprising the amino acid sequence of SEQ ID NO: 361,
(14) a heavy chain CDR1 comprising the amino acid sequence of SEQ ID NO: 368,
a heavy chain CDR2 comprising the amino acid sequence of SEQ ID NO: 369,
a heavy chain CDR3 comprising the amino acid sequence of SEQ ID NO: 370,
a light chain CDR1 comprising the amino acid sequence of SEQ ID NO: 371,
a light chain CDR2 comprising the amino acid sequence of SEQ ID NO: 372, and
a light chain CDR3 comprising the amino acid sequence of SEQ ID NO: 373,
(15) a heavy chain CDR1 comprising the amino acid sequence of SEQ ID NO: 385,
a heavy chain CDR2 comprising the amino acid sequence of SEQ ID NO: 386,
a heavy chain CDR3 comprising the amino acid sequence of SEQ ID NO: 387,
a light chain CDR1 comprising the amino acid sequence of SEQ ID NO: 388,
a light chain CDR2 comprising the amino acid sequence of SEQ ID NO: 389, and
a light chain CDR3 comprising the amino acid sequence of SEQ ID NO: 390,
(16) a heavy chain CDR1 comprising the amino acid sequence of SEQ ID NO: 391,
a heavy chain CDR2 comprising the amino acid sequence of SEQ ID NO: 392,
a heavy chain CDR3 comprising the amino acid sequence of SEQ ID NO: 393,
a light chain CDR1 comprising the amino acid sequence of SEQ ID NO: 394,
a light chain CDR2 comprising the amino acid sequence of SEQ ID NO: 395, and
a light chain CDR3 comprising the amino acid sequence of SEQ ID NO: 396,
(17) a heavy chain CDR1 comprising the amino acid sequence of SEQ ID NO: 403,
a heavy chain CDR2 comprising the amino acid sequence of SEQ ID NO: 404,
a heavy chain CDR3 comprising the amino acid sequence of SEQ ID NO: 405,
a light chain CDR1 comprising the amino acid sequence of SEQ ID NO: 406,
a light chain CDR2 comprising the amino acid sequence of SEQ ID NO: 407, and
a light chain CDR3 comprising the amino acid sequence of SEQ ID NO: 408,
(18) a heavy chain CDR1 comprising the amino acid sequence of SEQ ID NO: 415,
a heavy chain CDR2 comprising the amino acid sequence of SEQ ID NO: 416,
a heavy chain CDR3 comprising the amino acid sequence of SEQ ID NO: 417,
a light chain CDR1 comprising the amino acid sequence of SEQ ID NO: 418,
a light chain CDR2 comprising the amino acid sequence of SEQ ID NO: 419, and
a light chain CDR3 comprising the amino acid sequence of SEQ ID NO: 420,
(19) a heavy chain CDR1 comprising the amino acid sequence of SEQ ID NO: 427,
a heavy chain CDR2 comprising the amino acid sequence of SEQ ID NO: 428,
a heavy chain CDR3 comprising the amino acid sequence of SEQ ID NO: 429,
a light chain CDR1 comprising the amino acid sequence of SEQ ID NO: 430,
a light chain CDR2 comprising the amino acid sequence of SEQ ID NO: 431, and
a light chain CDR3 comprising the amino acid sequence of SEQ ID NO: 432,
(20) a heavy chain CDR1 comprising the amino acid sequence of SEQ ID NO: 433,
a heavy chain CDR2 comprising the amino acid sequence of SEQ ID NO: 434,
a heavy chain CDR3 comprising the amino acid sequence of SEQ ID NO: 435,
a light chain CDR1 comprising the amino acid sequence of SEQ ID NO: 436,
a light chain CDR2 comprising the amino acid sequence of SEQ ID NO: 437, and
a light chain CDR3 comprising the amino acid sequence of SEQ ID NO: 438,
(21) a heavy chain CDR1 comprising the amino acid sequence of SEQ ID NO: 439,
a heavy chain CDR2 comprising the amino acid sequence of SEQ ID NO: 440,
a heavy chain CDR3 comprising the amino acid sequence of SEQ ID NO: 441,
a light chain CDR1 comprising the amino acid sequence of SEQ ID NO: 442,
a light chain CDR2 comprising the amino acid sequence of SEQ ID NO: 443, and
a light chain CDR3 comprising the amino acid sequence of SEQ ID NO: 444,
(22) a heavy chain CDR1 comprising the amino acid sequence of SEQ ID NO: 445,
a heavy chain CDR2 comprising the amino acid sequence of SEQ ID NO: 446,
a heavy chain CDR3 comprising the amino acid sequence of SEQ ID NO: 447,
a light chain CDR1 comprising the amino acid sequence of SEQ ID NO: 448,
a light chain CDR2 comprising the amino acid sequence of SEQ ID NO: 449, and
a light chain CDR3 comprising the amino acid sequence of SEQ ID NO: 450,
(23) a heavy chain CDR1 comprising the amino acid sequence of SEQ ID NO: 451,
a heavy chain CDR2 comprising the amino acid sequence of SEQ ID NO: 452,
a heavy chain CDR3 comprising the amino acid sequence of SEQ ID NO: 453,
a light chain CDR1 comprising the amino acid sequence of SEQ ID NO: 454,
a light chain CDR2 comprising the amino acid sequence of SEQ ID NO: 455, and
a light chain CDR3 comprising the amino acid sequence of SEQ ID NO: 456,
(24) a heavy chain CDR1 comprising the amino acid sequence of SEQ ID NO: 457,
a heavy chain CDR2 comprising the amino acid sequence of SEQ ID NO: 458,
a heavy chain CDR3 comprising the amino acid sequence of SEQ ID NO: 459,
a light chain CDR1 comprising the amino acid sequence of SEQ ID NO: 460,
a light chain CDR2 comprising the amino acid sequence of SEQ ID NO: 461, and
a light chain CDR3 comprising the amino acid sequence of SEQ ID NO: 462,
(25) a heavy chain CDR1 comprising the amino acid sequence of SEQ ID NO: 463,
a heavy chain CDR2 comprising the amino acid sequence of SEQ ID NO: 464,
a heavy chain CDR3 comprising the amino acid sequence of SEQ ID NO: 465,
a light chain CDR1 comprising the amino acid sequence of SEQ ID NO: 466,
a light chain CDR2 comprising the amino acid sequence of SEQ ID NO: 467, and
a light chain CDR3 comprising the amino acid sequence of SEQ ID NO: 468,
(26) a heavy chain CDR1 comprising the amino acid sequence of SEQ ID NO: 469,
a heavy chain CDR2 comprising the amino acid sequence of SEQ ID NO: 470,
a heavy chain CDR3 comprising the amino acid sequence of SEQ ID NO: 471,
a light chain CDR1 comprising the amino acid sequence of SEQ ID NO: 472,
a light chain CDR2 comprising the amino acid sequence of SEQ ID NO: 473, and
a light chain CDR3 comprising the amino acid sequence of SEQ ID NO: 474,
(27) a heavy chain CDR1 comprising the amino acid sequence of SEQ ID NO: 475,
a heavy chain CDR2 comprising the amino acid sequence of SEQ ID NO: 476,
a heavy chain CDR3 comprising the amino acid sequence of SEQ ID NO: 477,
a light chain CDR1 comprising the amino acid sequence of SEQ ID NO: 478,
a light chain CDR2 comprising the amino acid sequence of SEQ ID NO: 479, and
a light chain CDR3 comprising the amino acid sequence of SEQ ID NO: 480,
(28) a heavy chain CDR1 comprising the amino acid sequence of SEQ ID NO: 481,
a heavy chain CDR2 comprising the amino acid sequence of SEQ ID NO: 482,
a heavy chain CDR3 comprising the amino acid sequence of SEQ ID NO: 483,
a light chain CDR1 comprising the amino acid sequence of SEQ ID NO: 484,
a light chain CDR2 comprising the amino acid sequence of SEQ ID NO: 485, and
a light chain CDR3 comprising the amino acid sequence of SEQ ID NO: 486,
(29) a heavy chain CDR1 comprising the amino acid sequence of SEQ ID NO: 487,
a heavy chain CDR2 comprising the amino acid sequence of SEQ ID NO: 488,
a heavy chain CDR3 comprising the amino acid sequence of SEQ ID NO: 489,
a light chain CDR1 comprising the amino acid sequence of SEQ ID NO: 490,
a light chain CDR2 comprising the amino acid sequence of SEQ ID NO: 491, and
a light chain CDR3 comprising the amino acid sequence of SEQ ID NO: 492,
(30) a heavy chain CDR1 comprising the amino acid sequence of SEQ ID NO: 499,
a heavy chain CDR2 comprising the amino acid sequence of SEQ ID NO: 500,
a heavy chain CDR3 comprising the amino acid sequence of SEQ ID NO: 501,
a light chain CDR1 comprising the amino acid sequence of SEQ ID NO: 502,
a light chain CDR2 comprising the amino acid sequence of SEQ ID NO: 503, and
a light chain CDR3 comprising the amino acid sequence of SEQ ID NO: 504,
(31) a heavy chain CDR1 comprising the amino acid sequence of SEQ ID NO: 511,
a heavy chain CDR2 comprising the amino acid sequence of SEQ ID NO: 512,
a heavy chain CDR3 comprising the amino acid sequence of SEQ ID NO: 513,
a light chain CDR1 comprising the amino acid sequence of SEQ ID NO: 514,
a light chain CDR2 comprising the amino acid sequence of SEQ ID NO: 515, and
a light chain CDR3 comprising the amino acid sequence of SEQ ID NO: 516,
(32) a heavy chain CDR1 comprising the amino acid sequence of SEQ ID NO: 517,
a heavy chain CDR2 comprising the amino acid sequence of SEQ ID NO: 518,
a heavy chain CDR3 comprising the amino acid sequence of SEQ ID NO: 519,
a light chain CDR1 comprising the amino acid sequence of SEQ ID NO: 520,
a light chain CDR2 comprising the amino acid sequence of SEQ ID NO: 521, and
a light chain CDR3 comprising the amino acid sequence of SEQ ID NO: 522, or
(33) a heavy chain CDR1 comprising the amino acid sequence of SEQ ID NO: 523,
a heavy chain CDR2 comprising the amino acid sequence of SEQ ID NO: 524,
a heavy chain CDR3 comprising the amino acid sequence of SEQ ID NO: 525,
a light chain CDR1 comprising the amino acid sequence of SEQ ID NO: 526,
a light chain CDR2 comprising the amino acid sequence of SEQ ID NO: 527, and
a light chain CDR3 comprising the amino acid sequence of SEQ ID NO: 528.

70. The ROR antigen binding molecule of claim 67, wherein the third antigen binding site comprises:
(1) a heavy chain CDR1 comprising the amino acid sequence of SEQ ID NO: 314,
a heavy chain CDR2 comprising the amino acid sequence of SEQ ID NO: 315,
a heavy chain CDR3 comprising the amino acid sequence of SEQ ID NO: 316,
a light chain CDR1 comprising the amino acid sequence of SEQ ID NO: 317,
a light chain CDR2 comprising the amino acid sequence of SEQ ID NO: 318, and
a light chain CDR3 comprising the amino acid sequence of SEQ ID NO: 319,
(2) a heavy chain CDR1 comprising the amino acid sequence of SEQ ID NO: 165.
a heavy chain CDR2 comprising the amino acid sequence of SEQ ID NO: 166,
a heavy chain CDR3 comprising the amino acid sequence of SEQ ID NO: 167,
a light chain CDR1 comprising the amino acid sequence of SEQ ID NO: 168,
a light chain CDR2 comprising the amino acid sequence of SEQ ID NO: 169, and
a light chain CDR3 comprising the amino acid sequence of SEQ ID NO: 170,
(3) a heavy chain CDR1 comprising the amino acid sequence of SEQ ID NO: 171.
a heavy chain CDR2 comprising the amino acid sequence of SEQ ID NO: 172,
a heavy chain CDR3 comprising the amino acid sequence of SEQ ID NO: 173,
a light chain CDR1 comprising the amino acid sequence of SEQ ID NO: 174,
a light chain CDR2 comprising the amino acid sequence of SEQ ID NO: 175, and
a light chain CDR3 comprising the amino acid sequence of SEQ ID NO: 176,
(4) a heavy chain CDR1 comprising the amino acid sequence of SEQ ID NO: 183
a heavy chain CDR2 comprising the amino acid sequence of SEQ ID NO: 184,
a heavy chain CDR3 comprising the amino acid sequence of SEQ ID NO: 185,
a light chain CDR1 comprising the amino acid sequence of SEQ ID NO: 186,
a light chain CDR2 comprising the amino acid sequence of SEQ ID NO: 187, and
a light chain CDR3 comprising the amino acid sequence of SEQ ID NO: 188,
(5) a heavy chain CDR1 comprising the amino acid sequence of SEQ ID NO: 195,
a heavy chain CDR2 comprising the amino acid sequence of SEQ ID NO: 196,
a heavy chain CDR3 comprising the amino acid sequence of SEQ ID NO: 197,
a light chain CDR1 comprising the amino acid sequence of SEQ ID NO: 198,
a light chain CDR2 comprising the amino acid sequence of SEQ ID NO: 199, and
a light chain CDR3 comprising the amino acid sequence of SEQ ID NO: 200;
(6) a heavy chain CDR1 comprising the amino acid sequence of SEQ ID NO: 225,
a heavy chain CDR2 comprising the amino acid sequence of SEQ ID NO: 226,
a heavy chain CDR3 comprising the amino acid sequence of SEQ ID NO: 227,
a light chain CDR1 comprising the amino acid sequence of SEQ ID NO: 228,
a light chain CDR2 comprising the amino acid sequence of SEQ ID NO: 229, and
a light chain CDR3 comprising the amino acid sequence of SEQ ID NO: 230;
(7) a heavy chain CDR1 comprising the amino acid sequence of SEQ ID NO: 272,
a heavy chain CDR2 comprising the amino acid sequence of SEQ ID NO: 273,
a heavy chain CDR3 comprising the amino acid sequence of SEQ ID NO: 274,
a light chain CDR1 comprising the amino acid sequence of SEQ ID NO: 275,
a light chain CDR2 comprising the amino acid sequence of SEQ ID NO: 276, and
a light chain CDR3 comprising the amino acid sequence of SEQ ID NO: 277;
(8) a heavy chain CDR1 comprising the amino acid sequence of SEQ ID NO: 302,
a heavy chain CDR2 comprising the amino acid sequence of SEQ ID NO: 303,
a heavy chain CDR3 comprising the amino acid sequence of SEQ ID NO: 304,
a light chain CDR1 comprising the amino acid sequence of SEQ ID NO: 305,
a light chain CDR2 comprising the amino acid sequence of SEQ ID NO: 306, and
a light chain CDR3 comprising the amino acid sequence of SEQ ID NO: 307;
(9) a heavy chain CDR1 comprising the amino acid sequence of SEQ ID NO: 308,
a heavy chain CDR2 comprising the amino acid sequence of SEQ ID NO: 309,
a heavy chain CDR3 comprising the amino acid sequence of SEQ ID NO: 310,
a light chain CDR1 comprising the amino acid sequence of SEQ ID NO: 311,
a light chain CDR2 comprising the amino acid sequence of SEQ ID NO: 312, and
a light chain CDR3 comprising the amino acid sequence of SEQ ID NO: 313;
(10) a heavy chain CDR1 comprising the amino acid sequence of SEQ ID NO: 332,
a heavy chain CDR2 comprising the amino acid sequence of SEQ ID NO: 333,
a heavy chain CDR3 comprising the amino acid sequence of SEQ ID NO: 334,
a light chain CDR1 comprising the amino acid sequence of SEQ ID NO: 335,
a light chain CDR2 comprising the amino acid sequence of SEQ ID NO: 336, and
a light chain CDR3 comprising the amino acid sequence of SEQ ID NO: 337;
(11) a heavy chain CDR1 comprising the amino acid sequence of SEQ ID NO: 344,
a heavy chain CDR2 comprising the amino acid sequence of SEQ ID NO: 345,
a heavy chain CDR3 comprising the amino acid sequence of SEQ ID NO: 346,
a light chain CDR1 comprising the amino acid sequence of SEQ ID NO: 347,
a light chain CDR2 comprising the amino acid sequence of SEQ ID NO: 348, and
a light chain CDR3 comprising the amino acid sequence of SEQ ID NO: 349;
(12) a heavy chain CDR1 comprising the amino acid sequence of SEQ ID NO: 350,
a heavy chain CDR2 comprising the amino acid sequence of SEQ ID NO: 351,
a heavy chain CDR3 comprising the amino acid sequence of SEQ ID NO: 352,
a light chain CDR1 comprising the amino acid sequence of SEQ ID NO: 353,
a light chain CDR2 comprising the amino acid sequence of SEQ ID NO: 354, and
a light chain CDR3 comprising the amino acid sequence of SEQ ID NO: 355; or
(13) a heavy chain CDR1 comprising the amino acid sequence of SEQ ID NO: 374,
a heavy chain CDR2 comprising the amino acid sequence of SEQ ID NO: 375,
a heavy chain CDR3 comprising the amino acid sequence of SEQ ID NO: 376,
a light chain CDR1 comprising the amino acid sequence of SEQ ID NO: 377,
a light chain CDR2 comprising the amino acid sequence of SEQ ID NO: 378, and
a light chain CDR3 comprising the amino acid sequence of SEQ ID NO: 379.

71. The ROR antigen binding molecule of claim 67, wherein the third antigen binding site comprises:
(1) a heavy chain CDR1 comprising the amino acid sequence of SEQ ID NO: 505,
a heavy chain CDR2 comprising the amino acid sequence of SEQ ID NO: 506,
a heavy chain CDR3 comprising the amino acid sequence of SEQ ID NO: 507,
a light chain CDR1 comprising the amino acid sequence of SEQ ID NO: 508,
a light chain CDR2 comprising the amino acid sequence of SEQ ID NO: 509, and
a light chain CDR3 comprising the amino acid sequence of SEQ ID NO: 510;
(2) a heavy chain CDR1 comprising the amino acid sequence of SEQ ID NO: 397,
a heavy chain CDR2 comprising the amino acid sequence of SEQ ID NO: 398,
a heavy chain CDR3 comprising the amino acid sequence of SEQ ID NO: 399,
a light chain CDR1 comprising the amino acid sequence of SEQ ID NO: 400,
a light chain CDR2 comprising the amino acid sequence of SEQ ID NO: 401, and
a light chain CDR3 comprising the amino acid sequence of SEQ ID NO: 402;
(3) a heavy chain CDR1 comprising the amino acid sequence of SEQ ID NO: 409,
a heavy chain CDR2 comprising the amino acid sequence of SEQ ID NO: 410,
a heavy chain CDR3 comprising the amino acid sequence of SEQ ID NO: 411,
a light chain CDR1 comprising the amino acid sequence of SEQ ID NO: 412,
a light chain CDR2 comprising the amino acid sequence of SEQ ID NO: 413, and
a light chain CDR3 comprising the amino acid sequence of SEQ ID NO: 414;
(4) a heavy chain CDR1 comprising the amino acid sequence of SEQ ID NO: 421,
a heavy chain CDR2 comprising the amino acid sequence of SEQ ID NO: 422,
a heavy chain CDR3 comprising the amino acid sequence of SEQ ID NO: 423,
a light chain CDR1 comprising the amino acid sequence of SEQ ID NO: 424,
a light chain CDR2 comprising the amino acid sequence of SEQ ID NO: 425, and
a light chain CDR3 comprising the amino acid sequence of SEQ ID NO: 426;
(5) a heavy chain CDR1 comprising the amino acid sequence of SEQ ID NO: 493,
a heavy chain CDR2 comprising the amino acid sequence of SEQ ID NO: 494,
a heavy chain CDR3 comprising the amino acid sequence of SEQ ID NO: 495,
a light chain CDR1 comprising the amino acid sequence of SEQ ID NO: 496,
a light chain CDR2 comprising the amino acid sequence of SEQ ID NO: 497, and
a light chain CDR3 comprising the amino acid sequence of SEQ ID NO: 498;
(6) a heavy chain CDR1 comprising the amino acid sequence of SEQ ID NO: 529,
a heavy chain CDR2 comprising the amino acid sequence of SEQ ID NO: 530,
a heavy chain CDR3 comprising the amino acid sequence of SEQ ID NO: 531,
a light chain CDR1 comprising the amino acid sequence of SEQ ID NO: 532,
a light chain CDR2 comprising the amino acid sequence of SEQ ID NO: 533, and
a light chain CDR3 comprising the amino acid sequence of SEQ ID NO: 534; or
(7) a heavy chain CDR1 comprising the amino acid sequence of SEQ ID NO: 535,
a heavy chain CDR2 comprising the amino acid sequence of SEQ ID NO: 536,
a heavy chain CDR3 comprising the amino acid sequence of SEQ ID NO: 537,
a light chain CDR1 comprising the amino acid sequence of SEQ ID NO: 538,
a light chain CDR2 comprising the amino acid sequence of SEQ ID NO: 539, and
a light chain CDR3 comprising the amino acid sequence of SEQ ID NO: 540.

72-156. (canceled)

157. A pharmaceutical composition comprising the ROR antigen binding molecule of claim 2 and a pharmaceutically acceptable diluent.

158. A method for treating a subject with cancer, the method comprising administering a therapeutically effective amount of the pharmaceutical composition of claim 157 to the subject.

159. The method of claim 158, wherein the cancer is an ROR antigen expressing cancer.

160. The method of claim 159, wherein the cancer expresses an ROR1 antigen.

161-162. (canceled)

163. The method of claim 158, wherein the cancer is selected from the group consisting of: pancreatic cancer, ovarian cancer, breast cancer, lung cancer, gastric cancer, melanoma, Ewing sarcoma, chronic lymphocytic leukemia, mantle cell lymphoma, B-ALL, hematological cancer, prostate cancer, colon cancer, renal cancer, thyroid cancer, liver cancer, urothelial carcinoma, melanoma, endometrial cancer, clear cell renal cell carcinoma, clear cell carcinoma, and uterine cancer.

164. The method of claim 158, wherein the pharmaceutical composition is administered in combination with an additional therapy.

165. The method of claim 164, wherein the additional therapy is surgery, radiotherapy, endocrine therapy, immunotherapy, or chemotherapy.

166. The method of claim 165, wherein the immunotherapy is an immunotherapeutic agent.

167. The method of claim 166, wherein the immunotherapeutic agent is an immune checkpoint inhibitor.

168. The method of claim 166, wherein the immunotherapeutic agent is a vaccine.

169. The method of claim 165, wherein the chemotherapy is a cytotoxic agent or a chemotherapeutic agent.

170. The ROR antigen binding molecule of claim 2, wherein the first antigen binding site comprises:
a heavy chain CDR1 comprising SEQ ID NO: 314,
a heavy chain CDR2 comprising SEQ ID NO: 315,
a heavy chain CDR3 comprising SEQ ID NO: 316,
a light chain CDR1 comprising SEQ ID NO: 317,
a light chain CDR2 comprising SEQ ID NO: 318, and
a light chain CDR3 comprising SEQ ID NO: 319.

171. The ROR antigen binding molecule of claim 2, wherein the VH region of the first antigen binding site comprises the amino acid sequence of SEQ ID NO: 104 and the VL region of the first antigen binding site comprises the amino acid sequence of SEQ ID NO: 100.


SEQUENCE LISTING (540 sequences)

SEQ ID NO: 1 - protein, 338 aa, synthetic construct
  source 1..338: note = Example 1, bivalent monospecific construct CHAIN 1, VL
VEIKRTPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 112 aa, synthetic construct
  source 1..112: note = Example 1, bivalent monospecific construct CHAIN 2, VH
VTVSSASPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 338 aa, synthetic construct; identical to SEQ ID NO: 1
  source 1..338: note = Example 1, bivalent, bispecific construct CHAIN 1, VL

SEQ ID NO: 4 - protein, 112 aa, synthetic construct; identical to SEQ ID NO: 2
  source 1..112: note = Example 1, bivalent, bispecific construct CHAIN 2, VH

SEQ ID NO: 5 - protein, 338 aa, synthetic construct
  source 1..338: note = Example 1, bivalent, bispecific construct CHAIN 3, VL
VEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 109 aa, synthetic construct
  source 1..109: note = Example 1, bivalent, bispecific construct CHAIN 4, VH
VTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 7 - protein, 105 aa, synthetic construct
  source 1..105: note = Fc Fragment of Human IgG1
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 441 aa, synthetic construct
  source 1..441: note = BC1 chain 1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 227 aa, synthetic construct
  source 1..227: note = BC1 chain 2
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 10 - protein, 441 aa, synthetic construct
  source 1..441: note = BC1 chain 3 and BC28 bivalent chain 3
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 217 aa, synthetic construct
  source 1..217: note = BC1 chain 4 and BC28 bivalent chain 4
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 12 - protein, 109 aa, synthetic construct
  source 1..109: note = BC1 Domain A
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 13 - protein, 105 aa, synthetic construct
  source 1..105: note = BC1 Domain B
PREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSC

SEQ ID NO: 14 - protein, 110 aa, synthetic construct
  source 1..110: note = BC1 Domain D
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 107 aa, synthetic construct
  source 1..107: note = BC1 Domain E
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 122 aa, synthetic construct
  source 1..122: note = BC1 Domain F
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSAS

SEQ ID NO: 17 - protein, 105 aa, synthetic construct
  source 1..105: note = BC1 Domain G
PREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 18 - protein, 107 aa, synthetic construct [sequence omitted]

SEQ ID NO: 19 - protein, 107 aa, synthetic construct [sequence omitted]

SEQ ID NO: 20 - protein, 110 aa, synthetic construct; identical to SEQ ID NO: 14
  source 1..110: note = BC1 Domain J

SEQ ID NO: 21 - protein, 107 aa, synthetic construct
  source 1..107: note = BC1 Domain K
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 113 aa, synthetic construct [sequence omitted]

SEQ ID NO: 23 - protein, 104 aa, synthetic construct
  source 1..104: note = BC1 Domain M
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 24 - protein, 441 aa, synthetic construct
  source 1..441: note = BC28 chain 1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 227 aa, synthetic construct
  source 1..227: note = BC28 chain 2
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASPREPQVYTLPPCRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 109 aa, synthetic construct; identical to SEQ ID NO: 12
  source 1..109: note = BC28 domain A

SEQ ID NO: 27 - protein, 105 aa, synthetic construct
  source 1..105: note = BC28 domain B
PREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 110 aa, synthetic construct; identical to SEQ ID NO: 14
  source 1..110: note = BC28 domain D

SEQ ID NO: 29 - protein, 107 aa, synthetic construct; identical to SEQ ID NO: 15
  source 1..107: note = BC28 domain E

SEQ ID NO: 30 - protein, 122 aa, synthetic construct; identical to SEQ ID NO: 16
  source 1..122: note = BC28 domain F

SEQ ID NO: 31 - protein, 105 aa, synthetic construct
  source 1..105: note = BC28 domain G
PREPQVYTLPPCRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 441 aa, synthetic construct
  source 1..441: note = BC44 chain 1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 109 aa, synthetic construct; identical to SEQ ID NO: 12
  source 1..109: note = BC44 Domain A

SEQ ID NO: 34 - protein, 105 aa, synthetic construct
  source 1..105: note = BC44 Domain B
VREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 110 aa, synthetic construct; identical to SEQ ID NO: 14
  source 1..110: note = BC44 Domain D

SEQ ID NO: 36 - protein, 107 aa, synthetic construct; identical to SEQ ID NO: 15
  source 1..107: note = BC44 Domain E

SEQ ID NO: 37 - protein, 661 aa, synthetic construct
  source 1..661: note = BC28 1x2 chain 3
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 109 aa, synthetic construct; identical to SEQ ID NO: 12
  source 1..109: note = BC28 1x2 domain R

SEQ ID NO: 39 - protein, 105 aa, synthetic construct; identical to SEQ ID NO: 27
  source 1..105: note = BC28 1x2 domain S

SEQ ID NO: 40 - protein, 6 aa, synthetic construct
  source 1..6: note = BC28 1x2 linker
GSGSGS

SEQ ID NO: 41 - protein, 107 aa, synthetic construct [sequence omitted]

SEQ ID NO: 42 - protein, 107 aa, synthetic construct [sequence omitted]

SEQ ID NO: 43 - protein, 110 aa, synthetic construct; identical to SEQ ID NO: 14
  source 1..110: note = BC28 1x2 domain J

SEQ ID NO: 44 - protein, 107 aa, synthetic construct; identical to SEQ ID NO: 21
  source 1..107: note = BC28 1x2 domain K

SEQ ID NO: 45 - protein, 661 aa, synthetic construct
  source 1..661: note = BC28-1x1x1a chain 3
DIQMTQSPSSLSASVGDRVTITCRASQSVSSAVAWYQQKPGKAPKLLIYSASSLYSGVPSRFSGSRSGTDFTLTISSLQPEDFATYYCQQRDSYLWTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCGSGSGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 109 aa, synthetic construct
  source 1..109: note = BC28-1x1x1a domain R
DIQMTQSPSSLSASVGDRVTITCRASQSVSSAVAWYQQKPGKAPKLLIYSASSLYSGVPSRFSGSRSGTDFTLTISSLQPEDFATYYCQQRDSYLWTFGQGTKVEIKRT

SEQ ID NO: 47 - protein, 105 aa, synthetic construct; identical to SEQ ID NO: 13
  source 1..105: note = BC28-1x1x1a domain S

SEQ ID NO: 48 - protein, 6 aa, synthetic construct
  source 1..6: note = BC28-1x1x1a linker
GSGSGS

SEQ ID NO: 49 - protein, 107 aa, synthetic construct [sequence omitted]

SEQ ID NO: 50 - protein, 107 aa, synthetic construct [sequence omitted]

SEQ ID NO: 51 - protein, 110 aa, synthetic construct; identical to SEQ ID NO: 14
  source 1..110: note = BC28-1x1x1a domain J

SEQ ID NO: 52 - protein, 107 aa, synthetic construct; identical to SEQ ID NO: 21
  source 1..107: note = BC28-1x1x1a domain K

SEQ ID NO: 53 - protein, 224 aa, synthetic construct
  source 1..224: note = hCTLA4-4.chain 2
EVQLVESGGGLVQPGGSLRLSCAASGFTFSTYYIHWVRQAPGKGLEWVAVIYPYTGFTYYADSVKGRFTISADTSKNTAYLQMNSLRAEDTAVYYCARGEYTVLDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 54 - protein, 119 aa, synthetic construct
  source 1..119: note = hCTLA4-4 domain F
EVQLVESGGGLVQPGGSLRLSCAASGFTFSTYYIHWVRQAPGKGLEWVAVIYPYTGFTYYADSVKGRFTISADTSKNTAYLQMNSLRAEDTAVYYCARGEYTVLDYWGQGTLVTVSSAS

SEQ ID NO: 55 - protein, 105 aa, synthetic construct; identical to SEQ ID NO: 17
  source 1..105: note = hCTLA4-4 domain G

SEQ ID NO: 56 - protein, 10 aa, synthetic construct
  source 1..10: note = Hinge
DKTHTCPPCP

SEQ ID NO: 57 - protein, 8 aa, synthetic construct
  source 1..8: note = BC1, BC5, BC6, BC13, BC14, BC24, BC25, BC28, BC30, and BC45, Polypeptide 1 Domain Junction
IKRTPREP

SEQ ID NO: 58 - protein, 8 aa, synthetic construct
  source 1..8: note = BC15 and BC44 Polypeptide 1 Domain Junction
IKRTVREP

SEQ ID NO: 59 - protein, 7 aa, synthetic construct
  source 1..7: note = BC16-Polypeptide 1 Domain Junction
IKRTREP

SEQ ID NO: 60 - protein, 9 aa, synthetic construct
  source 1..9: note = BC17-Polypeptide 1 Domain Junction
IKRTVPREP

SEQ ID NO: 61 - protein, 8 aa, synthetic construct
  source 1..8: note = BC26-Polypeptide 1 Domain Junction
IKRTVAEP

SEQ ID NO: 62 - protein, 10 aa, synthetic construct
  source 1..10: note = BC27-Polypeptide 1 Domain Junction
IKRTVAPREP

SEQ ID NO: 63 - protein, 8 aa, synthetic construct
  source 1..8: note = BC1, BC5, BC6, BC15, BC16, BC17, BC26, BC27, BC28, BC30, BC44, and BC45, Polypeptide 2 Domain Junction
SSASPREP

SEQ ID NO: 64 - protein, 8 aa, synthetic construct
  source 1..8: note = BC13-Polypeptide 2 Domain Junction
SSASTREP

SEQ ID NO: 65 - protein, 9 aa, synthetic construct
  source 1..9: note = BC14-Polypeptide 2 Domain Junction
SSASTPREP

SEQ ID NO: 66 - protein, 9 aa, synthetic construct
  source 1..9: note = BC24-Polypeptide 2 Domain Junction
SSASTKGEP

SEQ ID NO: 67 - protein, 10 aa, synthetic construct
  source 1..10: note = BC25-Polypeptide 2 Domain Junction
SSASTKGREP

SEQ ID NO: 68 - protein, 123 aa, synthetic construct
  source 1..123: note = SP34-89 VH
EVQLVESGGGLVQPGGSLRLSCAASGFTFNTYAMNWVRQAPGKGLEWVARIRSKYNNYATYYADSVKGRFSISRDDSKNTAYLQMNSLRAEDTAVYYCVRHGNFGNSYVSWFAYWGQGTLVTV

SEQ ID NO: 69 - protein, 109 aa, synthetic construct
  source 1..109: note = SP34-89 VL
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 70 - protein, 123 aa, synthetic construct
  source 1..123: note = SP34-89 VH-N30S VH, lower case denotes mutation
EVQLVESGGGLVQPGGSLRLSCAASGFTFSTYAMNWVRQAPGKGLEWVARIRSKYNNYATYYADSVKGRFSISRDDSKNTAYLQMNSLRAEDTAVYYCVRHGNFGNSYVSWFAYWGQGTLVTV

SEQ ID NO: 71 - protein, 123 aa, synthetic construct
  source 1..123: note = SP34-89 VH-G65D VH, lower case denotes mutation
EVQLVESGGGLVQPGGSLRLSCAASGFTFNTYAMNWVRQAPGKGLEWVARIRSKYNNYATYYADSVKDRFSISRDDSKNTAYLQMNSLRAEDTAVYYCVRHGNFGNSYVSWFAYWGQGTLVTV

SEQ ID NO: 72 - protein, 123 aa, synthetic construct
  source 1..123: note = SP34-89 VH-S68T VH, lower case denotes mutation
EVQLVESGGGLVQPGGSLRLSCAASGFTFNTYAMNWVRQAPGKGLEWVARIRSKYNNYATYYADSVKGRFTISRDDSKNTAYLQMNSLRAEDTAVYYCVRHGNFGNSYVSWFAYWGQGTLVTV

SEQ ID NO: 73 - protein, 109 aa, synthetic construct
  source 1..109: note = SP34-89 VL-W57G VL, lower case denotes mutation
QAVVTQEPSLTVSPGGTVTLTCRSSTGAVTTSNYANWVQQKPGQAPRGLIGGTNKRAPGTPARFSGSLLGGKAALTITGAQAEDEADYYCALWYSNLWVFGGGTKLTVL

SEQ ID NO: 74 - protein, 455 aa, synthetic construct
  source 1..455: note = Phage display heavy chain
  VARIANT 30..33: note = Xaa can be any naturally occurring amino acid
  REGION 34..35: note = CDR sequences that were constant
  VARIANT 50..60: note = Xaa can be any naturally occurring amino acid
  REGION 61..67: note = CDR sequences that were constant
  VARIANT 100..112: note = Xaa can be any naturally occurring amino acid
  REGION 113..114: note = CDR sequences that were constant
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, synthetic construct
  source 1..214: note = Phage display light chain
  REGION 24..32: note = CDR sequences that were constant
  REGION 50..56: note = CDR sequences that were constant
  REGION 89..90: note = CDR sequences that were constant
  REGION 97..98: note = CDR sequences that were constant
  VARIANT 91..96: note = Xaa can be any naturally occurring amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 109 aa, synthetic construct
  REGION 24..32: note = CDR sequences that were constant
  REGION 50..56: note = CDR sequences that were constant
  REGION 89..90: note = CDR sequences that were constant
  VARIANT 91..96: note = Xaa can be any naturally occurring amino acid
  REGION 97..98: note = CDR sequences that were constant
  source 1..109: note = B-Body Domain A/H Scaffold
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRT

SEQ ID NO: 77 - protein, 127 aa, synthetic construct
  VARIANT 30..33: note = Xaa can be any naturally occurring amino acid
  REGION 34..35: note = CDR sequences that were constant
  VARIANT 50..60: note = Xaa can be any naturally occurring amino acid
  REGION 61..67: note = CDR sequences that were constant
  VARIANT 100..112: note = Xaa can be any naturally occurring amino acid
  REGION 113..114: note = CDR sequences that were constant
  source 1..127: note = B-Body Domain F/L Scaffold
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSAS

SEQ ID NO: 78 - protein, 441 aa, synthetic construct
  REGION 24..32: note = CDR sequences that were constant
  REGION 50..56: note = CDR sequences that were constant
  REGION 89..90: note = CDR sequences that were constant
  VARIANT 91..96: note = Xaa can be any naturally occurring amino acid
  REGION 97..98: note = CDR sequences that were constant
  source 1..441: note = BC1 Chain 1 Scaffold
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 232 aa, synthetic construct
  VARIANT 30..33: note = Xaa can be any naturally occurring amino acid
  REGION 34..35: note = CDR sequences that were constant
  VARIANT 50..60: note = Xaa can be any naturally occurring amino acid
  REGION 61..67: note = CDR sequences that were constant
  VARIANT 100..112: note = Xaa can be any naturally occurring amino acid
  REGION 113..114: note = CDR sequences that were constant
  source 1..232: note = BC1 Chain 2 Scaffold
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 80 - protein, 441 aa, synthetic construct
  REGION 24..32: note = CDR sequences that were constant
  REGION 50..56: note = CDR sequences that were constant
  REGION 89..90: note = CDR sequences that were constant
  VARIANT 91..96: note = Xaa can be any naturally occurring amino acid
  REGION 97..98: note = CDR sequences that were constant
  source 1..441: note = BC1 Chain 3 Scaffold
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 81 - protein, 229 aa, synthetic construct
  VARIANT 30..33: note = Xaa can be any naturally occurring amino acid
  REGION 34..35: note = CDR sequences that were constant
  VARIANT 50..60: note = Xaa can be any naturally occurring amino acid
  REGION 61..67: note = CDR sequences that were constant
  VARIANT 100..112: note = Xaa can be any naturally occurring amino acid
  REGION 113..114: note = CDR sequences that were constant
  source 1..229: note = BC1 Chain 4 Scaffold
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 82 - protein, 668 aa, synthetic construct
  REGION 24..32: note = CDR sequences that were constant
  REGION 50..56: note = CDR sequences that were constant
  REGION 89..90: note = CDR sequences that were constant
  VARIANT 91..96: note = Xaa can be any naturally occurring amino acid
  REGION 97..98: note = CDR sequences that were constant
  source 1..668: note = BC1 Chain 3 1(A)x2(B-A) SP34-89 Scaffold
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 10 aa, synthetic construct
  source 1..10: note = BC1 Chain 3 1(A)x2(B-A) SP34-89 S-H Junction
TASSGGSSSG

SEQ ID NO: 84 - protein, 441 aa, synthetic construct
  source 1..441: note = I2A-10 Chain 1 BC1 1x1
DIQMTQSPSSLSASVGDRVTITCRASQSVSSAVAWYQQKPGKAPKLLIYSASSLYSGVPSRFSGSRSGTDFTLTISSLQPEDFATYYCQQYYYYPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 223 aa, synthetic construct
  source 1..223: note = I2A-10 Chain 2 BC1 1x1
EVQLVESGGGLVQPGGSLRLSCAASGFTFSSYFIHWVRQAPGKGLEWVAGIYPSDGYTSYADSVKGRFTISADTSKNTAYLQMNSLRAEDTAVYYCARYYSGMDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 86 - protein, 444 aa, synthetic construct
  source 1..444: note = I2A-10 Chain 3 BC1 1x1
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 229 aa, synthetic construct
  source 1..229: note = I2A-10 Chain 4 BC1 1x1
EVQLVESGGGLVQPGGSLRLSCAASGFTFNTYAMNWVRQAPGKGLEWVARIRSKYNNYATYYADSVKGRFSISRDDSKNTAYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 88 - protein, 668 aa, synthetic construct
  source 1..668: note = I2A-10 Chain 3 BC1 1x2
DIQMTQSPSSLSASVGDRVTITCRASQSVSSAVAWYQQKPGKAPKLLIYSASSLYSGVPSRFSGSRSGTDFTLTISSLQPEDFATYYCQQYYYYPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 89 - protein, 441 aa, synthetic construct
  source 1..441: note = I2A-27 Chain 1 BC1 1x1
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPRTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 225 aa, synthetic construct
  source 1..225: note = I2A-27 Chain 2 BC1 1x1
EVQLVESGGGLVQPGGSLRLSCAASGFTFKGYYIHWVRQAPGKGLEWVAAIYPYGGSTDYADSVKGRFTISADTSKNTAYLQMNSLRAEDTAVYYCARVYIYGVFDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 91 - protein, 444 aa, synthetic construct; identical to SEQ ID NO: 86
  source 1..444: note = I2A-27 Chain 3 BC1 1x1

SEQ ID NO: 92 - protein, 229 aa, synthetic construct; identical to SEQ ID NO: 87
  source 1..229: note = I2A-27 Chain 4 BC1 1x1

SEQ ID NO: 93 - protein, 668 aa, synthetic construct
  source 1..668: note = I2A-27 Chain 3 BC1 1x2
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPRTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 937 aa, Homo sapiens
  source 1..937: note = Human ROR1 (UniProt accession #Q01973)
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 95 - protein, 943 aa, Homo sapiens
  source 1..943: note = Human ROR2 (UniProt accession #Q01974)
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 96 - protein, 441 aa, synthetic construct
  source 1..441: note = I2A-27 Chain 1
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPRTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 225 aa, synthetic construct; identical to SEQ ID NO: 90
  source 1..225: note = I2A-27 Chain 2 or Chain 5

SEQ ID NO: 98 - protein, 668 aa, synthetic construct
  source 1..668: note = I2A-27 Chain 3
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPRTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 229 aa, synthetic construct; identical to SEQ ID NO: 87
  source 1..229: note = I2A-27 Chain 4

SEQ ID NO: 100 - protein, 109 aa, synthetic construct
  source 1..109: note = I2A-27 Domain A
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPRTFGQGTKVEIKRT

SEQ ID NO: 101 - protein, 105 aa, synthetic construct; identical to SEQ ID NO: 13
  source 1..105: note = I2A-27 Domain B

SEQ ID NO: 102 - protein, 110 aa, synthetic construct
  source 1..110: note = I2A-27 Domain D
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAK

SEQ ID NO: 103 - protein, 107 aa, synthetic construct; identical to SEQ ID NO: 21
  source 1..107: note = I2A-27 Domain E

SEQ ID NO: 104 - protein, 120 aa, synthetic construct
  source 1..120: note = I2A-27 Domain F or Domain T
EVQLVESGGGLVQPGGSLRLSCAASGFTFKGYYIHWVRQAPGKGLEWVAAIYPYGGSTDYADSVKGRFTISADTSKNTAYLQMNSLRAEDTAVYYCARVYIYGVFDYWGQGTLVTVSSAS

SEQ ID NO: 105 - protein, 105 aa, synthetic construct; identical to SEQ ID NO: 17
  source 1..105: note = I2A-27 Domain G or Domain U

SEQ ID NO: 106 - protein, 109 aa, synthetic construct; identical to SEQ ID NO: 100
  source 1..109: note = I2A-27 Domain R

SEQ ID NO: 107 - protein, 105 aa, synthetic construct; identical to SEQ ID NO: 13
  source 1..105: note = I2A-27 Domain S

SEQ ID NO: 108 - protein, 113 aa, synthetic construct
  source 1..113: note = I2A-27 Domain H
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTV

SEQ ID NO: 109 - protein, 104 aa, synthetic construct
  source 1..104: note = I2A-27 Domain I
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 110 aa, synthetic construct; identical to SEQ ID NO: 102
  source 1..110: note = I2A-27 Domain J

SEQ ID NO: 111 - protein, 107 aa, synthetic construct; identical to SEQ ID NO: 15
  source 1..107: note = I2A-27 Domain K

SEQ ID NO: 112 - protein, 127 aa, synthetic construct
  source 1..127: note = I2A-27 Domain L
EVQLVESGGGLVQPGGSLRLSCAASGFTFNTYAMNWVRQAPGKGLEWVARIRSKYNNYATYYADSVKGRFSISRDDSKNTAYLQMNSLRAEDTAVYYCVRHGNFGNSYVSWFAYWGQGTLVTVSSAS

SEQ ID NO: 113 - protein, 102 aa, synthetic construct
  source 1..102: note = I2A-27 Domain M
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 114 - protein, 441 aa, synthetic construct
  source 1..441: note = I2A-10 Chain 1
DIQMTQSPSSLSASVGDRVTITCRASQSVSSAVAWYQQKPGKAPKLLIYSASSLYSGVPSRFSGSRSGTDFTLTISSLQPEDFATYYCQQYYYYPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - protein, 224 aa, synthetic construct
  source 1..224: note = I2A-10 Chain 2 or Chain 5
EVQLVESGGGLVQPGGSLRLSCAASGFTFSSYFIHWVRQAPGKGLEWVAGIYPSDGYTSYADSVKGRFTISADTSKNTAYLQMNSLRAEDTAVYYCARYYVSGMDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 116 - protein, 668 aa, synthetic construct
  source 1..668: note = I2A-10 Chain 3
DIQMTQSPSSLSASVGDRVTITCRASQSVSSAVAWYQQKPGKAPKLLIYSASSLYSGVPSRFSGSRSGTDFTLTISSLQPEDFATYYCQQYYYYPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 229 aa, synthetic construct; identical to SEQ ID NO: 87
  source 1..229: note = I2A-10 Chain 4

SEQ ID NO: 118 - protein, 109 aa, synthetic construct
  source 1..109: note = I2A-10 Domain A
DIQMTQSPSSLSASVGDRVTITCRASQSVSSAVAWYQQKPGKAPKLLIYSASSLYSGVPSRFSGSRSGTDFTLTISSLQPEDFATYYCQQYYYYPGTFGQGTKVEIKRT

SEQ ID NO: 119 - protein, 105 aa, synthetic construct; identical to SEQ ID NO: 13
  source 1..105: note = I2A-10 Domain B

SEQ ID NO: 120 - protein, 110 aa, synthetic construct; identical to SEQ ID NO: 102
  source 1..110: note = I2A-10 Domain D

SEQ ID NO: 121 - protein, 107 aa, synthetic construct; identical to SEQ ID NO: 21
  source 1..107: note = I2A-10 Domain E

SEQ ID NO: 122 - protein, 119 aa, synthetic construct
  source 1..119: note = I2A-10 Domain F or Domain T
EVQLVESGGGLVQPGGSLRLSCAASGFTFSSYFIHWVRQAPGKGLEWVAGIYPSDGYTSYADSVKGRFTISADTSKNTAYLQMNSLRAEDTAVYYCARYYVSGMDYWGQGTLVTVSSAS

SEQ ID NO: 123 - protein, 105 aa, synthetic construct; identical to SEQ ID NO: 17
  source 1..105: note = I2A-10 Domain G or Domain U

SEQ ID NO: 124 - protein, 109 aa, synthetic construct; identical to SEQ ID NO: 118
  source 1..109: note = I2A-10 Domain R

SEQ ID NO: 125 - protein, 105 aa, synthetic construct; identical to SEQ ID NO: 13
  source 1..105: note = I2A-10 Domain S

SEQ ID NO: 126 - protein, 113 aa, synthetic construct; identical to SEQ ID NO: 108
  source 1..113: note = I2A-10 Domain H

SEQ ID NO: 127 - protein, 104 aa, synthetic construct; identical to SEQ ID NO: 109
  source 1..104: note = I2A-10 Domain I

SEQ ID NO: 128 - protein, 110 aa, synthetic construct; identical to SEQ ID NO: 102
  source 1..110: note = I2A-10 Domain J

SEQ ID NO: 129 - protein, 107 aa, synthetic construct; identical to SEQ ID NO: 15
  source 1..107: note = I2A-10 Domain K

SEQ ID NO: 130 - protein, 127 aa, synthetic construct; identical to SEQ ID NO: 112
  source 1..127: note = I2A-10 Domain L

SEQ ID NO: 131 - protein, 102 aa, synthetic construct; identical to SEQ ID NO: 113
  source 1..102: note = I2A-10 Domain M

SEQ ID NO: 132 - protein, 441 aa, synthetic construct
  source 1..441: note = I2A-10 D54E Y55Q Chain 1
DIQMTQSPSSLSASVGDRVTITCRASQSVSSAVAWYQQKPGKAPKLLIYSASSLQSGVPSRFSGSRSGTDFTLTISSLQPEDFATYYCQQYYYYPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 133 - protein, 224 aa, synthetic construct
  source 1..224: note = I2A-10 D54E Y55Q Chain 2 or Chain 5
EVQLVESGGGLVQPGGSLRLSCAASGFTFSSYFIHWVRQAPGKGLEWVAGIYPSEGYTSYADSVKGRFTISADTSKNTAYLQMNSLRAEDTAVYYCARYYVSGMDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 134 - protein, 668 aa, synthetic construct
  source 1..668: note = I2A-10 D54E Y55Q Chain 3
DIQMTQSPSSLSASVGDRVTITCRASQSVSSAVAWYQQKPGKAPKLLIYSASSLQSGVPSRFSGSRSGTDFTLTISSLQPEDFATYYCQQYYYYPGTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 229 aa, synthetic construct; identical to SEQ ID NO: 87
  source 1..229: note = I2A-10 D54E Y55Q Chain 4

SEQ ID NO: 136 - protein, 109 aa, synthetic construct
  source 1..109: note = I2A-10 D54E Y55Q Domain A
DIQMTQSPSSLSASVGDRVTITCRASQSVSSAVAWYQQKPGKAPKLLIYSASSLQSGVPSRFSGSRSGTDFTLTISSLQPEDFATYYCQQYYYYPGTFGQGTKVEIKRT

SEQ ID NO: 137 - protein, 105 aa, synthetic construct; identical to SEQ ID NO: 13
  source 1..105: note = I2A-10 D54E Y55Q Domain B

SEQ ID NO: 138 - protein, 110 aa, synthetic construct; identical to SEQ ID NO: 102
  source 1..110: note = I2A-10 D54E Y55Q Domain D

SEQ ID NO: 139 - protein, 107 aa, synthetic construct; identical to SEQ ID NO: 21
  source 1..107: note = I2A-10 D54E Y55Q Domain E

SEQ ID NO: 140 - protein, 119 aa, synthetic construct
  source 1..119: note = I2A-10 D54E Y55Q Domain F or Domain T
EVQLVESGGGLVQPGGSLRLSCAASGFTFSSYFIHWVRQAPGKGLEWVAGIYPSEGYTSYADSVKGRFTISADTSKNTAYLQMNSLRAEDTAVYYCARYYVSGMDYWGQGTLVTVSSAS

SEQ ID NO: 141 - protein, 105 aa, synthetic construct; identical to SEQ ID NO: 17
  source 1..105: note = I2A-10 D54E Y55Q Domain G or Domain U

SEQ ID NO: 142 - protein, 109 aa, synthetic construct; identical to SEQ ID NO: 136
  source 1..109: note = I2A-10 D54E Y55Q Domain R

SEQ ID NO: 143 - protein, 105 aa, synthetic construct; identical to SEQ ID NO: 13
  source 1..105: note = I2A-10 D54E Y55Q Domain S

SEQ ID NO: 144 - protein, 113 aa, synthetic construct; identical to SEQ ID NO: 108
  source 1..113: note = I2A-10 D54E Y55Q Domain H

SEQ ID NO: 145 - protein, 104 aa, synthetic construct; identical to SEQ ID NO: 109
  source 1..104: note = I2A-10 D54E Y55Q Domain I

SEQ ID NO: 146 - protein, 110 aa, synthetic construct; identical to SEQ ID NO: 102
  source 1..110: note = I2A-10 D54E Y55Q Domain J

SEQ ID NO: 147 - protein, 107 aa, synthetic construct; identical to SEQ ID NO: 15
  source 1..107: note = I2A-10 D54E Y55Q Domain K

SEQ ID NO: 148 - protein, 127 aa, synthetic construct; identical to SEQ ID NO: 112
  source 1..127: note = I2A-10 D54E Y55Q Domain L

SEQ ID NO: 149 - protein, 102 aa, synthetic construct; identical to SEQ ID NO: 113
  source 1..102: note = I2A-10 D54E Y55Q Domain M

SEQ ID NO: 150 - protein, 19 aa, synthetic construct
  source 1..19: note = Exemplary signal sequence for I2A-27, I2A-10 and I2A-10 D54E Y55Q, e.g., Chain 1
MGWSLILLFLVAVATRVLS

SEQ ID NO: 151 - protein, 22 aa, synthetic construct
  source 1..22: note = Exemplary signal sequence for I2A-27, I2A-10 and I2A-10 D54E Y55Q , e.g., Chain 2, 3, 4 or 5
MDFQVQIISFLLISASVIMSRG

SEQ ID NO: 152 - protein, 23 aa, synthetic construct
  source 1..23: note = Exemplary signal sequence for I2A-27, I2A-10 and I2A-10 D54E Y55Q, e.g., Chain 3
MDFQVQIISFLLISASVIMSRGS

SEQ ID NO: 153 - protein, 6 aa, synthetic construct
  source 1..6: note = CDR1 VH of I2A-1
FSSYFI

SEQ ID NO: 154 - protein, 10 aa, synthetic construct
  source 1..10: note = CDR2 VH of I2A-1
AIYPEGGYTY

SEQ ID NO: 155 - protein, 8 aa, synthetic construct
  source 1..8: note = CDR3 VH of I2A-1
DYKYVGAL

SEQ ID NO: 156 - protein, 11 aa, synthetic construct
  source 1..11: note = CDR1 VL of I2A-1
RASQSVSSAVA

SEQ ID NO: 157 - protein, 7 aa, synthetic construct
  source 1..7: note = CDR2 VL of I2A-1
SASSLYS

SEQ ID NO: 158 - protein, 6 aa, synthetic construct
  source 1..6: note = CDR3 VL of I2A-1
YYYFPG

SEQ ID NO: 159 - protein, 6 aa, synthetic construct
  source 1..6: note = CDR1 VH of I2A-2
FSYYGI

SEQ ID NO: 160 - protein, 10 aa, synthetic construct
  source 1..10: note = CDR2 VH of I2A-2
FIYSRGGYTI

SEQ ID NO: 161 - protein, 6 aa, synthetic construct
  source 1..6: note = CDR3 VH of I2A-2
YIGAGL

SEQ ID NO: 162 - protein, 11 aa, synthetic construct
  source 1..11: note = CDR1 VL of I2A-2
RASQSVSSAVA

SEQ ID NO: 163 - protein, 7 aa, synthetic construct
  source 1..7: note = CDR2 VL of I2A-2
SASSLYS

SEQ ID NO: 164 - protein, 6 aa, synthetic construct
  source 1..6: note = CDR3 VL of I2A-2
YYWDPI

SEQ ID NO: 165 - protein, 6 aa, synthetic construct
  source 1..6: note = CDR1 VH of I2A-3
FTSYEI

SEQ ID NO: 166 - protein, 10 aa, synthetic construct
  source 1..10: note = CDR2 VH of I2A-3
HIDPYGGYTQ

SEQ ID NO: 167 - protein, 6 aa, synthetic construct
  source 1..6: note = CDR3 VH of I2A-3
RGVAVF

SEQ ID NO: 168 - protein, 11 aa, synthetic construct
  source 1..11: note = CDR1 VL of I2A-3
RASQSVSSAVA

SEQ ID NO: 169 - protein, 7 aa, synthetic construct
  source 1..7: note = CDR2 VL of I2A-3
SASSLYS

SEQ ID NO: 170 - protein, 6 aa, synthetic construct
  source 1..6: note = CDR3 VL of I2A-3
WAYAPV

SEQ ID NO: 171 - protein, 6 aa, synthetic construct
  source 1..6: note = CDR1 VH of I2A-4
FYSYDI

SEQ ID NO: 172 - protein, 10 aa, synthetic construct
  source 1..10: note = CDR2 VH of I2A-4
YISPYWGITT

SEQ ID NO: 173 - protein, 10 aa, synthetic construct
  source 1..10: note = CDR3 VH of I2A-4
YIGSSYWDAL

SEQ ID NO: 174 - protein, 11 aa, synthetic construct
  source 1..11: note = CDR1 VL of I2A-4
RASQSVSSAVA

SEQ ID NO: 175 - protein, 7 aa, synthetic construct
  source 1..7: note = CDR2 VL of I2A-4
SASSLYS

SEQ ID NO: 176 - protein, 6 aa, synthetic construct
  source 1..6: note = CDR3 VL of I2A-4
SDSSLV

SEQ ID NO: 177 - protein, 6 aa, synthetic construct
  source 1..6: note = CDR1 VH of I2A-5
FSSYGI

SEQ ID NO: 178 - protein, 10 aa, synthetic construct
  source 1..10: note = CDR2 VH of I2A-5
WISPTGSITI

SEQ ID NO: 179 - protein, 8 aa, synthetic construct
  source 1..8: note = CDR3 VH of I2A-5
SYMIYGGL

SEQ ID NO: 180 - protein, 11 aa, synthetic construct
  source 1..11: note = CDR1 VL of I2A-5
RASQSVSSAVA

SEQ ID NO: 181 - protein, 7 aa, synthetic construct
  source 1..7: note = CDR2 VL of I2A-5
SASSLYS

SEQ ID NO: 182 - protein, 6 aa, synthetic construct
  source 1..6: note = CDR3 VL of I2A-5
RVSSPW

SEQ ID NO: 183 - protein, 6 aa, synthetic construct
  source 1..6: note = CDR1 VH of I2A-6
FSLYAI

SEQ ID NO: 184 - protein, 10 aa, synthetic construct
  source 1..10: note = CDR2 VH of I2A-6
EIDSWLGYTY

SEQ ID NO: 185 - protein, 11 aa, synthetic construct
  source 1..11: note = CDR3 VH of I2A-6
RPVTEVYYSAL

SEQ ID NO: 186 - protein, 11 aa, synthetic construct
  source 1..11: note = CDR1 VL of I2A-6
RASQSVSSAVA

SEQ ID NO: 187 - protein, 7 aa, synthetic construct
  source 1..7: note = CDR2 VL of I2A-6
SASSLYS

SEQ ID NO: 188 - protein, 6 aa, synthetic construct
  source 1..6: note = CDR3 VL of I2A-6
YDRSLH

SEQ ID NO: 189 - protein, 6 aa, synthetic construct
  source 1..6: note = CDR1 VH of I2A-7
FSRYYI

SEQ ID NO: 190 - protein, 10 aa, synthetic construct
  source 1..10: note = CDR2 VH of I2A-7
DIDSYGGFTY

SEQ ID NO: 191 - protein, 11 aa, synthetic construct
  source 1..11: note = CDR3 VH of I2A-7
AHRFLQGGYVL

SEQ ID NO: 192 - protein, 11 aa, synthetic construct
  source 1..11: note = CDR1 VL of I2A-7
RASQSVSSAVA

SEQ ID NO: 193 - protein, 7 aa, synthetic construct
  source 1..7: note = CDR2 VL of I2A-7
SASSLYS

SEQ ID NO: 194 - protein, 6 aa, synthetic construct
  source 1..6: note = CDR3 VL of I2A-7
YSWGLW

SEQ ID NO: 195 - protein, 6 aa, synthetic construct
  source 1..6: note = CDR1 VH of I2A-8
FYGYYI

SEQ ID NO: 196 - protein, 10 aa, synthetic construct
  source 1..10: note = CDR2 VH of I2A-8
GIRPGGTYTY

SEQ ID NO: 197 - protein, 6 aa, synthetic construct
  source 1..6: note = CDR3 VH of I2A-8
YRYPAF

SEQ ID NO: 198 - protein, 11 aa, synthetic construct
  source 1..11: note = CDR1 VL of I2A-8
RASQSVSSAVA

SEQ ID NO: 199 - protein, 7 aa, synthetic construct
  source 1..7: note = CDR2 VL of I2A-8
SASSLYS

SEQ ID NO: 200 - protein, 6 aa, synthetic construct
  source 1..6: note = CDR3 VL of I2A-8
RRQHLW

SEQ ID NO: 201 - protein, 6 aa, synthetic construct
  source 1..6: note = CDR1 VH of I2A-9
FSSYTI

SEQ ID NO: 202 - protein, 10 aa, synthetic construct
  source 1..10: note = CDR2 VH of I2A-9
AIDSGWSYTD

SEQ ID NO: 203 - protein, 6 aa, synthetic construct
  source 1..6: note = CDR3 VH of I2A-9
AYGGVM

SEQ ID NO: 204 - protein, 11 aa, synthetic construct
  source 1..11: note = CDR1 VL of I2A-9
RASQSVSSAVA

SEQ ID NO: 205 - protein, 7 aa, synthetic construct
  source 1..7: note = CDR2 VL of I2A-9
SASSLYS

SEQ ID NO: 206 - protein, 5 aa, synthetic construct
  source 1..5: note = CDR3 VL of I2A-9
YWWPG

SEQ ID NO: 207 - protein, 6 aa, synthetic construct
  source 1..6: note = CDR1 VH of I2A-10
FSSYFI

SEQ ID NO: 208 - protein, 10 aa, synthetic construct
  source 1..10: note = CDR2 VH of I2A-10
GIYPSDGYTS

SEQ ID NO: 209 - protein, 6 aa, synthetic construct
  source 1..6: note = CDR3 VH of I2A-10
YYVSGM

SEQ ID NO: 210 - protein, 11 aa, synthetic construct
  source 1..11: note = CDR1 VL of I2A-10
RASQSVSSAVA

SEQ ID NO: 211 - protein, 7 aa, synthetic construct
  source 1..7: note = CDR2 VL of I2A-10
SASSLYS

SEQ ID NO: 212 - protein, 6 aa, synthetic construct
  source 1..6: note = CDR3 VL of I2A-10
YYYYPG

SEQ ID NO: 213 - protein, 6 aa, synthetic construct
  source 1..6: note = CDR1 VH of I2A-10 D54E Y55Q
FSSYFI

SEQ ID NO: 214 - protein, 10 aa, synthetic construct
  source 1..10: note = CDR2 VH of I2A-10 D54E Y55Q
GIYPSEGYTS

SEQ ID NO: 215 - protein, 6 aa, synthetic construct
  source 1..6: note = CDR3 VH of I2A-10 D54E Y55Q
YYVSGM

SEQ ID NO: 216 - protein, 11 aa, synthetic construct
  source 1..11: note = CDR1 VL of I2A-10 D54E Y55Q
RASQSVSSAVA

SEQ ID NO: 217 - protein, 7 aa, synthetic construct
  source 1..7: note = CDR2 VL of I2A-10 D54E Y55Q
SASSLQS

SEQ ID NO: 218 - protein, 6 aa, synthetic construct
  source 1..6: note = CDR3 VL of I2A-10 D54E Y55Q
YYYYPG

SEQ ID NO: 219 - protein, 6 aa, synthetic construct
  source 1..6: note = CDR1 VH of I2A-11
FSSYVI

SEQ ID NO: 220 - protein, 10 aa, synthetic construct
  source 1..10: note = CDR2 VH of I2A-11
AIYPYTSSTQ

SEQ ID NO: 221 - protein, 7 aa, synthetic construct
  source 1..7: note = CDR3 VH of I2A-11
SYGTGGF

SEQ ID NO: 222 - protein, 11 aa, synthetic construct
  source 1..11: note = CDR1 VL of I2A-11
RASQSVSSAVA

SEQ ID NO: 223 - protein, 7 aa, synthetic construct
  source 1..7: note = CDR2 VL of I2A-11
SASSLYS

SEQ ID NO: 224 - protein, 6 aa, synthetic construct
  source 1..6: note = CDR3 VL of I2A-11
WYSYPL

SEQ ID NO: 225 - protein, 6 aa, synthetic construct
  source 1..6: note = CDR1 VH of I2A-12
FTTYYI

SEQ ID NO: 226 - protein, 10 aa, synthetic construct
  source 1..10: note = CDR2 VH of I2A-12
YISPEDGYTS

SEQ ID NO: 227 - protein, 7 aa, synthetic construct
  source 1..7: note = CDR3 VH of I2A-12
AYYSAVM

SEQ ID NO: 228 - protein, 11 aa, synthetic construct
  source 1..11: note = CDR1 VL of I2A-12
RASQSVSSAVA

SEQ ID NO: 229 - protein, 7 aa, synthetic construct
  source 1..7: note = CDR2 VL of I2A-12
SASSLYS

SEQ ID NO: 230 - protein, 6 aa, synthetic construct
  source 1..6: note = CDR3 VL of I2A-12
SWSPAT

SEQ ID NO: 231 - protein, 6 aa, synthetic construct
  source 1..6: note = CDR1 VH of I2A-13
FSYYFI

SEQ ID NO: 232 - protein, 10 aa, synthetic construct
  source 1..10: note = CDR2 VH of I2A-13
VIYPDGGYTL

SEQ ID NO: 233 - protein, 8 aa, synthetic construct
  source 1..8: note = CDR3 VH of I2A-13
IYYPSGAM

SEQ ID NO: 234 - protein, 11 aa, synthetic construct
  source 1..11: note = CDR1 VL of I2A-13
RASQSVSSAVA

SEQ ID NO: 235 - protein, 7 aa, synthetic construct
  source 1..7: note = CDR2 VL of I2A-13
SASSLYS

SEQ ID NO: 236 - protein, 6 aa, synthetic construct
  source 1..6: note = CDR3 VL of I2A-13
TYWYPG

SEQ ID NO: 237 - protein, 6 aa, synthetic construct
  source 1..6: note = CDR1 VH of I2A-14
FDSYVI

SEQ ID NO: 238 - protein, 10 aa, synthetic construct
  source 1..10: note = CDR2 VH of I2A-14
YIFSFGGYTY

SEQ ID NO: 239 - protein, 8 aa, synthetic construct
  source 1..8: note = CDR3 VH of I2A-14
SPYGTFAL

SEQ ID NO: 240 - protein, 11 aa, synthetic construct
  source 1..11: note = CDR1 VL of I2A-14
RASQSVSSAVA

SEQ ID NO: 241 - protein, 7 aa, synthetic construct
  source 1..7: note = CDR2 VL of I2A-14
SASSLYS

SEQ ID NO: 242 - protein, 6 aa, synthetic construct
  source 1..6: note = CDR3 VL of I2A-14
YYYTPG

SEQ ID NO: 243 - protein, 6 aa, synthetic construct
  source 1..6: note = CDR1 VH of I2A-15
FWGYVI

SEQ ID NO: 244 - protein, 10 aa, synthetic construct
  source 1..10: note = CDR2 VH of I2A-15
AIDSWDGDTD

SEQ ID NO: 245 - protein, 8 aa, synthetic construct
  source 1..8: note = CDR3 VH of I2A-15
SFYYIYVM

SEQ ID NO: 246 - protein, 11 aa, synthetic construct
  source 1..11: note = CDR1 VL of I2A-15
RASQSVSSAVA

SEQ ID NO: 247 - protein, 7 aa, synthetic construct
  source 1..7: note = CDR2 VL of I2A-15
SASSLYS

SEQ ID NO: 248 - protein, 6 aa, synthetic construct
  source 1..6: note = CDR3 VL of I2A-15
LYSTLV

SEQ ID NO: 249 - protein, 6 aa, synthetic construct
  source 1..6: note = CDR1 VH of I2A-16
FSGYFI

SEQ ID NO: 250 - protein, 10 aa, synthetic construct
  source 1..10: note = CDR2 VH of I2A-16
AIFPYRGGTS

SEQ ID NO: 251 - protein, 8 aa, synthetic construct
  source 1..8: note = CDR3 VH of I2A-16
GGVSPGGF

SEQ ID NO: 252 - protein, 11 aa, synthetic construct
  source 1..11: note = CDR1 VL of I2A-16
RASQSVSSAVA

SEQ ID NO: 253 - protein, 7 aa, synthetic construct
  source 1..7: note = CDR2 VL of I2A-16
SASSLYS

SEQ ID NO: 254 - protein, 6 aa, synthetic construct
  source 1..6: note = CDR3 VL of I2A-16
YYLYPG

SEQ ID NO: 255 - protein, 6 aa, synthetic construct
  source 1..6: note = CDR1 VH of I2A-17
FESYDI

SEQ ID NO: 256 - protein, 10 aa, synthetic construct
  source 1..10: note = CDR2 VH of I2A-17
AIFSYGGYTT

SEQ ID NO: 257 - protein, 9 aa, synthetic construct
  source 1..9: note = CDR3 VH of I2A-17
GSYGDGRGM

SEQ ID NO: 258 - protein, 11 aa, synthetic construct
  source 1..11: note = CDR1 VL of I2A-17
RASQSVSSAVA

SEQ ID NO: 259 - protein, 7 aa, synthetic construct
  source 1..7: note = CDR2 VL of I2A-17
SASSLYS

SEQ ID NO: 260 - protein, 6 aa, synthetic construct
  source 1..6: note = CDR3 VL of I2A-17
YYYWPG

SEQ ID NO: 261 - protein, 6 aa, synthetic construct
  source 1..6: note = CDR1 VH of I2A-18
FSSYFI

SEQ ID NO: 262 - protein, 10 aa, synthetic construct
  source 1..10: note = CDR2 VH of I2A-18
AIHPAFSFTY

SEQ ID NO: 263 - protein, 9 aa, synthetic construct
  source 1..9: note = CDR3 VH of I2A-18
PRLSSAVVL

SEQ ID NO: 264 - protein, 11 aa, synthetic construct
  source 1..11: note = CDR1 VL of I2A-18
RASQSVSSAVA

SEQ ID NO: 265 - protein, 7 aa, synthetic construct
  source 1..7: note = CDR2 VL of I2A-18
SASSLYS

SEQ ID NO: 266 - protein, 6 aa, synthetic construct
  source 1..6: note = CDR1 VH of I2A-19
FSSYFI

SEQ ID NO: 267 - protein, 10 aa, synthetic construct
  source 1..10: note = CDR2 VH of I2A-19
WIYPSGSYTY

SEQ ID NO: 268 - protein, 10 aa, synthetic construct
  source 1..10: note = CDR3 VH of I2A-19
EMDRVGYSGM

SEQ ID NO: 269 - protein, 11 aa, synthetic construct
  source 1..11: note = CDR1 VL of I2A-19
RASQSVSSAVA

SEQ ID NO: 270 - protein, 7 aa, synthetic construct
  source 1..7: note = CDR2 VL of I2A-19
SASSLYS

SEQ ID NO: 271 - protein, 6 aa, synthetic construct
  source 1..6: note = CDR3 VL of I2A-19
YRTPLG

SEQ ID NO: 272 - protein, 6 aa, synthetic construct
  source 1..6: note = CDR1 VH of I2A-20
FSDYGI

SEQ ID NO: 273 - protein, 10 aa, synthetic construct
  source 1..10: note = CDR2 VH of I2A-20
EIDSWLGYTY

SEQ ID NO: 274 - protein, 10 aa, synthetic construct
  source 1..10: note = CDR3 VH of I2A-20
SPYHYLYYGL

SEQ ID NO: 275 - protein, 11 aa, synthetic construct
  source 1..11: note = CDR1 VL of I2A-20
RASQSVSSAVA

SEQ ID NO: 276 - protein, 7 aa, synthetic construct
  source 1..7: note = CDR2 VL of I2A-20
SASSLYS

SEQ ID NO: 277 - protein, 6 aa, synthetic construct
  source 1..6: note = CDR3 VL of I2A-20
LSSSLG

SEQ ID NO: 278 - protein, 6 aa, synthetic construct
  source 1..6: note = CDR1 VH of I2A-21
FSGYFI

SEQ ID NO: 279 - protein, 10 aa, synthetic construct
  source 1..10: note = CDR2 VH of I2A-21
GISPWAGYTS

SEQ ID NO: 280 - protein, 6 aa, synthetic construct
  source 1..6: note = CDR3 VH of I2A-21
GGGRAF

SEQ ID NO: 281 - protein, 11 aa, synthetic construct
  source 1..11: note = CDR1 VL of I2A-21
RASQSVSSAVA

SEQ ID NO: 282 - protein, 7 aa, synthetic construct
  source 1..7: note = CDR2 VL of I2A-21
SASSLYS

SEQ ID NO: 283 - protein, 6 aa, synthetic construct
  source 1..6: note = CDR3 VL of I2A-21
YYWYPG

SEQ ID NO: 284 - protein, 6 aa, synthetic construct
  source 1..6: note = CDR1 VH of I2A-22
FSSYFI

SEQ ID NO: 285 - protein, 10 aa, synthetic construct
  source 1..10: note = CDR2 VH of I2A-22
AIYPSGWYTS

SEQ ID NO: 286 - protein, 6 aa, synthetic construct
  source 1..6: note = CDR3 VH of I2A-22
VQAGVF

SEQ ID NO: 287 - protein, 11 aa, synthetic construct
  source 1..11: note = CDR1 VL of I2A-22
RASQSVSSAVA

SEQ ID NO: 288 - protein, 7 aa, synthetic construct
  source 1..7: note = CDR2 VL of I2A-22
SASSLYS

SEQ ID NO: 289 - protein, 6 aa, synthetic construct
  source 1..6: note = CDR3 VL of I2A-22
YYYYPG

SEQ ID NO: 290 - protein, 6 aa, synthetic construct
  source 1..6: note = CDR1 VH of I2A-23
FDDYFI

SEQ ID NO: 291 - protein, 10 aa, synthetic construct
  source 1..10: note = CDR2 VH of I2A-23
AISSEGGYTD

SEQ ID NO: 292 - protein, 6 aa, synthetic construct
  source 1..6: note = CDR3 VH of I2A-23
AYRGVF

SEQ ID NO: 293 - protein, 11 aa, synthetic construct
  source 1..11: note = CDR1 VL of I2A-23
RASQSVSSAVA

SEQ ID NO: 294 - protein, 7 aa, synthetic construct
  source 1..7: note = CDR2 VL of I2A-23
SASSLYS

SEQ ID NO: 295 - protein, 6 aa, synthetic construct
  source 1..6: note = CDR3 VL of I2A-23
YYYFPG

SEQ ID NO: 296 - protein, 6 aa, synthetic construct
  source 1..6: note = CDR1 VH of I2A-24
FSTYGI

SEQ ID NO: 297 - protein, 10 aa, synthetic construct
  source 1..10: note = CDR2 VH of I2A-24
AIYPGTSYTG

SEQ ID NO: 298 - protein, 6 aa, synthetic construct
  source 1..6: note = CDR3 VH of I2A-24
EYFMGM

SEQ ID NO: 299 - protein, 11 aa, synthetic construct
  source 1..11: note = CDR1 VL of I2A-24
RASQSVSSAVA

SEQ ID NO: 300 - protein, 7 aa, synthetic construct
  source 1..7: note = CDR2 VL of I2A-24
SASSLYS

SEQ ID NO: 301 - protein, 6 aa, synthetic construct
  source 1..6: note = CDR3 VL of I2A-24
YYYWPG

SEQ ID NO: 302 - protein, 6 aa, synthetic construct
  source 1..6: note = CDR1 VH of I2A-25
FYGYTI

SEQ ID NO: 303 - protein, 10 aa, synthetic construct
  source 1..10: note = CDR2 VH of I2A-25
AIYPYTDSTR

SEQ ID NO: 304 - protein, 6 aa, synthetic construct
  source 1..6: note = CDR3 VH of I2A-25
DYRRAL

SEQ ID NO: 305 - protein, 11 aa, synthetic construct
  source 1..11: note = CDR1 VL of I2A-25
RASQSVSSAVA

SEQ ID NO: 306 - protein, 7 aa, synthetic construct
  source 1..7: note = CDR2 VL of I2A-25
SASSLYS

SEQ ID NO: 307 - protein, 6 aa, synthetic construct
  source 1..6: note = CDR3 VL of I2A-25
YTDFPW

SEQ ID NO: 308 - protein, 6 aa, synthetic construct
  source 1..6: note = CDR1 VH of I2A-26
FQSYDI

SEQ ID NO: 309 - protein, 10 aa, synthetic construct
  source 1..10: note = CDR2 VH of I2A-26
AIDPTGRSTA

SEQ ID NO: 310 - protein, 5 aa, synthetic construct
  source 1..5: note = CDR3 VH of I2A-26
DYGVF

SEQ ID NO: 311 - protein, 11 aa, synthetic construct
  source 1..11: note = CDR1 VL of I2A-26
RASQSVSSAVA

SEQ ID NO: 312 - protein, 7 aa, synthetic construct
  source 1..7: note = CDR2 VL of I2A-26
SASSLYS

SEQ ID NO: 313 - protein, 6 aa, synthetic construct
  source 1..6: note = CDR3 VL of I2A-26
FYRSPA

SEQ ID NO: 314 - protein, 6 aa, synthetic construct
  source 1..6: note = CDR1 VH of I2A-27
FKGYYI

SEQ ID NO: 315 - protein, 10 aa, synthetic construct
  source 1..10: note = CDR2 VH of I2A-27
AIYPYGGSTD

SEQ ID NO: 316 - protein, 7 aa, synthetic construct
  source 1..7: note = CDR3 VH of I2A-27
VYIYGVF

SEQ ID NO: 317 - protein, 11 aa, synthetic construct
  source 1..11: note = CDR1 VL of I2A-27
RASQSVSSAVA

SEQ ID NO: 318 - protein, 7 aa, synthetic construct
  source 1..7: note = CDR2 VL of I2A-27
SASSLYS

SEQ ID NO: 319 - protein, 6 aa, synthetic construct
  source 1..6: note = CDR3 VL of I2A-27
YYSSPR

SEQ ID NO: 320 - protein, 6 aa, synthetic construct
  source 1..6: note = CDR1 VH of I2A-28
FSSYWI

SEQ ID NO: 321 - protein, 10 aa, synthetic construct
  source 1..10: note = CDR2 VH of I2A-28
WIYPGTRYTE

SEQ ID NO: 322 - protein, 8 aa, synthetic construct
  source 1..8: note = CDR3 VH of I2A-28
DYVWPYGF

SEQ ID NO: 323 - protein, 11 aa, synthetic construct
  source 1..11: note = CDR1 VL of I2A-28
RASQSVSSAVA

SEQ ID NO: 324 - protein, 7 aa, synthetic construct
  source 1..7: note = CDR2 VL of I2A-28
SASSLYS

SEQ ID NO: 325 - protein, 6 aa, synthetic construct
  source 1..6: note = CDR3 VL of I2A-28
ASWSPV

SEQ ID NO: 326 - protein, 6 aa, synthetic construct
  source 1..6: note = CDR1 VH of I2A-29
FSSYWI

SEQ ID NO: 327 - protein, 10 aa, synthetic construct
  source 1..10: note = CDR2 VH of I2A-29
WIYSSGGYTF

SEQ ID NO: 328 - protein, 8 aa, synthetic construct
  source 1..8: note = CDR3 VH of I2A-29
EYFLYTGF

SEQ ID NO: 329 - protein, 11 aa, synthetic construct
  source 1..11: note = CDR1 VL of I2A-29
RASQSVSSAVA

SEQ ID NO: 330 - protein, 7 aa, synthetic construct
  source 1..7: note = CDR2 VL of I2A-29
SASSLYS

SEQ ID NO: 331 - protein, 6 aa, synthetic construct
  source 1..6: note = CDR3 VL of I2A-29
YSSGPV

SEQ ID NO: 332 - protein, 6 aa, synthetic construct
  source 1..6: note = CDR1 VH of I2A-30
FDSYFI

SEQ ID NO: 333 - protein, 10 aa, synthetic construct
  source 1..10: note = CDR2 VH of I2A-30
YIYSWGSYTH

SEQ ID NO: 334 - protein, 8 aa, synthetic construct
  source 1..8: note = CDR3 VH of I2A-30
GHRRYFAL

SEQ ID NO: 335 - protein, 11 aa, synthetic construct
  source 1..11: note = CDR1 VL of I2A-30
RASQSVSSAVA

SEQ ID NO: 336 - protein, 7 aa, synthetic construct
  source 1..7: note = CDR2 VL of I2A-30
SASSLYS

SEQ ID NO: 337 - protein, 6 aa, synthetic construct
  source 1..6: note = CDR3 VL of I2A-30
VYFTPG

SEQ ID NO: 338 - protein, 6 aa, synthetic construct
  source 1..6: note = CDR1 VH of I2A-31
FSSYWI

SEQ ID NO: 339 - protein, 10 aa, synthetic construct
  source 1..10: note = CDR2 VH of I2A-31
FIGPSGGYTY

SEQ ID NO: 340 - protein, 8 aa, synthetic construct
  source 1..8: note = CDR3 VH of I2A-31
ETDSYTGF

SEQ ID NO: 341 - protein, 11 aa, synthetic construct
  source 1..11: note = CDR1 VL of I2A-31
RASQSVSSAVA

SEQ ID NO: 342 - protein, 7 aa, synthetic construct
  source 1..7: note = CDR2 VL of I2A-31
SASSLYS

SEQ ID NO: 343 - protein, 6 aa, synthetic construct
  source 1..6: note = CDR3 VL of I2A-31
YYSWLG

SEQ ID NO: 344 - protein, 6 aa, synthetic construct
  source 1..6: note = CDR1 VH of I2A-32
FQSYVI

SEQ ID NO: 345 - protein, 10 aa, synthetic construct
  source 1..10: note = CDR2 VH of I2A-32
AIYPYSSSTI

SEQ ID NO: 346 - protein, 8 aa, synthetic construct
  source 1..8: note = CDR3 VH of I2A-32
SWSVYLGM

SEQ ID NO: 347 - protein, 11 aa, synthetic construct
  source 1..11: note = CDR1 VL of I2A-32
RASQSVSSAVA

SEQ ID NO: 348 - protein, 7 aa, synthetic construct
  source 1..7: note = CDR2 VL of I2A-32
SASSLYS

SEQ ID NO: 349 - protein, 6 aa, synthetic construct
  source 1..6: note = CDR3 VL of I2A-32
SYDSPR

SEQ ID NO: 350 - protein, 6 aa, synthetic construct
  source 1..6: note = CDR1 VH of I2A-33
FDDYYI

SEQ ID NO: 351 - protein, 10 aa, synthetic construct
  source 1..10: note = CDR2 VH of I2A-33
WIDSYGGYTS

SEQ ID NO: 352 - protein, 9 aa, synthetic construct
  source 1..9: note = CDR3 VH of I2A-33
SSYYYPGGF

SEQ ID NO: 353 - protein, 11 aa, synthetic construct
  source 1..11: note = CDR1 VL of I2A-33
RASQSVSSAVA

SEQ ID NO: 354 - protein, 7 aa, synthetic construct
  source 1..7: note = CDR2 VL of I2A-33
SASSLYS

SEQ ID NO: 355 - protein, 6 aa, synthetic construct
  source 1..6: note = CDR3 VL of I2A-33
WDSTLY

SEQ ID NO: 356 - protein, 6 aa, synthetic construct
  source 1..6: note = CDR1 VH of I2A-34
FSWYVI

SEQ ID NO: 357 - protein, 10 aa, synthetic construct
  source 1..10: note = CDR2 VH of I2A-34
YIAPYTGSTY

SEQ ID NO: 358 - protein, 9 aa, synthetic construct
  source 1..9: note = CDR3 VH of I2A-34
AFFGIRLGL

SEQ ID NO: 359 - protein, 11 aa, synthetic construct
  source 1..11: note = CDR1 VL of I2A-34
RASQSVSSAVA

SEQ ID NO: 360 - protein, 7 aa, synthetic construct
  source 1..7: note = CDR2 VL of I2A-34
SASSLYS

SEQ ID NO: 361 - protein, 6 aa, synthetic construct
  source 1..6: note = CDR3 VL of I2A-34
AISSPY

SEQ ID NO: 362 - protein, 6 aa, synthetic construct
  source 1..6: note = CDR1 VH of I2A-35
FSAYDI

SEQ ID NO: 363 - protein, 10 aa, synthetic construct
  source 1..10: note = CDR2 VH of I2A-35
WIDPYGGDTD

SEQ ID NO: 364 - protein, 10 aa, synthetic construct
  source 1..10: note = CDR3 VH of I2A-35
SPSYMQYGGL

SEQ ID NO: 365 - protein, 11 aa, synthetic construct
  source 1..11: note = CDR1 VL of I2A-35
RASQSVSSAVA

SEQ ID NO: 366 - protein, 7 aa, synthetic construct
  source 1..7: note = CDR2 VL of I2A-35
SASSLYS

SEQ ID NO: 367 - protein, 6 aa, synthetic construct
  source 1..6: note = CDR3 VL of I2A-35
YYSSLL

SEQ ID NO: 368 - protein, 6 aa, synthetic construct
  source 1..6: note = CDR1 VH of I2A-36
FSQYWI

SEQ ID NO: 369 - protein, 10 aa, synthetic construct
  source 1..10: note = CDR2 VH of I2A-36
AIYSSTKYTI

SEQ ID NO: 370 - protein, 10 aa, synthetic construct
  source 1..10: note = CDR3 VH of I2A-36
ESMYFYSYGL

SEQ ID NO: 371 - protein, 11 aa, synthetic construct
  source 1..11: note = CDR1 VL of I2A-36
RASQSVSSAVA

SEQ ID NO: 372 - protein, 7 aa, synthetic construct
  source 1..7: note = CDR2 VL of I2A-36
SASSLYS

SEQ ID NO: 373 - protein, 6 aa, synthetic construct
  source 1..6: note = CDR3 VL of I2A-36
LPSTPL

SEQ ID NO: 374 - protein, 6 aa, synthetic construct
  source 1..6: note = CDR1 VH of I2A-37
FSWYGI

SEQ ID NO: 375 - protein, 10 aa, synthetic construct
  source 1..10: note = CDR2 VH of I2A-37
YIDSYTSSTY

SEQ ID NO: 376 - protein, 10 aa, synthetic construct
  source 1..10: note = CDR3 VH of I2A-37
SHFGHYDYVM

SEQ ID NO: 377 - protein, 11 aa, synthetic construct
  source 1..11: note = CDR1 VL of I2A-37
RASQSVSSAVA

SEQ ID NO: 378 - protein, 7 aa, synthetic construct
  source 1..7: note = CDR2 VL of I2A-37
SASSLYS

SEQ ID NO: 379 - protein, 6 aa, synthetic construct
  source 1..6: note = CDR3 VL of I2A-37
AYDQLY

SEQ ID NO: 380 - protein, 6 aa, synthetic construct
  source 1..6: note = CDR1 VH of I2A-38
FDWYRI

SEQ ID NO: 381 - protein, 10 aa, synthetic construct
  source 1..10: note = CDR2 VH of I2A-38
WIDSYGSWTG

SEQ ID NO: 382 - protein, 10 aa, synthetic construct
  source 1..10: note = CDR3 VH of I2A-38
SYFGPYGYVL

SEQ ID NO: 383 - protein, 11 aa, synthetic construct
  source 1..11: note = CDR1 VL of I2A-38
RASQSVSSAVA

SEQ ID NO: 384 - protein, 7 aa, synthetic construct
  source 1..7: note = CDR2 VL of I2A-38
SASSLYS

SEQ ID NO: 385 - protein, 6 aa, synthetic construct
  source 1..6: note = CDR1 VH of I2C-1
FTSYGI

SEQ ID NO: 386 - protein, 10 aa, synthetic construct
  source 1..10: note = CDR2 VH of I2C-1
AIYPHSGFTS

SEQ ID NO: 387 - protein, 6 aa, synthetic construct
  source 1..6: note = CDR3 VH of I2C-1
TSYRGF

SEQ ID NO: 388 - protein, 11 aa, synthetic construct
  source 1..11: note = CDR1 VL of I2C-1
RASQSVSSAVA

SEQ ID NO: 389 - protein, 7 aa, synthetic construct
  source 1..7: note = CDR2 VL of I2C-1
SASSLYS

SEQ ID NO: 390 - protein, 6 aa, synthetic construct
  source 1..6: note = CDR3 VL of I2C-1
YYWYPG

SEQ ID NO: 391 - protein, 6 aa, synthetic construct
  source 1..6: note = CDR1 VH of I2C-2
FSDYFI

SEQ ID NO: 392 - protein, 10 aa, synthetic construct
  source 1..10: note = CDR2 VH of I2C-2
GIYPYSGYTK

SEQ ID NO: 393 - protein, 6 aa, synthetic construct
  source 1..6: note = CDR3 VH of I2C-2
DHSPVL

SEQ ID NO: 394 - protein, 11 aa, synthetic construct
  source 1..11: note = CDR1 VL of I2C-2
RASQSVSSAVA

SEQ ID NO: 395 - protein, 7 aa, synthetic construct
  source 1..7: note = CDR2 VL of I2C-2
SASSLYS

SEQ ID NO: 396 - protein, 6 aa, synthetic construct
  source 1..6: note = CDR3 VL of I2C-2
WYYWPG

SEQ ID NO: 397 - protein, 6 aa, synthetic construct
  source 1..6: note = CDR1 VH of I2C-3
FSHYWI

SEQ ID NO: 398 - protein, 10 aa, synthetic construct
  source 1..10: note = CDR2 VH of I2C-3
LIAPGGDYTS

SEQ ID NO: 399 - protein, 6 aa, synthetic construct
  source 1..6: note = CDR3 VH of I2C-3
SGLPGF

SEQ ID NO: 400 - protein, 11 aa, synthetic construct
  source 1..11: note = CDR1 VL of I2C-3
RASQSVSSAVA

SEQ ID NO: 401 - protein, 7 aa, synthetic construct
  source 1..7: note = CDR2 VL of I2C-3
SASSLYS

SEQ ID NO: 402 - protein, 6 aa, synthetic construct
  source 1..6: note = CDR3 VL of I2C-3
YKSSPL

SEQ ID NO: 403 - protein, 6 aa, synthetic construct
  source 1..6: note = CDR1 VH of I2C-4
FWSYFI

SEQ ID NO: 404 - protein, 10 aa, synthetic construct
  source 1..10: note = CDR2 VH of I2C-4
YIHPSSSYTD

SEQ ID NO: 405 - protein, 7 aa, synthetic construct
  source 1..7: note = CDR3 VH of I2C-4
TSRDGAM

SEQ ID NO: 406 - protein, 11 aa, synthetic construct
  source 1..11: note = CDR1 VL of I2C-4
RASQSVSSAVA

SEQ ID NO: 407 - protein, 7 aa, synthetic construct
  source 1..7: note = CDR2 VL of I2C-4
SASSLYS

SEQ ID NO: 408 - protein, 6 aa, synthetic construct
  source 1..6: note = CDR3 VL of I2C-4
WYSPPE

SEQ ID NO: 409 - protein, 6 aa, synthetic construct
  source 1..6: note = CDR1 VH of I2C-5
FSSYDI

SEQ ID NO: 410 - protein, 10 aa, synthetic construct
  source 1..10: note = CDR2 VH of I2C-5
WIYPYWGYTI

SEQ ID NO: 411 - protein, 7 aa, synthetic construct
  REGION 1..7: note = CDR3 VH of I2C-5
GTYAPAL

SEQ ID NO: 412 - protein, 11 aa, synthetic construct
  source 1..11: note = CDR1 VL of I2C-5
RASQSVSSAVA

SEQ ID NO: 413 - protein, 7 aa, synthetic construct
  source 1..7: note = CDR2 VL of I2C-5
SASSLYS

SEQ ID NO: 414 - protein, 6 aa, synthetic construct
  source 1..6: note = CDR3 VL of I2C-5
FYSYLS

SEQ ID NO: 415 - protein, 6 aa, synthetic construct
  source 1..6: note = CDR1 VH of I2C-6
FSWYFI

SEQ ID NO: 416 - protein, 10 aa, synthetic construct
  source 1..10: note = CDR2 VH of I2C-6
RIYSTGGYTE

SEQ ID NO: 417 - protein, 7 aa, synthetic construct
  source 1..7: note = CDR3 VH of I2C-6
SAFFGAL

SEQ ID NO: 418 - protein, 11 aa, synthetic construct
  source 1..11: note = CDR1 VL of I2C-6
RASQSVSSAVA

SEQ ID NO: 419 - protein, 7 aa, synthetic construct
  source 1..7: note = CDR2 VL of I2C-6
SASSLYS

SEQ ID NO: 420 - protein, 6 aa, synthetic construct
  source 1..6: note = CDR3 VL of I2C-6
YPSGPE

SEQ ID NO: 421 - protein, 6 aa, synthetic construct
  source 1..6: note = CDR1 VH of I2C-7
FDSYYI

SEQ ID NO: 422 - protein, 10 aa, synthetic construct
  source 1..10: note = CDR2 VH of I2C-7
WIDPYGLDTK

SEQ ID NO: 423 - protein, 7 aa, synthetic construct
  source 1..7: note = CDR3 VH of I2C-7
EPGDYGM

SEQ ID NO: 424 - protein, 11 aa, synthetic construct
  source 1..11: note = CDR1 VL of I2C-7
RASQSVSSAVA

SEQ ID NO: 425 - protein, 7 aa, synthetic construct
  source 1..7: note = CDR2 VL of I2C-7
SASSLYS

SEQ ID NO: 426 - protein, 6 aa, synthetic construct
  source 1..6: note = CDR3 VL of I2C-7
AYGSLL

SEQ ID NO: 427 - protein, 6 aa, synthetic construct
  source 1..6: note = CDR1 VH of I2C-8
FSGYFI

SEQ ID NO: 428 - protein, 10 aa, synthetic construct
  source 1..10: note = CDR2 VH of I2C-8
AIFPYRGGTS

SEQ ID NO: 429 - protein, 8 aa, synthetic construct
  source 1..8: note = CDR3 VH of I2C-8
GGVSPGGF

SEQ ID NO: 430 - protein, 11 aa, synthetic construct
  source 1..11: note = CDR1 VL of I2C-8
RASQSVSSAVA

SEQ ID NO: 431 - protein, 7 aa, synthetic construct
  source 1..7: note = CDR2 VL of I2C-8
SASSLYS

SEQ ID NO: 432 - protein, 6 aa, synthetic construct
  source 1..6: note = CDR3 VL of I2C-8
YYLYPG

SEQ ID NO: 433 - protein, 6 aa, synthetic construct
  source 1..6: note = CDR1 VH of I2C-9
FTDYDI

SEQ ID NO: 434 - protein, 10 aa, synthetic construct
  source 1..10: note = CDR2 VH of I2C-9
RIWPHGSYTF

SEQ ID NO: 435 - protein, 8 aa, synthetic construct
  source 1..8: note = CDR3 VH of I2C-9
SLTHSYGF

SEQ ID NO: 436 - protein, 11 aa, synthetic construct
  source 1..11: note = CDR1 VL of I2C-9
RASQSVSSAVA

SEQ ID NO: 437 - protein, 7 aa, synthetic construct
  source 1..7: note = CDR2 VL of I2C-9
SASSLYS

SEQ ID NO: 438 - protein, 6 aa, synthetic construct
  source 1..6: note = CDR3 VL of I2C-9
YYTWLI

SEQ ID NO: 439 - protein, 6 aa, synthetic construct
  source 1..6: note = CDR1 VH of I2C-10
FSSYFI

SEQ ID NO: 440 - protein, 10 aa, synthetic construct
  source 1..10: note = CDR2 VH of I2C-10
TIHSYFDGTS

SEQ ID NO: 441 - protein, 8 aa, synthetic construct
  source 1..8: note = CDR3 VH of I2C-10
TRPTGGAF

SEQ ID NO: 442 - protein, 11 aa, synthetic construct
  source 1..11: note = CDR1 VL of I2C-10
RASQSVSSAVA

SEQ ID NO: 443 - protein, 7 aa, synthetic construct
  source 1..7: note = CDR2 VL of I2C-10
SASSLYS

SEQ ID NO: 444 - protein, 6 aa, synthetic construct
  source 1..6: note = CDR3 VL of I2C-10
AYWSPA

SEQ ID NO: 445 - protein, 6 aa, synthetic construct
  source 1..6: note = CDR1 VH of I2C-11
FGSYFI

SEQ ID NO: 446 - protein, 10 aa, synthetic construct
  source 1..10: note = CDR2 VH of I2C-11
AIFPAGGYTY

SEQ ID NO: 447 - protein, 8 aa, synthetic construct
  source 1..8: note = CDR3 VH of I2C-11
YGSMGGAF

SEQ ID NO: 448 - protein, 11 aa, synthetic construct
  source 1..11: note = CDR1 VL of I2C-11
RASQSVSSAVA

SEQ ID NO: 449 - protein, 7 aa, synthetic construct
  source 1..7: note = CDR2 VL of I2C-11
SASSLYS

SEQ ID NO: 450 - protein, 6 aa, synthetic construct
  source 1..6: note = CDR3 VL of I2C-11
YYWFPG

SEQ ID NO: 451 - protein, 6 aa, synthetic construct
  source 1..6: note = CDR1 VH of I2C-12
FSSYFI

SEQ ID NO: 452 - protein, 10 aa, synthetic construct
  source 1..10: note = CDR2 VH of I2C-12
AIHPAFSFTY

SEQ ID NO: 453 - protein, 9 aa, synthetic construct
  source 1..9: note = CDR3 VH of I2C-12
PRLSSAVVL

SEQ ID NO: 454 - protein, 11 aa, synthetic construct
  source 1..11: note = CDR1 VL of I2C-12
RASQSVSSAVA

SEQ ID NO: 455 - protein, 7 aa, synthetic construct
  source 1..7: note = CDR2 VL of I2C-12
SASSLYS

SEQ ID NO: 456 - protein, 6 aa, synthetic construct
  source 1..6: note = CDR3 VL of I2C-12
GYYFPG

SEQ ID NO: 457 - protein, 6 aa, synthetic construct
  source 1..6: note = CDR1 VH of I2C-13
FSSYGI

SEQ ID NO: 458 - protein, 10 aa, synthetic construct
  source 1..10: note = CDR2 VH of I2C-13
YIHSGIGYTI

SEQ ID NO: 459 - protein, 9 aa, synthetic construct
  source 1..9: note = CDR3 VH of I2C-13
TSSTSTSVM

SEQ ID NO: 460 - protein, 11 aa, synthetic construct
  source 1..11: note = CDR1 VL of I2C-13
RASQSVSSAVA

SEQ ID NO: 461 - protein, 7 aa, synthetic construct
  source 1..7: note = CDR2 VL of I2C-13
SASSLYS

SEQ ID NO: 462 - protein, 6 aa, synthetic construct
  source 1..6: note = CDR3 VL of I2C-13
TYWSPG

SEQ ID NO: 463 - protein, 6 aa, synthetic construct
  source 1..6: note = CDR1 VH of I2C-14
FESYDI

SEQ ID NO: 464 - protein, 10 aa, synthetic construct
  source 1..10: note = CDR2 VH of I2C-14
AIFSYGGYTT

SEQ ID NO: 465 - protein, 9 aa, synthetic construct
  source 1..9: note = CDR3 VH of I2C-14
GSYGDGRGM

SEQ ID NO: 466 - protein, 11 aa, synthetic construct
  source 1..11: note = CDR1 VL of I2C-14
RASQSVSSAVA

SEQ ID NO: 467 - protein, 7 aa, synthetic construct
  source 1..7: note = CDR2 VL of I2C-14
SASSLYS

SEQ ID NO: 468 - protein, 6 aa, synthetic construct
  source 1..6: note = CDR3 VL of I2C-14
YYYWPG

SEQ ID NO: 469 - protein, 6 aa, synthetic construct
  source 1..6: note = CDR1 VH of I2C-15
FSSYFI

SEQ ID NO: 470 - protein, 10 aa, synthetic construct
  source 1..10: note = CDR2 VH of I2C-15
LIYPDTDDTY

SEQ ID NO: 471 - protein, 9 aa, synthetic construct
  source 1..9: note = CDR3 VH of I2C-15
YGYLGVGAF

SEQ ID NO: 472 - protein, 11 aa, synthetic construct
  source 1..11: note = CDR1 VL of I2C-15
RASQSVSSAVA

SEQ ID NO: 473 - protein, 7 aa, synthetic construct
  source 1..7: note = CDR2 VL of I2C-15
SASSLYS

SEQ ID NO: 474 - protein, 6 aa, synthetic construct
  source 1..6: note = CDR3 VL of I2C-15
LYWTPG

SEQ ID NO: 475 - protein, 6 aa, synthetic construct
  source 1..6: note = CDR1 VH of I2C-16
FSSYWI

SEQ ID NO: 476 - protein, 10 aa, synthetic construct
  source 1..10: note = CDR2 VH of I2C-16
AIHPSGSYTY

SEQ ID NO: 477 - protein, 9 aa, synthetic construct
  source 1..9: note = CDR3 VH of I2C-16
VGLLVTSVM

SEQ ID NO: 478 - protein, 11 aa, synthetic construct
  source 1..11: note = CDR1 VL of I2C-16
RASQSVSSAVA

SEQ ID NO: 479 - protein, 7 aa, synthetic construct
  source 1..7: note = CDR2 VL of I2C-16
SASSLYS

SEQ ID NO: 480 - protein, 6 aa, synthetic construct
  source 1..6: note = CDR3 VL of I2C-16
YYYFPG

SEQ ID NO: 481 - protein, 6 aa, synthetic construct
  source 1..6: note = CDR1 VH of I2C-17
FSDYFI

SEQ ID NO: 482 - protein, 10 aa, synthetic construct
  source 1..10: note = CDR2 VH of I2C-17
YIYPASGGTC

SEQ ID NO: 483 - protein, 9 aa, synthetic construct
  source 1..9: note = CDR3 VH of I2C-17
GYIPHMAAL

SEQ ID NO: 484 - protein, 11 aa, synthetic construct
  source 1..11: note = CDR1 VL of I2C-17
RASQSVSSAVA

SEQ ID NO: 485 - protein, 7 aa, synthetic construct
  source 1..7: note = CDR2 VL of I2C-17
SASSLYS

SEQ ID NO: 486 - protein, 6 aa, synthetic construct
  source 1..6: note = CDR3 VL of I2C-17
YYWWPG

SEQ ID NO: 487 - protein, 6 aa, synthetic construct
  source 1..6: note = CDR1 VH of I2C-18
FAGYPI

SEQ ID NO: 488 - protein, 10 aa, synthetic construct
  source 1..10: note = CDR2 VH of I2C-18
AIDPDGGYTY

SEQ ID NO: 489 - protein, 10 aa, synthetic construct
  source 1..10: note = CDR3 VH of I2C-18
HTGFHRYRGM

SEQ ID NO: 490 - protein, 11 aa, synthetic construct
  source 1..11: note = CDR1 VL of I2C-18
RASQSVSSAVA

SEQ ID NO: 491 - protein, 7 aa, synthetic construct
  source 1..7: note = CDR2 VL of I2C-18
SASSLYS

SEQ ID NO: 492 - protein, 6 aa, synthetic construct
  source 1..6: note = CDR3 VL of I2C-18
YYWFPP

SEQ ID NO: 493 - protein, 6 aa, synthetic construct
  source 1..6: note = CDR1 VH of I2C-19
FTSYDI

SEQ ID NO: 494 - protein, 10 aa, synthetic construct
  source 1..10: note = CDR2 VH of I2C-19
WIDPGLSYTS

SEQ ID NO: 495 - protein, 10 aa, synthetic construct
  source 1..10: note = CDR3 VH of I2C-19
ASIGGGVPVM

SEQ ID NO: 496 - protein, 11 aa, synthetic construct
  source 1..11: note = CDR1 VL of I2C-19
RASQSVSSAVA

SEQ ID NO: 497 - protein, 7 aa, synthetic construct
  source 1..7: note = CDR2 VL of I2C-19
SASSLYS

SEQ ID NO: 498 - protein, 6 aa, synthetic construct
  source 1..6: note = CDR3 VL of I2C-19
YVTGPY

SEQ ID NO: 499 - protein, 6 aa, synthetic construct
  source 1..6: note = CDR1 VH of I2C-20
FSSYYI

SEQ ID NO: 500 - protein, 10 aa, synthetic construct
  source 1..10: note = CDR2 VH of I2C-20
IIIPYSGYTY

SEQ ID NO: 501 - protein, 10 aa, synthetic construct
  source 1..10: note = CDR3 VH of I2C-20
RGYFSLGTAM

SEQ ID NO: 502 - protein, 11 aa, synthetic construct
  source 1..11: note = CDR1 VL of I2C-20
RASQSVSSAVA

SEQ ID NO: 503 - protein, 7 aa, synthetic construct
  source 1..7: note = CDR2 VL of I2C-20
SASSLYS

SEQ ID NO: 504 - protein, 6 aa, synthetic construct
  source 1..6: note = CDR3 VL of I2C-20
VYWWPG

SEQ ID NO: 505 - protein, 6 aa, synthetic construct
  source 1..6: note = CDR1 VH of I2C-21
FSSYYI

SEQ ID NO: 506 - protein, 10 aa, synthetic construct
  source 1..10: note = CDR2 VH of I2C-21
WIDPYISYTY

SEQ ID NO: 507 - protein, 10 aa, synthetic construct
  source 1..10: note = CDR3 VH of I2C-21
DSELVSGYAM

SEQ ID NO: 508 - protein, 11 aa, synthetic construct
  source 1..11: note = CDR1 VL of I2C-21
RASQSVSSAVA

SEQ ID NO: 509 - protein, 7 aa, synthetic construct
  source 1..7: note = CDR2 VL of I2C-21
SASSLYS

SEQ ID NO: 510 - protein, 6 aa, synthetic construct
  source 1..6: note = CDR3 VL of I2C-21
GDSSLV

SEQ ID NO: 511 - protein, 6 aa, synthetic construct
  source 1..6: note = CDR1 VH of I2C-22
FSSYSI

SEQ ID NO: 512 - protein, 10 aa, synthetic construct
  source 1..10: note = CDR2 VH of I2C-22
AIYPYWGTTE

SEQ ID NO: 513 - protein, 10 aa, synthetic construct
  source 1..10: note = CDR3 VH of I2C-22
PSGVTYGYAL

SEQ ID NO: 514 - protein, 11 aa, synthetic construct
  source 1..11: note = CDR1 VL of I2C-22
RASQSVSSAVA

SEQ ID NO: 515 - protein, 7 aa, synthetic construct
  source 1..7: note = CDR2 VL of I2C-22
SASSLYS

SEQ ID NO: 516 - protein, 6 aa, synthetic construct
  source 1..6: note = CDR3 VL of I2C-22
YYYSPW

SEQ ID NO: 517 - protein, 6 aa, synthetic construct
  source 1..6: note = CDR1 VH of I2C-23
FSSYEI

SEQ ID NO: 518 - protein, 10 aa, synthetic construct
  source 1..10: note = CDR2 VH of I2C-23
SIYPFSGDTY

SEQ ID NO: 519 - protein, 10 aa, synthetic construct
  source 1..10: note = CDR3 VH of I2C-23
PGRAIYYAVM

SEQ ID NO: 520 - protein, 11 aa, synthetic construct
  source 1..11: note = CDR1 VL of I2C-23
RASQSVSSAVA

SEQ ID NO: 521 - protein, 7 aa, synthetic construct
  source 1..7: note = CDR2 VL of I2C-23
SASSLYS

SEQ ID NO: 522 - protein, 6 aa, synthetic construct
  source 1..6: note = CDR3 VL of I2C-23
AGSHLF

SEQ ID NO: 523 - protein, 6 aa, synthetic construct
  source 1..6: note = CDR1 VH of I2C-24
FSRYYI

SEQ ID NO: 524 - protein, 10 aa, synthetic construct
  source 1..10: note = CDR2 VH of I2C-24
DIDSYGGFTY

SEQ ID NO: 525 - protein, 11 aa, synthetic construct
  source 1..11: note = CDR3 VH of I2C-24
AHRFLQGGYVL

SEQ ID NO: 526 - protein, 11 aa, synthetic construct
  source 1..11: note = CDR1 VL of I2C-24
RASQSVSSAVA

SEQ ID NO: 527 - protein, 7 aa, synthetic construct
  source 1..7: note = CDR2 VL of I2C-24
SASSLYS

SEQ ID NO: 528 - protein, 6 aa, synthetic construct
  source 1..6: note = CDR3 VL of I2C-24
YSWGLW

SEQ ID NO: 529 - protein, 6 aa, synthetic construct
  source 1..6: note = CDR1 VH of I2C-25
FSDYYI

SEQ ID NO: 530 - protein, 10 aa, synthetic construct
  source 1..10: note = CDR2 VH of I2C-25
YIAPYGGFTY

SEQ ID NO: 531 - protein, 11 aa, synthetic construct
  source 1..11: note = CDR3 VH of I2C-25
DSYRRGYVSGF

SEQ ID NO: 532 - protein, 11 aa, synthetic construct
  source 1..11: note = CDR1 VL of I2C-25
RASQSVSSAVA

SEQ ID NO: 533 - protein, 7 aa, synthetic construct
  source 1..7: note = CDR2 VL of I2C-25
SASSLYS

SEQ ID NO: 534 - protein, 6 aa, synthetic construct
  source 1..6: note = CDR3 VL of I2C-25
RYSSPS

SEQ ID NO: 535 - protein, 6 aa, synthetic construct
  source 1..6: note = CDR1 VH of I2C-26
FSDYYI

SEQ ID NO: 536 - protein, 10 aa, synthetic construct
  source 1..10: note = CDR2 VH of I2C-26
DIDSYGGFTY

SEQ ID NO: 537 - protein, 11 aa, synthetic construct
  source 1..11: note = CDR3 VH of I2C-26
DPHFLDDVYVL

SEQ ID NO: 538 - protein, 11 aa, synthetic construct
  source 1..11: note = CDR1 VL of I2C-26
RASQSVSSAVA

SEQ ID NO: 539 - protein, 7 aa, synthetic construct
  source 1..7: note = CDR2 VL of I2C-26
SASSLYS

SEQ ID NO: 540 - protein, 6 aa, synthetic construct
  source 1..6: note = CDR3 VL of I2C-26
YSWGLW